SEQUENCE LISTING (58 sequences)

SEQ ID NO: 1 - DNA, 1671 bp, Arabidopsis thaliana
atgacgacacaagatgtgatagtcaatgatcagaatgatcagaaacagtgtagtaatgacgtcattttccgatcgagattgcctgatatatacatccctaaccacctcccactccacgactacatcttcgaaaatatctcagagttcgccgctaagccatgcttgatcaacggtcccaccggcgaagtatacacctacgccgatgtccacgtaacatctcggaaactcgccgccggtcttcataacctcggcgtgaagcaacacgacgttgtaatgatcctcctcccgaactctcctgaagtagtcctcactttccttgccgcctccttcatcggcgcaatcaccacctccgcgaacccgttcttcactccggcggagatttctaaacaagccaaagcctccgcggcgaaactcatcgtcactcaatcccgttacgtcgataaaatcaagaacctccaaaacgacggcgttttgatcgtcaccaccgactccgacgccatccccgaaaactgcctccgtttctccgagttaactcagtccgaagaaccacgagtggactcaataccggagaagatttcgccagaagacgtcgtggcgcttcctttctcatccggcacgacgggtctccccaaaggagtgatgctaacacacaaaggtctagtcacgagcgtggcgcagcaagtcgacggcgagaatccgaatctttacttcaacagagacgacgtgatcctctgtgtcttgcctatgttccatatatacgctctcaactccatcatgctctgtagtctcagagttggtgccacgatcttgataatgcctaagttcgaaatcactctcttgttagagcagatacaaaggtgtaaagtcacggtggctatggtcgtgccaccgatcgttttagctatcgcgaagtcgccggagacggagaagtatgatctgagctcggttaggatggttaagtctggagcagctcctcttggtaaggagcttgaagatgctattagtgctaagtttcctaacgccaagcttggtcagggctatgggatgacagaagcaggtccggtgctagcaatgtcgttagggtttgctaaagagccgtttccagtgaagtcaggagcatgtggtacggtggtgaggaacgccgagatgaagatacttgatccagacacaggagattctttgcctaggaacaaacccggcgaaatatgcatccgtggcaaccaaatcatgaaaggctatctcaatgaccccttggccacggcatcgacgatcgataaagatggttggcttcacactggagacgtcggatttatcgatgatgacgacgagcttttcattgtggatagattgaaagaactcatcaagtacaaaggatttcaagtggctccagctgagctagagtctctcctcataggtcatccagaaatcaatgatgttgctgtcgtcgccatgaaggaagaagatgctggtgaggttcctgttgcgtttgtggtgagatcgaaagattcaaatatatccgaagatgaaatcaagcaattcgtgtcaaaacaggttgtgttttataagagaatcaacaaagtgttcttcactgactctattcctaaagctccatcagggaagatattgaggaaggatctaagagcaagactagcaaatggattaatgaactag

SEQ ID NO: 2 - protein, 556 aa, Arabidopsis thaliana
MTTQDVIVNDQNDQKQCSNDVIFRSRLPDIYIPNHLPLHDYIFENISEFAAKPCLINGPTGEVYTYADVHVTSRKLAAGLHNLGVKQHDVVMILLPNSPEVVLTFLAASFIGAITTSANPFFTPAEISKQAKASAAKLIVTQSRYVDKIKNLQNDGVLIVTTDSDAIPENCLRFSELTQSEEPRVDSIPEKISPEDVVALPFSSGTTGLPKGVMLTHKGLVTSVAQQVDGENPNLYFNRDDVILCVLPMFHIYALNSIMLCSLRVGATILIMPKFEITLLLEQIQRCKVTVAMVVPPIVLAIAKSPETEKYDLSSVRMVKSGAAPLGKELEDAISAKFPNAKLGQGYGMTEAGPVLAMSLGFAKEPFPVKSGACGTVVRNAEMKILDPDTGDSLPRNKPGEICIRGNQIMKGYLNDPLATASTIDKDGWLHTGDVGFIDDDDELFIVDRLKELIKYKGFQVAPAELESLLIGHPEINDVAVVAMKEEDAGEVPVAFVVRSKDSNISEDEIKQFVSKQVVFYKRINKVFFTDSIPKAPSGKILRKDLRARLANGLMN

SEQ ID NO: 3 - DNA, 1095 bp, Malus domestica
atggctccagccactaccttaacctctattgcacatgaaaagacattacagcagaagttcgttagagatgaggatgaaaggcctaaggttgcctataacgacttttctaatgaaattccaataatctctttggctggtatagacgaagtagaaggtagaaggggagaaatatgtaagaagattgttgcagcttgcgaagattggggcattttccagatcgtagaccatggtgtagatgccgaattgatatcagaaatgacaggtttggctagagaattcttcgcattgccttcagaagagaagttaaggtttgatatgtccggtggtaagaaaggtggttttatagtctctagtcatttacagggtgaagccgttcaagattggagagaaatcgtaacatatttctcatacccaattagacacagagattactccaggtggcctgataagccagaagcctggagggaagttactaagaaatactcagatgagttgatgggattagcttgtaaattgttgggcgtgttgtcagaagccatgggattggatacagaggccttgaccaaagcatgtgttgatatggaccaaaaggtagttgtcaacttctaccctaaatgccctcaaccagacttgacattaggcttgaaaagacataccgaccccggcactatcactttattattacaagaccaagtcggtggtttgcaggctactagagacgacggtaaaacctggatcactgttcaacccgttgaaggagcattcgtcgttaatttgggcgatcatggacacttattgtccaatggtagatttaagaatgctgatcaccaagctgtggtcaactctaatagtagtagattatccattgctacatttcagaacccagcacaagaagcaattgtttatcctttatctgtgagagaaggagagaagcctattttagaggcaccaattacatatactgagatgtataagaagaagatgtctaaagatttggagttagcaagattgaagaaattagctaaagagcaacaaagtcaagatttagagaaggctaaagtggatactaaaccagtggatgatatcttcgcttaa

SEQ ID NO: 4 - protein, 364 aa, Malus domestica
MAPATTLTSIAHEKTLQQKFVRDEDERPKVAYNDFSNEIPIISLAGIDEVEGRRGEICKKIVAACEDWGIFQIVDHGVDAELISEMTGLAREFFALPSEEKLRFDMSGGKKGGFIVSSHLQGEAVQDWREIVTYFSYPIRHRDYSRWPDKPEAWREVTKKYSDELMGLACKLLGVLSEAMGLDTEALTKACVDMDQKVVVNFYPKCPQPDLTLGLKRHTDPGTITLLLQDQVGGLQATRDDGKTWITVQPVEGAFVVNLGDHGHLLSNGRFKNADHQAVVNSNSSRLSIATFQNPAQEAIVYPLSVREGEKPILEAPITYTEMYKKKMSKDLELARLKKLAKEQQSQDLEKAKVDTKPVDDIFA

SEQ ID NO: 5 - DNA, 1044 bp, Anthurium andraeanum
atgatgcacaaaggtacagtttgtgttactggtgctgccggcttcgtaggtagttggttaatcatgaggttattagaacaaggttactccgttaaggctacagtgagagatccttctaacatgaagaaagttaagcatttgttggatttacccggagcagcaaataggttgactttgtggaaggcagatttagttgatgaaggttcctttgatgaacctattcaaggttgcacaggtgtattccatgtcgcaactccaatggatttcgagtctaaagatcctgagagtgagatgattaaacctacaatcgagggcatgttaaacgttttgaggtcatgtgcaagagcatccagtactgtcagaagggtagttttcacttcctctgccggtactgttagtatccatgaaggcagaagacacttatacgatgaaaccagttggtcagacgtcgatttctgcagggccaagaagatgacaggttggatgtatttcgtctctaaaaccttagcagaaaaggccgcctgggatttcgcagaaaagaataacattgacttcatttctattatacccactttagtcaatggtccctttgttatgccaactatgccaccatcaatgttgtcagctttggctttaattaccagaaatgaacctcattactcaattttgaaccctgtgcaatttgtacatttggatgatttatgcaatgctcatattttcttgtttgaatgtccagatgctaagggtagatacatctgttcttcacacgatgtaacaatcgccggtttagctcaaatattgagacaaagatatccagagtttgacgtgccaacagaatttggagaaatggaggtgtttgacattatatcatattcttctaagaagttaactgacttgggatttgaatttaaatattctttagaggacatgtttgacggcgctatacagtcttgtagagaaaagggcttgttgcctccagctacaaaagaaccatcctatgctaccgaacaattgatagctaccggacaggacaatggacactaa

SEQ ID NO: 6 - protein, 347 aa, Anthurium andraeanum
MMHKGTVCVTGAAGFVGSWLIMRLLEQGYSVKATVRDPSNMKKVKHLLDLPGAANRLTLWKADLVDEGSFDEPIQGCTGVFHVATPMDFESKDPESEMIKPTIEGMLNVLRSCARASSTVRRVVFTSSAGTVSIHEGRRHLYDETSWSDVDFCRAKKMTGWMYFVSKTLAEKAAWDFAEKNNIDFISIIPTLVNGPFVMPTMPPSMLSALALITRNEPHYSILNPVQFVHLDDLCNAHIFLFECPDAKGRYICSSHDVTIAGLAQILRQRYPEFDVPTEFGEMEVFDIISYSSKKLTDLGFEFKYSLEDMFDGAIQSCREKGLLPPATKEPSYATEQLIATGQDNGH

SEQ ID NO: 7 - DNA, 1041 bp, Populus trichocarpa
atgggtactgaagctgaaaccgtttgtgttactggtgcttctggttttattggttcctggttgatcatgagattattggaaaaaggttacgctgttagagccactgttagagatccagataatatgaagaaggtcacccacttgttggaattgccaaaggcttctactcatttgactttgtggaaagccgatttgtctgttgaaggttcttacgatgaagctattcaaggttgtactggtgttttccatgttgctactccaatggatttcgaatctaaggatccagaaaacgaagttatcaagccaaccattaacggtgttttggatattatgagagcttgcgctaactctaagaccgttagaaagatcgttttcacttcttctgctggtactgttgatgtcgaagaaaaaagaaagccagtctacgatgaatcttgctggtctgatttggatttcgtccaatctattaagatgaccggttggatgtacttcgtttctaaaactttggctgaacaagctgcttggaagttcgctaaagaaaacaacttggacttcatctccattatcccaactttggttgttggtccattcatcatgcaatctatgccaccatctttgttgactgccttgtctttgattactggtaacgaagctcattacggtatcttgaaacaaggtcattacgttcacttggatgacttgtgtatgtcccatatcttcttgtacgaaaacccaaaagctgaaggtagatatatctgcaactctgatgatgccaacattcatgatttggctaagttgttgagagaaaagtacccagaatacaacgttccagctaagttcaaggatatcgacgaaaatttggcttgcgttgctttctcatctaagaagttgacagatttgggtttcgaattcaagtactccttggaagatatgtttgctggtgcagttgaaacctgtagagaaaagggtttgattccattgtcccacagaaaacaagtcgtcgaagaatgcaaagaaaatgaagttgttccagcttcttaa

SEQ ID NO: 8 - protein, 346 aa, Populus trichocarpa
MGTEAETVCVTGASGFIGSWLIMRLLEKGYAVRATVRDPDNMKKVTHLLELPKASTHLTLWKADLSVEGSYDEAIQGCTGVFHVATPMDFESKDPENEVIKPTINGVLDIMRACANSKTVRKIVFTSSAGTVDVEEKRKPVYDESCWSDLDFVQSIKMTGWMYFVSKTLAEQAAWKFAKENNLDFISIIPTLVVGPFIMQSMPPSLLTALSLITGNEAHYGILKQGHYVHLDDLCMSHIFLYENPKAEGRYICNSDDANIHDLAKLLREKYPEYNVPAKFKDIDENLACVAFSSKKLTDLGFEFKYSLEDMFAGAVETCREKGLIPLSHRKQVVEECKENEVVPAS

SEQ ID NO: 9 - DNA, 1293 bp, Petunia x hybrida
atggttaacgccgttgttactaccccatctagagttgaatctttggctaagtctggtattcaagccatcccaaaagaatacgttagaccacaagaagaattgaacggtatcggtaacattttcgaagaagaaaagaaagacgaaggtccacaagttccaaccatcgatttgaaagaaatcgactccgaagacaaagaaatcagagaaaagtgccaccaattgaaaaaggctgctatggaatggggtgttatgcatttggttaatcacggtatctccgacgaattgatcaacagagttaaggttgctggtgaaacctttttcgatcaaccagtcgaagaaaaagaaaagtacgctaacgatcaagccaacggtaatgttcaaggttacggttctaaattggctaactctgcttgtggtcaattggaatgggaagattactttttccattgcgctttcccagaagataagagagatttgtctatctggccaaagaacccaactgattatactccagctacttctgaatacgccaagcaaattagagctttggctactaagattttgaccgtcttgtctattggtttgggtttggaagaaggtagattggaaaaagaagttggtggtatggaagatttgttgttgcaaatgaagatcaactactacccaaagtgtccacaaccagaattggctttgggtgttgaagctcatactgatgtttctgctttgaccttcatcttgcataatatggtcccaggtttacaattattctacgaaggtcaatgggttaccgctaagtgtgttccaaattccattatcatgcatatcggtgacaccatcgaaatcttgtctaacggtaaatacaagtccatcttgcacagaggtgttgtcaacaaagaaaaggttagattctcctgggctattttctgtgaaccacctaaagaaaagatcatcttgaagccattgccagaaactgttactgaagctgaaccaccaagatttccaccaagaacttttgctcaacatatggcccataagttgttcagaaaggatgataaggatgctgccgttgaacataaggttttcaacgaagatgaattggatactgctgctgaacacaaagtcttgaagaaggataatcaagacgctgttgctgaaaacaaggacatcaaagaagatgaacaatgtggtccagcagaacacaaagatatcaaagaagatggtcaaggtgctgctgcagaaaacaaggttttcaaagaaaacaatcaagatgtcgccgccgaagaatctaagtaa

SEQ ID NO: 10 - protein, 430 aa, Petunia x hybrida
MVNAVVTTPSRVESLAKSGIQAIPKEYVRPQEELNGIGNIFEEEKKDEGPQVPTIDLKEIDSEDKEIREKCHQLKKAAMEWGVMHLVNHGISDELINRVKVAGETFFDQPVEEKEKYANDQANGNVQGYGSKLANSACGQLEWEDYFFHCAFPEDKRDLSIWPKNPTDYTPATSEYAKQIRALATKILTVLSIGLGLEEGRLEKEVGGMEDLLLQMKINYYPKCPQPELALGVEAHTDVSALTFILHNMVPGLQLFYEGQWVTAKCVPNSIIMHIGDTIEILSNGKYKSILHRGVVNKEKVRFSWAIFCEPPKEKIILKPLPETVTEAEPPRFPPRTFAQHMAHKLFRKDDKDAAVEHKVFNEDELDTAAEHKVLKKDNQDAVAENKDIKEDEQCGPAEHKDIKEDGQGAAAENKVFKENNQDVAAEESK

SEQ ID NO: 11 - DNA, 1380 bp, Dianthus caryophyllus
atgtcagcaaattctaactacatgaacaaaagtcgtctccatgtcgctgtgtttccattcccttttggaacacacgcgactccacttttcaacataacccaaaaactagcatcatttatgcctgatgtcgtcttctccttcttcaacatcccacaatccaacgctaagatatcttctgattttaaaaacgataccataaacatgtatgatgtgtgggacggggtgccggaaggatatgtcttcaagggtaagcctcaagaagacatcgagctcttcatgctggctgcacctcccacattgacagaggcgttggctaaagccgaggtggaaacagggaccaaggtgagctgcatacttggcgatgcctttttatggttcctggaggaactcgcccaacaaaaacaagttccctggattactacttatatgtctgaggagcattctcttttggctcatatttgcactgatcttatcagacaaactattggcattcatgagaaagcagaagagcggaaagatgaagagctagatttcattccaggattgtccaagattagagtccaagacttaccagagggaatcgtgatgggaaatttggattcgtattttgcgagaatgcttcaccaaatggggcgggcattaccgcgtgcatcagcagtttgcattagttcatgtcaagaactagaccctgttgcgactaatgagcttaacagaaaattgaataaattgattaatgttggacctctaagtctaattacgcaatcaaactcattaccttcaggcacaaacaagagtctgggttggcttgataaacaagaatctgaaaacagtgttgcgtacgttagttttgggtcagttgcacgccctgatgcaaccgagattacagccctggctcaagcattggaggcaagtcaggtcaaatttatctggtcgattagagacaatcttaaggtacatttgccaggtggatttattgagaatacaaaggataaagggatggtggtgtcgtgggtgccacagacagctgtgttggctcacaaggcagttggtgttttcataacccatttcggtcacaattccatcatggaaagtattgcaagtgaggttccaatgatagggcgaccattcatcggggaacaaaagttgaacggtagaatagtggaagccaaatggtgtatcggtttggttgtggaaggtggagttttcactaaagatggtgtactgagaagcttgaacaaaatactaggtagcacacaaggtgaagaaatgaggagaaatataagagacctacgactcatggttgacaaggcactcagtcctgacggaagctgcaatacaaacttgaaacatttggtcgacatgatcgtcacttctaactaa

SEQ ID NO: 12 - protein, 459 aa, Dianthus caryophyllus
MSANSNYMNKSRLHVAVFPFPFGTHATPLFNITQKLASFMPDVVFSFFNIPQSNAKISSDFKNDTINMYDVWDGVPEGYVFKGKPQEDIELFMLAAPPTLTEALAKAEVETGTKVSCILGDAFLWFLEELAQQKQVPWITTYMSEEHSLLAHICTDLIRQTIGIHEKAEERKDEELDFIPGLSKIRVQDLPEGIVMGNLDSYFARMLHQMGRALPRASAVCISSCQELDPVATNELNRKLNKLINVGPLSLITQSNSLPSGTNKSLGWLDKQESENSVAYVSFGSVARPDATEITALAQALEASQVKFIWSIRDNLKVHLPGGFIENTKDKGMVVSWVPQTAVLAHKAVGVFITHFGHNSIMESIASEVPMIGRPFIGEQKLNGRIVEAKWCIGLVVEGGVFTKDGVLRSLNKILGSTQGEEMRRNIRDLRLMVDKALSPDGSCNTNLKHLVDMIVTSN

SEQ ID NO: 13 - DNA, 669 bp, Medicago sativa
atggctgcttccattaccgctattaccgttgaaaatttggaatacccagctgttgttacttctccagttactggtaagtcttactttttgggtggtgctggtgaaagaggtttgactattgaaggtaacttcattaagttcaccgccatcggtgtttacttggaagatattgctgttgcttctttggctgctaaatggaagggtaaatcctccgaagaattattggaaaccttggacttctacagagacattatttctggtccattcgaaaagttgatcagaggttccaagatcagagaattgtctggtccagaatactccagaaaggttatggaaaattgcgttgcccatttgaagtctgttggtacttatggtgatgctgaagctgaagctatgcaaaaatttgctgaagcctttaagccagttaattttccaccaggtgcttccgttttttacagacaatctccagatggtatcttgggtttgtctttttcaccagatacctccatcccagaaaaagaagctgctttgattgaaaacaaggctgtttcttctgctgtcttggaaactatgattggtgaacatgctgtttccccagatttgaaaagatgtttagctgctagattgcctgccttgttgaatgaaggtgcttttaagattggtaactaa

SEQ ID NO: 14 - protein, 222 aa, Medicago sativa
MAASITAITVENLEYPAVVTSPVTGKSYFLGGAGERGLTIEGNFIKFTAIGVYLEDIAVASLAAKWKGKSSEELLETLDFYRDIISGPFEKLIRGSKIRELSGPEYSRKVMENCVAHLKSVGTYGDAEAEAMQKFAEAFKPVNFPPGASVFYRQSPDGILGLSFSPDTSIPEKEAALIENKAVSSAVLETMIGEHAVSPDLKRCLAARLPALLNEGAFKIGN

SEQ ID NO: 15 - DNA, 2112 bp, Zea mays
atggcgggcaacggcgccatcgtggagagcgacccgctgaactggggcgcggcggcggcggagctggccgggagccacctggacgaggtgaagcgcatggtggcgcaggcccggcagcccgtggtcaagatcgagggctccaccctccgcgtcggccaggtggccgccgtcgcctccgccaaggacgcgtccggcgtcgccgtcgagctcgacgaggaggcccgcccccgcgtcaaggccagcagcgagtggatcctcgactgcatcgcccacggcggcgacatctacggcgtcaccaccggcttcggcggcacctcccaccgccgcaccaaggacgggcccgcgctccaggtcgagctgctcaggcatctcaacgccggaatcttcggcaccggcagcgacgggcacacgctgccgtcggaggtcacccgcgcggcgatgctggtgcgcatcaacaccctcctccagggctactccggcatccgcttcgagatcctcgaggccatcacgaagctgctcaacaccggtgtcagcccctgcctgccgctccggggcaccatcaccgcgtcgggcgacctggtcccgctctcctacatcgccggcctcatcacgggccgccccaacgcgcaggccgtcaccgtcgacggaaggaaggtggacgccgccgaggcgttcaagatcgccggcatcgagggcggcttcttcaagctcaaccccaaggagggcctcgccatcgtcaacggcacgtccgtgggctccgcgctcgcggccaccgtgatgtacgacgccaacgtcctggccgtcctgtcggaggtcctgtccgccgtcttctgcgaggtcatgaacggcaagcccgagtacacggaccacctgacccacaagctgaagcaccacccggggtccatcgaggccgcggccatcatggagcacatcctggatggcagctccttcatgaagcaggccaagaaggtgaacgagctggacccgctgctgaagcccaagcaggacaggtacgcgctccgcacgtcgccgcagtggctgggcccccagatcgaggtcatccgcgccgccaccaagtccatcgagcgcgaggtcaactccgtgaacgacaacccggtcatcgacgtccaccgcggcaaggcgctgcacggcggcaacttccagggcacccccatcggcgtgtccatggacaacgcccgcctcgccatcgccaacatcggcaagctcatgttcgcgcagttctccgagctcgtcaacgagttctacaacaacgggctcacctccaacctggccggcagccgcaaccccagcctggactacggcttcaagggcaccgagatcgccatggcctcctactgctccgagctccagtacctgggcaaccccatcaccaaccacgtgcagagcgcggacgagcacaaccaggacgtgaactccctgggcctcgtctcggccaggaagaccgccgaggcgatcgacatcctgaagctcatgtcgtccacctacatcgtggcgctgtgccaggccgtggacctgcgccacctcgaggagaacatcaaggcgtcggtgaagaacaccgtgacccaggtggccaagaaggtgctgaccatgaacccctcgggcgagctctccagcgcccgcttcagcgagaaggagctgatcagcgccatcgaccgcgaggccgtgttcacgtacgcggaggacgcggccagcgccagcctgccgctgatgcagaagctgcgcgccgtgctggtggaccacgccctcagcagcggcgagcgcggagcgggagccctccgtgttctccaagatcaccaggttcgaggaggagctccgcgcggtgctgccccaggaggtggaggccgcccgcgtggcgtcgccgagggcaccgcccccgtggcgaaccggatcgcggacagccggtcgttcccgctgtaccgcttcgtgcgcgaggagctcggctgcgtgttcctgaccggcgagaggctcaagtcccccggcgaggagtgcaacaaggtgttcgtcggcatcagccagggcaagctcgtggaccccatgctcgagtgcctcaaggagtgggacggcaagccgctgcccatcaacatcaagtaa

SEQ ID NO: 16 - protein, 703 aa, Zea mays
MAGNGAIVESDPLNWGAAAAELAGSHLDEVKRMVAQARQPVVKIEGSTLRVGQVAAVASAKDASGVAVELDEEARPRVKASSEWILDCIAHGGDIYGVTTGFGGTSHRRTKDGPALQVELLRHLNAGIFGTGSDGHTLPSEVTRAAMLVRINTLLQGYSGIRFEILEAITKLLNTGVSPCLPLRGTITASGDLVPLSYIAGLITGRPNAQAVTVDGRKVDAAEAFKIAGIEGGFFKLNPKEGLAIVNGTSVGSALAATVMYDANVLAVLSEVLSAVFCEVMNGKPEYTDHLTHKLKHHPGSIEAAAIMEHILDGSSFMKQAKKVNELDPLLKPKQDRYALRTSPQWLGPQIEVIRAATKSIEREVNSVNDNPVIDVHRGKALHGGNFQGTPIGVSMDNARLAIANIGKLMFAQFSELVNEFYNNGLTSNLAGSRNPSLDYGFKGTEIAMASYCSELQYLGNPITNHVQSADEHNQDVNSLGLVSARKTAEAIDILKLMSSTYIVALCQAVDLRHLEENIKASVKNTVTQVAKKVLTMNPSGELSSARFSEKELISAIDREAVFTYAEDAASASLPLMQKLRAVLVDHALSSGERGAGALRVLQDHQVRGGAPRGAAPGGGGRPRGVAEGTAPVANRIADSRSFPLYRFVREELGCVFLTGERLKSPGEECNKVFVGISQGKLVDPMLECLKEWDGKPLPINIK

SEQ ID NO: 17 - DNA, 2154 bp, Arabidopsis thaliana
atggaccaaattgaagcaatgctatgcggtggtggtgaaaagaccaaggtggccgtaacgacaaaaactcttgcagatcctttgaattggggtctggcagctgaccagatgaaaggtagccatctggatgaagttaagaagatggttgaggaatacagaagaccagtcgtaaatctaggcggcgagacattgacgataggacaggtagctgctatttcgaccgttggcggttcagtgaaggtagaacttgcagaaacaagtagagccggagttaaggcttcatcagattgggtcatggaaagtatgaacaagggcacagattcctatggcgttaccacaggctttggtgctacctctcatagaagaactaaaaatggcactgctttgcaaacagaactgatcagattccttaacgccggtattttcggtaatacaaaggaaacttgccatacattaccccaatcggcaacaagagctgctatgcttgttagggtgaacactttgttgcaaggttactctggaataaggtttgaaattcttgaggccatcacttcactattgaaccacaacatttctccttcgttgcccttaagaggaacaataactgccagcggtgatttggttcccctttcatatatcgcaggcttattaacgggaagacctaattcaaaggccactggtccagacggagaatccttaaccgctaaggaagcatttgagaaagctggtatttcaactggtttctttgatttgcaacccaaggaaggtttagccctggtgaatggcaccgctgtcggcagcggtatggcatccatggtgttgtttgaagctaacgtacaagcagttttggccgaagttttgtccgcaatttttgccgaagtcatgagtggaaaacctgagtttactgatcacttgacccacaggttaaaacatcacccaggacaaattgaagcagcagctatcatggagcacattttggacggctctagctacatgaagttagcccagaaggttcatgaaatggaccctttgcaaaaacccaaacaagatagatatgctttaaggacatccccacaatggcttggccctcaaattgaagtaattagacaagctacaaagtctatagaaagagagatcaactctgttaacgataatccacttattgatgtgtcgaggaataaggcaatacatggaggcaatttccagggtacacccataggagtcagtatggataataccaggcttgccatagccgcaattggcaaattaatgtttgcccaattttctgaattggtcaatgacttctacaataacggtttgccttcgaatctgaccgcatcttctaaccctagtcttgattatggtttcaaaggtgctgagatagcaatggcaagctattgttcagagctgcaatatctagccaacccagtaacctctcatgtacaatcagccgaacaacacaatcaggatgttaattctttgggcctgatttcatcaagaaaaacaagcgaggccgttgatatccttaaattaatgtccacaacatttttagtgggtatatgccaggccgtagatttgagacacttggaagagaatttgagacagacagtgaaaaataccgtatcacaggttgcaaaaaaggttctaactacaggtatcaatggtgaattgcacccatcaagattctgtgaaaaagatttattaaaagttgtagatagagaacaagtatttacttacgttgacgatccatgtagcgctacttatccattgatgcagagattgagacaagttattgtagatcacgctttatccaatggtgaaactgagaaaaatgccgttacttcaatattccaaaagataggtgcctttgaagaagaactgaaggcagttttaccaaaggaagtcgaagctgctagagccgcatacggaaatggtactgcccctataccaaatagaatcaaagagtgtaggtcgtaccctttgtacagattcgttagagaagagttgggaaccaaattactaactggtgaaaaagtcgttagcccaggtgaagaatttgacaaggtattcacagctatgtgcgagggaaagttgatagatccacttatggattgcttgaaagagtggaatggtgcacctattccaatctgctaa

SEQ ID NO: 18 - protein, 717 aa, Arabidopsis thaliana
MDQIEAMLCGGGEKTKVAVTTKTLADPLNWGLAADQMKGSHLDEVKKMVEEYRRPVVNLGGETLTIGQVAAISTVGGSVKVELAETSRAGVKASSDWVMESMNKGTDSYGVTTGFGATSHRRTKNGTALQTELIRFLNAGIFGNTKETCHTLPQSATRAAMLVRVNTLLQGYSGIRFEILEAITSLLNHNISPSLPLRGTITASGDLVPLSYIAGLLTGRPNSKATGPDGESLTAKEAFEKAGISTGFFDLQPKEGLALVNGTAVGSGMASMVLFEANVQAVLAEVLSAIFAEVMSGKPEFTDHLTHRLKHHPGQIEAAAIMEHILDGSSYMKLAQKVHEMDPLQKPKQDRYALRTSPQWLGPQIEVIRQATKSIEREINSVNDNPLIDVSRNKAIHGGNFQGTPIGVSMDNTRLAIAAIGKLMFAQFSELVNDFYNNGLPSNLTASSNPSLDYGFKGAEIAMASYCSELQYLANPVTSHVQSAEQHNQDVNSLGLISSRKTSEAVDILKLMSTTFLVGICQAVDLRHLEENLRQTVKNTVSQVAKKVLTTGINGELHPSRFCEKDLLKVVDREQVFTYVDDPCSATYPLMQRLRQVIVDHALSNGETEKNAVTSIFQKIGAFEEELKAVLPKEVEAARAAYGNGTAPIPNRIKECRSYPLYRFVREELGTKLLTGEKVVSPGEEFDKVFTAMCEGKLIDPLMDCLKEWNGAPIPIC

SEQ ID NO: 19 - DNA, 1521 bp, Ammi majus
atgatggattttgttttgttagaaaaagctcttcttggtttgttcattgcaactatagtagccatcacaatctctaagctaaggggaaagaaacttaagttgcctccaggcccaatccctgtcccagtgtttggtaattggttacaagttggcgacgacttaaaccagaggaatttggtagagtatgctaaaaagttcggcgacttatttctacttaggatgggtcaaagaaacttggtcgtggtttcatcccctgacttagcaaaagacgtactacatacccagggtgtcgagttcggaagtagaactagaaatgttgtgtttgatattttcacaggcaaaggtcaagatatggtttttaccgtatacagcgagcactggaggaaaatgagaagaataatgactgtcccattctttacaaacaaagtggttcaacagtataggttcggatgggaggacgaagccgctagagtagtcgaggatgttaaggcaaatcctgaagccgctaccaacggtattgtgttgaggaatagattacaacttttgatgtacaacaatatgtatagaataatgtttgacaggagatttgaatctgttgatgatccattattcctaaaacttaaggcattgaatggcgagagatcaaggttagctcaatcctttgaatacaacttcggtgacttcattcctatattgaggccattcttgagaggatatcttaagttgtgtcaggaaatcaaggacaaaaggttaaagctattcaaggactacttcgtcgacgagagaaaaaagttggagagtatcaagagcgtaggtaataactccttaaagtgcgccatagatcatattatcgaggcacaagaaaaaggcgagataaacgaggataacgtgttatacatcgtcgagaatatcaacgtggctgccattgaaactacactttggtctattgaatggggtatagcagaactagtgaataaccctgaaatccagaaaaaattgagacacgaattagacaccgtacttggagctggtgttcaaatttgtgaaccagatgttcaaaaattgccttatctacaggccgtgataaaagagactttaaggtacaggatggcaattccattgttagtcccacatatgaatcttcacgaagccaaattggccggctatgatatccctgcagagagcaaaattttggtaaacgcttggtggttagccaataatccagcacattggaacaaacctgatgagtttagaccagaaagatttttggaggaagaatccaaggtcgaggctaatggaaacgactttaagtacatccctttcggtgttggcagaagatcttgcccaggtataattcttgctttaccaatccttggaatagtaattggtaggttggttcaaaacttcgagttacttccacctccaggccaaagcaaaatagatacagccgaaaaaggtggacagttttcattgcaaatcctaaagcattccactattgtgtgtaaacctagaagttcttaa

SEQ ID NO: 20 - protein, 506 aa, Ammi majus
MMDFVLLEKALLGLFIATIVAITISKLRGKKLKLPPGPIPVPVFGNWLQVGDDLNQRNLVEYAKKFGDLFLLRMGQRNLVVVSSPDLAKDVLHTQGVEFGSRTRNVVFDIFTGKGQDMVFTVYSEHWRKMRRIMTVPFFTNKVVQQYRFGWEDEAARVVEDVKANPEAATNGIVLRNRLQLLMYNNMYRIMFDRRFESVDDPLFLKLKALNGERSRLAQSFEYNFGDFIPILRPFLRGYLKLCQEIKDKRLKLFKDYFVDERKKLESIKSVGNNSLKCAIDHIIEAQEKGEINEDNVLYIVENINVAAIETTLWSIEWGIAELVNNPEIQKKLRHELDTVLGAGVQICEPDVQKLPYLQAVIKETLRYRMAIPLLVPHMNLHEAKLAGYDIPAESKILVNAWWLANNPAHWNKPDEFRPERFLEEESKVEANGNDFKYIPFGVGRRSCPGIILALPILGIVIGRLVQNFELLPPPGQSKIDTAEKGGQFSLQILKHSTIVCKPRSS

SEQ ID NO: 21 - DNA, 1200 bp, Hordeum vulgare
atggctgcagtaagattgaaagaagttagaatggcacagagggctgaaggtttagctacagttttagcaatcggtactgccgttccagctaattgtgtttatcaagctacctatccagattattattttagggttactaaaagtgagcacttggcagatttaaaggagaagtttcaaagaatgtgtgacaaatcaatgattagaaagagacacatgcacttgaccgaggaaatattgatcaagaacccaaagatctgtgcacacatggagacctcattggatgctagacacgccatcgcattagttgaagttcccaaattgggccaaggtgcagctgagaaggccattaaggagtggggccaacccttgtctaagattactcatttggtattttgcacaacatccggcgttgacatgcccggtgctgattaccaattaacaaagttgttaggtttgtcccctacagtcaaaaggttaatgatgtaccaacaaggttgctttggtggtgcaactgttttgagattggcaaaagatatcgctgaaaataatagaggtgccagagtgttagtcgtttgttccgagataactgctatggccttcagaggtccatgcaagagtcatttagattccttggtaggtcatgccttgttcggtgatggtgccgctgctgcaattataggcgctgacccagaccaattagacgaacaaccagttttccagttggtatcagcttctcagactatattaccagaatcagaaggtgccatagatggccatttaacagaagctggtttaactatacatttattaaaagatgttcctggtttaatttcagagaacattgaacaggctttggaggatgcctttgaacctttaggtattcataactggaattcaattttctggattgcacatcctggtggccctgccattttagacagagttgaagatagagtaggattggataagaagagaatgagggcttctagggaagtgttatctgaatacggaaatatgtctagtgcctctgtgttgtttgtgttagatgtcatgaggaaaagttctgctaaagacggattggcaaccacaggagaaggaaaagattggggagtgttgtttggattcggaccaggcttgactgtagaaaccttagtgttgcatagtgtcccagtccctgtccctactgcagcttctgcatga

SEQ ID NO: 22 - protein, 399 aa, Hordeum vulgare
MAAVRLKEVRMAQRAEGLATVLAIGTAVPANCVYQATYPDYYFRVTKSEHLADLKEKFQRMCDKSMIRKRHMHLTEEILIKNPKICAHMETSLDARHAIALVEVPKLGQGAAEKAIKEWGQPLSKITHLVFCTTSGVDMPGADYQLTKLLGLSPTVKRLMMYQQGCFGGATVLRLAKDIAENNRGARVLVVCSEITAMAFRGPCKSHLDSLVGHALFGDGAAAAIIGADPDQLDEQPVFQLVSASQTILPESEGAIDGHLTEAGLTIHLLKDVPGLISENIEQALEDAFEPLGIHNWNSIFWIAHPGGPAILDRVEDRVGLDKKRMRASREVLSEYGNMSSASVLFVLDVMRKSSAKDGLATTGEGKDWGVLFGFGPGLTVETLVLHSVPVPVPTAASA

SEQ ID NO: 23 - DNA, 2076 bp, Saccharomyces cerevisiae
atgccgtttggaatagacaacaccgacttcactgtcctggcggggctagtgcttgccgtgctactgtacgtaaagagaaactccatcaaggaactgctgatgtccgatgacggagatatcacagctgtcagctcgggcaacagagacattgctcaggtggtgaccgaaaacaacaagaactacttggtgttgtatgcgtcgcagactgggactgccgaggattacgccaaaaagttttccaaggagctggtggccaagttcaacctaaacgtgatgtgcgcagatgttgagaactacgactttgagtcgctaaacgatgtgcccgtcatagtctcgatttttatctctacatatggtgaaggagacttccccgacggggcggtcaactttgaagactttatttgtaatgcggaagcgggtgcactatcgaacctgaggtataatatgtttggtctgggaaattctacttatgaattctttaatggtgccgccaagaaggccgagaagcatctctccgctgcgggcgctatcagactaggcaagctcggtgaagctgatgatggtgcaggaactacagacgaagattacatggcctggaaggactccatcctggaggttttgaaagacgaactgcatttggacgaacaggaagccaagttcacctctcaattccagtacactgtgttgaacgaaatcactgactccatgtcgcttggtgaaccctctgctcactatttgccctcgcatcagttgaaccgcaacgcagacggcatccaattgggtcccttcgatttgtctcaaccgtatattgcacccatcgtgaaatctcgcgaactgttctcttccaatgaccgtaattgcatccactctgaatttgacttgtccggctctaacatcaagtactccactggtgaccatcttgctgtttggccttccaacccattggaaaaggtcgaacagttcttatccatattcaacctggaccctgaaaccatttttgacttgaagcccctggatcccaccgtcaaagtgcccttcccaacgccaactactattggcgctgctattaaacactatttggaaattacaggacctgtctccagacaattgttttcatctttgattcagttcgcccccaacgctgacgtcaaggaaaaattgactctgctttcgaaagacaaggaccaattcgccgtcgagataacctccaaatatttcaacatcgcagatgctctgaaatatttgtctgatggcgccaaatgggacaccgtacccatgcaattcttggtcgaatcagttccccaaatgactcctcgttactactctatctcttcctcttctctgtctgaaaagcaaaccgtccatgtcacctccattgtggaaaactttcctaacccagaattgcctgatgctcctccagttgttggtgttacgactaacttgttaagaaacattcaattggctcaaaacaatgttaacattgccgaaactaacctacctgttcactacgatttaaatggcccacgtaaacttttcgccaattacaaattgcccgtccacgttcgtcgttctaacttcagattgccttccaacccttccaccccagttatcatgatcggtccaggtaccggtgttgccccattccgtgggtttatcagagagcgtgtcgcgttcctcgaatcacaaaagaagggcggtaacaacgtttcgctaggtaagcatatactgttttatggatcccgtaacactgatgatttcttgtaccaggacgaatggccagaatacgccaaaaaattggatggttcgttcgaaatggtcgtggcccattccaggttgccaaacaccaaaaaagtttatgttcaagataaattaaaggattacgaagaccaagtatttgaaatgattaacaacggtgcatttatctacgtctgtggtgatgcaaagggtatggccaagggtgtgtcaaccgcattggttggcatcttatcccgtggtaaatccattaccactgatgaagcaacagagctaatcaagatgctcaagacttcaggtagataccaagaagatgtctggtaa

SEQ ID NO: 24 - protein, 691 aa, Saccharomyces cerevisiae
MPFGIDNTDFTVLAGLVLAVLLYVKRNSIKELLMSDDGDITAVSSGNRDIAQVVTENNKNYLVLYASQTGTAEDYAKKFSKELVAKFNLNVMCADVENYDFESLNDVPVIVSIFISTYGEGDFPDGAVNFEDFICNAEAGALSNLRYNMFGLGNSTYEFFNGAAKKAEKHLSAAGAIRLGKLGEADDGAGTTDEDYMAWKDSILEVLKDELHLDEQEAKFTSQFQYTVLNEITDSMSLGEPSAHYLPSHQLNRNADGIQLGPFDLSQPYIAPIVKSRELFSSNDRNCIHSEFDLSGSNIKYSTGDHLAVWPSNPLEKVEQFLSIFNLDPETIFDLKPLDPTVKVPFPTPTTIGAAIKHYLEITGPVSRQLFSSLIQFAPNADVKEKLTLLSKDKDQFAVEITSKYFNIADALKYLSDGAKWDTVPMQFLVESVPQMTPRYYSISSSSLSEKQTVHVTSIVENFPNPELPDAPPVVGVTTNLLRNIQLAQNNVNIAETNLPVHYDLNGPRKLFANYKLPVHVRRSNFRLPSNPSTPVIMIGPGTGVAPFRGFIRERVAFLESQKKGGNNVSLGKHILFYGSRNTDDFLYQDEWPEYAKKLDGSFEMVVAHSRLPNTKKVYVQDKLKDYEDQVFEMINNGAFIYVCGDAKGMAKGVSTALVGILSRGKSITTDEATELIKMLKTSGRYQEDVW

SEQ ID NO: 25 - DNA, 1383 bp, Arabidopsis thaliana
atgaccaagccatctgatccaaccagagattctcatgttgctgttttggcttttccatttggtactcatgctgctccattattgactgttactagaagattggcttctgcttctccatctaccgttttttcttttttcaacaccgcccaatccaactcctctttgttttcatctggtgatgaagctgatagaccagccaatattagagtttacgatattgctgatggtgtcccagaaggttacgttttttcaggtagaccacaagaagccatcgaattattcttgcaagctgctccagaaaacttcagaagagaaattgctaaggctgaaaccgaagttggtactgaagttaagtgtttgatgaccgatgcttttttttggttcgctgctgatatggctactgaaatcaatgcttcttggattgctttttggactgctggtgctaattctttgtctgctcacttgtacaccgatttgattagagaaaccatcggtgtcaaagaagtcggtgaaagaatggaagaaactattggtgttatttccggtatggaaaagatcagagttaaggatactccagaaggtgttgttttcggtaacttggattctgttttctccaagatgttgcaccaaatgggtttggctttgccaagagctactgctgtttttatcaactccttcgaagatttggatcctaccttgactaacaacttgagatccagattcaagagatacttgaacattggtccattgggtttgttgtcctctacattgcaacaattggttcaagatccacatggttgtttggcttggatggaaaaaagatcatctggttccgttgcctacatttcttttggtactgttatgactccaccaccaggtgaattggctgctattgctgaaggtttggaatcttctaaggttccatttgtttggtccttgaaagaaaagtccttggtccaattgccaaagggttttttggatagaactagagaacaaggtatcgttgttccatgggctccacaagttgaattattgaaacatgaagctaccggtgttttcgttactcattgtggttggaattctgtcttggaatcagtttctggtggtgttccaatgatctgtagaccattttttggtgaccaaagattgaacggtagagccgttgaagttgtttgggaaattggtatgaccatcatcaatggtgttttcaccaaggatggtttcgaaaagtgtttggataaggttttggtccaagacgacggtaaaaagatgaagtgtaatgccaagaagttgaaagaattggcttacgaagctgtctcctctaaaggtagatcatccgaaaatttcagaggtttgttggatgccgttgtcaacattatctga

SEQ ID NO: 26 - protein, 460 aa, Arabidopsis thaliana
MTKPSDPTRDSHVAVLAFPFGTHAAPLLTVTRRLASASPSTVFSFFNTAQSNSSLFSSGDEADRPANIRVYDIADGVPEGYVFSGRPQEAIELFLQAAPENFRREIAKAETEVGTEVKCLMTDAFFWFAADMATEINASWIAFWTAGANSLSAHLYTDLIRETIGVKEVGERMEETIGVISGMEKIRVKDTPEGVVFGNLDSVFSKMLHQMGLALPRATAVFINSFEDLDPTLTNNLRSRFKRYLNIGPLGLLSSTLQQLVQDPHGCLAWMEKRSSGSVAYISFGTVMTPPPGELAAIAEGLESSKVPFVWSLKEKSLVQLPKGFLDRTREQGIVVPWAPQVELLKHEATGVFVTHCGWNSVLESVSGGVPMICRPFFGDQRLNGRAVEVVWEIGMTIINGVFTKDGFEKCLDKVLVQDDGKKMKCNAKKLKELAYEAVSSKGRSSENFRGLLDAVVNII

SEQ ID NO: 27 - DNA, 1539 bp, Petunia x hybrida
atggagattttaagtttaattttgtatacagttatcttcagtttcttattgcaatttattttgagatctttctttaggaaaagatatccattaccattacctccaggtccaaaaccatggccaataataggcaacttagtacacttgggacccaaaccacaccagtctaccgccgctatggcccaaacatatggtccattgatgtacttaaagatgggcttcgtagacgtcgttgtcgctgcatctgcaagtgttgctgcacaattcttgaagactcacgatgctaacttctcttctagacctccaaatagtggcgctgagcatatggcctataattaccaagacttggttttcgccccatacggccctaggtggagaatgttaaggaaaatatgttctgtgcacttgttctctacaaaagcattggatgatttcagacatgtcagacaagacgaagtaaagactttaaccagagcattagcttcagcaggtcagaagcccgtgaagttaggccaattattaaacgtctgtactactaatgctttagccagagtaatgttaggtaaaagagtcttcgctgacggttcaggcgatgttgacccacaagccgcagaattcaaatctatggtagttgagatgatggtcgtcgccggtgtatttaacataggagatttcattcctcaattaaattggttggacattcaaggtgtggccgctaaaatgaagaagttacatgctagattcgatgctttcttgacagacatattggaagaacataaaggtaaaatctttggtgaaatgaaggatttattaagtaccttaatctccttgaagaatgatgatgccgacaatgatggtggaaaattgacagatacagagattaaagcattattattaaacttgtttgttgcaggaactgatacttcatcctcaactgttgaatgggcaattgccgaattgatcagaaatccaaagattttggctcaggctcaacaagagatcgacaaagtggtaggtagagacaggttggtgggcgaattagatttagcacaattaacctacttggaagcaattgttaaggaaacctttagattgcatccctccactccattatcattgccaagaatagcatcagaatcatgtgaaatcaacggttactttatcccaaaaggatccactttattattgaatgtttgggctatagccagggatcctaatgcttgggccgatcctttagaatttagacctgaaagattcttgcctggtggtgaaaagcctaaggtggatgtaaggggaaatgattttgaggtgattccctttggagcaggtaggaggatttgcgctggaatgaatttgggtattaggatggttcagttaatgatcgcaacattgatacatgcatttaactgggatttggtttccggtcagttgcctgaaatgttgaacatggaagaggcttatggtttgacattgcagagagctgatcctttggttgttcatcccagacccagattggaagctcaggcttatatcggttga

SEQ ID NO: 28 - protein, 512 aa, Petunia x hybrida
MEILSLILYTVIFSFLLQFILRSFFRKRYPLPLPPGPKPWPIIGNLVHLGPKPHQSTAAMAQTYGPLMYLKMGFVDVVVAASASVAAQFLKTHDANFSSRPPNSGAEHMAYNYQDLVFAPYGPRWRMLRKICSVHLFSTKALDDFRHVRQDEVKTLTRALASAGQKPVKLGQLLNVCTTNALARVMLGKRVFADGSGDVDPQAAEFKSMVVEMMVVAGVFNIGDFIPQLNWLDIQGVAAKMKKLHARFDAFLTDILEEHKGKIFGEMKDLLSTLISLKNDDADNDGGKLTDTEIKALLLNLFVAGTDTSSSTVEWAIAELIRNPKILAQAQQEIDKVVGRDRLVGELDLAQLTYLEAIVKETFRLHPSTPLSLPRIASESCEINGYFIPKGSTLLLNVWAIARDPNAWADPLEFRPERFLPGGEKPKVDVRGNDFEVIPFGAGRRICAGMNLGIRMVQLMIATLIHAFNWDLVSGQLPEMLNMEEAYGLTLQRADPLVVHPRPRLEAQAYIG

SEQ ID NO: 29 - DNA, 1053 bp, Fragaria x ananassa
atgactgttagtccatctatcgctagtgcagccaaatctggcagagtattaattatcggtgccaccggctttataggtaaatttgttgctgaagcatctttggatagtggcttgccaacatatgtcttagtaagaccaggtccttcaagaccaagtaaaagtgatacaattaaatctttaaaagacaggggcgcaataattttacacggtgtcatgtctgataaaccattgatggaaaaattgttaaaggagcatgaaatcgagattgttatttcagctgtgggtggtgctactattttagatcaaatcaccttggtagaagctatcacctcagtaggaacagtcaagagatttttgccctccgaatttggccatgacgtagatagagccgaccctgttgaacccggtttgaccatgtatttggaaaagagaaaggtcagaagggccatagaaaagtctggtgtaccatacacttacatatgctgtaactcaatcgcctcatggccatactatgataataagcacccttctgaagtggtgccacctttggatcaattccagatctatggcgatggaaccgttaaggcatactttgtggatggacctgatattggtaaatttactatgaagactgtcgatgatatcaggactatgaacaaaaacgttcatttcagaccatcctccaatttatatgatattaatggattggcctcattgtgggaaaagaagattggaagaactttgccaaaggtgactataaccgagaatgacttgttaacaatggcagctgaaaacagaattcctgaatctatagttgcatccttcacacatgatattttcataaaaggttgccaaactaattttcccatagaaggtcctaatgacgttgacattggaacattatatcctgaggaatcctttaggactttagacgaatgtttcaatgatttcttagttaaagttggtggtaaattagagacagacaaattagcagctaaaaacaaagcagcagttggtgtcgagcccatggctattacagctacatgtgcttaa

SEQ ID NO: 30 - protein, 350 aa, Fragaria x ananassa
MTVSPSIASAAKSGRVLIIGATGFIGKFVAEASLDSGLPTYVLVRPGPSRPSKSDTIKSLKDRGAIILHGVMSDKPLMEKLLKEHEIEIVISAVGGATILDQITLVEAITSVGTVKRFLPSEFGHDVDRADPVEPGLTMYLEKRKVRRAIEKSGVPYTYICCNSIASWPYYDNKHPSEVVPPLDQFQIYGDGTVKAYFVDGPDIGKFTMKTVDDIRTMNKNVHFRPSSNLYDINGLASLWEKKIGRTLPKVTITENDLLTMAAENRIPESIVASFTHDIFIKGCQTNFPIEGPNDVDIGTLYPEESFRTLDECFNDFLVKVGGKLETDKLAAKNKAAVGVEPMAITATCA

SEQ ID NO: 31 - DNA, 2079 bp, Arabidopsis thaliana
atgacttctgcactttatgcctccgatcttttcaaacaattgaaaagtatcatgggaacggattctttgtccgatgatgttgtattagttattgctacaacttctctggcactggttgctggtttcgttgtcttattgtggaaaaagaccacggcagatcgttccggcgagctaaagccactaatgatccctaagtctctgatggcgaaagatgaggatgatgacttagatctaggttctggaaaaacgagagtctctatcttcttcggcacacaaaccggaacagccgaaggattcgctaaagcactttcagaagagatcaaagcaagatacgaaaaggcggctgtaaaagtaatcgatttggatgattacgctgccgatgatgaccaatatgaggaaaagttgaaaaaggaaacattggctttcttttgtgtagccacgtatggtgatggtgaaccaaccgataacgccgcaagattctacaagtggtttactgaagagaacgaaagagatatcaagttgcagcaacttgcttacggcgtttttgccttaggtaacagacaatacgagcactttaacaagataggtattgtcttagatgaagagttatgcaaaaagggtgcgaagagattgattgaagtcggtttaggagatgatgatcaatctatcgaggatgactttaatgcatggaaggaatctttgtggtctgaattagataagttacttaaggacgaagatgataaatccgttgccactccatacacagccgtcattccagaatatagagtagttactcatgatccaagattcacaacacagaaatcaatggaaagtaatgtggctaatggtaatactaccatcgatattcatcatccatgtagagtagacgttgcagttcaaaaggaattgcacactcatgaatcagacagatcttgcatacatcttgaatttgatatatcacgtactggtatcacttacgaaacaggtgatcacgtgggtgtctacgctgaaaaccatgttgaaattgtagaggaagctggaaagttgttgggccatagtttagatcttgttttctcaattcatgccgataaagaggatggctcaccactagaaagtgcagtgcctccaccatttccaggaccatgcaccctaggtaccggtttagctcgttacgcggatctgttaaatcctccacgtaaatcagctctagtggccttggctgcgtacgccacagaaccttctgaggcagaaaaactgaaacatctaacttcaccagatggtaaggatgaatactcacaatggatagtagctagtcaacgttctttactagaagttatggctgctttcccatccgctaaacctcctttgggtgttttcttcgccgcaatagcgcctagactgcaaccaagatactattcaatttcatcctcacctagactggcaccatcaagagttcatgtcacatccgctttagtgtacggtccaactcctactggtagaatccataagggcgtttgttcaacatggatgaaaaacgcggttccagcagagaagtctcacgaatgttctggtgctccaatctttatcagagcctccaacttcaaactgccttccaatccttctactcctattgtcatggtcggtcctggtacaggtcttgctccattcagaggtttcttacaagagagaatggccttaaaggaggatggtgaagagttgggatcttctttgttgtttttcggctgtagaaacagacaaatggatttcatctacgaagatgaactgaataactttgtagatcaaggagttatttcagagttgataatggctttttctagagaaggtgctcagaaggagtacgtccaacacaaaatgatggaaaaggccgcacaagtttgggacttaatcaaagaggaaggctatctatatgtctgtggtgatgcaaagggtatggcaagagatgttcacagaacacttcatactatagtccaggaacaggaaggcgttagttcttctgaagcggaagcaattgtgaaaaagttacaaacagagggaagatacttgagagatgtgtggtaa

SEQ ID NO: 32 - protein, 692 aa, Arabidopsis thaliana [sequence omitted]

SEQ ID NO: 33 - DNA, 1521 bp, Viola tricolor
atggcaattctagtcaccgacttcgttgtcgcggctataattttcttgatcactcggttcttagttcgttctcttttcaagaaaccaacccgaccgctccccccgggtcctctcggttggcccttggtgggcgccctccctctcctaggcgccatgcctcacgtcgcactagccaaactcgctaagaagtatggtccgatcatgcacctaaaaatgggcacgtgcgacatggtggtcgcgtccacccccgagtcggctcgagccttcctcaaaacgctagacctcaacttctccaaccgcccacccaacgcgggcgcatcccacctagcgtacggcgcgcaggacttagtcttcgccaagtacggtccgaggtggaagactttaagaaaattgagcaacctccacatgctaggcgggaaggcgttggatgattgggcaaatgtgagggtcaccgagctaggccacatgcttaaagccatgtgcgaggcgagccggtgcggggagcccgtggtgctggccgagatgctcacgtacgccatggcgaacatgatcggtcaagtgatactcagccggcgcgtgttcgtgaccaaagggaccgagtctaacgagttcaaagacatggtggtcgagttgatgacgtccgccgggtacttcaacatcggtgacttcataccctcgatcgcttggatggatttgcaagggatcgagcgagggatgaagaagctgcacacgaagtttgatgtgttattgacgaagatggtgaaggagcatagagcgacgagtcatgagcgcaaagggaaggcagatttcctcgacgttctcttggaagaatgcgacaatacaaatggggagaagcttagtattaccaatatcaaagctgtccttttgaatctattcacggcgggcacggacacatcttcgagcataatcgaatgggcgttaacggagatgatcaagaatccgacgatcttaaaaaaggcgcaagaggagatggatcgagtcatcggtcgtgatcggaggctgctcgaatcggacatatcgagcctcccgtacctacaagccattgctaaagaaacgtatcgcaaacacccgtcgacgcctctcaacttgccgaggattgcgatccaagcatgtgaagttgatggctactacatccctaaggacgcgaggcttagcgtgaacatttgggcgatcggtcgggacccgaatgtttgggagaatccgttggagttcttgccggaaagattcttgtctgaagagaatgggaagatcaatcccggtgggaatgattttgagctgattccgtttggagccgggaggagaatttgtgcggggacaaggatgggaatggtccttgtaagttatattttgggcactttggtccattcttttgattggaaattaccaaatggtgtcgctgagcttaatatggatgaaagttttgggcttgcattgcaaaaggccgtgccgctctcggccttggtcagcccacggttggcctcaaacgcgtacgcaacctga

SEQ ID NO: 34 - protein, 506 aa, Viola tricolor
MAILVTDFVVAAIIFLITRFLVRSLFKKPTRPLPPGPLGWPLVGALPLLGAMPHVALAKLAKKYGPIMHLKMGTCDMVVASTPESARAFLKTLDLNFSNRPPNAGASHLAYGAQDLVFAKYGPRWKTLRKLSNLHMLGGKALDDWANVRVTELGHMLKAMCEASRCGEPVVLAEMLTYAMANMIGQVILSRRVFVTKGTESNEFKDMVVELMTSAGYFNIGDFIPSIAWMDLQGIERGMKKLHTKFDVLLTKMVKEHRATSHERKGKADFLDVLLEECDNTNGEKLSITNIKAVLLNLFTAGTDTSSSIIEWALTEMIKNPTILKKAQEEMDRVIGRDRRLLESDISSLPYLQAIAKETYRKHPSTPLNLPRIAIQACEVDGYYIPKDARLSVNIWAIGRDPNVWENPLEFLPERFLSEENGKINPGGNDFELIPFGAGRRICAGTRMGMVLVSYILGTLVHSFDWKLPNGVAELNMDESFGLALQKAVPLSALVSPRLASNAYAT

SEQ ID NO: 35 - DNA, 3561 bp, Artificial sequence
  DNA sequence of pEVE4745 -ZA for HRT integration into XI-3 site
ctaaattgtaagcgttaatattttgttaaaattcgcgttaaatttttgttaaatcagctcattttttaaccaataggccgaaatcggcaaaatcccttataaatcaaaagaatagaccgagatagggttgagtggccgctacagggcgctcccattcgccattcaggctgcgcaactgttgggaagggcgtttcggtgcgggcctcttcgctattacgccagctggcgaaagggggatgtgctgcaaggcgattaagttgggtaacgccagggttttcccagtcacgacgttgtaaaacgacggccagtgagcgcgacgtaatacgactcactatagggcgaattgaaggaaggccgtcaaggccgcatgtcgacggcgcgccagttacttgctctatgcgtttgcgcatcctctttttactttttttttttcagtaaagcctaagcataaatcgttttatacgtacgacacgttcaacttttcttggttagtagtggcaatctctgcaatacatacagggagtcatggtctatcatcttgtccaatcaaagaagcatcggttcagatcgagcaaactgtagggagaaaggaaagtagaaatgcagagtgtgctatatgtccaatctcggttttgtagtttggatgtcattagagatctaccacccaaccggctgctttcatgtggaacagaaaagaaatcggggcgcttcctcttctgtattcctttaattaacgtttttattcagccatctaaccatcatacccccatacggtaacaaaacctcttctaagaaaagaagtctctgctcctccgccatcttatttttattcgctgcgcgcgtttattgtcgcatcgctagccagcaaaaagttggttgcctttttttacctaaaaaagacacatctaactgattagttttccgttttaggatattgacgccaagcgtgcgtctgattcccgggtcatcgtccacctccggagaacaggccaccatcacgcatctgtgtctgaatttcatcacgaggcgcgccttttcccgtctttcagtgccttgttcagttcttcctgacgggcggtatatttctccagcttactagtttacgtggattgagccagcaatacagatcattattaaactgttttgtacatgatgttagtatataatcgtaaagcttttctaatatgtataccttatacatggaactccacagaacttgcaaacataccaaaaatcctttattcttgttcactcattttacatcaaaaaataatatttcagttattaaggaaaataaaaaaatagattagagaagcattttgaagaaatagtatattcttttattgaacctaagagcgtgatatttttactcgaaataaaatacgaaaaatctatacactcatctttccgactactattggctcctgctcaaaaaaagagggaaaaaaagctccaaaattctatcttttcctatcgctcctgtcctatccttattacgttcattactattttaatactatccattcttttattttcagtctaaaaaaaacatttctcataacgggaaaagcaaaaaaatgtcaagcttatacatcaaaacaccactgcatgcattatctgctggtccggattctcaggcgcgcccctgcaggctgggcctcatgggccttcctttcactgcccgctttccagtcgggaaacctgtcgtgccagctgcattaacatggtcatagctgtttccttgcgtattgggcgctctccgcttcctcgctcactgactcgctgcgctcggtcgttcgggtaaagcctggggtgcctaatgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttattagaaaaattcatccagcagacgataaaacgcaatacgctggctatccggtgccgcaatgccatacagcaccagaaaacgatccgcccattcgccgcccagttcttccgcaatatcacgggtggccagcgcaatatcctgataacgatccgccacgcccagacggccgcaatcaataaagccgctaaaacggccattttccaccataatgttcggcaggcacgcatcaccatgggtcaccaccagatcttcgccatccggcatgctcgctttcagacgcgcaaacagctctgccggtgccaggccctgatgttcttcatccagatcatcctgatccaccaggcccgcttccatacgggtacgcgcacgttcaatacgatgtttcgcctgatgatcaaacggacaggtcgccgggtccagggtatgcagacgacgcatggcatccgccataatgctcactttttctgccggcgccagatggctagacagcagatcctgacccggcacttcgcccagcagcagccaatcacggcccgcttcggtcaccacatccagcaccgccgcacacggaacaccggtggtggccagccagctcagacgcgccgcttcatcctgcagctcgttcagcgcaccgctcagatcggttttcacaaacagcaccggacgaccctgcgcgctcagacgaaacaccgccgcatcagagcagccaatggtctgctgcgcccaatcatagccaaacagacgttccacccacgctgccgggctacccgcatgcaggccatcctgttcaatcatactcttcctttttcaatattattgaagcatttatcagggttattgtctcatgagcggatacatatttgaatgtatttagaaaaataaacaaataggggttccgcgcacatttccccgaaaagtgccac

SEQ ID NO: 36 - DNA, 4595 bp, Artificial Sequence
  DNA sequence of pEVE3169 -AB with URA3 marker flanked by LoxP sites
cggtgcgggcctcttcgctattacgccagctggcgaaagggggatgtgctgcaaggcgattaagttgggtaacgccagggttttcccagtcacgacgttgtaaaacgacggccagtgaattgtaatacgactcactatagggcgacccttaggatcctatggcgcgcctcatcgtccacctccggagaacaggccaccatcacgcatctgtgtctgaatttcatcacgacgcgccgctgcaggtcgacaacccttaatataacttcgtataatgtatgctatacgaagttattaggtctagagatcccaatacaacagatcacgtgatcttttgtaagatgaagttgaagtgagtgttgcaccgtgccaatgcaggtggctattagattaaatatgtgatttgttctattaagtttcctgtataattaatggggagcgctgattctcttttggtacgcttcccatccagcatttctgtatctttcaccttcaaccttaggatctctacccttggcgaaaagtcctctgccaacaatgatgatatctgatccaccacttacaacttcgtcgacggttctgtactgctgacccaatgcatcgcctttgtcgtctaaacctacacctggggtcatgattagccaatcaaacccttcttctcttcctcccatatcgttctgagcaatgaacccaataacgaaatctttatcactctttgcaatatcaacggtacccttagtatattcaccgtgtgctagagaacccttggaagacaattcagcaagcatcaataatccccttggttctttggtgacctcttgcgcaccttgtttcaagccagcaacaataccagcaccagtaaccccgtgggcgttggtgatatcagaccattctgcgatacggtaaacgcccgatgtatattgtaatttgactgtgttaccgatatcggcgaattttctgtcctcaaatatcaagaacttgtatttctctgccaatgctttcaatggaacgacagtaccctcataactgaaatcatccaagatatcaacgtgtgttttcaaaaggcaaatgtatggacccaacgtttcaacaagtttcaatagctcatcagtcgaacgaacgtcaagagaagcacacaaattggtcttcttttcatccattaaacgtaaaagtttcgatgcaaccggacttgcatgagtctcagctctactggtatatgattttgtggacatggtgcaactaattgacgggagtgtattgacgctggcgtactggctttcacaaaatggcccaatcacaaccacatcttagatagttgaaatgactttagataacatcaattgagatgagcttaatcatgtcaaagctaaaagtgtcaccatgaacgacaattcttaagcaaatcacgtgatatagatccacgaataaccaccatttgatgctcgaggcaagtaatgtgtgtaaaaaaatgcgttaccaccatccaatgcagaccgatcttctacccagaatcacatatatttatgtaccgagtaccttttttctatcttccaattgcttctcccatatgattgtctccgtaagctcgaaatttctaagttggattttaatcttcacgcaggatgacagttcgatgagcttctgaggagtgtttagaacataatcagtttatccatggtctatctcttcttgtcgctttttctcctcgatagaacctaaataaaacgagctctcgagaacccttaatataacttcgtataatgtatgctatacgaagttattaggtgatatcagatccggcgcgtggcacccttgcgggccatgtcatacaccgccttcagagcagccggacctatctgcccgttggcgcgcctattgaaagatcttaaggggatatcctcgaggttccctttagtgagggttaattgcgagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttgccattcgccattcaggctgcgcaactgttgggaagggcgat

SEQ ID NO: 37 - DNA, 3633 bp, Artificial Sequence
  DNA sequence of pEVE1919 - Closing linker HZ for 6 gene plasmid or integration
cggtgcgggcctcttcgctattacgccagctggcgaaagggggatgtgctgcaaggcgattaagttgggtaacgccagggttttcccagtcacgacgttgtaaaacgacggccagtgaattgtaatacgactcactatagggcgaccctaggatcctatggcgcgccgccaccaacagccccgccaatggcgctgccgatactcccgacaatccccaccattgcctgacgcgtccagtatcccagcagatacgggatatcgacatttctgcaccattccggcgggtataggttttattgatggcctcatccacacgcagcagcgtctgttcatcgtcgtggcggcccataataatctgccggtcaatcagccagctttcctcacccggcccccatccccatacgcgcatttcgtagcggtccagctgggagtcgataccggcggtcaggtaagccacacggtcaggaacgggcgctgaataatgctctttccgctctgccatcacttcagcatccggacgttcgccaattttcgcctcccacgtctcaccgagcgtggtgtttacgaaggttttacgttttcccgtatcccctttcgttttcatccagtctttgacaatctgcacccaggtggtgaacgggctgtacgctgtccagatgtgaaaggtcacactgtcaggtggctcaatctcttcaccggatgacgaaaaccagagaatgccatcacgggtccagatcccggtcttttcgcagatataacgggcatcagtaaagtccagctcctgctggcggatgacgcaggcattatgctcgcagagataaaacacgctggagacgcgttttcccgtctttcagtgccttgttcagttcttcctgacgggcggtatatttctccagcttggcgcgcctaagacttagatcttaaggggatatcctcgaggttccctttagtgagggttaattgcgagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttgccattcgccattcaggctgcgcaactgttgggaagggcgat

SEQ ID NO: 38 - DNA, 6308 bp, Artificial Sequence
  DNA sequence of pEVE4729 - ZA with HIS3 marker and pSC101 ORI for HRT plasmids
cggccgcctgcacggtcctgttccctagcatgtacgtgagcgtatttccttttaaaccacgacgctttgtcttcattcaacgtttcccattgtttttttctactattgctttgctgtgggaaaaacttatcgaaagatgacgactttttcttaattctcgttttaagagcttggtgagcgctaggagtcactgccaggtatcgtttgaacacggcattagtcagggaagtcataacacagtcctttcccgcaattttctttttctattactcttggcctcctctagtacactctatatttttttatgcctcggtaatgattttcatttttttttttccacctagcggatgactctttttttttcttagcgattggcattatcacataatgaattatacattatataaagtaatgtgatttcttcgaagaatatactaaaaaatgagcaggcaagataaacgaaggcaaagatgacagagcagaaagccctagtaaagcgtattacaaatgaaaccaagattcagattgcgatctctttaaagggtggtcccctagcgatagagcactcgatcttcccagaaaaagaggcagaagcagtagcagaacaggccacacaatcgcaagtgattaacgtccacacaggtatagggtttctggaccatatgatacatgctctggccaagcattccggctggtcgctaatcgttgagtgcattggtgacttacacatagacgaccatcacaccactgaagactgcgggattgctctcggtcaagcttttaaagaggccctaggggccgtgcgtggagtaaaaaggtttggatcaggatttgcgcctttggatgaggcactttccagagcggtggtagatctttcgaacaggccgtacgcagttgtcgaacttggtttgcaaagggagaaagtaggagatctctcttgcgagatgatcccgcattttcttgaaagctttgcagaggctagcagaattaccctccacgttgattgtctgcgaggcaagaatgatcatcaccgtagtgagagtgcgttcaaggctcttgcggttgccataagagaagccacctcgcccaatggtaccaacgatgttccctccaccaaaggtgttcttatgtagtgacaccgattatttaaagctgcagcatacgatatatatacatgtgtatatatgtatacctatgaatgtcagtaagtatgtatacgaacagtatgatactgaagatgacaaggtaatgcatcattctatacgtgtcattctgaacgaggcgcgctttccttttttctttttgctttttctttttttttctcttgaactcgatcgagaaaaaaaatataaaagagatggaggaacgggaaaaagttagttgtggtgataggtggcaagtggtattccgtaagaacaacaagaaaagcatttcatattatggctgaactgagcgaacaagtgcaaaatttaagcatcaacgacaacaacgagaatggttatgttcctcctcacttaagaggaaaaccaagaagtgccagaaataacagtagcaactacaataacaacaacggcggctacaacggtggccgtggcggtggcagcttctttagcaacaaccgtcgtggtggttacggcaacggtggtttcttcggtggaaacaacggtggcagcagatctaacggccgttctggtggtagatggatcgatggcaaacatgtcccagctccaagaaacgaaaaggccgagatcgccatatttggtgtggcggccgcacgcgttcatcgtccacctccggagaacaggccaccatcacgcatctgtgtctgaatttcatcacgggcgcgccctgggcctcatgggccttccgctcactgcccgctttccagtcgggaaacctgtcgtgccagctgcattaacatggtcatagctgtttccttgcgtattgggcgctctccgcttcctcgctcactgactcgctgcgctcggtcgttcgggtaaagcctggggtgcctaatgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagaaccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctaaattgtaagcgttaatattttgttaaaattcgcgttaaatttttgttaaatcagctcattttttaaccaataggccgaaatcggcaaaatcccttataaatcaaaagaatagaccgagatagggttgagtggccgctacagggcgctcccattcgccattcaggctgcgcaactgttgggaagggcgtttcggtgcgggcctcttcgctattacgccagctggcgaaagggggatgtgctgcaaggcgattaagttgggtaacgccagggttttcccagtcacgacgttgtaaaacgacggccagtgagcgcgacgtaatacgactcactatagggcgaattggcggaaggccgtcaaggccgcatggcgcgcctttcccgtctttcagtgccttgttcagttcttcctgacgggcggtatatttctccagcttggcctatgcggccctgtcagaccaagtttacgagctcgcttggactcctgttgatagatccagtaatgacctcagaactccatctggatttgttcagaacgctcggttgccgccgggcgttttttattggtgagaatccaagcactagggacagtaagacgggtaagcctgttgatgataccgctgccttactgggtgcattagccagtctgaatgacctgtcacgggataatccgaagtggtcagactggaaaatcagagggcaggaactgctgaacagcaaaaagtcagatagcaccacatagcagacccgccataaaacgccctgagaagcccgtgacgggcttttcttgtattatgggtagtttccttgcatgaatccataaaaggcgcctgtagtgccatttacccccattcactgccagagccgtgagcgcagcgaactgaatgtcacgaaaaagacagcgactcaggtgcctgatggtcggagacaaaaggaatattcagcgatttgcccgagcttgcgagggtgctacttaagcctttagggttttaaggtctgttttgtagaggagcaaacagcgtttgcgacatccttttgtaatactgcggaactgactaaagtagtgagttatacacagggctgggatctattctttttatctttttttattctttctttattctataaattataaccacttgaatataaacaaaaaaaacacacaaaggtctagcggaatttacagagggtctagcagaatttacaagttttccagcaaaggtctagcagaatttacagatacccacaactcaaaggaaaaggacatgtaattatcattgactagcccatctcaattggtatagtgattaaaatcacctagaccaattgagatgtatgtctgaattagttgttttcaaagcaaatgaactagcgattagtcgctatgacttaacggagcatgaaaccaagctaattttatgctgtgtggcactactcaaccccacgattgaaaaccctacaaggaaagaacggacggtatcgttcacttataaccaatacgctcagatgatgaacatcagtagggaaaatgcttatggtgtattagctaaagcaaccagagagctgatgacgagaactgtggaaatcaggaatcctttggttaaaggctttgagattttccagtggacaaactatgccaagttctcaagcgaaaaattagaattagtttttagtgaagagatattgccttatcttttccagttaaaaaaattcataaaatataatctggaacatgttaagtcttttgaaaacaaatactctatgaggatttatgagtggttattaaaagaactaacacaaaagaaaactcacaaggcaaatatagagattagccttgatgaatttaagttcatgttaatgcttgaaaataactaccatgagtttaaaaggcttaaccaatgggttttgaaaccaataagtaaagatttaaacacttacagcaatatgaaattggtggttgataagcgaggccgcccgactgatacgttgattttccaagttgaactagatagacaaatggatctcgtaaccgaacttgagaacaaccagataaaaatgaatggtgacaaaataccaacaaccattacatcagattcctacctacataacggactaagaaaaacactacacgatgctttaactgcaaaaattcagctcaccagttttgaggcaaaatttttgagtgacatgcaaagtaagtatgatctcaatggttcgttctcatggctcacgcaaaaacaacgaaccacactagagaacatactggctaaatacggaaggatctgaggttcttatggctcttgtatctatcagtgaagcatcaagactaacaaacaaaagtagaacaactgttcaccgttacatatcaaagggaaaactgtccatatgcacagatgaaaacggtgtaaaaaagatagatacatcagagcttttacgagtttttggtgcattcaaagctgttcaccatgaacagatcgacaatgtaacg

SEQ ID NO: 39 - DNA, 4756 bp, Artificial Sequence
  DNA sequence of pEVE1968 - AB with ARS/CEN origin and CmR marker for HRT plasmids
cggtgcgggcctcttcgctattacgccagctggcgaaagggggatgtgctgcaaggcgattaagttgggtaacgccagggttttcccagtcacgacgttgtaaaacgacggccagtgaattgtaatacgactcactatagggcgacccttaggatcctatggcgcgcctcatcgtccacctccggagaacaggccaccatcacgcatctgtgtctgaatttcatcacgacgcgccttaagggcaccaataactgccttaaaaaaattacgccccgccctgccactcatcgcagtactgttgtaattcattaagcattctgccgacatggaagccatcacagacggcatgatgaacctgaatcgccagcggcatcagcaccttgtcgccttgcgtataatatttgcccatggtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacggaattccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgataactcaaaaaatacgcccggtagtgatcttatttcattatggtgaaagttggaacctcttacgtgccgatcaacgtctcattttcgccaaaagttggcccagggcttcccggtatcaacagggacaccaggatttatttattctgcgaagtgatcttccgtcacaggtattggaccaccctgtgggtttataagcgcgctgctggcgtgtaaggcggtgacggcgaaggaagggtccttttcatcacgtgctataaaaataattataatttaaattttttaatataaatatataaattaaaaatagaaagtaaaaaaagaaattaaagaaaaaatagtttttgttttccgaagatgtaaaagactctagggggatcgccaacaaatactaccttttatcttgctcttcctgctctcaggtattaatgccgaattgtttcatcttgtctgtgtagaagaccacacacgaaaatcctgtgattttacattttacttatcgttaatcgaatgtatatctatttaatctgcttttcttgtctaataaatatatatgtaaagtacgctttttgttgaaattttttaaacctttgtttatttttttttcttcattccgtaactcttctaccttctttatttactttctaaaatccaaatacaaaacataaaaataaataaacacagagtaaattcccaaattattccatcattaaaagatacgaggcgcgtgtaagttacaggcaagcgatccgtcctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccacggcgcgtggcacccttgcgggccatgtcatacaccgccttcagagcagccggacctatctgcccgttggcgcgcctattgaaagatcttaaggggatatcctcgaggttccctttagtgagggttaattgcgagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttgccattcgccattcaggctgcgcaactgttgggaagggcgat

SEQ ID NO: 40 - DNA, 3634 bp, Artificial Sequence
  DNA sequence of pEVE1917 - Closing linker FZ for 4 gene HRT plasmid
cggtgcgggcctcttcgctattacgccagctggcgaaagggggatgtgctgcaaggcgattaagttgggtaacgccagggttttcccagtcacgacgttgtaaaacgacggccagtgaattgtaatacgactcactatagggcgacccttaggatcctatggcgcgccaccacggtgaacaatccccgctggctcatatttgccgccggttcccgtaaatcctccggtacgcgtccagtatcccagcagatacgggatatcgacatttctgcaccattccggcgggtataggttttattgatggcctcatccacacgcagcagcgtctgttcatcgtcgtggcggcccataataatctgccggtcaatcagccagctttcctcacccggcccccatccccatacgcgcatttcgtagcggtccagctgggagtcgataccggcggtcaggtaagccacacggtcaggaacgggcgctgaataatgctctttccgctctgccatcacttcagcatccggacgttcgccaattttcgcctcccacgtctcaccgagcgtggtgtttacgaaggttttacgttttcccgtatcccctttcgttttcatccagtctttgacaatctgcacccaggtggtgaacgggctgtacgctgtccagatgtgaaaggtcacactgtcaggtggctcaatctcttcaccggatgacgaaaaccagagaatgccatcacgggtccagatcccggtcttttcgcagatataacgggcatcagtaaagtccagctcctgctggcggatgacgcaggcattatgctcgcagagataaaacacgctggagacgcgttttcccgtctttcagtgccttgttcagttcttcctgacgggcggtatatttctccagcttggcgcgcctaagacttagatcttaaggggatatcctcgaggttccctttagtgagggttaattgcgagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttgccattcgccattcaggctgcgcaactgttgggaagggcgat

SEQ ID NO: 41 - DNA, 5254 bp, Artificial Sequence
  DNA sequence of pEVE1765 - ZA with LEU2 marker and pMB1 ORI for HRT plasmids
cggtgcgggcctcttcgctattacgccagctggcgaaagggggatgtgctgcaaggcgattaagttgggtaacgccagggttttcccagtcacgacgttgtaaaacgacggccagtgaattgtaatacgactcactatagggcgacccttaggcgcgcctttcccgtctttcagtgccttgttcagttcttcctgacgggcggtatatttctccagcttacgcgccatgcagggatatcagatcttcgaggagaacttctagtatatccacatacctaatattattgccttattaaaaatggaatcccaacaattacatcaaaatccacattctcttcaaaatcaattgtcctgtacttccttgttcatgtgtgttcaaaaacgttatatttataggataattatactctatttctcaacaagtaattggttgtttggccgagcggtctaaggcgcctgattcaagaaatatcttgaccgcagttaactgtgggaatactcaggtatcgtaagatgcaagagttcgaatctcttagcaaccattatttttttcctcaacataacgagaacacacaggggcgctatcgcacagaatcaaattcgatgactggaaattttttgttaatttcagaggtcgcctgacgcatatacctttttcaactgaaaaattgggagaaaaaggaaaggtgagaggccggaaccggcttttcatatagaatagagaagcgttcatgactaaatgcttgcatcacaatacttgaagttgacaatattatttaaggacctattgttttttccaataggtggttagcaatcgtcttactttctaacttttcttaccttttacatttcagcaatatatatatatatttcaaggatataccattctaatgtctgcccctatgtctgcccctaagaagatcgtcgttttgccaggtgaccacgttggtcaagaaatcacagccgaagccattaaggttcttaaagctatttctgatgttcgttccaatgtcaagttcgatttcgaaaatcatttaattggtggtgctgctatcgatgctacaggtgtcccacttccagatgaggcgctggaagcctccaagaaggttgatgccgttttgttaggtgctgtggctggtcctaaatggggtaccggtagtgttagacctgaacaaggtttactaaaaatccgtaaagaacttcaattgtacgccaacttaagaccatgtaactttgcatccgactctcttttagacttatctccaatcaagccacaatttgctaaaggtactgacttcgttgttgtcagagaattagtgggaggtatttactttggtaagagaaaggaagacgatggtgatggtgtcgcttgggatagtgaacaatacaccgttccagaagtgcaaagaatcacaagaatggccgctttcatggccctacaacatgagccaccattgcctatttggtccttggataaagctaatcttttggcctcttcaagattatggagaaaaactgtggaggaaaccatcaagaacgaattccctacattgaaggttcaacatcaattgattgattctgccgccatgatcctagttaagaacccaacccacctaaatggtattataatcaccagcaacatgtttggtgatatcatctccgatgaagcctccgttatcccaggttccttgggtttgttgccatctgcgtccttggcctctttgccagacaagaacaccgcatttggtttgtacgaaccatgccacggttctgctccagatttgccaaagaataaggttgaccctatcgccactatcttgtctgctgcaatgatgttgaaattgtcattgaacttgcctgaagaaggtaaggccattgaagatgcagttaaaaaggttttggatgcaggtatcagaactggtgatttaggtggttccaacagtaccaccgaagtcggtgatgctgtcgccgaagaagttaagaaaatccttgcttaaaaagattctctttttttatgatatttgtacataaactttataaatgaaattcataatagaaacgacacgaaattacaaaatggaatatgttcatagggtagacgaaactatatacgcaatctacatacatttatcaagaaggagaaaaaggaggatagtaaaggaatacaggtaagcaaattgatactaatggctcaacgtgataaggaaaaagaattgcactttaacattaatattgacaaggaggagggcaccacacaaaaagttaggtgtaacagaaaatcatgaaactacgattcctaatttgatattggaggattttctctaaaaaaaaaaaaatacaacaaataaaaaacactcaatgacctgaccatttgatggagtttaagtcaataccttcttgaagcatttcccataatggtgaaagttccctcaagaattttactctgtcagaaacggccttacgacgtagtcgagcatgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaacggcgcgttcatcgtccacctccggagaacaggccaccatcacgcatctgtgtctgaatttcatcacgggcgcgcctaaggggatatcctcgaggttccctttagtgagggttaattgcgagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaacatcataccgtataggctatccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttgccattcgccattcaggctgcgcaactgttgggaagggcgat

SEQ ID NO: 42 - DNA, 3638 bp, Artificial Sequence
  DNA sequence of pEVE1915 - Closing linker DZ for 2 gene HRT plasmid
cggtgcgggcctcttcgctattacgccagctggcgaaagggggatgtgctgcaaggcgattaagttgggtaacgccagggttttcccagtcacgacgttgtaaaacgacggccagtgaattgtaatacgactcactatagggcgacccttaggatctaagcattggcgcgccccggctgtctgccatgctgcccggtgtaccgacataaccgccggtggcatagccgcgcatacgcgtctccagcgtgttttatctctgcgagcataatgcctgcgtcatccgccagcaggagctggactttactgatgcccgttatatctgcgaaaagaccgggatctggacccgtgatggcattctctggttttcgtcatccggtgaagagattgagccacctgacagtgtgacctttcacatctggacagcgtacagcccgttcaccacctgggtgcagattgtcaaagactggatgaaaacgaaaggggatacgggaaaacgtaaaaccttcgtaaacaccacgctcggtgagacgtgggaggcgaaaattggcgaacgtccggatgctgaagtgatggcagagcggaaagagcattattcagcgcccgttcctgaccgtgtggcttacctgaccgccggtatcgactcccagctggaccgctacgaaatgcgcgtatggggatgggggccgggtgaggaaagctggctgattgaccggcagattattatgggccgccacgacgatgaacagacgctgctgcgtgtggatgaggccatcaataaaacctatacccgccggaatggtgcagaaatgtcgatatcccgtatctgctgggatactggacgcgttttcccgtctttcagtgccttgttcagttcttcctgacgggcggtatatttctccagcttggcgcgcctaagacttagatcttaaggggatatcctcgaggttccctttagtgagggttaattgcgagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttgccattcgccattcaggctgcgcaactgttgggaagggcgat

SEQ ID NO: 43 - DNA, 9 bp, Artificial Sequence
  DNA sequence of 5′-end including HindIII restriction site and Kozak sequence
aagcttaaa

SEQ ID NO: 44 - DNA, 6 bp, Artificial Sequence
  DNA sequence of 3′-end including a SacII recognition site
ccgcgg

SEQ ID NO: 45 - DNA, 5356 bp, Vitis amurensis
cggtgcgggcctcttcgctattacgccagctggcgaaagggggatgtgctgcaaggcgattaagttgggtaacgccagggttttcccagtcacgacgttgtaaaacgacggccagtgaattgtaatacgactcactatagggcgacccttaggatcctatggcgcgccaccacggtgaacaatccccgctggctcatatttgccgccggttcccgtaaatcctccggtacgcgccgggccgtatacttacatatagtagatgtcaagcgtaggcgcttcccctgccggctgtgagggcgccataaccaaggtatctatagaccgccaatcagcaaactacctccgtacattcatgttgcacccacacatttatacacccagaccgcgacaaattacccataaggttgtttgtgacggcgtcgtacaagagaacgtgggaactttttaggctcaccaaaaaagaaagaaaaaatacgagttgctgacagaagcctcaagaaaaaaaaaattcttcttcgactatgctggaggcagagatgatcgagccggtagttaactatatatagctaaattggttccatcaccttcttttctggtgtcgctccttctagtgctatttctggcttttcctatttttttttttccatttttctttctctctttctaatatataaattctcttgcattttctatttttctctctatctattctacttgtttattcccttcaaggtttttttttaaggagtacttgtttttagaatatacggtcaacgaactataattaactaaacaagcttaaaatggctaacccacacccacatttcttgattattacttttccagcccaaggtcatattaacccagctttggaattggccaaaagattgattggtgttggtgctgatgttactttcgctactactattcatgccaagtccagattggttaagaacccaactgttgatggtttgagattctctactttctccgatggtcaagaagaaggtgttaagagaggtccaaacgaattgccagtttttcaaagattggcctccgaaaacttgtccgaattgattatggcttctgctaatgaaggtagaccaatctcttgtttgatctactccattttgattccaggtgctgctgaattggctagatcattcaatattccatctgctttcttgtggattcaaccagctactgttttggacatctattactactacttcaacggtttcggtgacttgatcagatccaaatcttctgatccatccttctccattgaattaccaggtttgccatctttgtccagacaagatttgccatcctttttcgttggttccgaccaaaatcaagaaaaccatgctttggctgcctttcaaaagcacttggaaattttggaacaagaagaaaacccaaaggtcttggttaacactttcgatgctttagaaccagaagccttgagagctgttgaaaagttgaaattgactgctgttggtccattggttccatctggtttttctgatggtaaagatgcttctgatacaccatctggtggtgatttgtctgatggttctagagattatatggaatggttgaagtccaagccagaatctactgttgtttacgtttccttcggttccatcagtatgttctctatgcaacaaatggaagaaatcgccagaggtttgttggaatctggtagaccatttttgtgggttatcagagctaaagaaaacggtgaagaaaacaaagaagaagataagttgtcctgccaagaagaattggaaaagcaaggtatgttgatccaatggtgctctcaaatggaagttttgtctcatccatctttgggttgtttcgttactcattgtggttggaactcctctattgaatctttagcttctggtgttccaatgattgcatttccacaatgggctgatcaaggtactaataccaagttgattaaggacgtttggaaaaccggtgttagattgatggttaacgaagaagaaattgtcacctccgacgaattgagaagatgcttggaattagttatgggtgatggtgaaaagggtcaagaaatgagaaagaatgctaagaagtggaagattttggctaaagaagccttaaaagaaggtggttcctctcacaagaatttgaagaacttcgttgacgaagtcatccaaggttactgaccgcggacaaatcgctcttaaatatatacctaaagaacattaaagctatattataagcaaagatacgtaaattttgcttatattattatacacatatcatatttctatatttttaagatttggttatataatgtacgtaatgcaaaggaaataaattttatacattattgaacagcgtccaagtaactacattatgtgcactaatagtttagcgtcgtgaagactttattgtgtcgcgaaaagtaaaaattttaaaaattagagcaccttgaacttgcgaaaaaggttctcatcaactgtttaaaaggaggatatcaggtcctatttctgacaaacaatatacaaatttagtttcaaaggcgcgttgcaaaatggaatttcgccgcagcggcctgaatggctgtaccgcctgacgcggatgcgccggcgcgcctattgaaagatcttaaggggatatcctcgaggttccctttagtgagggttaattgcgagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttgccattcgccattcaggctgcgcaactgttgggaagggcgat

SEQ ID NO: 46 - DNA, 4709 bp, Artificial Sequence
  DNA sequence of pEVE2176 - empty HRT plasmid with BC tags
cggtgcgggcctcttcgctattacgccagctggcgaaagggggatgtgctgcaaggcgattaagttgggtaacgccagggttttcccagtcacgacgttgtaaaacgacggccagtgaattgtaatacgactcactatagggcgacccttaggatcctatggcgcgccggcacccttgcgggccatgtcatacaccgccttcagagcagccggacctatctgcccgttacgcgccagcttgcaaattaaagccttcgagcgtcccaaaaccttctcaagcaaggttttcagtataatgttacatgcgtacacgcgtctgtacagaaaaaaaagaaaaatttgaaatataaataacgttcttaatactaacataactataaaaaaataaatagggacctagacttcaggttgtctaactccttccttttcggttagagcggatgtggggggagggcgtgaatgtaagcgtgacataactaattacatgatatcgacaaaggaaaagggggacggatctccgaggcctcggacccgtcgggccgccgtcggacgtgccgcggatccccgggtcgagcctgaacggcctcgaggcctgaacggcctcgacgaattcattatttgtagagctcatccatgccatgtgtaatcccagcagcagttacaaactcaagaaggaccatgtggtcacgcttttcgttgggatctttcgaaagggcagattgtgtcgacaggtaatggttgtctggtaaaaggacagggccatcgccaattggagtattttgttgataatggtctgctagttgaacggatccatcttcaatgttgtggcgaattttgaagttagctttgattccattcttttgtttgtctgccgtgatgtatacattgtgtgagttatagttgtactcgagtttgtgtccgagaatgtttccatcttctttaaaatcaataccttttaactcgatacgattaacaagggtatcaccttcaaacttgacttcagcacgcgtcttgtagttcccgtcatctttgaaagatatagtgcgttcctgtacataaccttcgggcatggcactcttgaaaaagtcatgccgtttcatatgatccggataacgggaaaagcattgaacaccataagagaaagtagtgacaagtgttggccatggaacaggtagttttccagtagtgcaaataaatttaagggtaagctggccctgcaggccaagctttttgtttgtttatgtgtgtttattcgaaactaagttcttggtgttttaaaactaaaaaaaagactaactataaaagtagaatttaagaagtttaagaaatagatttacagaattacaatcaatacctaccgtctttatatacttattagtcaagtaggggaataatttcagggaactggtttcaaccttttttttcagctttttccaaatcagagagagcagaaggtaatagaaggtgtaagaaaatgagatagatacatgcgtgggtcaattgccttgtgtcatcatttactccaggcaggttgcatcactccattgaggttgtgtccgttttttgcctgtttgtgcccctgttctctgtagttgcgctaagagaatggacctatgaactgatggttggtgaagaaaacaatattttggtgctgggattctttttttttctggatgccagcttaaaaagcgggctccattatatttagtggatgccaggaataaactgttcacccagacacctacgatgttatatattctgtgtaacccgccccctattttgggcatgtacgggttacagcagaattaaaaggctaattttttgactaaataaagttaggaaaatcactactattaattatttacgtattctttgaaatggcagtattgataatgataaactcgaactgggcgcgtcgtgccgtcgttgttaatcaccacatggttattctgctcaaacgtcccggacgcctgcgaggcgcgcctattgaaagatcttaaggggatatcctcgaggttccctttagtgagggttaattgcgagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttgccattcgccattcaggctgcgcaactgttgggaagggcgat

SEQ ID NO: 47 - DNA, 5642 bp, Solanum lycopersicum
cggtgcgggcctcttcgctattacgccagctggcgaaagggggatgtgctgcaaggcgattaagttgggtaacgccagggttttcccagtcacgacgttgtaaaacgacggccagtgaattgtaatacgactcactatagggcgacccttaagatctgtaatggcgcgccatgcgcggctatgccaccggcggttatgtcggtacaccgggcagcatggcagacagccggacgcgccacgcacagatattataacatctgcataataggcatttgcaagaattactcgtgagtaaggaaagagtgaggaactatcgcatacctgcatttaaagatgccgatttgggcgcgaatcctttattttggcttcaccctcatactattatcagggccagaaaaaggaagtgtttccctccttcttgaattgatgttaccctcataaagcacgtggcctcttatcgagaaagaaattaccgtcgctcgtgatttgtttgcaaaaagaacaaaactgaaaaaacccagacacgctcgacttcctgtcttcctattgattgcagcttccaatttcgtcacacaacaaggtcctagcgacggctcacaggttttgtaacaagcaatcgaaggttctggaatggcgggaaagggtttagtaccacatgctatgatgcccactgtgatctccagagcaaagttcgttcgatcgtactgttactctctctctttcaaacagaattgtccgaatcgtgtgacaacaacagcctgttctcacacactcttttcttctaaccaagggggtggtttagtttagtagaacctcgtgaaacttacatttacatatatataaacttgcataaattggtcaatgcaagaaatacatatttggtcttttctaattcgtagtttttcaagttcttagatgctttctttttctcttttttacagatcatcaaggaagtaattatctactttttacaacaaatataaaacaaagcttaaaatggccttgagaatcaacgaattattcgtcgctgccatcatctacatcatcgttcatattatcatctccaagttgatcaccaccgttagagaaagaggtagaagattgccattgccaccaggtccaactggttggccagttattggtgctttgccattattgggttctatgccacatgttgctttggctaaaatggctaagaaatacggtccaatcatgtacttgaaggttggtacttgtggtatggttgttgcttctactccaaatgctgctaaggctttcttgaaaaccttggacattaacttctctaacagaccacctaatgctggtgctactcatttggcttataatgcccaagatatggtttttgctccatatggtccaagatggaagttgttgagaaagttgtctaacttgcatatgttgggtggtaaggctttggaaaattgggctaatgttagagctaacgaattgggtcatatgttgaagtctatgttcgatgcttctcaagatggtgaatgcgttgttattgctgatgttttgactttcgctatggctaacatgatcggtcaagttatgttgtccaagagagttttcgttgaaaagggtgtcgaagttaacgaattcaagaacatggttgtcgaattgatgactgttgctggttactttaacatcggtgatttcattccaaagttggcctggatggatattcaaggtattgaaaaaggtatgaagaacttgcacaagaagttcgacgatttgttgaccaagatgtttgatgaacatgaagccacctccaacgaaagaaaagaaaatccagatttcttggatgtcgtcatggccaatagagataattctgaaggtgaaagattgtccaccaccaatattaaggccttgttgttgaatttgttcaccgctggtactgatacctcctcttctgttattgaatgggctttagctgaaatgatgaagaacccaaaaatcttcaaaaaggcccaacaagaaatggaccaagttatcggtaaaaacagaagattgatcgaatccgacattccaaacttgccatatttgagagctatctgcaaagaaactttcagaaagcacccatctactccattgaatttgccaagagtttcttctgaaccatgtaccgttgatggttactacatcccaaaaaacactagattgtccgttaacatttgggccattggtagagatccagatgtttgggaaaatccattggaattcactccagaaagattcttgtctggtaagaacgctaagattgaacctagaggtaacgactttgaattgattccatttggtgccggtagaagaatttgtgctggtactagaatgggtatcgttgtcgttgaatatatcttaggtactttggtccactccttcgattggaaattgccaaacaacgttatcgacatcaacatggaagaatcatttggtttggccttgcaaaaagctgttccattagaagctatggttaccccaagattgtctttggatgtttacagatgctaaccgcggatctcttatgtctttacgatttatagttttcattatcaagtatgcctatattagtatatagcatctttagatgacagtgttcgaagtttcacgaataaaagataatattctactttttgctcccaccgcgtttgctagcacgagtgaacaccatccctcgcctgtgagttgtacccattcctctaaactgtagacatggtagcttcagcagtgttcgttatgtacggcatcctccaacaaacagtcggttatagtttgtcctgctcctctgaatcgtctccctcgatatttctcattttccttcggcgcgttcgcaggcgtccgggacgtttgagcagaataaccatgtggtgattaacaacgacggcacgggcgcgccaatgcttagatcttaaggggatatcctcgaggttccctttagtgagggttaattgcgagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttgccattcgccattcaggctgcgcaactgttgggaagggcgat

SEQ ID NO: 48 - DNA, 5893 bp, Arabidopsis thaliana
cggtgcgggcctcttcgctattacgccagctggcgaaagggggatgtgctgcaaggcgattaagttgggtaacgccagggttttcccagtcacgacgttgtaaaacgacggccagtgaattgtaatacgactcactatagggcgacccttaggatctaagcattggcgcgccccggctgtctgccatgctgcccggtgtaccgacataaccgccggtggcatagccgcgcatacgcgccatttccttccatcttgtgattcatgctatccatcttttttgagtatccaattaacgaagacgttaccagctgattgaaggttctcaaagtgactgtactccatgttttcttatcatccatgtagttatttttcaaactgcaaattcaagaaaaagccacgcgtgtgcaccttttttttccccttccagtgcattatgcaatagacagcacgagtctttgaaaaagtaacttataaaactgtatcaatttttaaacctaaatagattcataaactattcgttaatataaagtgttctaaactatgatgaaaaaataagcagaaaagactaataattcttagttaaaagcactccgcggttaccacacatctctcaagtatcttccctctgtttgtaactttttcacaattgcttccgcttcagaagaactaacgccttcctgttcctggactatagtatgaagtgttctgtgaacatctcttgccataccctttgcatcaccacagacatatagatagccttcctctttgattaagtcccaaacttgtgcggccttttccatcattttgtgttggacgtactccttctgagcaccttctctagaaaaagccattatcaactctgaaataactccttgatctacaaagttattcagttcatcttcgtagatgaaatccatttgtctgtttctacagccgaaaaacaacaaagaagatcccaactcttcaccatcctcctttaaggccattctctcttgtaagaaacctctgaatggagcaagacctgtaccaggaccgaccatgacaataggagtagaaggattggaaggcagtttgaagttggaggctctgataaagattggagcaccagaacattcgtgagacttctctgctggaaccgcgtttttcatccatgttgaacaaacgcccttatggattctaccagtaggagttggaccgtacactaaagcggatgtgacatgaactcttgatggtgccagtctaggtgaggatgaaattgaatagtatcttggttgcagtctaggcgctattgcggcgaagaaaacacccaaaggaggtttagcggatgggaaagcagccataacttctagtaaagaacgttgactagctactatccattgtgagtattcatccttaccatctggtgaagttagatgtttcagtttttctgcctcagaaggttctgtggcgtacgcagccaaggccactagagctgatttacgtggaggatttaacagatccgcgtaacgagctaaaccggtacctagggtgcatggtcctggaaatggtggaggcactgcactttctagtggtgagccatcctctttatcggcatgaattgagaaaacaagatctaaactatggcccaacaactttccagcttcctctacaatttcaacatggttttcagcgtagacacccacgtgatcacctgtttcgtaagtgataccagtacgtgatatatcaaattcaagatgtatgcaagatctgtctgattcatgagtgtgcaattccttttgaactgcaacgtctactctacatggatgatgaatatcgatggtagtattaccattagccacattactttccattgatttctgtgttgtgaatcttggatcatgagtaactactctatattctggaatgacggctgtgtatggagtggcaacggatttatcatcttcgtccttaagtaacttatctaattcagaccacaaagattccttccatgcattaaagtcatcctcgatagattgatcatcatctcctaaaccgacttcaatcaatctcttcgcaccctttttgcataactcttcatctaagacaatacctatcttgttaaagtgctcgtattgtctgttacctaaggcaaaaacgccgtaagcaagttgctgcaacttgatatctctttcgttctcttcagtaaaccacttgtagaatcttgcggcgttatcggttggttcaccatcaccatacgtggctacacaaaagaaagccaatgtttcctttttcaacttttcctcatattggtcatcatcggcagcgtaatcatccaaatcgattacttttacagccgccttttcgtatcttgctttgatctcttctgaaagtgctttagcgaatccttcggctgttccggtttgtgtgccgaagaagatagagactctcgtttttccagaacctagatctaagtcatcatcctcatctttcgccatcagagacttagggatcattagtggctttagctcgccggaacgatctgccgtggtctttttccacaataagacaacgaaaccagcaaccagtgccagagaagttgtagcaataactaatacaacatcatcggacaaagaatccgttcccatgatacttttcaattgtttgaaaagatcggaggcataaagtgcagaagtcattttaagctttttgtaattaaaacttagattagattgctatgctttctttctaatgagcaagaagtaaaaaaagttgtaatagaacaagaaaaatgaaactgaaacttgagaaattgaagaccgtttattaacttaaatatcaatgggaggtcatcgaaagagaaaaaaatcaaaaaaaaaaattttcaagaaaaagaaacgtgataaaaatttttattgcctttttcgacgaagaaaaagaaacgaggcggtctcttttttcttttccaaacctttagtacgggtaattaacgacaccctagaggaagaaagaggggaaatttagtatgctgtgcttgggtgttttgaagtggtacggcgatgcgcggagtccgagaaaatctggaagagtaaaaaaggagtagaaacattttgaagctaggcgcgtcagccggtaaagattccccacgccaatccggctggttgcctccttcgtgaagacaaactcggcgcgccattacagatcttaaggggatatcctcgaggttccctttagtgagggttaattgcgagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttgccattcgccattcaggctgcgcaactgttgggaagggcgat

SEQ ID NO: 49 - DNA, 3634 bp, Artificial Sequence
  DNA sequence of pEVE1916 - Closing linker EZ for 3 gene HRT plasmid
cggtgcgggcctcttcgctattacgccagctggcgaaagggggatgtgctgcaaggcgattaagttgggtaacgccagggttttcccagtcacgacgttgtaaaacgacggccagtgaattgtaatacgactcactatagggcgacccttaggatcctatggcgcgcccagccggtaaagattccccacgccaatccggctggttgcctccttcgtgaagacaaactcacgcgtccagtatcccagcagatacgggatatcgacatttctgcaccattccggcgggtataggttttattgatggcctcatccacacgcagcagcgtctgttcatcgtcgtggcggcccataataatctgccggtcaatcagccagctttcctcacccggcccccatccccatacgcgcatttcgtagcggtccagctgggagtcgataccggcggtcaggtaagccacacggtcaggaacgggcgctgaataatgctctttccgctctgccatcacttcagcatccggacgttcgccaattttcgcctcccacgtctcaccgagcgtggtgtttacgaaggttttacgttttcccgtatcccctttcgttttcatccagtctttgacaatctgcacccaggtggtgaacgggctgtacgctgtccagatgtgaaaggtcacactgtcaggtggctcaatctcttcaccggatgacgaaaaccagagaatgccatcacgggtccagatcccggtcttttcgcagatataacgggcatcagtaaagtccagctcctgctggcggatgacgcaggcattatgctcgcagagataaaacacgctggagacgcgttttcccgtctttcagtgccttgttcagttcttcctgacgggcggtatatttctccagcttggcgcgcctaagacttagatcttaaggggatatcctcgaggttccctttagtgagggttaattgcgagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttgccattcgccattcaggctgcgcaactgttgggaagggcgat

SEQ ID NO: 50 - DNA, 4685 bp, Artificial Sequence
  DNA sequence of pEVE2177 - empty HRT plasmid with CD tags
tttcccagtcacgacgttgtaaaacgacggccagtgaattgtaatacgactcactatagggcgacccttaagatctgtaatggcgcgccatgcgcggctatgccaccggcggttatgtcggtacaccgggcagcatggcagacagccggacgcgccacgcacagatattataacatctgcataataggcatttgcaagaattactcgtgagtaaggaaagagtgaggaactatcgcatacctgcatttaaagatgccgatttgggcgcgaatcctttattttggcttcaccctcatactattatcagggccagaaaaaggaagtgtttccctccttcttgaattgatgttaccctcataaagcacgtggcctcttatcgagaaagaaattaccgtcgctcgtgatttgtttgcaaaaagaacaaaactgaaaaaacccagacacgctcgacttcctgtcttcctattgattgcagcttccaatttcgtcacacaacaaggtcctagcgacggctcacaggttttgtaacaagcaatcgaaggttctggaatggcgggaaagggtttagtaccacatgctatgatgcccactgtgatctccagagcaaagttcgttcgatcgtactgttactctctctctttcaaacagaattgtccgaatcgtgtgacaacaacagcctgttctcacacactcttttcttctaaccaagggggtggtttagtttagtagaacctcgtgaaacttacatttacatatatataaacttgcataaattggtcaatgcaagaaatacatatttggtcttttctaattcgtagtttttcaagttcttagatgctttctttttctcttttttacagatcatcaaggaagtaattatctactttttacaacaaatataaaacaaagcttggcctgcagggccagcttacccttaaatttatttgcactactggaaaactacctgttccatggccaacacttgtcactactttctcttatggtgttcaatgcttttcccgttatccggatcatatgaaacggcatgactttttcaagagtgccatgcccgaaggttatgtacaggaacgcactatatctttcaaagatgacgggaactacaagacgcgtgctgaagtcaagtttgaaggtgatacccttgttaatcgtatcgagttaaaaggtattgattttaaagaagatggaaacattctcggacacaaactcgagtacaactataactcacacaatgtatacatcacggcagacaaacaaaagaatggaatcaaagctaacttcaaaattcgccacaacattgaagatggatccgttcaactagcagaccattatcaacaaaatactccaattggcgatggccctgtccttttaccagacaaccattacctgtcgacacaatctgccctttcgaaagatcccaacgaaaagcgtgaccacatggtccttcttgagtttgtaactgctgctgggattacacatggcatggatgagctctacaaataatgaattcgtcgaggccgttcaggcctcgaggccgttcaggctcgacccggggatccgcggatctcttatgtctttacgatttatagttttcattatcaagtatgcctatattagtatatagcatctttagatgacagtgttcgaagtttcacgaataaaagataatattctactttttgctcccaccgcgtttgctagcacgagtgaacaccatccctcgcctgtgagttgtacccattcctctaaactgtagacatggtagcttcagcagtgttcgttatgtacggcatcctccaacaaacagtcggttatagtttgtcctgctcctctgaatcgtctccctcgatatttctcattttccttcggcgcgttcgcaggcgtccgggacgtttgagcagaataaccatgtggtgattaacaacgacggcacgggcgcgccaatgcttagatcttaaggggatatcctcgaggttccctttagtgagggttaattgcgagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttgccattcgccattcaggctgcgcaactgttgggaagggcgat

SEQ ID NO: 51 - DNA, 5459 bp, Arabidopsis thaliana
cggtgcgggcctcttcgctattacgccagctggcgaaagggggatgtgctgcaaggcgattaagttgggtaacgccagggttttcccagtcacgacgttgtaaaacgacggccagtgaattgtaatacgactcactatagggcgacccttaggatcctatggcgcgccggcacccttgcgggccatgtcatacaccgccttcagagcagccggacctatctgcccgttacgcgccagcttgcaaattaaagccttcgagcgtcccaaaaccttctcaagcaaggttttcagtataatgttacatgcgtacacgcgtctgtacagaaaaaaaagaaaaatttgaaatataaataacgttcttaatactaacataactataaaaaaataaatagggacctagacttcaggttgtctaactccttccttttcggttagagcggatgtggggggagggcgtgaatgtaagcgtgacataactaattacatgatatcgacaaaggaaaagggggacggatctccgaggcctcggacccgtcgggccgccgtcggacgtgccgcggtcaggtggcgaacttcttaataccttgttgcaagatagagtcgaaaacgtccatctttttcttttccaaggcaataccaatttcaacaccgttagaaccatctctagattcagagaaggcaatggaaccaccagtttcaatatgaacgatttccatcttgcatggcttacccaaaccaaaatccatatcgtacaaacccaattttggagcaccagcaatagaggttgggtaatgagacataacccattttctaacaccttgaccccatcttggagcagttttcaacaaatcggaggacaacatatccttgattctagcagtaatagcatcagaagcagccaaaacgcacttttcacccaacaaatcatgttttttgacagagactatacctggagccatacagttaccgaagtaagtttgtggaataggttgggtgtacttcaatctgtttctacagtcaacgttaatcatcaagtggaaaacttcgtccttatcttcttcgttagccttagtttcagaatcttggaccaaggtcttaatcaaggaaacccagataaaagccaaggtaacaacgaaggtagaaactggagattgattttcggattgttcggtgacccaagacttcaagttatcgatttgctttctggacaaggtgaaagtagctctaaccatgttttctggagtaacatgagaagagtgcttggcggaattttgtgaccaaaatctttccaaatgaccagcaccaacttcacctggatccttgatcatgtttctgcaagaatgaattggcaaagatggcaacaaaacagtagctggatctttaccagaagatttggtcaaggacatccagtacttcatgaaatgtgagaaagtaacaccatcagcaacaacatgagtagcagagttaccaatacagataccagcacctggaaaaatagtgacttgcatagccataattggtctcatttgaataccttcaggtgaaacatgtggtggtggcaattttggcaaaacaccatgtaaaacggaaatatcctttggggaatcggacttcaattgatcgaaatcggtttcagtagattcagcaacggtgaaaaccaaagagtcttgaccatcattgtaatgcaagtatggtggatctggtcttggtggaataatcaacttaccggcgtatggaaaaaaatgttgcaaggtaatagacaaggagtgcttcaagtttgggacgaaatcttgtaagaaagattcggtggagttttggtaggagaagaagaacaaagaatcagccaatggtaaagacaaccatggggcatcaaaaaaagtcaatggcaaagtagtagatggaacagtaccctttggtggagaaatatggcaggtttcaataatctttggtggttgcaagtgagcaaccattttaagctttttgtttgtttatgtgtgtttattcgaaactaagttcttggtgttttaaaactaaaaaaaagactaactataaaagtagaatttaagaagtttaagaaatagatttacagaattacaatcaatacctaccgtctttatatacttattagtcaagtaggggaataatttcagggaactggtttcaaccttttttttcagctttttccaaatcagagagagcagaaggtaatagaaggtgtaagaaaatgagatagatacatgcgtgggtcaattgccttgtgtcatcatttactccaggcaggttgcatcactccattgaggttgtgtccgttttttgcctgtttgtgcccctgttctctgtagttgcgctaagagaatggacctatgaactgatggttggtgaagaaaacaatattttggtgctgggattctttttttttctggatgccagcttaaaaagcgggctccattatatttagtggatgccaggaataaactgttcacccagacacctacgatgttatatattctgtgtaacccgccccctattttgggcatgtacgggttacagcagaattaaaaggctaattttttgactaaataaagttaggaaaatcactactattaattatttacgtattctttgaaatggcagtattgataatgataaactcgaactgggcgcgtcgtgccgtcgttgttaatcaccacatggttattctgctcaaacgtcccggacgcctgcgaggcgcgcctattgaaagatcttaaggggatatcctcgaggttccctttagtgagggttaattgcgagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttgccattcgccattcaggctgcgcaactgttgggaagggcgat

SEQ ID NO: 52 - DNA, 5432 bp, Dahlia variabilis
cggtgcgggcctcttcgctattacgccagctggcgaaagggggatgtgctgcaaggcgattaagttgggtaacgccagggttttcccagtcacgacgttgtaaaacgacggccagtgaattgtaatacgactcactatagggcgacccttaggatcctatggcgcgccggcacccttgcgggccatgtcatacaccgccttcagagcagccggacctatctgcccgttacgcgccagcttgcaaattaaagccttcgagcgtcccaaaaccttctcaagcaaggttttcagtataatgttacatgcgtacacgcgtctgtacagaaaaaaaagaaaaatttgaaatataaataacgttcttaatactaacataactataaaaaaataaatagggacctagacttcaggttgtctaactccttccttttcggttagagcggatgtggggggagggcgtgaatgtaagcgtgacataactaattacatgatatcgacaaaggaaaagggggacggatctccgaggcctcggacccgtcgggccgccgtcggacgtgccgcggttaagaagcaatagcggattccaaaccgtcgttaaagattttaccaaaggcttccatttgcatggatgggaaacaaacaccaatttcaaaatcttgggcggattctttacaagctgacaaagaaacagaggcggagtagtcaatagaaacaacttcgtacttcatagccttaccccaaccgaaatcaatatcgtagaagttcaactttggagtaccagaaatacccatctttctagctggaatcttaaaaccatcgtaccatctatcagcgtattccaaaataccacccttcttgttaaccatcttagagataccttcaccaatcaacttagcagccataacaaaaccgttttcacccttcaagacaccgttcttaatagtgacaatacatggagcagaacagttaccgaagtagttttctggtaatggtggatctaatcttgatctgcaaccgacagaaacgatgaattgttccaattcatcttcaccctttttttcacccatgttgaccaaggacttaacgatacaagaccaaatgtaaccgcaggtaacagtgaaagaagaagtgtattccaacattggcaattgagtcaagacttgcttcttcaaaccggaaatatgagttctggccaaaacgaaagtagctctaactctatcagatgaagaaccaaccaaagaaggagcttggtagaaagtacccaatctggtttgattcaatctgttttcgtataattgtgggttaacaacaactctatcgaaaactggtggggaaccatttttcaagaatggttgatcttcaccagtttcacaaacagaagcccaagccttcaaaaaaccgaatctagtgttagcatcagacaaagagtgatggttggtcaaaccaatagaaataccggagtttgggaagtaagtaacttgaacagagaaaactggcaaggtaacgtaatcagattcttttacagcgttacccaatggtggaaccaatggatagaaattttcgcactttcttggatggttagcagacaaatcgttgaaatccaaggtagtttcagcgaaagtcaaagcaacagaatcaccttcaacatgtctgatttctggctttctggtagaatcatgtggatttgggtaaacgatcaacttaccgacgaatggaaagtaatgttgcaaggtaatggacaaggagtgcttcaaatttgggataacagtttcggtgaaatgggacttggagtatggaaaatggtagaagtacaagtgatgaactggtggaaacaacaaccaggcaatatcgaagaaagtcaatggcaatgatctatgaccaatagtagatggtggtggagaaattctagagtgttccaagatggtcaagtttgggatgttgtccattttaagctttttgtttgtttatgtgtgtttattcgaaactaagttcttggtgttttaaaactaaaaaaaagactaactataaaagtagaatttaagaagtttaagaaatagatttacagaattacaatcaatacctaccgtctttatatacttattagtcaagtaggggaataatttcagggaactggtttcaaccttttttttcagctttttccaaatcagagagagcagaaggtaatagaaggtgtaagaaaatgagatagatacatgcgtgggtcaattgccttgtgtcatcatttactccaggcaggttgcatcactccattgaggttgtgtccgttttttgcctgtttgtgcccctgttctctgtagttgcgctaagagaatggacctatgaactgatggttggtgaagaaaacaatattttggtgctgggattctttttttttctggatgccagcttaaaaagcgggctccattatatttagtggatgccaggaataaactgttcacccagacacctacgatgttatatattctgtgtaacccgccccctattttgggcatgtacgggttacagcagaattaaaaggctaattttttgactaaataaagttaggaaaatcactactattaattatttacgtattctttgaaatggcagtattgataatgataaactcgaactgggcgcgtcgtgccgtcgttgttaatcaccacatggttattctgctcaaacgtcccggacgcctgcgaggcgcgcctattgaaagatcttaaggggatatcctcgaggttccctttagtgagggttaattgcgagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttgccattcgccattcaggctgcgcaactgttgggaagggcgat

SEQ ID NO: 53 - DNA, 3638 bp, Artificial Sequence
  DNA sequence of pEVE1918 - Closing linker GZ for 5 gene plasmid
cggtgcgggcctcttcgctattacgccagctggcgaaagggggatgtgctgcaaggcgattaagttgggtaacgccagggttttcccagtcacgacgttgtaaaacgacggccagtgaattgtaatacgactcactatagggcgacccttaagatctaagtcttaggcgcgccaagctggagaaatataccgcccgtcaggaagaactgaacaaggcactgaaagacgggaaaacgcgtccagtatcccagcagatacgggatatcgacatttctgcaccattccggcgggtataggttttattgatggcctcatccacacgcagcagcgtctgttcatcgtcgtggcggcccataataatctgccggtcaatcagccagctttcctcacccggcccccatccccatacgcgcatttcgtagcggtccagctgggagtcgataccggcggtcaggtaagccacacggtcaggaacgggcgctgaataatgctctttccgctctgccatcacttcagcatccggacgttcgccaattttcgcctcccacgtctcaccgagcgtggtgtttacgaaggttttacgttttcccgtatcccctttcgttttcatccagtctttgacaatctgcacccaggtggtgaacgggctgtacgctgtccagatgtgaaaggtcacactgtcaggtggctcaatctcttcaccggatgacgaaaaccagagaatgccatcacgggtccagatcccggtcttttcgcagatataacgggcatcagtaaagtccagctcctgctggcggatgacgcaggcattatgctcgcagagataaaacacgctggagacgcgtggcgcatccgcgtcaggcggtacagccattcaggccgctgcggcgaaattccattttgcaggcgcgccaatgcttagatcctaaggggatatcctcgaggttccctttagtgagggttaattgcgagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttgccattcgccattcaggctgcgcaactgttgggaagggcgat

SEQ ID NO: 54 - protein, 464 aa, Vitis amurensis
MANPHPHFLIITFPAQGHINPALELAKRLIGVGADVTFATTIHAKSRLVKNPTVDGLRFSTFSDGQEEGVKRGPNELPVFQRLASENLSELIMASANEGRPISCLIYSILIPGAAELARSFNIPSAFLWIQPATVLDIYYYYFNGFGDLIRSKSSDPSFSIELPGLPSLSRQDLPSFFVGSDQNQENHALAAFQKHLEILEQEENPKVLVNTFDALEPEALRAVEKLKLTAVGPLVPSGFSDGKDASDTPSGGDLSDGSRDYMEWLKSKPESTVVYVSFGSISMFSMQQMEEIARGLLESGRPFLWVIRAKENGEENKEEDKLSCQEELEKQGMLIQWCSQMEVLSHPSLGCFVTHCGWNSSIESLASGVPMIAFPQWADQGTNTKLIKDVWKTGVRLMVNEEEIVTSDELRRCLELVMGDGEKGQEMRKNAKKWKILAKEALKEGGSSHKNLKNFVDEVIQGY

SEQ ID NO: 55 - protein, 511 aa, Solanum lycopersicum
MALRINELFVAAIIYIIVHIIISKLITTVRERGRRLPLPPGPTGWPVIGALPLLGSMPHVALAKMAKKYGPIMYLKVGTCGMVVASTPNAAKAFLKTLDINFSNRPPNAGATHLAYNAQDMVFAPYGPRWKLLRKLSNLHMLGGKALENWANVRANELGHMLKSMFDASQDGECVVIADVLTFAMANMIGQVMLSKRVFVEKGVEVNEFKNMVVELMTVAGYFNIGDFIPKLAWMDIQGIEKGMKNLHKKFDDLLTKMFDEHEATSNERKENPDFLDVVMANRDNSEGERLSTTNIKALLLNLFTAGTDTSSSVIEWALAEMMKNPKIFKKAQQEMDQVIGKNRRLIESDIPNLPYLRAICKETFRKHPSTPLNLPRVSSEPCTVDGYYIPKNTRLSVNIWAIGRDPDVWENPLEFTPERFLSGKNAKIEPRGNDFELIPFGAGRRICAGTRMGIVVVEYILGTLVHSFDWKLPNNVIDINMEESFGLALQKAVPLEAMVTPRLSLDVYRC

SEQ ID NO: 56 - protein, 692 aa, Arabidopsis thaliana [sequence omitted]

SEQ ID NO: 57 - protein, 469 aa, Arabidopsis thaliana
MVAHLQPPKIIETCHISPPKGTVPSTTLPLTFFDAPWLSLPLADSLFFFSYQNSTESFLQDFVPNLKHSLSITLQHFFPYAGKLIIPPRPDPPYLHYNDGQDSLVFTVAESTETDFDQLKSDSPKDISVLHGVLPKLPPPHVSPEGIQMRPIMAMQVTIFPGAGICIGNSATHVVADGVTFSHFMKYWMSLTKSSGKDPATVLLPSLPIHSCRNMIKDPGEVGAGHLERFWSQNSAKHSSHVTPENMVRATFTLSRKQIDNLKSWVTEQSENQSPVSTFVVTLAFIWVSLIKTLVQDSETKANEEDKDEVFHLMINVDCRNRLKYTQPIPQTYFGNCMAPGIVSVKKHDLLGEKCVLAASDAITARIKDMLSSDLLKTAPRWGQGVRKWVMSHYPTSIAGAPKLGLYDMDFGLGKPCKMEIVHIETGGSIAFSESRDGSNGVEIGIALEKKKMDVFDSILQQGIKKFAT

SEQ ID NO: 58 - protein, 460 aa, Dahlia variabilis
MDNIPNLTILEHSRISPPPSTIGHRSLPLTFFDIAWLLFPPVHHLYFYHFPYSKSHFTETVIPNLKHSLSITLQHYFPFVGKLIVYPNPHDSTRKPEIRHVEGDSVALTFAETTLDFNDLSANHPRKCENFYPLVPPLGNAVKESDYVTLPVFSVQVTYFPNSGISIGLTNHHSLSDANTRFGFLKAWASVCETGEDQPFLKNGSPPVFDRVVVNPQLYENRLNQTRLGTFYQAPSLVGSSSDRVRATFVLARTHISGLKKQVLTQLPMLEYTSSFTVTCGYIWSCIVKSLVNMGEKKGEDELEQFIVSVGCRSRLDPPLPENYFGNCSAPCIVTIKNGVLKGENGFVMAAKLIGEGISKMVNKKGGILEYADRWYDGFKIPARKMGISGTPKLNFYDIDFGWGKAMKYEVVSIDYSASVSLSACKESAQDFEIGVCFPSMQMEAFGKIFNDGLESAIAS

(19) United States
(12) Patent Application Publication
(10) Pub. No.: US 2018/0371513 A1
(43) Pub. Date: Dec. 27, 2018

(54) Production of Anthocyanin from Simple Sugars

(71) Applicant: Evolva SA, Reinach (CH)

(72) Inventors: Michael Naesby, Huningue (FR); Zina Zokouri, Zurich (CH); David Fischer, Arlesheim (CH); Michael Eichenberger, Basel (CH); Anders Hansson, Basel (CH)

(21) Appl. No.: 15/761,654

(22) PCT Filed: Sep. 21, 2016
(86) PCT No.: PCT/EP2016/072474

§ 371 Date: Mar. 30, 2018

Related U.S. Application Data
(60) Provisional application No. 62/222,919, filed on Sep. 24, 2015.

(51) Int. Cl. C12P 17/06

(52) U.S. Cl. CPC C12P 17/06; C12Y 602/01012; C12Y 203/01074; C12Y 114/11009; C12Y 403/01025; C12Y 114/11019; C12Y 204/01115; C12Y 505/01006; C12Y 403/01023; C12Y 101/01021

(57) ABSTRACT

Methods for producing anthocyanin by expression in a microorganism are disclosed including culturing of the microorganism under anthocyanin producing conditions, wherein the microorganism has an operative metabolic pathway including at least one heterologous enzyme activity, the pathway producing anthocyanin from simple sugars or other simple carbon sources.

### BACKGROUND OF THE INVENTION

### Field of the Invention

Provided are methods for producing anthocyanins in recombinant host cells.

### Description of Related Art

Over the last decade there have been several reports of heterologous production of flavonoids, including anthocyanins, using unicellular hosts, particularly in the prokaryote, Escherichia coli, and the eukaryote, Saccharomyces cerevisiae. Especially in E. coli there has been some success, predominantly after feeding intermediates of the flavonoid pathway to the bacteria. This has allowed several flavanones, flavones, and flavonols to be produced from phenyl propanoid precursors (see e.g., Yan 2005; Jiang 2005; Leonard 2007, respectively). In addition, several other flavonoids were made by intermediate feeding, such as isoflavonoids from liquiritigenin; flavan-3-ols and flavan-4-ols from flavanones; and anthocyanins from either flavanones or from (+)-catechin. However, there are no reports of anthocyanins being produced from basal medium components such as sugar or from the natural precursors phenylalanine or tyrosine.

The anthocyanin biosynthetic pathway is shown in FIG. 1. As shown, in this pathway the flavonoid intermediate coumaroyl-CoA is produced via the plant phenylpropanoid pathway. Phenylalanine is deaminated by the action of phenylalanine ammonia lyase (PAL), an enzyme of the ammonia lyase family, to form cinnamic acid. Cinnamic acid is then hydroxylated to p-coumaric acid (also called 4-coumaric acid) by cinnamate 4-hydroxylase (C4H), a CYP450 enzyme. Alternatively, p-coumaric acid is formed directly from tyrosine by the action of tyrosine ammonia lyase (TAL). Some enzymes have both PAL and TAL activity. The enzyme 4-coumarate-CoA-ligase (4CL) activates p-coumaric acid to p-coumaroyl CoA by attachment of a CoA group.

Chalcone synthase (CHS), a polyketide synthase, is the first committed enzyme in the flavonoid pathway, and catalyzes synthesis of naringenin chalcone from one molecule of p-coumaroyl CoA and three molecules of malonyl CoA. Naringenin chalcone is rapidly and stereospecifically isomerized to the colorless (2S)-naringenin by chalcone isomerase (CHI). (2S)-Naringenin is hydroxylated at the 3-position by flavanone 3-hydroxylase (F3H) to yield (2R,3R)-dihydrokaempferol, a dihydroflavonol. F3H belongs to the 2-oxoglutarate-dependent dioxygenase (2ODD) family. Flavonoid 3′-hydroxylase (F3′H) and flavonoid 3′,5′-hydroxylase (F3′5′H), which are P450 enzymes, catalyze hydroxylation of dihydrokaempferol (DHK) to form (2R,3R)-dihydroquercetin and dihydromyricetin, respectively. F3′H and F3′5′H determine the hydroxylation pattern of the B-ring of flavonoids and anthocyanins and are necessary for cyanidin and deiphinidin production, respectively. They are the key enzymes that determine the structures of anthocyanins and thus their color. Dihydroflavonols are reduced to corresponding 3,4-cis leucoanthocyanidins by the action of dihydroflavonol 4-reductase (DFR). Anthocyanidin synthase (ANS, also called leucoanthocyanidin dioxygenase or LDOX), which belongs to the 2ODD family, catalyzes synthesis of corresponding colored anthocyanidins. In contrast to the well-conserved main pathway of flavonoid biosynthesis described above, modification of anthocyanidins is family- or species-dependent and can be very diverse. Additionally, in order to form more stable anthocyanins, anthocyanidins can be 3-glucosylated by the action of UDP-glucose:flavonoid (or anthocyanidin) 3GT.

In yeast (e.g., S. cerevisiae), some of the same molecules (flavanones, flavones, and flavonols) have been made from phenyl propanoids. In addition, a few examples have been reported of production of flavonoids from sugar, e.g., naringenin (Koopman et al. 2012) and various flavanones and flavonols (Naesby 2009). However, production of anthocyanins has never been reported.

Therefore, new approaches are required for producing anthocyanins via heterologous biosynthetic pathways in microbes.

### SUMMARY OF THE INVENTION

It is against the above background that the present invention provides certain advantages and advancements over the prior art. Set forth herein are methods developed by selection of highly active heterologous genes, and by balancing the expression thereof, that produce anthocyanins from glucose in a microorganism host cell. Specifically provided herein are operative metabolic pathways for producing anthocyanins from glucose or other simple sugars.

In a first aspect, the invention provides a microorganism including an operative metabolic pathway capable of producing an anthocyanin from glucose. The operative metabolic pathway includes at least a 4-coumaric acid-CoA ligase (4CL), a chalcone synthase (CHS), a flavanone 3-hydroxylase (F3H), a dihydroflavonol-4-reductase (DFR), an anthocyanidin synthase (ANS), an anthocyanidin 3-O-glycosyltransferase (A3GT), a chalcone isomerase (CHI), and at least one of a) a tyrosine ammonia lyase; or b) a phenylalanine ammonia lyase (PAL) and a trans-cinnamate 4-monooxygenase (C4H). At least one enzyme of the operative metabolic pathway is encoded by a gene heterologous to the microorganism is encoded by a gene heterologous to the microorganism. In particular embodiments, the anthocyanin is produced in a ratio of at least 1:1 to its anthocyanidin precursor by the operative metabolic pathway.

In a second aspect, the invention provides a fermentation vessel including a microorganism having an operative metabolic pathway producing an anthocyanin from glucose. The operative metabolic pathway includes a 4-coumaric acid-CoA ligase (4CL), a chalcone synthase (CHS), a flavanone 3-hydroxylase (F3H), a dihydroflavonol-4-reductase (DFR), an anthocyanidin synthase (ANS), an anthocyanidin 3-O-glycosyltransferase (A3GT), a chalcone isomerase (CHI), and a tyrosine ammonia lyase or a phenylalanine ammonia lyase (PAL) and a trans-cinnamate 4-monooxygenase (C4H), wherein at least one enzyme of the operative metabolic pathway is encoded by a gene heterologous to the microorganism.

In a third aspect, the invention provides a microorganism including an operative metabolic pathway producing an anthocyanin from glucose. The operative metabolic pathway includes a 4-coumaric acid-CoA ligase (4CL) encoded by the nucleic acid sequence set forth in SEQ ID NO: 1, a chalcone synthase (CHS) encoded by the nucleic acid sequence set forth in SEQ ID NO: 21, a flavanone 3-hydroxylase (F3H) encoded by the nucleic acid sequence set forth in SEQ ID NO: 3, a dihydroflavonol-4-reductase (DFR) encoded by the nucleic acid sequence set forth in SEQ ID NO: 5 or SEQ ID NO: 7, an anthocyanidin synthase (ANS) encoded by the nucleic acid sequence set forth in SEQ ID NO: 9, an anthocyanidin 3-O-glycosyltransferase (A3GT) encoded by the nucleic acid sequence set forth in SEQ ID NO: 11, a chalcone isomerase (CHI) encoded by the nucleic acid sequence set forth in SEQ ID NO: 13, and at least one of a) a tyrosine ammonia lyase (TAL) encoded by the nucleic acid sequence set forth in SEQ ID NO: 15 or b) a phenylalanine ammonia lyase (PAL) encoded by the nucleic acid sequence set forth in SEQ ID NO: 17 and a trans-cinnamate 4-monooxygenase (C4H) encoded by the nucleic acid sequence set forth in SEQ ID NO: 19.

In a fourth aspect, a microorganism includes an operative metabolic pathway capable of producing an anthocyanin from a simple sugar. The operative metabolic pathway includes a 4-coumaric acid-CoA ligase (4CL), a chalcone synthase (CHS), a flavanone 3-hydroxylase (F3H), a dihydroflavonol-4-reductase (DFR), an anthocyanidin synthase (ANS), an anthocyanidin 3-O-glycosyltransferase (A3GT), a chalcone isomerase (CHI), at least one of a) a tyrosine ammonia lyase (TAL) or b) a phenylalanine ammonia lyase (PAL) and a trans-cinnamate 4-monooxygenase (C4H), and an anthocyanin-5-O-glycosyl transferase (A5GT), an anthocyanin-3-O-aromatic acyl transferase (A3AAT), or an anthocyanin-3-O-malonyl acyl transferase (A3MAT). At least one enzyme of the operative metabolic pathway is encoded by a gene heterologous to the microorganism. In one embodiment, the anthocyanin is pelargonidin-3,5-O-diglucoside, cyanidin-3,5-O-diglucoside, delphinidin-3,5-O-diglucoside, pelargonidin-3-O-coumaroyl-glucoside, pelargonidin-3-O-coumaroyl glucoside-5-O-glucoside, pelargonidin-3-O-malonyl glucoside, or pelargonidin-3-O-malonyl glucoside-5-O-glucoside.

In a fifth aspect, a method of producing an anthocyanin includes the steps of a) culturing a microorganism comprising an operative metabolic pathway producing an anthocyanin from a simple sugar, the operative metabolic pathway comprising: a 4-coumaric acid-CoA ligase (4CL); a chalcone synthase (CHS); a flavanone 3-hydroxylase (F3H); a dihydroflavonol-4-reductase (DFR); an anthocyanidin synthase (ANS); an anthocyanidin 3-O-glycosyltransferase (A3GT); a chalcone isomerase (CHI); at least one of a) a tyrosine ammonia lyase (TAL) or b) a phenylalanine ammonia lyase (PAL) and a trans-cinnamate 4-monooxygenase (C4H), and an anthocyanin-5-O-glycosyl transferase (A5GT), an anthocyanin-3-O-aromatic acyl transferase (A3AAT), or an anthocyanin-3-O-malonyl acyl transferase (A3MAT), at least one enzyme of the operative metabolic pathway is encoded by a gene heterologous to the microorganism, b) producing an anthocyanin by the microorganism, and c) optionally isolating the anthocyanin. In one embodiment, the anthocyanin is pelargonidin-3,5-O-diglucoside, cyanidin-3,5-O-glucoside, delphinidin-3,5-O-diglucoside, pelargonidin-3-O-coumaroyl-glucoside, pelargonidin-3-O-coumaroyl glucoside-5-O-glucoside, pelargonidin-3-O-malonyl glucoside, or pelargonidin-3-O-malonyl glucoside-5-O-glucoside.

These and other features and advantages of the present invention will be more fully understood from the following detailed description of the invention taken together with the accompanying claims. It is noted that the scope of the claims is defined by the recitations therein and not by the specific discussion of features and advantages set forth in the present description.

### DESCRIPTION OF DRAWINGS

FIG. 1. Anthocyanin biosynthetic pathway overview.

FIGS. 2(a) and 2(b). FIG. 2(a) depicts DNA fragments used for assembling, by in vivo homologous recombination, the plasmid shown in FIG. 2(b). Each DNA fragment is amplified in a bacterial vector from which it is released by a restriction enzyme digest (only the released fragments are shown). The DNA fragments contain elements for stable maintenance and replication in yeast, or they contain a yeast expression cassette (promoter-gene coding sequence-terminator) for expressing one of the genes of the desired biosynthetic pathway. Finally, one fragment contains the tags necessary for closing the circle: All fragments have so-called HRTs (Homologous Recombination Tag) at the ends, where the 3′-end of one fragment is identical to the 5′-end of the next fragment, etc. When introduced into yeast, the repair mechanism of this host will assemble the fragments into the full plasmid shown in FIG. 2(b).

FIG. 3 depicts DNA fragments used for assembling and integrating, by in vivo homologous recombination, the expression cassettes (as described in FIGS. 2(a) and 2(b) for assembly of a desired biosynthetic pathway. Instead of sequences for plasmid replication, the first and the last fragment have sequences (Integration Tags) which are homologous to the integration site in the host genome.

FIG. 4. Chromatogram of the anthocyanidin pelargonidin detected by LC/MS.

FIG. 5. Chromatogram of anthocyanin pelargonidin-3-O-glucoside (P3G) detected by LC/MS.

FIG. 6. Chromatogram of pelargonidin-3,5-O-diglucoside detected by LC/MS.

FIG. 7. Chromatogram of the cyanidin detected by LC/MS.

FIG. 8. Chromatogram of cyanidin-3-O-glucoside (C3G) detected by LC/MS.

FIG. 9. Chromatogram of cyanidin-3,5-O-diglucoside detected by LC/MS.

FIG. 10. Chromatogram of the delphinidin detected by LC/MS.

FIG. 11. Chromatogram of the delphinidin-3-O-glucoside detected by LC/MS.

FIG. 12. Chromatogram of delphinidin-3,5-O-diglucoside detected by LC/MS.

FIG. 13. Chromatogram of the pelargonidin-3-O-coumaroyl-glucoside detected by LC/MS.

FIG. 14. Chromatogram of the pelargonidin-3-O-coumaroyl-glucoside-5-O-glucoside detected by LC/MS.

FIG. 15. Chromatogram of the pelargonidin-3-O-malonyl-glucoside detected by LC/MS.

FIG. 16. Chromatogram of the pelargonidin-3-O-malonyl-glucoside-5-O-glucoside detected by LC/MS.

FIG. 17. A photograph of methanol extracted P3G producing cells. Cell samples were adjusted to pH 2 with HCl. Cells in the left tube contain the full P3G pathway, and as can be seen, express the P3G molecule. The cells in the right tube contain the full P3G pathway but lack DFR, and therefore, have no color.

FIG. 18. A photograph of methanol extracted P3G producing cells. Cell samples were pH adjusted with HCl to a pH of <2 (left tube=a first shade), ˜5 (center tube=no color), or about 10 (right tube=a second shade).

### DETAILED DESCRIPTION

All publications, patents and patent applications cited herein are hereby expressly incorporated by reference in their entirety for all purposes.

Before describing the present invention in detail, a number of terms will be defined. As used herein, the singular forms “a,” “an,” and “the” include plural referents unless the context clearly dictates otherwise. For example, reference to “a compound” means one or more compounds.

It is noted that terms like “preferably,” “commonly,” and “typically” are not utilized herein to limit the scope of the claimed invention or to imply that certain features are critical, essential, or even important to the structure or function of the claimed invention. Rather, these terms are merely intended to highlight alternative or additional features that can or cannot be utilized in a particular embodiment of the present invention.

For the purposes of describing and defining the present invention it is noted that the term “substantially” is utilized herein to represent the inherent degree of uncertainty that can be attributed to any quantitative comparison, value, measurement, or other representation. The term “substantially” is also utilized herein to represent the degree by which a quantitative representation can vary from a stated reference without resulting in a change in the basic function of the subject matter at issue.

As used herein, the term “about” refers to ±10% of a given value unless otherwise specified.

As used herein, the terms “or” and “and/or” are utilized to describe multiple components in combination or exclusive of one another. For example, “x, y, and/or z” can refer to “x” alone, “y” alone, “z” alone, “x, y, and z,” “(x and y) or z,” “x or (y and z),” or “x or y or z.”

Methods well known to those skilled in the art can be used to construct genetic expression constructs and recombinant cells according to this invention. These methods include in vitro recombinant DNA techniques, synthetic techniques, in vivo recombination techniques, and polymerase chain reaction (PCR) techniques. See, for example, techniques as described in Green & Sambrook, 2012, MOLECULAR CLONING: A LABORATORY MANUAL, Fourth Edition, Cold Spring Harbor Laboratory, New York; Ausubel et al., 1989, CURRENT PROTOCOLS IN MOLECULAR BIOLOGY, Greene Publishing Associates and Wiley Interscience, New York, and PCR Protocols: A Guide to Methods and Applications (Innis et al., 1990, Academic Press, San Diego, Calif.).

As used herein, the terms “polynucleotide,” “nucleotide,” “oligonucleotide,” and “nucleic acid” can be used interchangeably to refer to nucleic acid comprising DNA, RNA, derivatives thereof, or combinations thereof.

As used herein, the terms “microorganism,” “microorganism host,” “microorganism host cell,” “recombinant host,” and “recombinant host cell” can be used interchangeably. As used herein, the term “recombinant host” is intended to refer to a host, the genome of which has been augmented by at least one DNA sequence. Such DNA sequences include but are not limited to genes that are not naturally present, DNA sequences that are not normally transcribed into RNA or translated into a protein (“expressed”), and other genes or DNA sequences which one desires to introduce into the non-recombinant host. It will be appreciated that typically the genome of a recombinant host described herein is augmented through stable introduction of one or more recombinant genes that may be inserted into the host genome and/or by way of an episomal vector (e.g., plasmid, YAC, etc.). Generally, introduced DNA is not originally resident in the host that is the recipient of the DNA, but it is within the scope of this disclosure to isolate a DNA segment from a given host, and to subsequently introduce one or more additional copies of that DNA into the same host, e.g., to enhance production of the product of a gene or alter the expression pattern of a gene. In some instances, the introduced DNA will modify or even replace an endogenous gene or DNA sequence by, e.g., homologous recombination or site-directed mutagenesis. Suitable recombinant hosts include microorganisms.

As used herein, the term “recombinant gene” refers to a gene or DNA sequence that is introduced into a recipient host, regardless of whether the same or a similar gene or DNA sequence may already be present in such a host. “Introduced,” or “augmented” in this context, is known in the art to mean introduced or augmented by the hand of man. Thus, a recombinant gene can be a DNA sequence from another species, or can be a DNA sequence that originated from or is present in the same species, but has been incorporated into a host by recombinant methods to form a recombinant host. It will be appreciated that a recombinant gene that is introduced into a host can be identical to a DNA sequence that is normally present in the host being transformed. For any recombinant gene, one or more additional copies of the DNA can be introduced, to thereby permit overexpression or modified expression of the gene product of that DNA. Said recombinant genes are particularly encoded by cDNA.

As used herein, the terms “codon optimization” and “codon optimized” refer to a technique to maximize protein expression in fast-growing microorganisms such as E. coli or S. cerevisiae by increasing the translation efficiency of a particular gene. Codon optimization can be achieved, for example, by converting a nucleotide sequence of one species into a genetic sequence which better reflects the translation machinery of a different, host species. Optimal codons help to achieve faster translation rates and high accuracy.

As used herein, the term “engineered biosynthetic pathway” or “operative metabolic pathway” refers to a biosynthetic pathway that occurs in a recombinant host, as described herein, and does not naturally occur in the host. Further, an “engineered microorganism” refers to a recombinant host that contains an engineered biosynthetic pathway or operative metabolic pathway.

As used herein, the terms “heterologous sequence,” “heterologous coding sequence,” and “heterologous gene” are used to describe a sequence or gene derived from a species other than the recombinant host. For example, if the recombinant host is an S. cerevisiae cell, then the cell would include a heterologous sequence derived from an organism other than S. cerevisiae. A heterologous coding sequence or gene, for example, can be from a prokaryotic microorganism, a eukaryotic microorganism, a plant, an animal, an insect, or a fungus different than the recombinant host expressing the heterologous sequence.

As used herein, “highly efficient enzyme” refers to an enzyme that when expressed in a recombinant host exhibits a rate of enzymatic catalysis more efficient than a second enzyme (e.g., a functional homolog or another embodiment of the first enzyme) expressed in the same host under the same conditions and that catalyzes the same reaction as the highly efficient enzyme. For example, the highly efficient enzyme and second enzyme could both be glycosyltransferases but from different species. By way of illustration, said highly efficient enzyme would have an enzymatic activity that is two-fold, or four-fold, or ten-fold, or twenty-fold, or one hundred-fold, or one thousand-fold higher than said second heterologous enzyme.

As used herein, “functional homolog” refers to a polypeptide that has sequence similarity to a reference polypeptide, and that carries out one or more of the biochemical or physiological function(s) of the reference polypeptide. A functional homolog and the reference polypeptide can be a natural occurring polypeptide, and the sequence similarity can be due to convergent or divergent evolutionary events. As such, functional homologs are sometimes designated in the literature as homologs, or orthologs, or paralogs. Variants of a naturally occurring functional homolog, such as polypeptides encoded by mutants of a wild type coding sequence, can themselves be functional homologs. Functional homologs can also be created via site-directed mutagenesis of the coding sequence for a polypeptide, or by combining domains from the coding sequences for different naturally-occurring polypeptides (“domain swapping”). Techniques for modifying genes encoding functional polypeptides described herein are known and include, inter alia, directed evolution techniques, site-directed mutagenesis techniques and random mutagenesis techniques, and can be useful to increase specific activity of a polypeptide, alter substrate specificity, alter expression levels, alter subcellular location, or modify polypeptide-polypeptide interactions in a desired manner. Such modified polypeptides are considered functional homologs. The term “functional homolog” is sometimes applied to the nucleic acid that encodes a functionally homologous polypeptide.

As used herein, “optimal conditions,” in reference to an enzyme, refers to reaction conditions in which an expressed enzyme is able to operate at its maximum efficiency. For example, an enzyme of a biosynthetic pathway operating under optimal conditions would have a non-rate-limiting supply of substrate for its reaction step. Further, the enzyme would have little to no feedback inhibition caused by, for example, an overabundance of product accumulation downstream of the enzyme in the biosynthetic pathway.

Also, as used herein “optimal conditions,” in reference to a biosynthetic pathway, refers to a biosynthetic pathway in which each enzyme is operating under optimal conditions for a given host taking into account side-reactions that sap initial substrates and intermediates between enzymes of the pathway.

In one embodiment, optimal conditions for a biosynthetic pathway may be achieved by balancing the rate of a single catalytic step or the rate of flow through a single step of the pathway. In another embodiment, optimal conditions for a biosynthetic pathway may be achieved by balancing the rate of two or more catalytic steps or the rates of flow through two or more steps of the pathway. For example, if substrate availability and intermediate accumulation are non-limiting, then pathway flow rate may be optimized by choosing highly efficient enzymes. Where less efficient enzymes are used, the resultant decreased flow rate may be compensated for by increasing their expression levels to provide a greater number of the less efficient enzyme to increase overall flow volume. This may be achieved, for example, by pairing a gene promoter with a high rate (e.g., 2× expression rate) of gene expression with a relatively less efficient enzyme and a gene promoter with a lower rate (e.g., 1× expression rate) of gene expression with a relatively more efficient enzyme. As a result, on average, the flow through the step catalyzed by the less efficient, but more abundant enzyme and that catalyzed by the more efficient, but less abundant enzyme can be balanced or made relatively equal. Such an approach may be used to “balance” biosynthetic pathways having multiple enzymes with varying levels of efficiency relative to one another by choosing the appropriate promoter/gene combination that results in an equivalent level of catalytic activity for each step. Another approach is to integrate multiple gene copies encoding of a less efficient enzyme into the genome of the host cell to increase the expression levels of the less efficient enzyme.

A recombinant gene encoding a polypeptide described herein comprises the coding sequence for that polypeptide, operably linked in sense orientation to one or more regulatory regions suitable for expressing the polypeptide. Because many microorganisms, particularly prokaryotes, are capable of expressing multiple gene products from a polycistronic mRNA, multiple polypeptides can be expressed under the control of a single regulatory region for those microorganisms, if desired. A coding sequence and a regulatory region are considered to be operably-linked when the regulatory region and coding sequence are positioned so that the regulatory region is effective for regulating transcription or translation of the sequence.

In many cases, the coding sequence for a polypeptide described herein is identified in a species other than the recombinant host, i.e., is a heterologous nucleic acid. Thus, if the recombinant host is a microorganism, the coding sequence can be from other prokaryotic or eukaryotic microorganisms, from plants or from animals. In some case, however, the coding sequence is a sequence that is native to the host and is being reintroduced into that organism. A native sequence can often be distinguished from the naturally occurring sequence by the presence of non-natural sequences linked to the exogenous nucleic acid, e.g., non-native regulatory sequences flanking a native sequence in a recombinant nucleic acid construct. In addition, stably transformed exogenous nucleic acids typically are integrated at positions other than the position where the native sequence is found. “Regulatory region” refers to a nucleic acid having nucleotide sequences that influence transcription or translation initiation and rate, and stability and/or mobility of a transcription or translation product. Regulatory regions include, without limitation, promoter sequences, enhancer sequences, response elements, protein recognition sites, inducible elements, protein binding sequences, 5′ and 3′ untranslated regions (UTRs), transcriptional start sites, termination sequences, polyadenylation sequences, introns, and combinations thereof. A regulatory region typically comprises at least a core (basal) promoter. A regulatory region also can include at least one control element, such as an enhancer sequence, an upstream element or an upstream activation region (UAR). A regulatory region is operably linked to a coding sequence by positioning the regulatory region and the coding sequence so that the regulatory region is effective for regulating transcription or translation of the sequence. A regulatory region can, however, be positioned as much as about 5,000 nucleotides upstream of the translation initiation site or about 2,000 nucleotides upstream of the transcription start site.

The choice of regulatory regions to be included depends upon several factors, including, but not limited to, efficiency, selectability, inducibility, desired expression level, and preferential expression during certain culture stages. It is a routine matter for one of skill in the art to modulate the expression of a coding sequence by appropriately selecting and positioning regulatory regions relative to the coding sequence. It will be understood that more than one regulatory region can be present, e.g., introns, enhancers, upstream activation regions, transcription terminators, and inducible elements.

As used herein, the term “detectable concentration” refers to a level of anthocyanin measured in mg/L, nM, μM, or mM. Anthocyanin production can be detected and/or analyzed by techniques generally available to one skilled in the art, for example, but not limited to, thin layer chromatography (TLC), high-performance liquid chromatography (HPLC), ultraviolet-visible spectroscopy/spectrophotometry (UV-Vis), mass spectrometry (MS), and nuclear magnetic resonance spectroscopy (NMR).

Anthocyanins

Anthocyanins are multi-glycosylated anthocyanidins, which, in turn, are derived from flavonoids such as naringenin. The anthocyanins are often further acylated in a process where moieties from aromatic or non-aromatic acids are transferred to hydroxyl groups of the anthocyanin-resident sugars. The aromatic acylation of anthocyanins increases stability and shifts their color.

Anthocyanins are pigments, which naturally appear red, purple, or blue, Frequently, the color of anthocyanins is dependent on pH. Anthocyanins are naturally found in flowers, where they provide bright-red and -purple colors. Anthocyanins are also found in vegetables and fruits. Anthocyanins are useful as dyes or coloring agents, and furthermore, anthocyanins have caught attention for their antioxidant properties.

There could be any number of reasons for the observed lack of previous demonstration of anthocyanin production from sugar in unicellular organisms. For instance, in E. coli, one impediment could have been a lack of sufficient precursors such as UDP-sugar, and malonyl-CoA, as well as the amino acids phenylalanine and tyrosine. In addition, expression of plant monooxygenases (CYP450s) in bacteria is a recognized challenge, because these enzymes depend on cofactors such as NAD(P)H dependent reductases, as well as co-localization to the ER membrane. In yeast, however, precursors and co-factors are relatively abundant, and most plant enzymes can readily be expressed. Yet, the art contained a surprising lack of attempts or examples for producing anthocyanins in yeast.

In addition, some of the later intermediates in the anthocyanin biosynthetic pathway, in particular leucoanthocyanins and anthocyanidins, are relatively unstable at physiological pH. In plants, this instability is thought to be circumvented by channeling these intermediates between enzymes that form close association or aggregates in the cytosol, possibly anchored on the ER surface. It is not known whether this channeling is taking place between enzymes heterologously expressed in bacteria and yeast. An attempt of channeling was made by Yan 2005 with some success by fusing the anthocyanidin synthase (ANS) and anthocyanidin 3-O-glycosyltransferase (A3GT) enzymes, but it was later suggested that the more important factor is to have efficient expression of A3GT (Lim 2015).

Another issue that has hampered heterologous expression is the promiscuity of several enzymes regarding substrate specificity, and the ability of such enzymes to catalyze more than one reaction. This is particularly the case with a group of 2-oxoglutarate dependent dioxygenases (2ODDs) including flavanone 3-hydroxylase (F3H) and ANS. ANS has very high similarity to flavonol synthase (FLS) and has been shown to catalyze many of the same reactions normally associated with FLS and flavonol synthesis. Hence, after expression of biosynthetic pathways directed to anthocyanin production, the result has been high amounts of flavonols (both aglycones and their 3-O-glycosides). Several ANS enzymes have been tested with similar results, and this has hampered production of anthocyanins from their precursors, e.g., flavanones and dihydroflavonols. It is also likely to be one of the major reasons why anthocyanin production from glucose has not been previously demonstrated in bacteria and yeast.

Further, heterologous compound production via heterologous biosynthetic pathways often faces competition from host enzymes capable of degrading or modifying intermediates, or otherwise shunting them away from the main pathway. In yeast, this includes degradation of phenyl propanoids, as well as cleavage of the final glucoside to revert anthocyanins to the unstable anthocyanidins. Such issues are further exacerbated when the heterologous synthetic pathways compete for primary substrates for host metabolism, such as glucose.

Despite these previous challenges, this invention demonstrates that unexpectedly, it is possible to produce anthocyanins from simple sugars, such as glucose, or other simple carbon sources such as glycerol, ethanol, or easily fermentable raw materials in microorganisms such as yeast, by careful selection and expression of highly efficient heterologous enzymes.

In one embodiment, the invention discloses a recombinant host cell including an operative metabolic pathway capable of producing an anthocyanidin of the formula I:

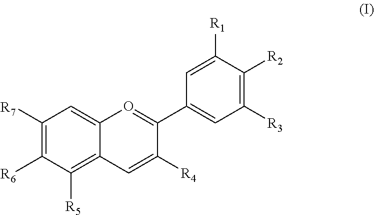

wherein
R₁ is selected from the group consisting of —H, —OH and —OCH₃; and
R₂ is selected from the group consisting of —H and —OH; and
R₃ is selected from the group consisting of —H, —OH and —OCH₃; and
R₄ is selected from the group consisting of —H and —OH; and
R₅ is selected from the group consisting of —OH and —OCH₃; and
R₆ is selected from the group consisting of —H and —OH; and
R₇ is selected from the group consisting of —OH and —OCH₃ [0015] In certain aspects, the anthocyanidin is selected from the group consisting of aurantinidin, cyanidin, deiphinidin, europinidin, luteolinidin, pelargonidin, malvidin, peonidin, petunidin and rosinidin.

In one embodiment, a recombinant host cell is provided that is genetically engineered to include an operative metabolic pathway for producing anthocyanins from glucose. In another embodiment, a microorganism is provided that is engineered to include an operative metabolic pathway for producing anthocyanins including only heterologous genes in the operative metabolic pathway. For example, in the case of a yeast host, the operative metabolic pathway may include genes from plants, archaea, bacteria, animals, and other fungi. In one embodiment, each of the heterologous genes in the operative metabolic pathway is from one or more plants.

In another embodiment, a recombinant host cell is provided that includes one or more heterologous nucleic acid molecules that encode enzymes of the aurantinidin, cyanidin, deiphinidin, europinidin, luteolinidin, pelargonidin, malvidin, peonidin, petunidin and/or rosinidin biosynthesis pathways. In certain aspects, the host cells are capable of producing cyanidin. In other aspects, the host cells comprise one or more heterologous enzyme nucleic acid molecules each encoding an enzyme of the cyanidin biosynthesis pathway.

As will be understood by a person skilled in the art, any enzyme of the anthocyanin synthetic pathway can be a target for optimization by genetic modifications, such as specific deletions, insertions, alterations, e.g., by mutagenesis, to improve both the specificity and turn-over rate of that enzyme. Moreover, while specific enzymes are disclosed herein, the skilled worker will appreciate that each disclosed enzyme represents its enzymatic function rather than only the listed enzyme and should not be considered to be limited to the particular enzyme exemplified herein by name or sequence.

In certain embodiments, the heterologous enzymes can be selected from any one or a combination of organisms. For example, organisms from which heterologous enzymes for use herein may be selected include one or more of the following genera: Petunia, Malus, Anthurium, Zea, Arabidopsis, Ammi, Glycine, Hordeum, Medicago, Populus, Fragaria, Dianthus, Saccharomyces, and the like. Representative species from these genera that may be used include Petunia x hybrida, Malus domestica, Anthurium andraeanum, Arabidopsis thaliana, Ammi majus, Hordeum vulgare, Medicago sativa, Populus trichocarpa, Fragaria x ananassa, Dianthus caryuphyllus, and Saccharomyces cerevisiae.

Orthogonal enzymes from other organisms may also be substituted. Hence, there may be many options for constructing anthocyanin or catechin pathways by identifying a set of enzymes that will work well together in a given microorganism.

Host optimization to improve expression of the heterologous pathways described is also possible. This may, for example, be done in such a way as to improve the ability of the host to provide higher levels of precursor molecules, tolerate higher levels of product, or to eliminate unwanted host enzyme activity which interferes with the heterologous anthocyanin-producing pathway.

In another embodiment, enzymes that may be used herein include any enzymes involved in anthocyanidin synthesis or anthocyanin synthesis. For example, enzymes contemplated for use herein include those listed in Table No. 1 below and homologs and variants thereof, including host-specific codon optimized variants.

TABLE NO. 1
Enzymes.

| Gene | Gene product |
|---|---|
| ANS | Anthocyanidin synthase |
| A3GT | Anthocyanidin-3-O-glycosyl transferase |
| DFR | Dihydroflavonol-4-reductase |
| PAL | Phenylalanine ammonia lyase |
| C4H | Trans-cinnamate 4-monooxygenase |
| 4CL | 4-coumaric acid-CoA ligase |
| CHS | Chalcone synthase |
| CHI | Chalcone isomerase |
| F3H | Flavanone 3-hydroxylase |
| F3′H | Flavonoid 3′-hydroxylase |
| F3′5′H | Flavonoid 3′-5′-hydroxylase |
| FLS | Flavonol synthase |
| LAR | Leucoanthocyanidin reductase |
| TAL | Tyrosine ammonia lyase |
| A5GT | Anthocyanin-5-O-glycosyl transferase |
| A3AAT | Anthocyanin-3-O-aromatic acyl transferase |
| A3MAT | Anthocyanin-3-O-malonyl acyl transferase |

In another embodiment, the recombinant host cell may further include anthocyanidin synthase (AIMS (I_DOX)), flavonol synthase (FLS), leucoanthocyanidin reductase (LAR), and anthocyanidin reductase (ANR).

In other aspects, the invention provides a recombinant host cell that is capable of producing a compound selected from the group consisting of coumaroyl-CoA, benzoyl-CoA, sinapoyl-CoA, feruloyl-CoA, malonyl-CoA, cinnamoyl-CoA, and caffeoyl-CoA. In further aspects, the recombinant host comprises one or more heterologous enzyme nucleic acid molecules each encoding an enzyme of the coumaoryl-CoA biosynthesis pathway.

In one embodiment, a recombinant host cell is provided that is capable of producing one or more anthocyanins, wherein the host cell expresses at least one anthocyanidin, and wherein the host cell includes one or more heterologous GT nucleic acid molecules and one or more heterologous AT nucleic acid molecules.

In a further embodiment, a recombinant host cell is provided that includes a glycosyltransferase that is a UDP-glucose dependent glucosyltransferase. For example, the glycosyltransferase can be a UDP-glucose dependent glucosyltransferase of family 1.

In another embodiment, a recombinant host cell is provided that includes an acyltransferase, for example, a BAHD acyltransferase.

The term “anthocyanin” as used herein refers to any anthocyanidin, which have been glycosylated and/or acylated at least once. However, an anthocyanin may also have been glycosylated and/or acylated several times. Thus, in principle, an anthocyanidin may also be an anthocyanin, which has been glycosylated and/or acylated at least once.

Thus, an anthocyanin may be any of the anthocyanidins described herein, wherein the anthocyanidin is substituted with one or more selected from the group consisting of glycosyl, acyl, substituents consisting of more than one glycosyl, substituents consisting of more than one acyl and substituents consisting of one or more glycosyl(s) and one or more acyl(s).

The anthocyanidin can be substituted at any useful position. Frequently, the anthocyanidin is substituted at one or more of the following positions: the 3 position on the C-ring, the 5 position on the A-ring, the 7 position on the A ring, the 3′ position of the B ring, the 4′ position of the B-ring or the 5′ position of the B-ring.

Accordingly, in one embodiment of the invention the anthocyanin is a compound of the formula I:

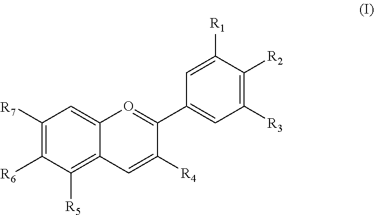

wherein
R₁ is selected from the group consisting of —H, —OH, —OCH₃ and O—R₈; and
R₂ is selected from the group consisting of —H, —OH and O—R₈; and
R₃ is selected from the group consisting of —H, —OH, —OCH₃ and O—R₈; and
R₄ is selected from the group consisting of —H, —OH and O—R₈; and
R₅ is selected from the group consisting of —OH, —OCH₃ and O—R₈; and
R₆ is selected from the group consisting of —H and —OH; and
R₇ is selected from the group consisting of —OH, —OCH₃ and O—R₈ and
R₈ is selected from the group consisting of glycosyl, acyl, substituents consisting of more than one glycosyl, substituents consisting of more than one acyl and substituents consisting of one or more glycosyl(s) and one or more acyl(s); and wherein at least one of R₁, R₂, R₃, R₄, R₅ and R₇ is —O—R₈.

The acyl may be any acyl. In one embodiment, one or more acyls are selected from the group consisting of the acyl moiety of a fatty acid. In another embodiment one or more acyls are selected from the group consisting of coumaroyl, benzoyl, sinapoyl, feruloyl and caffeoyl, malonyl and hydroxybenzoyl.

The glycoside can be any sugar residue. For example, one or more glycosides may be selected from the group consisting of glucoside, rhamnoside, xyloside, galactoside and arabinoside.

The substituent consisting of one or more glycosides can be, for example, a monosaccharide, disaccharide, or a trisaccharide. The monosaccharide can be, for example, selected from the group consisting of glucoside, rhamnoside, xyloside, galactoside and arabinoside. The disaccharide and the trisaccharide can, for example, consist of glycosides selected from the group consisting of glucoside, rhamnoside, xyloside, galactoside and arabinoside.

The substituent consisting of one or more glycosides and one or more acyl can be, for example, a monosaccharide, disaccharide or a trisaccharide substituted at one or more positions with an acyl. The substituent consisting of one or more glycosides and one or more acyl can be, for example, a monosaccharide selected from the group consisting of glucoside, rhamnoside, xyloside, galactoside and arabinoside, wherein any of the aforementioned can be substituted at one or more positions with an acyl selected from the group consisting of coumaroyl, benzoyl, sinapoyl, feruloyl and caffeoyl, malonyl and hydroxybenzoyl. The substituent consisting of one or more glycosides and one or more acyl can also be, for example, a disaccharide or a trisaccharide consisting of glycosides selected from the group consisting of glucoside, rhamnoside, xyloside, galactoside and arabinoside, wherein any of the aforementioned can be substituted at one or more positions with an acyl selected from the group consisting of coumaroyl, benzoyl, sinapoyl, feruloyl and caffeoyl, malonyl and hydroxybenzoyl.

In one embodiment, an anthocyanin can have multiple glycosylations. Such anthocyanins exhibit improved systemic bioavailability (compared to the aglycon (a non-glycosylated molecule) alone or an anthocyanin with fewer glycosylations). The sugars can be removed in the GI tract. Such multiply glycosylated anthocyanins (one or more glycosylations) also have improved aqueous solubility. The anthocyanin with no sugars or fewer sugars than when ingested can then cross through the GI wall.

The improvement of bioavailability or solubility or a combination thereof can be 2, 5, 10, 50, 100, 200 or more fold.

Sugars can be added to the anthocyanin by an enzyme or by a metabolic process within a cell. The sugars can be any sugar, for example, glucose, galactose, lactose, fructose, maltose, and can be added to more than one site on the anthocyanin. There can be more than one sugar per site, or 2, 3, 4, 5, or more sugars per site. The anthocyanin can first be derivatized with a functional group (using e.g. a P450 or other enzyme) that the sugar is subsequently added to.

Co-pigmentation can affect stability, color, and hue. This can be an intramolecular interaction e.g. of the acyl group with the rest of the anthocyanin molecule or intermolecular interactions with other molecules in solution. The effect of acyl group variation protects intramolecular but not intermolecular co-pigmentation.

For processing, formulation and storage of products containing anthocyanins, stabilization of the intact anthocyanin is desired. However, in vivo therapeutic effects of anthocyanins can be due to one of more of native anthocyanin, degradation products, metabolites or anthocyanin derivatives. Notably, the amount of native anthocyanin in plasma has been quoted as less than 1% of the consumed quantities. This has been considered to be due to limited intestinal absorption, high rates of cellular uptake, metabolism and excretion.

Therefore, for therapeutic applications of anthocyanins, it can be advantageous to use anthocyanins with instability at the relevant stage of the digestive tract, or derivatization for maximum adsorption at the relevant stage of the digestive tract. Colonic metabolism of anthocyanins can also be considered. Therefore, in some instances “improved stability” of an anthocyanin may actually be a decrease in stability for delivery to a specific stage of the digestive tract or colon. The chemical forms of anthocyanins ingested in the diet may not be the ones that reach microbiota but instead their respective metabolites that were excreted in the bile and/or from the enterohepatic circulation.

Glycosyl Transferases

Glycosyltransferases that can be used with the present invention can be any enzymes that are capable of catalyzing transfer of one monosaccharide residue to an acceptor molecule. In particular, useful glycosyltransferases are any enzymes that can catalyze transfer of one monosaccharide residue from a sugar donor to an acceptor molecule. In particular, glycosyltransferases useful in the present invention are capable of catalyzing transfer of one monosaccharide residue selected from the group consisting of glucose, rhamnose, xylose, galactose and arabinose to an acceptor molecule selected from the group consisting of anthocyanins and anthocyanidins.

The sugar donor can be any moiety having a monosaccharide, such as any donor moiety covalently coupled to a glycoside, such as a glycoside selected from the group consisting of glucoside, rhamnoside, xyloside, galactoside and arabinoside. The donor moiety can be, for example, a nucleotide, such as a nucleoside diphosphosphate, for example, UDP. Thus, the sugar donor can be, for example, a UDP-glycoside, wherein glycoside for example may be selected from the group consisting of glucoside, rhamnoside, xyloside, galactoside and arabinoside.

The sugar donor can also be a molecule consisting of a sugar moiety and an acyl moiety, e.g., an aromatic acyl moiety, such as a phenyl propanoid moiety. Such donors are described in, e.g., Sasaki et al. (“The Role of Acyl-Glucose in Anthocyanin Modifications,” Molecules 19: 18747-66, 2014).

The art describes a number of glycosyltransferases that can glycosylate compounds of interest. Based on DNA sequence homology of the sequenced genome of the plant Arabidopsis thaliana, it is believed to contain around 100 different glycosyltransferases. These and numerous others have been analyzed in Paquette et al., (Phytochemistry 62: 399-413, 2003). WO2001/07631, WO2001/40491, and Arend et al., (Biotech. & Bioeng 78: 126-131, 2001) also describe useful glycosyltransferases, which may be employed with the present invention.

Furthermore, numerous suitable glycosyltransferases may be found in the Carbohydrate-Active enZYmes (CAZY) database (http://www.cazy.org/). In the CAZY database, suitable glycosyltransferase molecules from virtually all species including, animal, insects, plants and microorganisms can be found. Furthermore, a type of glycosyl transferase of the glycoside hydrolase family 1 (GH1), as described e.g. in Sasaki et al. that uses acyl-glucosides as donors, may be used in the present invention.

In one embodiment, at least 50% of the glycosyltransferases, such as at least 75% of the glycosyltransferases, to be used with the methods of the invention belong to the CAZy family GT1. The skilled person will be able to identify whether a given glycosyltransferase belong to a particular CAZy family using conventional, computer-aided methods based mainly on sequence information. The GT1 family has at least 5217 genes coding for glycosyltransferases. They are referred to as UGTs and are numbered UGT<family numberxgroup letter><enzyme number>.

Glycosyltransferases that are more than 40% identical to a given GT1 member in amino acid sequence are classified to the same UGT-family within GT1. Those that are 60% or more identical receive the same group letter, and the individual glycosyltransferase is then assigned an enzyme number.

In one embodiment, it may be advantageous to include Nucleotide-Sugar Interconversion enzymes, such as RHM2, to improve availability of the desired sugar donor, by converting UDP-glucose to UDP-rhamnose. Several of such enzymes are known in the art. (See e.g., Yin et al. (“Evolution of plant nucleotide-sugar interconversion enzymes,” PLoS One. 6(11): e27995, 2011).

Acyl Transferases

Acyltransferases that can be used with the present invention can be any enzyme that is capable of catalyzing transfer of an acyl residue to an acceptor molecule. In particular, the acyltransferase to be used with the present invention can be any enzymes that are capable of catalyzing transfer of one acyl residue from an acyl donor to an acceptor molecule selected from the group consisting of anthocyanins and anthocyanidins.

Useful acyltransferases include that capable of catalyzing transfer of one acyl residue from coenzyme A-derivative of an organic acid to an acceptor molecule selected from the group consisting of anthocyanins and anthocyanidins.

The acyltransferase can be any enzyme that is capable of catalysing transfer of one acyl residue from any of the acyl donors described herein below in the section “Acyl donor” to an anthocyanin and/or an anthocyanidin.

In one embodiment, the acyltransferase is of the BAHD type. Nucleic acid molecules encoding BAHD acyltransferases can be identified by screening gene transcripts present in anthocyanin-producing tissues of plants having a high level of anthocyanin production. The screening can use homology searching with known BAHD genes to identify additional nucleic acid molecules encoding BADH acyltransferases. For these enzymes, certain protein motifs are conserved well enough to allow easy identification. The identified nucleic acid molecules can then be transferred to host cells or be used for in vitro production of acyltransferases to be used with the methods of the invention.

In another embodiment, the acyltransferase can belong to the EC 2.3.1.—class of enzymes, including EC 2.3.1.18; EC 2.3.1.153; EC 2.3.1.171; EC 2.3.1.172; EC 2.3.1.173; EC 2.3.1.213; EC 2.3.1.214; EC 2.3.1.215; and similar enzymes.

In yet another embodiment, the acyltransferase can belong to the class of AHCT (anthocyanin o-hydroxy cinnamoyl transferase) enzymes. An exemplary GenBank Accession Number for an AHCT nucleic acid molecule includes, but is not limited to, AY395719.1.

In yet another embodiment, the acyltransferase can be a serine carboxypeptidase-like (SCPL) protein family type, which uses acyl-glycosides as donors to transfer the acyl to the target molecule. Such acyltransferases and their donor molecules are described, e.g., in Sasaki et al.

According to the invention, enzymes of any of the above mentioned classes can be used individually or as mixtures.

The acyl donor can be any useful acyl donor. In particular, the acyl donor may be any moiety including an acyl residue, such as any donor moiety covalently coupled to an acyl residue. The acyl residue can be the acyl part of an organic acid. The donor moiety can be coenzyme A, and thus, the acyl donor can be a coenzyme A-derivative of an organic acid including aromatic phenolic acids or phenylpropanoic acids. Further, the acyl donor can be a compound selected from the group consisting of acetyl-CoA, malyl-CoA, malonyl-CoA, coumaroyl-CoA, benzoyl-CoA, sinapoyl-CoA, feruloyl-CoA and caffeoyl-CoA. In particular, the acyl donor can be coumaroyl-CoA.

Further, the acyl donor can be an acyl-glucoside of the type described in Sasaki et al.

In certain embodiments of the invention, the acyl donor can be added directly to the fermentation broth. However, in a preferred embodiment of the invention, the recombinant host cell can be capable of producing the acyl donor. Many host cells are capable of producing one or more acyl donors. For example, yeast cells are capable of producing malonyl-CoA.

Frequently, however, host cells are not capable of producing all desired acyl donors, in which case the host cells can include one or more heterologous enzyme nucleic acid molecules each encoding enzymes of the biosynthetic pathway of the specific acyl donor.

Several biosynthesis pathways for conversion of a sugar into an acyl donor are known. Where the host cell is a yeast or bacterial cell, the cell can include a heterologous enzyme nucleic acid molecule encoding one or more enzymes of the biosynthetic pathway for conversion of a sugar into an acyl donor, even though some of the required enzymatic activities typically are present in the host cell. Thus, frequently the acyl donor can be prepared using phenyl alanine or tyrosine as a substrate. Typically host cells, such as yeast or bacterial cells, are capable of producing phenyl alanine or tyrosine.

Thus, the host cell can include heterologous nucleic acid molecules encoding one or more enzymes of the biosynthesis pathway for conversion of phenyl alanine or tyrosine to phenylpropanoyl-CoA. For example, the host cell can include heterologous nucleic acid molecules encoding all the enzymes of the biosynthesis pathway for conversion of phenylalanine or tyrosine to e.g. feruloyl-CoA.

The host cell can also include heterologous nucleic acid molecules encoding one or more enzymes of the biosynthesis pathway for conversion of phenylalanine or tyrosine to p-hydroxybenzoyl-CoA. For example, the host cell can include heterologous nucleic acid molecules encoding all the enzymes of the biosynthesis pathway for conversion of phenylalanine or tyrosine to p-hydroxybenzoyl-CoA.

Host cells may include any suitable cell for expression of the biosynthetic pathway proteins disclosed herein, including, but not limited to, prokaryotic and eukaryotic species, such as yeast cells, plant cells, mammalian cells, insect cells, fungal cells, bacterial cells. If the cells are human cells, they are isolated or cultured.

Suitable host cells include yeast, such as those belonging to the genera Saccharomyces, Ashbya, Arxula, Klyuveromyces, Gibberella, Aspergillus, Candida, Pichia, Debaromyces, Hansenula, Yarrowia, Zygosaccharomyces, Cyberlindnera, Hansenula, Xanthophyllomyces, or Schizosaccharomyces. For example, a suitable yeast species may be Saccharomyces cerevisiae, Schizosaccharomyces pombe, Yarrowia lipolytica, Candida glabrata, Ashbya gossypii, Gibberella fujikuroi, Aspergillus niger, Cyberlindnera jadinii, Pichia pastoris, Kluyveromyces lactis, Hansenula polymorpha, Candida boidinii, Arxula adeninivorans, Xanthophyllomyces dendrorhous, or Candida albicans.

Suitable bacterial cells include Escherichia bacteria cells, Lactobacillus bacteria cells, Lactococcus bacteria cells, Cornebacterium bacteria cells, Acetobacter bacteria cells, Acinetobacter bacteria cells, Pseudomonas bacterial cells, or Rhodobacter sphaeroides, Rhodobacter capsulatus, or Rhodotorula toruloides cells.

In some embodiments, a microorganism can be an algal cell such as Blakeslea trispora, Dunaliella salina, Haematococcus pluvialis, Chlorella sp., Undaria pinnatifida, Sargassum, Laminaria japonica, or Scenedesmus almeriensis species.

In some embodiments, a microorganism can be a cyanobacterial cell such as Blakeslea trispora, Dunaliella salina, Haematococcus pluvialis, Chlorella sp., Undaria pinnatifida, Sargassum, Laminaria japonica, or Scenedesmus almeriensis.

The genetically engineered microorganisms disclosed herein can be cultivated using conventional cell culture or fermentation processes, including, inter alia, chemostat, batch, fed-batch cultivations, continuous perfusion fermentation, and continuous perfusion cell culture.

After the microorganism has been grown in culture for a desired period of time, anthocyanin and/or one or more anthocyanin derivatives or anthocyanidin can then be recovered from the culture using various techniques known in the art.

Once isolated, anthocyanins produced according to the current disclosure may be used, as is known in the art, as colorants (such as dyes or pigments that may have a predetermined color and/or hue), pH indicators, food additives, antioxidants, for medicinal purposes, or for any other use, including food and nutritional supplements.

The invention will be further described in the following examples, which do not limit the scope of the invention described in the claims.

### EXAMPLES

The Examples that follow are illustrative of specific embodiments of the invention, and various uses thereof. They are set forth for explanatory purposes only and are not taken as limiting the invention.

Overview

The following Examples demonstrate successful anthocyanin production in yeast via a heterologous full-length biosynthetic pathway. Successful production was achieved by combining highly efficient enzymes and expressing them under near optimal conditions to achieve sufficient flow through the pathway (and to overcome deleterious side-reactions) to produce useful amounts of anthocyanin products. As listed in the tables below, the gene sequences disclosed in SEQ ID NOS: 1, 3, 5, 7, 9, 11, 13, 15, 17, 19, 21, 23, 25, 27, 29, 31, 33, 45, 47, 48, 51, and 52 encode the protein sequences of SEQ ID NOS: 2, 4, 6, 8, 10, 12, 14, 16, 18, 20, 22, 24, 26, 28, 30, 32, 34, 54, 55, 56, 57, and 58, respectively.

All flavonoids, anthocyanidins, anthocyanins, and their derivatives in the examples below were analyzed using the method set forth in Example No. 10.

### Example No. 1: Production of Naringenin in Yeast

Materials and Methods

The naringenin pathway was assembled by in vivo homologous recombination and simultaneous integration in a background S. cerevisiae strain to make a naringenin producing strain. The S. cerevisiae strains used were based on the S288c strain.

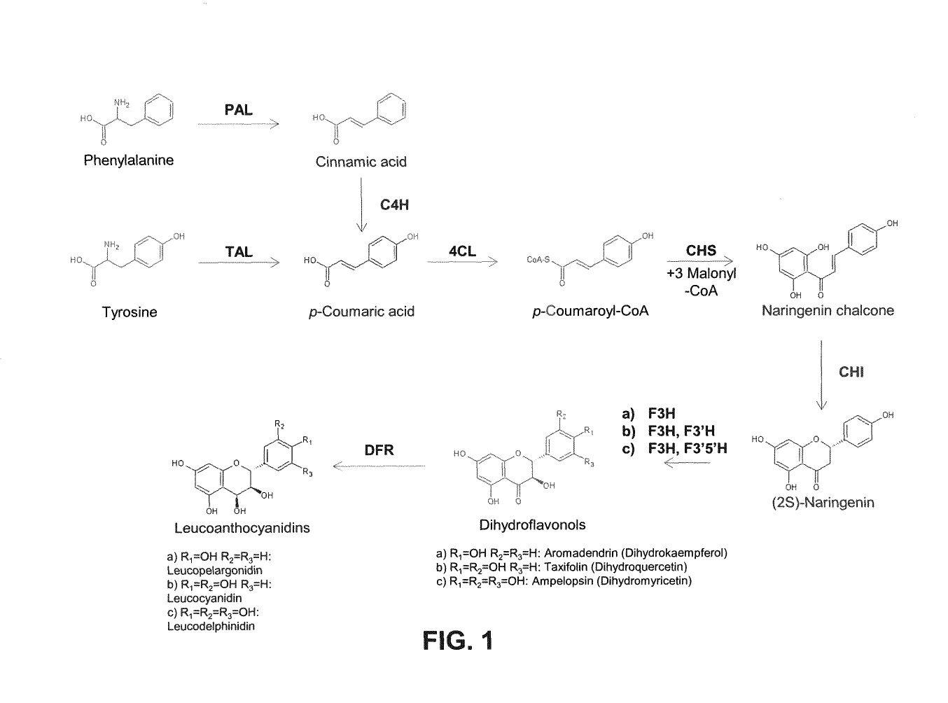

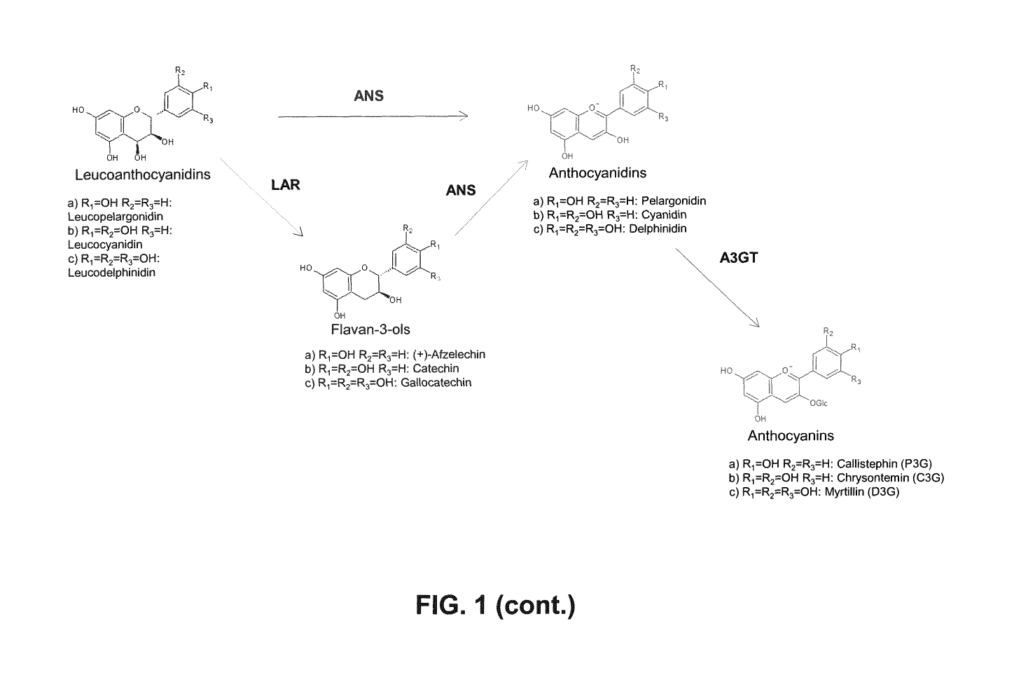

The naringenin pathway genes used in this example are listed in Table No. 2 below, though a tyrosine ammonia lyase (TAL), such as that encoded by SEQ ID NO: 15 may be used in place of or in addition to PAL2 and C4H (as illustrated in FIG. 1) to provide the intermediate, p-coumaric acid, in the pathway.

TABLE NO. 2
Naringenin Pathway Genes used in Example No. 1.

| Plasmid | | | SEQ ID | |
|---|---|---|---|---|
| (pEVE) | Cassette | Content | NO | Species |
| 4745 | ZA | Integration tag | 35 | |
| | | for XI-3 | | |
| 3169 | AB | URA3 and | 36 | |
| | | LoxP | | |
| | BC | PAL2 At | 17 | Arabidopsis thaliana |
| | CD | C4H Am | 19 | Ammi majus |
| | DE | 4CL2 At | 1 | Arabidopsis thaliana |
| | EF | CHS2 Hv | 21 | Hordeum vulgare |
| | FG | CHI Ms | 13 | Medicago sativa |
| | GH | CPR1 Sc | 23 | Saccharomyces cerevisiae |
| 1919 | HZ | 600 bp stuffer | 37 | |

All genes were manufactured based on sequences from public databases, except CPR1 Sc (SEQ ID NO: 23) and 4CL2 At (SEQ ID NO: 1), which were amplified from yeast genomic DNA and plant cDNA, respectively. Synthetic genes, codon-optimized for expression in yeast, were manufactured by DNA 2.0, Inc. (Menlo Park, Calif., USA) or GeneArt AG (Regensburg, Germany). During synthesis, all genes except PAL2 At were provided, at the 5′-end, with the DNA sequence AAGCTTAAA (SEQ ID NO: 43) including a Hind III restriction recognition site and a Kozak sequence, and at the 3′-end the DNA sequence CCGCGG (SEQ ID NO: 44) including a SacII recognition site. By PCR, PAL2 At was provided, at the 5′-end, with the DNA sequence AAGCTTAAA (SEQ ID NO: 43), including a HindIII restriction recognition site and a Kozak sequence, and at the 3′-end with the DNA sequence CCGCGG (SEQ ID NO: 44) including a SacII recognition site. The A. thaliana gene 4CL2 (SEQ ID NO: 1) was amplified by PCR from first strand cDNA. The 4CL2 sequence has one internal HindIII site and one internal SacII site, and was therefore cloned, using the In-Fusion® HD Cloning Plus kit (Clontech, Inc.), into HindIII and SacII, according to manufacturers' instructions.

The S. cerevisiae gene CPR1 was amplified from genomic DNA by PCR (SEQ ID NO: 23). During PCR, the gene was provided, at the 5′-end, with the DNA sequence AAGCTTAAA (SEQ ID NO: 43), including a HindIII restriction recognition site and a Kozak sequence, and at the 3′-end with the DNA sequence CCGCGG (SEQ ID NO: 44) including a SacII recognition site. An internal SacII site of SEQ ID NO: 23 was removed with a silent point mutation (C519T) by site directed mutagenesis. Yeast CPR1 was overexpressed to allow efficient regeneration of the CYP450 enzyme C4H. All genes were cloned into HindIII and SacII of pUC18 based vectors containing yeast expression cassettes derived from native yeast promoters and terminators.

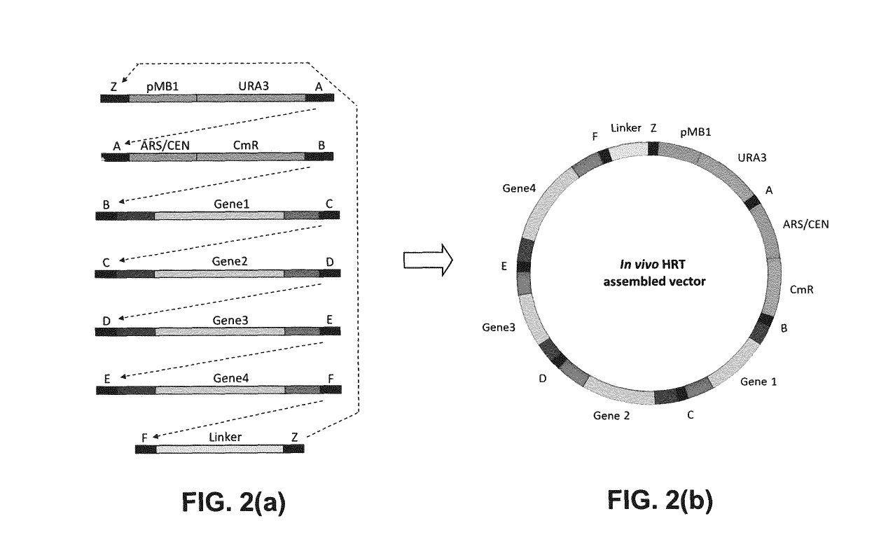

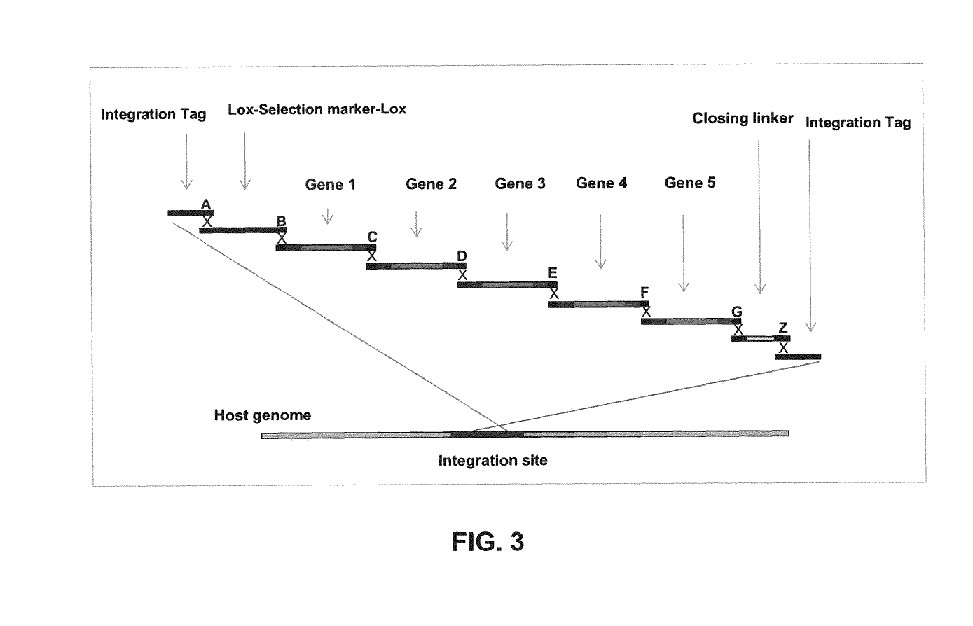

Promoters and terminators, described by Shao et at (Nucl. Acids Res. 2009, 37(2):e16), had been prepared by PCR from yeast genomic DNA. Each expression cassette was flanked by 60 bp homologous recombination tag (HRT) sequences, on both sides, and the cassettes including these HRTs were, in turn, flanked by AscI recognition sites (see FIGS. 2(a), 2(b), and 3). The HRTs were designed such that the 3′-end tag of the first expression cassette fragment is identical to the 5′-end tag of the second expression cassette fragment, and so forth. Three helper fragments were used to integrate multiple expression cassettes into the yeast genome by homologous recombination. One helper fragment (ZA in pEVE4745, SEQ ID NO: 35), included the two recombination tags for integration into the site XI-3, each of which was homologous to sequences in the yeast genome. These were both flanked by a HRT and separated with an AscI site. The second helper fragment (AB in pEVE3169, SEQ ID NO: 36) included a yeast auxotrophic marker (URA3) flanked by LoxP sites. This fragment also had flanking HRTs. The third helper fragment (HZ in pEVE1919, SEQ ID NO: 37) was designed only with HRTs separated by a short 600 bp spacer sequence. All helper fragments had been cloned in a pUC18 based backbone for amplification in E. coli. All fragments were cloned in AscI sites from where they could be excised. FIGS. 2(a) and (b) and FIG. 3 depict how the DNA assembler technology, based on Shao et al. 2009, can be used to assemble biosynthetic pathways by homologous recombination, for stable maintenance on a plasmid (FIGS. 2(a) and (b)) or after integration into the host genome (FIG. 3).

To integrate the naringenin pathway into the background strain, plasmid DNA from the three helper plasmids (pEVE4745, pEVE3169, and pEVE1919, SEQ ID NOS: 35-37, respectively) was mixed with plasmid DNA from each of the plasmids containing the expression cassettes. The mix of plasmid DNA was digested with AscI. This treatment released all fragments from the plasmid backbone and created fragments with HRTs at the ends, these being sequentially overlapping with the HRT of the next fragment. The background strain was transformed with the digested mix, and the naringenin pathway was integrated in vivo by homologous recombination essentially as described by Shao et al. 2009.

Following integration, the genes were transcribed and translated into the enzymes of the naringenin biosynthetic pathway, plus the additional yeast CPR1. Naringenin production was confirmed by LC/MS.

### Example No. 2: Production of Pelargonidin-3-O-Glucoside (P3G) in Yeast

The pelargonidin-3-O-glucoside (P3G)-pathway from naringenin was assembled on HRT vectors according to Table No. 3 below. Each yeast expression cassette BC, CD, DE and EF contained a gene encoding one enzyme of the P3G pathway. The BC cassette encoded an anthocyanidin synthase (ANS) from Petunia×hybrida, the CD cassette contained an anthocyanidin-3-O-glycosyl transferase (A3GT) from Arabidopsis thaliana, the DE cassette encoded a flavanone-3-hydroxylase (F3H) from Malus domestica, and the EF cassette encoded a dihydroflavonol-4-reductase (DFR) from Anthurium andraeanum. See FIGS. 2(a) and 2(b) depicting pathway assembly on a plasmid, and FIG. 3 depicting assembly by genomic integration.

The backbone of the HRT vectors was formed by the DNA fragments ZA, AB and FZ, which contained a yeast selection marker, an autonomously replicating sequence (ARS), a yeast centromere (CEN) and a 600 bp stuffer sequence (see Table No. 3 below). Expression of each cassette was driven by a yeast native promoter as described in Example No. 1 above. The DNA helper fragments, as well as the gene expression cassettes, were flanked by 60 bp homologous recombination tags (HRT), where each terminal tag was identical to the first tag of the following cassette. Each HRT cassette included terminal AscI restriction sites to allow excision from the vector backbone.

TABLE NO. 3
P3G Pathway Gene Cassettes.*

| Plasmid | | | SEQ ID | Plasmid size | Amount |
|---|---|---|---|---|---|
| (pEVE) | Cassette | Content | NO | (kb) | (ng) |
| 4729 | ZA | HIS3, pSC101 | 38 | 6.3 | 252 |
| 1968 | AB | ARS/CEN, | 39 | 4.8 | 192 |
| | | CmR | | | |
| 4134 | BC | ANS Ph | 9 | 5.3 | 318 |
| 4005 | CD | A3GT At | 25 | 5.5 | 330 |
| 4015 | DE | F3H-1 Md | 3 | 4.9 | 294 |
| 4024 | EF | DFR Aa | 5 | 5.2 | 312 |
| 1917 | FZ | 600 bp stuffer | 40 | 3.6 | 216 |

*Summary of the plasmids containing the cassettes included in the final HRT vector for P3G production in yeast. Approximate sizes of the undigested donor plasmids are indicated, as well as the amounts of DNA that were mixed and digested with Ascl before being used to transform the yeast.

Plasmids (from Table No. 3) containing the described helper fragments and gene expression cassettes were digested with AscI in a 20 μL reaction volume. The digest was performed for 2 h at 37° C.

For transformation of a naringenin producing yeast strain (described in Example No. 1) with the HRT reaction, a 5 mL pre-culture of the naringenin producing strain was inoculated the day before transformation. After transformation of the naringenin producing strain by the LiAC/SS carrier DNA/PEG method (see e.g., Gietz et al., Nat Protoc. 2007; 2(1):35-7), cells were grown at 30° C. for 72 h. Next, four clones were re-streaked onto fresh plates and grown for 72 h at 30° C.

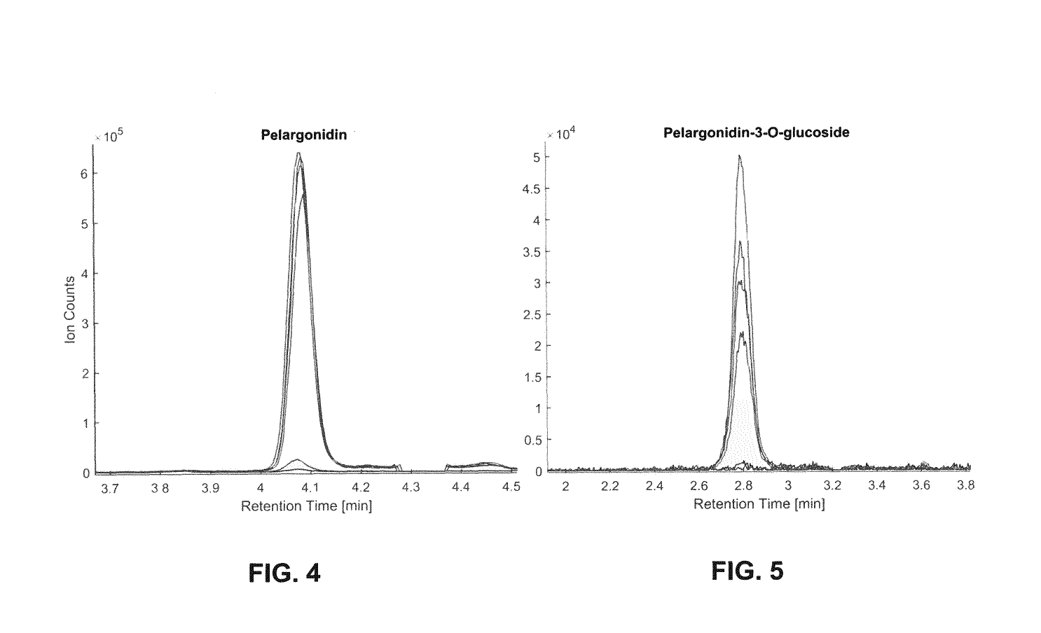

The clones were then grown in 2 mL liquid cultures until the cultures turned red (96 h to 120 h). Subsequently, 1 volume of acidified methanol was added, and after ½ hour of shaking at 30° C. cell debris was spun down by centrifugation and the cleared supernatant was collected for analysis by LC/MS. Analysis demonstrated the presence of pelargonidin (FIG. 4) and pelargonidin-3-O-glucoside (FIG. 5).

### Example No. 3: Production of Pelargonidin-3,5-O-Diglucoside (P35G) in Yeast

The pelargonidin-3-5-O-diglucoside pathway, starting from naringenin, was assembled in yeast by utilization of the HRT technique, described in Example No. 1 above and shown in FIGS. 2(a) and 2(b). Genes used for P35G production are summarized Table No. 4 below. Each yeast expression cassette BC, CD, DE, EF and FG contained a gene encoding one enzyme of the P35G pathway. The BC cassette encoded an anthocyanidin synthase (ANS) from Petunia×hybrida, the CD cassette contained an anthocyanidin-3-O-glycosyl transferase (A3GT) from Arabidopsis thaliana, the DE cassette encoded a flavanone-3-hydroxylase (F3H) from Malus domestica, the EF cassette encoded a dihydroflavonol-4-reductase (DFR) from Anthurium andraeanum, and the FG cassette encoded an anthocyanin-5-O-glucosyltransferase from Vitis amurensis. All genes were manufactured based on sequences from public databases, codon-optimized for expression in yeast, and manufactured by DNA 2.0, Inc. (Menlo Park, Calif., USA) or GeneArt AG (Regensburg, Germany).

The backbone of the P35G HRT vector was formed by the DNA fragments ZA, AB and GZ, which contained an auxotrophic yeast selection marker (HIS3), an autonomously replicating sequence (ARS), a yeast centromere (CEN) and a 600 bp stuffier sequence (see Table No. 4 below). Expression of each cassette was driven by a yeast native promoter as described in Example 1 above. The DNA backbone fragments, as well as the gene expression cassettes were flanked by 60 bp homologous recombination tags (HRT), where each terminal tag was identical to the first tag of the following cassette. Each HRT cassette included terminal AscI restriction sites to allow excision from the vector backbone.

TABLE NO. 4
P35G Pathway Gene Cassettes.*

| Plasmid | | | SEQ ID |
|---|---|---|---|
| (pEVE) | Cassette | Content | NO |
| 4729 | ZA | HIS3, pSC101 | 38 |
| 1968 | AB | ARS/CEN, CmR | 39 |
| 4134 | BC | ANS Ph | 9 |
| 4005 | CD | A3GT At | 25 |
| 4015 | DE | F3H-1 Md | 3 |
| 4024 | EF | DFR Aa | 5 |
| 25163 | FG | A5GT Va | 45 |
| 1918 | GZ | 600 bp stuffer | 40 |

*Summary of the plasmids containing the cassettes included in the final HRT vector for P35G production in yeast.

Plasmids (from Table No. 4) containing the described DNA helper fragments and gene expression cassettes were digested with AscI in a 20 μL reaction volume. The digest was performed for 2 h at 37° C.

For transformation of a naringenin producing yeast strain (described in Example 1) with the HRT reaction, a 3 mL pre-culture of the naringenin producing strain was inoculated the day before transformation and used to inoculate a fresh yeast culture the following day which was transformed after 3-4 hours of growth. After transformation of the naringenin producing strain by the LiAC method (see e.g., Gietz et al., Nat Protoc. 2007; 2(1):35-7), cells were grown at 30° C. for 72 h.

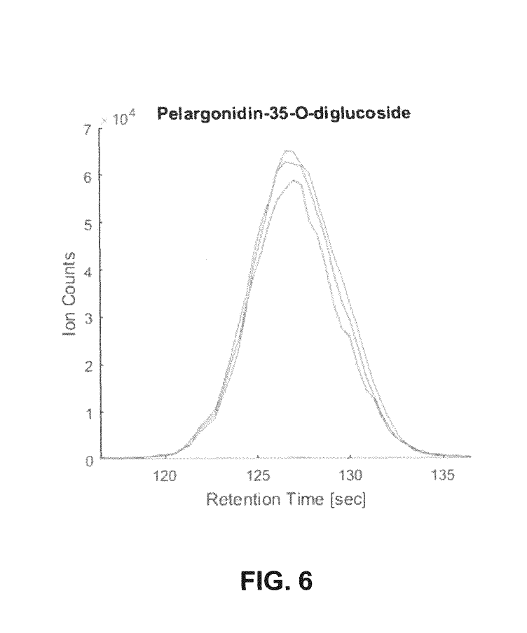

Individual yeast clones were subsequently grown in 2 mL liquid cultures for 96 hours, after which, the cultures were extracted with acidified Methanol (1% HCL) at 30° C., 300 rpm for 30 min. Following extraction, the cell debris was precipitated by centrifugation, and the supernatants were collected for analysis by LC/MS. Analysis demonstrated the presence of pelargonidin-3,5-O-glucoside (FIG. 6).

### Example 4: Production of Cyanidin-3-O-Glucoside (C3G) in Yeast

The cyanidin-3-O-glucoside (C3G)-pathway from naringenin was assembled in two steps including assembly of two HRT plasmids, as described below in reference to Table Nos. 5 and 6. In a first step a (+)-catechin (CAT)-producing strain was created by combining the genes listed in Table. No. 5. The CAT pathway was assembled on an HRT vector containing the genes F3′H from Petunia×hybrida, F3H-1 from Malus domestica, and a CPR (ATR1) from Arabidopsis thaliana cloned into yeast expression cassettes CD, DE, and GH, respectively. In addition, the expression cassettes EF and FG containing a DFR variant and a LAR variant, respectively, were included. The DNA fragment BC was empty, meaning no expression cassette was inserted between the HRTs. The plasmid backbone was formed by the DNA fragments ZA, AB, and HZ (see Table No. 5). The HRT reaction was performed as described above, but in a 50 μL reaction volume.

The naringenin producing strain (Example No. 1) was transformed with the HRT reaction. After transformation and growth of the cells for 72 h, clones were cultured in 96-well plates and screened for CAT production. A clone, with confirmed production of CAT was chosen for further engineering in a second step.

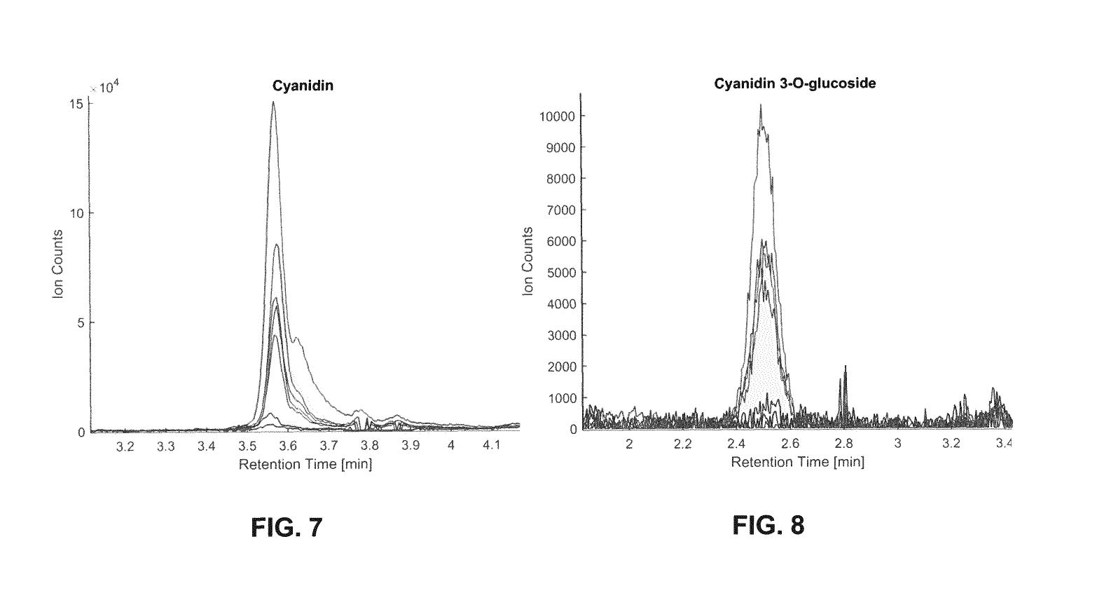

In the second step, a cyanidin-3-O-glucoside producing yeast strain was created from a combination of ANS and A3GT genes transformed into the CAT producing clone described above. The expression cassettes BC and CD of the second HRT vector contained one of eight tested ANS variants and one of eight tested A3GT variants, respectively. Note, that for the purpose of this example only one specific ANS and A3GT gene, respectively, are listed in Table No. 6. HRT reaction, transformation, and cell culture were performed as above. Clones were isolated and grown as described above, and analyzed for anthocyanin production. Several clones were shown to produce cyanidin (FIG. 7) and cyanidin-3-O-glucoside (FIG. 8). The highest concentrations were seen with the specific ANS and A3GT listed in Table No. 6.

TABLE NO. 5
Summary of a plasmid containing the cassettes included in a
HRT vector which exhibited (+)-catechin production in yeast.

| Plasmid | | | PI size | SEQ ID | PI amount |
|---|---|---|---|---|---|
| (pEVE) | Cassette | Content | (kb) | NO | (ng) |
| 1765 | ZA | LEU2, | 5.3 | 41 | 530 |
| | | pMB1 | | | |
| 1968 | AB | ARS/CEN, | 4.8 | 39 | 480 |
| | | CmR | | | |
| 2176 | BC | Empty BC | 4.7 | 46 | 705 |
| | | linker | | | |
| 3999 | CD | F3′H Ph | 5.6 | 27 | 840 |
| 4015 | DE | F3H-1 Md | 4.9 | 3 | 735 |
| 4026 | EF | DFR Pt | 5.2 | 7 | 97.5 |
| 4028 | FG | LAR-1 Fa | 5 | 29 | 250 |
| 3975 | GH | ATR-1 At | 6.5 | 31 | 975 |
| 1919 | HZ | 600 bp | 3.6 | 37 | 540 |
| | | stuffer | | | |

TABLE NO. 6
Summary of one plasmid containing the cassettes
included in the HRT vector for C3G production.

| Plasmid | | | PI size | SEQ ID | PI amount |
|---|---|---|---|---|---|
| (pEVE) | Cassette | Content | (kb) | NO | (ng) |
| 4729 | ZA | HIS3, | 6.3 | 38 | 1260 |
| | | pSC101 | | | |
| 1968 | AB | ARS/CEN, | 4.8 | 39 | 960 |
| | | CmR | | | |
| 4134 | BC | ANS Ph | 5.2 | 9 | 195 |
| 4438 | CD | A3GT Dc | 5.5 | 11 | 236 |
| 1915 | DZ | 600 bp stuffer | 3.6 | 42 | 1080 |

### Example No. 5: Production of Cyanidin-3,5-O-Diglucoside (C35G) in Yeast

The cyanidin-3,5-O-diglucoside (C35G) pathway was done in two steps including assembly of two HRT plasmids. In a first step, an eriodictyol strain was created from the naringenin strain (see Example No. 1 above) by the introduction and assembly of HRT expression fragments consisting of a flavonoid 3′-hydroxylase (F3′H) from Petunia hybrida and a cytochrome P450 reductase (CPR-1) gene from Arabidopsis thaliana, cloned into yeast expression cassettes CD and DE, respectively. The DNA fragment BC was empty, meaning no expression cassette was inserted between the HRTs. The plasmid backbone was formed by the DNA fragments ZA, AB, and EZ (see Table No. 7).

Plasmids containing the described helper fragments and gene expression cassettes were digested with AscI in a 20 μL reaction volume. The digest was performed for 2 h at 37° C.

The naringenin producing strain was transformed with the HRT reaction using the LiAC method (see e.g., Gietz et al., Nat Protoc. 2007; 2(1):35-7). After transformation, the cells were grown at 30° C. for 72 h.

Individual yeast clones were then grown in 2 mL liquid cultures for 96 h. Subsequently, the cultures were extracted with acidified methanol (1% HCL) at 30° C., 300 rpm for 30 min. Following extraction, the cell debris was precipitated by centrifugation, and the cleared supernatants were collected for analysis by LC/MS. Analysis showed that introduction of the listed genes (Table No. 7) resulted in the production of eriodictyol.

TABLE NO. 7
Eriodictyol Pathway Gene Cassettes.*

| Plasmid | | | SEQ ID |
|---|---|---|---|
| (pEVE) | Cassette | Content | NO |
| 4728 | ZA | LEU2, | 41 |
| | | pSC101 | |
| 1968 | AB | ARS/CEN, | 39 |
| | | CmR | |
| 2176 | BC | Empty BC | 46 |
| | | linker | |
| 3999 | CD | F3′H Ph | 27 |
| 4012 | DE | CPR-1 At | 48 |
| 1916 | EZ | 600 bp | 49 |
| | | stuffer | |

*Summary of the plasmids containing the cassettes included in the final HRT vector for eriodictyol production in yeast.

In the second step, a cyanidin-3,5-O-glucoside producing yeast strain was created from a combination of ANS, DFR, F3H, A3GT and A5GT genes transformed into the eriodictyol producing strain described above. Each yeast expression cassette BC, CD, DE and EF contained a gene encoding one enzyme of the C35G pathway. The BC cassette encoded an anthocyanidin synthase (ANS) from Petunia×hybrida, the CD cassette contained an anthocyanidin-3-O-glycosyl transferase (A3GT) from Arabidopsis thaliana, the DE cassette encoded a flavanone-3-hydroxylase (F3H) from Malus domestica, the EF cassette encoded a dihydroflavonol-4-reductase (DFR) from Anthurium andraeanum and the FG cassette contained an anthocyanin-5-O-glycosyl transferase (A5GT) from Vitis amurensis.

The backbone of the HRT vector was formed by the DNA helper fragments ZA, AB and GZ, which contained an auxotrophic yeast selection marker (HIS3), an autonomously replicating sequence (ARS), a yeast centromere (CEN) and a 600 bp stuffer sequence (see Table No. 8 below). Expression of each cassette was driven by a yeast native promoter. The DNA helper fragments, as well as the gene expression cassettes were flanked by 60 bp homologous recombination tags (HRT), where each terminal tag was identical to the first tag of the following cassette. Each HRT cassette included terminal AscI restriction sites to allow excision from the vector backbone.

TABLE NO. 8
C35G Pathway Gene Cassettes.*

| Plasmid | | | SEQ ID |
|---|---|---|---|
| (pEVE) | Cassette | Content | NO |
| 4729 | ZA | HIS, pSC101 | 38 |
| 1968 | AB | ARS/CEN, | 39 |
| | | CmR | |
| 4134 | BC | ANS Ph | 9 |
| 4005 | CD | A3GT At | 25 |
| 4015 | DE | F3H-1 Md | 3 |
| 4024 | EF | DFR Aa | 5 |
| 25163 | FG | A5GT Va | 45 |
| 1918 | GZ | 600 bp stuffer | |

*Summary of the plasmids containing the cassettes included in the final HRT vector for C35G production in yeast.

Plasmids containing the described helper fragments and gene expression cassettes were digested with AscI in a 20 μL reaction volume. The digest was performed for 2 h at 37° C.

The eriodictyol producing yeast strain was transformed with the HRT digest reaction using the LiAC method (see e.g., Gietz et al., Nat Protoc. 2007; 2(1):35-7). After transformation, the cells were grown at 30° C. for 72 h.

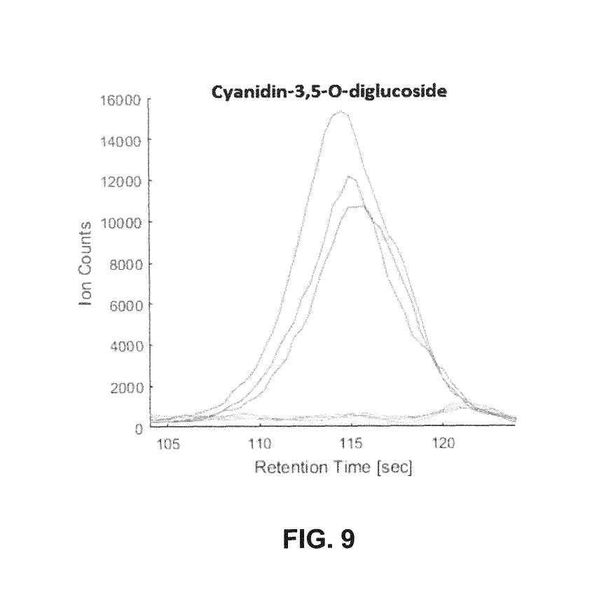

Individual yeast clones were then grown in 2 mL liquid cultures for 96 h. Subsequently, the cultures were extracted with acidified methanol (1% HCL) at 30° C., 300 rpm for 30 min. Following extraction, the cell debris was precipitated by centrifugation, and the cleared supernatants were collected for analysis by LC/MS. The analysis demonstrated the presence of cyanidin-3,5-O-glucoside (FIG. 9).

### Example No. 6: Production of Delphinidin and Delphinidin-3-O-Glucoside (D3G) in Yeast

The delphinidin-3-O-glucoside (D3G) pathway was done in two steps including assembly of two HRT plasmids. In a first step, a 5,7,3′,4′,5′ pentahydroxyflavone (PHF) strain was created from the naringenin strain (see Example No. 1 above) by the introduction and assembly of HRT expression fragments consisting of a flavonoid-3′5′-hydroxylase gene (F3′5′H) from Solanum lycopersicum and a cytochrome P450 reductase (CPR-1) gene from Arabidopsis thaliana, cloned into HRT yeast expression cassettes CD and DE, respectively. The DNA fragment BC was empty, meaning no expression cassette was inserted between the HRTs. The plasmid backbone was formed by the DNA fragments ZA, AB, and EZ, which contained an auxotrophic yeast selection marker (LEU2), an autonomously replicating sequence (ARS), a yeast centromere (CEN) and a 600 bp stuffer sequence (see Table No. 9). Expression of each cassette was driven by a yeast native promoter as described in Example No. 1. The DNA backbone fragments, as well as the gene expression cassettes were flanked by 60 bp homologous recombination tags (HRT). Each HRT cassette included terminal AscI restriction sites to allow excision from the vector backbone.

TABLE NO. 9
PHF Pathway Gene Cassettes.

| Plasmid | | | SEQ ID |
|---|---|---|---|
| (pEVE) | Cassette | Content | NO |
| 4728 | ZA | LEU2, pSC101 | 41 |
| 1968 | AB | ARS/CEN, | 39 |
| | | CmR | |
| 2176 | BC | Empty BC | 46 |
| | | linker | |
| 24070 | CD | F3′5′H SI | 47 |
| 4012 | DE | CPR-1 At | 48 |
| 1916 | EZ | 600 bp stuffer | 49 |

*Summary of the plasmids containing the cassettes included in the final HRT vector for PHF production in yeast.

Plasmids containing the described helper fragments and gene expression cassettes were digested with AscI in a 20 μL reaction volume. The digest was performed for 2 h at 37° C.

The naringenin producing yeast strain was transformed with the HRT digest reaction using the LiAC method (see e.g., Gietz et al., Nat Protoc. 2007; 2(1):35-7). After transformation, the cells were grown at 30° C. for 72 h.

Individual yeast clones were then grown in 2 mL liquid cultures for 96 h. Subsequently, the cultures were extracted with acidified methanol (1% HCL) at 30° C., 300 rpm for 30 min. Following extraction, the cell debris was precipitated by centrifugation, and the cleared supernatants were collected for analysis by LC/MS and production of PHF was confirmed.

In the second step, a delphinidin-3-O-glucoside producing yeast strain was created from a combination of ANS, DFR, F3H and A3GT genes transformed into the PHF producing strain described above. Each yeast expression cassette BC, CD, DE and EF contained a gene encoding one enzyme of the D3G pathway. The BC cassette encoded an anthocyanidin synthase (ANS) from Petunia×hybrida, the CD cassette contained an anthocyanidin-3-O-glycosyl transferase (A3GT) from Arabidopsis thaliana, the DE cassette encoded a flavanone-3-hydroxylase (F3H) from Malus domestica, the EF cassette encoded a dihydroflavonol-4-reductase (DFR) from Anthurium andraeanum.

The backbone of the HRT vector was formed by the DNA helper fragments ZA, AB and FZ, which contained an auxotrophic yeast selection marker (HIS3), an autonomously replicating sequence (ARS), a yeast centromere (CEN) and a 600 bp stuffer sequence (see Table No. 10 below). Expression of each cassette was driven by a yeast native promoter. The DNA helper fragments, as well as the gene expression cassettes were flanked by 60 bp homologous recombination tags (HRT), where each terminal tag was identical to the first tag of the following cassette. Each HRT cassette included terminal AscI restriction sites to allow excision from the vector backbone.

TABLE NO. 10
D3G Pathway Gene Cassettes.*

| Plasmid | | | SEQ ID |
|---|---|---|---|
| (pEVE) | Cassette | Content | NO |
| 4729 | ZA | HIS3, pSC101 | 38 |
| 1968 | AB | ARS/CEN, CmR | 39 |
| 4134 | BC | ANS Ph | 9 |
| 4005 | CD | A3GT At | 25 |
| 4015 | DE | F3H-1 Md | 3 |
| 4024 | EF | DFR Aa | 5 |
| 1917 | FZ | 600 bp stuffer | 40 |

*Summary of the plasmids containing the cassettes included in the final HRT vector for D3G production in yeast.

Plasmids containing the described helper fragments and gene expression cassettes were digested with AscI in a 20 μL reaction volume. The digest was performed for 2 h at 37° C.

Yeast was transformed with the HRT digest reaction using the LiAC method (see e.g., Gietz et al., Nat Protoc. 2007; 2(1):35-7). After transformation, the cells were grown at 30° C. for 72 h.

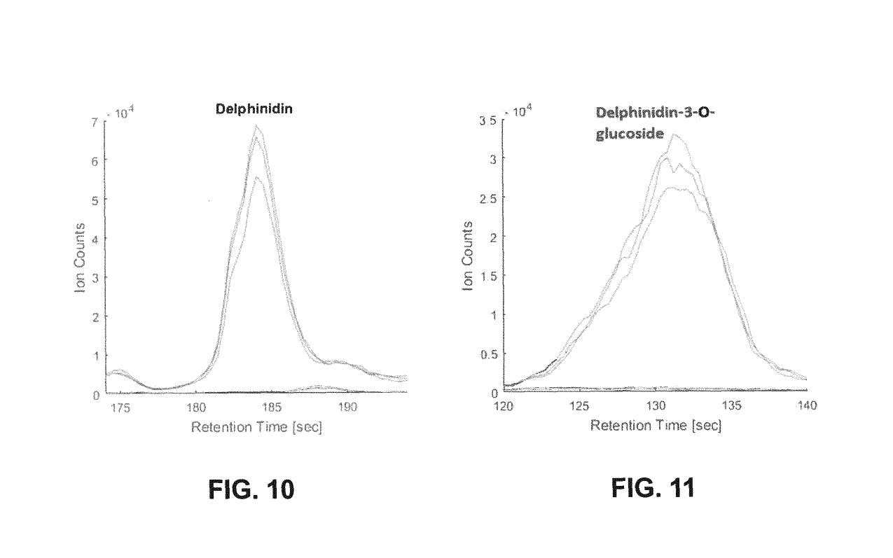

Individual yeast clones were then grown in 2 mL liquid cultures for 96 h. Subsequently, the cultures were extracted with acidified methanol (1% HCL) at 30° C., 300 rpm for 30 min. Following extraction, the cell debris was precipitated by centrifugation, and the cleared supernatants were collected for analysis by LC/MS. Analysis showed that introduction of the listed genes (Table No. 10) resulted in the production of delphinidin (see FIG. 10) and delphinidin-3-O-glucoside (see FIG. 11).

### Example No. 7: Production of Delphinidin-3,5-O-Diglucoside (D35G) in Yeast

The delphinidin-3,5-O-diglucoside (D35G) pathway was assembled in the 5,7,3′,4′,5′ pentahydroxyflavone (PHF) strain described in Example No. 6 above. Specifically, a delphinidin-3,5-O-diglucoside producing yeast strain was created from a combination of ANS, DFR, F3H, A3GT, and A5GT genes transformed into the PHF producing strain. Each yeast expression cassette BC, CD, DE and EF contained a gene encoding one enzyme of the D35G pathway. The BC cassette encoded an anthocyanidin synthase (ANS) from Petunia×hybrida, the CD cassette contained an anthocyanidin-3-O-glycosyl transferase (A3GT) from Arabidopsis thaliana, the DE cassette encoded a flavanone-3-hydroxylase (F3H) from Malus domestica, the EF cassette encoded a dihydroflavonol-4-reductase (DFR) from Anthurium andraeanum and the FG cassette contained an anthocyanin-5-O-glycosyl transferase (A5GT) from Vitis amurensis.

The backbone of the HRT vector was formed by the DNA helper fragments ZA, AB and GZ, which contained an auxotrophic yeast selection marker (HIS3), an autonomously replicating sequence (ARS), a yeast centromere (CEN) and a 600 bp stuffer sequence (see Table No. 11 below). Expression of each cassette was driven by a yeast native promoter. The DNA helper fragments, as well as the gene expression cassettes were flanked by 60 bp homologous recombination tags (HRT), where each terminal tag was identical to the first tag of the following cassette. Each HRT cassette included terminal AscI restriction sites to allow excision from the vector backbone.

TABLE NO. 11
D35G Pathway Gene Cassettes.*

| Plasmid | | | SEQ ID |
|---|---|---|---|
| (pEVE) | Cassette | Content | NO |
| 4729 | ZA | HIS3, pSC101 | 38 |
| 1968 | AB | ARS/CEN, CmR | 39 |
| 4134 | BC | ANS Ph | 9 |
| 4005 | CD | A3GT At | 25 |
| 4015 | DE | F3H-1 Md | 3 |
| 4024 | EF | DFR Aa | 5 |
| 25163 | FG | A5GT Va | 45 |
| 1918 | GZ | 600 bp stuffer | 53 |

*Summary of the plasmids containing the cassettes included in the final HRT vector for D35G production in yeast.

Plasmids containing the described helper fragments and gene expression cassettes were digested with AscI in a 20 μL reaction volume. The digest was performed for 2 h at 37° C.

The PHF producing yeast strain was transformed with the HRT digest reaction using the LiAC method (see e.g., Gietz et al., Nat Protoc. 2007; 2(1):35-7). After transformation, cells were grown at 30° C. for 72 h.

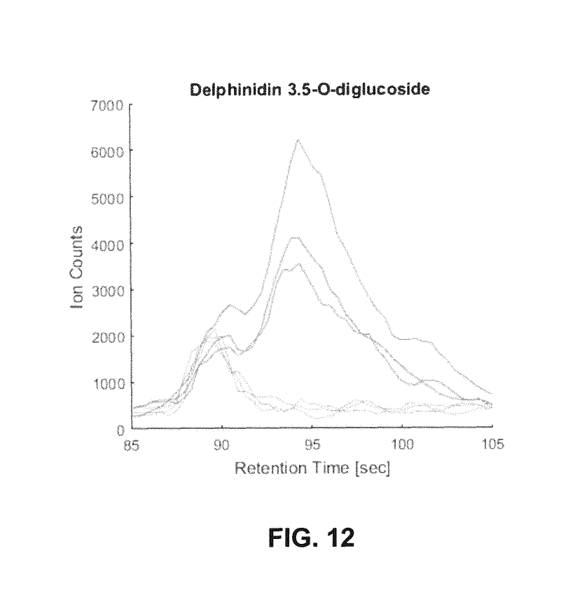

Individual yeast clones were then grown in 2 mL liquid cultures for 96 h. Subsequently, the cultures were extracted with acidified methanol (1% HCL) at 30° C., 300 rpm for 30 min. Following extraction, the cell debris was precipitated by centrifugation, and the cleared supernatants were collected for analysis by LC/MS. Analysis showed that introduction of the listed genes of Table No. 11 resulted in the production of delphinidin-3,5-O-diglucoside (FIG. 12).

### Example No. 8: Production of Pelargonidin-3-O-Coumaroyl-Glucoside (P3CG) and Pelargonidin-3-O-Coumaroyl Glucoside-5-O-Glucoside (P35CG) in Yeast

The assembly of the P3CG and P35CG pathways were done in the pelargonidin-3-O-glucoside and pelargonidin-3,5-O-diglucoside producing strains, respectively. The gene for an anthocyanin 3-O-glucoside:6″-O-p-coumaroyl transferase (A3AAT) from Arabidopsis thaliana, which had been codon-optimized for expression in yeast and manufactured by GeneArt AG (Regensburg, Germany), was introduced on a plasmid using the HRT technology. Table No. 12 lists the gene cassettes that were used for pathway assembly.

The DNA fragment CD was empty, meaning no expression cassette was inserted between the HRTs. The plasmid backbone was formed by the DNA fragments ZA, AB, and DZ which contained an auxotrophic yeast selection marker (LEU2), an autonomously replicating sequence (ARS), a yeast centromere (CEN) and a 600 bp stuffier sequence (see Table No. 12).

TABLE NO. 12
P3CG and P35CG Pathway Gene Cassettes.*

| Plasmid | | | SEQ ID |
|---|---|---|---|
| (pEVE) | Cassette | Content | NO |
| 4728 | ZA | LEU2, pSC101 | 41 |
| 1968 | AB | ARS/CEN, CmR | 39 |
| 27294 | BC | A3AAT | 51 |
| 2177 | CD | empty | 50 |
| 1915 | DZ | 600 bp stuffer | 42 |

*Summary of the plasmids containing the cassettes included in the final HRT vector for P3CG and P35CG production in yeast.

Plasmids containing the described helper fragments and gene expression cassettes were digested with AscI in a 20 μL reaction volume. The digest was performed for 2 h at 37° C.

The two yeast strains producing P3G and P35G, respectively, were transformed separately with the digested HRT fragments using the LiAC transformation method (see e.g., Gietz et al., Nat Protoc. 2007; 2(1):35-7). After transformation, the cells were grown at 30° C. for 72 h.

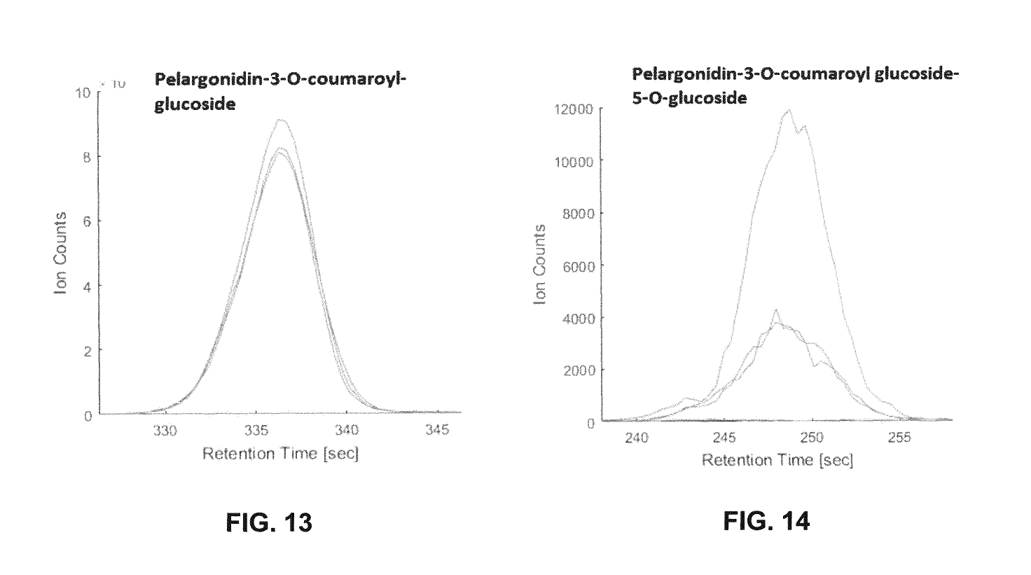

Individual yeast clones from both transformations were then grown in 2 mL liquid cultures for 96 h. Subsequently, the cultures were extracted with acidified methanol (1% HCL) at 30° C., 300 rpm for 30 min. Following extraction, the cell debris was precipitated by centrifugation, and the cleared supernatants were collected for analysis by LC/MS. Analysis showed that introduction of the gene encoding the anthocyanin 3-O-glucoside:6″-O-p-coumaroyl transferase resulted in the production of pelargonidin-3-O-coumaroyl glucoside (FIG. 13) and pelargonidin-3-O-coumaroyl glucoside-5-O-glucoside (FIG. 14).

### Example No. 9: Production of Pelargonidin-3-O-Malonyl Glucoside (P3MG) and Pelargonidin-3-O-Malonyl Glucoside-5-O-Glucoside (P35MG) in Yeast

The assembly of the P3MG and P35MG pathways were done in the pelargonidin-3-O-glucoside and pelargonidin-3,5-O-diglucoside producing strains, respectively. The gene encoding an anthocyanin 3-O-glucoside:6″-O-malonyl transferase (A3MAT) from Dahlia variabilis, which had been codon-optimized for expression in yeast and manufactured by GeneArt AG (Regensburg, Germany), was introduced on a plasmid using the HRT technology. Table No. 13 lists the gene cassettes that were used for pathway assembly.

The DNA fragment CD was empty, meaning no expression cassette was inserted between the HRTs. The plasmid backbone was formed by the DNA fragments ZA, AB, and DZ which contained an auxotrophic yeast selection marker (LEU2), an autonomously replicating sequence (ARS), a yeast centromere (CEN) and a 600 bp stuffer sequence (see Table No. 13).

TABLE NO. 13
P3MG and M35MG Pathway Gene Cassettes*

| Plasmid | | | SEQ ID |
|---|---|---|---|
| (pEVE) | Cassette | Content | NO |
| 4728 | ZA | LEU2, pSC101 | 41 |
| 1968 | AB | ARS/CEN, CmR | 39 |
| 27296 | BC | A3MAT | 52 |
| 2177 | CD | empty | 50 |
| 1915 | DZ | 600 bp stuffer | 42 |

Plasmids containing the described helper fragments and gene expression cassettes were digested with AscI in a 20 μL reaction volume. The digest was performed for 2 h at 37° C.

The two yeast strains producing P3G and P35G, respectively, were transformed separately with the digested HRT fragments using the LiAC transformation method (see e.g., Gietz et al., Nat Protoc. 2007; 2(1):35-7). After transformation, the cells were grown at 30° C. for 72 h.

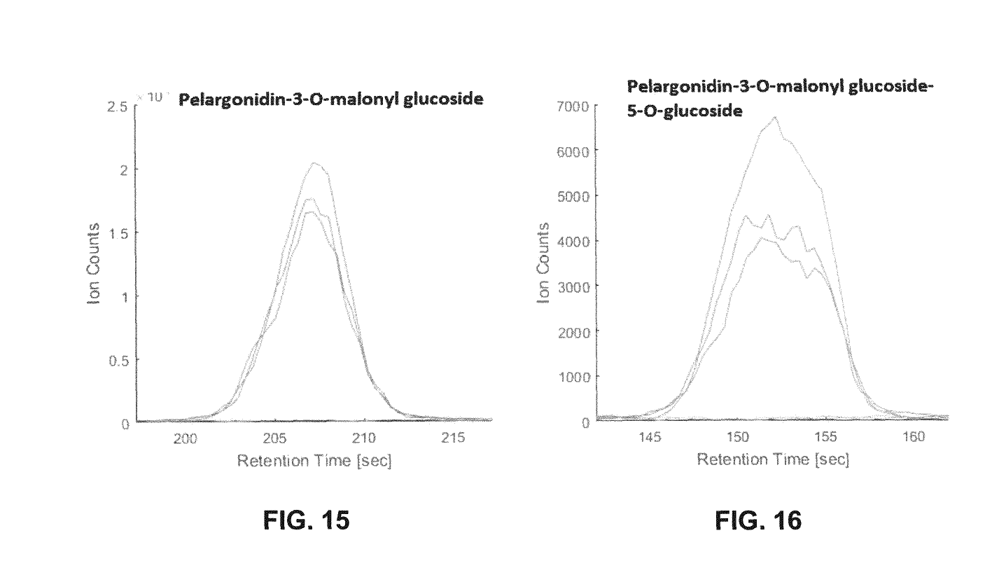

Individual yeast clones from both transformations were then grown in 2 mL liquid cultures for 96 h. Subsequently, the cultures were extracted with acidified methanol (1% HCL) at 30° C., 300 rpm for 30 min. Following extraction, the cell debris was precipitated by centrifugation, and the cleared supernatants were collected for analysis by LC/MS. Analysis showed that introduction of the gene encoding the anthocyanin 3-O-glucoside:6″-O-malonyl transferase resulted in the production of pelargonidin-3-O-malonyl glucoside (see FIG. 15) and pelargonidin-3-O-malonyl glucoside-5-O-glucoside (see FIG. 16).

### Example No. 10: Analysis of Flavonoids and Flavonoid Derivatives

LC Parameters

Flavonoids and derivatives were analyzed using liquid-chromatography coupled to mass spectrometry (LC/MS). An HSS T3 column, 130 Å, 1.7 μm, 2.1 mm×100 mm was employed using the conditions indicated in Table No. 14 below. A=0.1% formic acid, B=acetonitrile with 0.1% formic acid.

TABLE NO. 14
Chromatographic gradient for LCMS analysis
of flavonoids and flavonoid-derivatives.

| Time (min) | Flow (mL/min) | % A | % B |
|---|---|---|---|
| initial | 0.400 | 95.0 | 5.0 |
| 3.00 | 0.400 | 80.0 | 20.0 |
| 4.30 | 0.400 | 80.0 | 20.0 |
| 9.00 | 0.400 | 55.0 | 45.0 |
| 11.00 | 0.400 | 0.0 | 100.0 |
| 13.00 | 0.400 | 0.0 | 100.0 |
| 13.01 | 0.400 | 95.0 | 5.0 |
| 15.00 | 0.400 | 95.0 | 5.0 |

MS Parameters

For mass spectrum analysis, full scan spectrum data were recorded using a Xevo® G2-XS (Waters Cooperation, Milford, USA) with the parameters indicated in Table No. 15 below.

TABLE NO. 15
Mass spectrometry parameters.

| Source Parameter | Value | |
|---|---|---|
| Ion Source | Electrospray Positive Mode (ESI−) | |
| Capillary Voltage | 2.0 | kV |
| Sampling Cone | 40 | V |
| Source Offset | 80 | V |
| Source Temperature | 150° | C. |
| Desolvation Temperature | 500° | C. |
| Cone gas flow | 100 | L/h |
| Desolvation gas flow | 1000 | L/h |

| | |
|---|---|
| Mass Range | From 50 to 1200 m/z |
| Lock Mass | Leucin Enkephalin (ESI+) |

Data Processing and Quantification

For each compound, an extracted ion chromatogram within a mass window of 0.01 Da was calculated. Peak areas and compound quantities were calculated according to the retention time and linear calibration curve of the respective standard compounds (Sigma-Aldrich, Switzerland) (see Table No. 16 below).

TABLE NO. 16
Mass spectrometry standards

| Compound | Retention Time [min] |
|---|---|
| Cyanidin | 3.7 |
| Cyanidin-3-glucoside | 2.6 |
| Cyanidin-3,5-diglucoside | 1.9 |
| Pelargonidin | 4.2 |
| Pelargonidin-3-glucoside | 2.9 |
| Pelargonidin-3,5-diglucoside | 2.2 |
| Delphinidin | 3.1 |
| Delphinidin-3-glucoside | 2.3 |
| Delphinidin 3,5-diglucoside | 1.6 |

### Example No. 11: Characterization of Isolated Anthocyanins

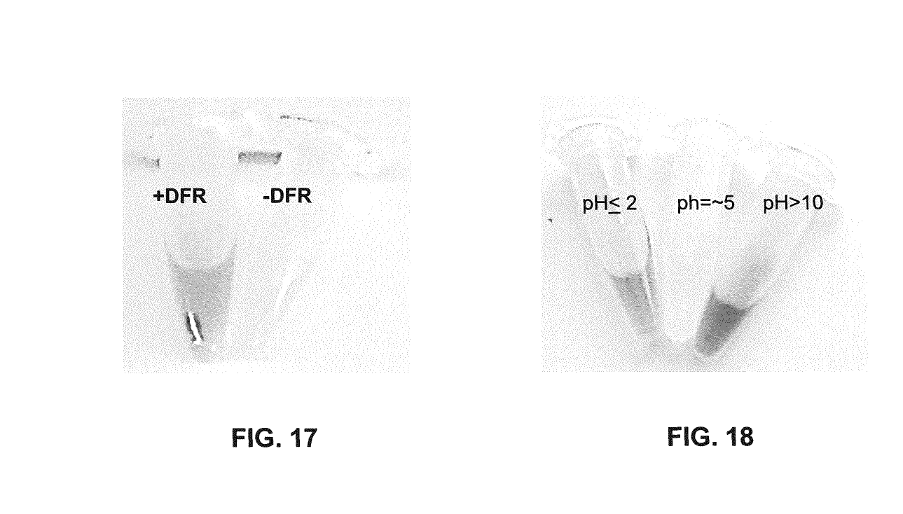

A yeast strain was constructed as described in Example No. 2, but leaving out the DFR gene. This strain was used as negative control for P3G production. After culturing this strain and the strain from Example No. 2, the broth was acidified with HCl to pH<2 and visually inspected. As seen in FIG. 17, the development of color, corresponding to the presence of P3G, was only achieved when DFR was included in the strain. The control strain without DFR did not produce any color. This shows that the compound(s) giving rise to the color is downstream from dihydroflavonols, in this case the dihydrokaempferol, and is consistent with the detection of P3G in this strain.

Further, the P3G-producing strain from Example No. 2 was grown, as described, and the broth was adjusted to various pH values: pH<2, pH=5, and pH>10. As seen in FIG. 18, the color observed at the different pH corresponds to the expected pH-dependent color changes, as reported in literature for P3G.

Having described the invention in detail and by reference to specific embodiments thereof, it will be apparent that modifications and variations are possible without departing from the scope of the invention defined in the appended claims. More specifically, although some aspects of the present invention are identified herein as particularly advantageous, it is contemplated that the present invention is not necessarily limited to these particular aspects of the invention.

Sequence IDs of genes/enzymes used in Examples.

| | |
|---|---|
| SEQ ID NO: 1 | DNA sequence encoding 4-coumarate- |
| | CoA ligase 2 (4CL2) of Arabidopsis |
| | thaliana |
| SEQ ID NO: 2 | Protein sequence of 4CL2 of Arabidopsis |
| | thaliana |
| SEQ ID NO: 3 | DNA sequence encoding F3H-1 of |
| | Malus domestica (pEVE 4015) |
| SEQ ID NO: 4 | Protein sequence of F3H-1 of Malus |
| | domestica |
| SEQ ID NO: 5 | DNA sequence encoding DFR of |
| | Anthurium andraeanum (pEVE 4024) |
| SEQ ID NO: 6 | Protein sequence of DFR of Anthurium |
| | andreanum |
| SEQ ID NO: 7 | DNA sequence encoding DFR of |
| | Populus trichocarpa (pEVE 4026) |
| SEQ ID NO: 8 | Protein sequence of DFR of Populus |
| | trichocarpa |
| SEQ ID NO: 9 | DNA sequence encoding ANS of Petunia |
| | x hybrida (pEVE 4134) |
| SEQ ID NO: 10 | Protein sequence of ANS of Petunia x |
| | hybrida |
| SEQ ID NO: 11 | DNA sequence encoding A3GT of |
| | Dianthus caryophyllus |
| SEQ ID NO: 12 | Protein sequence of A3GT of Dianthus |
| | caryophyllus |
| SEQ ID NO: 13 | DNA sequence encoding chalcone |
| | isomerase (CHI) of Medicago sativa |
| SEQ ID NO: 14 | Protein sequence of CHI of Medicago |
| | sativa |
| SEQ ID NO: 15 | DNA sequence encoding tyrosine |
| | ammonia lyase (TAL) of Zea mays |
| SEQ ID NO: 16 | Protein sequence of tyrosine ammonia |
| | lyase (TAL) of Zea mays |
| SEQ ID NO: 17 | DNA sequence encoding phenylalanine |
| | ammonia lyase (PAL2) of Arabidopsis |
| | thaliana |
| SEQ ID NO: 18 | Protein sequence of PAL2 of |
| | Arabidopsis thaliana |
| SEQ ID NO: 19 | DNA sequence encoding cinnamate 4- |
| | hydroxylase (C4H) of Ammi majus |
| SEQ ID NO: 20 | Protein sequence of C4H of Ammi majus |
| SEQ ID NO: 21 | DNA sequence encoding chalcone |
| | synthase (CHS2) of Hordeum vulgare |
| SEQ ID NO: 22 | Protein sequence of CHS2 of Hordeum |
| | vulgare |
| SEQ ID NO: 23 | DNA sequence encoding cytochrome |
| | p450 CPR1 (Ncp1) of Saccharomyces |
| | cerevisiae |
| SEQ ID NO: 24 | Protein sequence of CPR1 of |
| | Saccharomyces cerevisiae |
| SEQ ID NO: 25 | DNA sequence encoding A3GT of |
| | Arabidopsis thaliana (pEVE 4005) |
| SEQ ID NO: 26 | Protein sequence of A3GT of |
| | Arabidopsis thaliana |
| SEQ ID NO: 27 | DNA sequence encoding F3′H of |
| | Petunia x hybrida (pEVE 3999) |
| SEQ ID NO: 28 | Protein sequence of F3′H of |
| | Petunia x hybrida |
| SEQ ID NO: 29 | DNA sequence encoding LAR-1 of |
| | Fragaria x ananassa (pEVE 4028) |
| SEQ ID NO: 30 | Protein sequence of LAR-1 of |
| | Fragaria x ananassa |
| SEQ ID NO: 31 | DNA sequence encoding ATR-1 of |
| | Arabidopsis thaliana (pEVE 3975) |
| SEQ ID NO: 32 | Protein sequence of ATR-1 of |
| | Arabidopsis thaliana |
| SEQ ID NO: 33 | DNA sequence encoding F3′5′H of Viola |
| | tricolor |
| SEQ ID NO: 34 | Protein sequence of F3′5′H of Viola |
| | tricolor |
| SEQ ID NO: 35 | DNA sequence of pEVE4745-ZA for |
| | HRT integration into XI-3 site |
| SEQ ID NO: 36 | DNA sequence of pEVE3169-AB with |
| | URA3 marker flanked by LoxP sites |
| SEQ ID NO: 37 | DNA sequence of pEVE1919-Closing |
| | linker HZ for 6 gene plasmid or |
| | integration |
| SEQ ID NO: 38 | DNA sequence of pEVE4729-ZA with |
| | HIS3 marker and pSC101 ORI for HRT |
| | plasmids |
| SEQ ID NO: 39 | DNA sequence of pEVE1968-AB with |
| | ARS/CEN origin and CmR marker for |
| | HRT plasmids |
| SEQ ID NO: 40 | DNA sequence of pEVE1917-Closing |
| | linker FZ for 4 gene HRT plasmid |
| SEQ ID NO: 41 | DNA sequence of pEVE-1765-ZA with |
| | LEU2 marker and pMB1 ORI for HRT |
| | plasmids |
| SEQ ID NO: 42 | DNA sequence of pEVE1915-Closing |
| | linker DZ for 2 gene HRT plasmid |
| SEQ ID NO: 43 | DNA sequence of 5′-end including |
| | HindIII restriction site and Kozak |
| | sequence |
| SEQ ID NO: 44 | DNA sequence of 3′-end including a |
| | SacII recognition site. |
| SEQ ID NO: 45 | DNA sequence encoding anthocyanin-5- |
| | O-glycosyl transferase from Vitis |
| | amurensis |
| SEQ ID NO: 46 | DNA sequence of pEVE2176-empty |
| | HRT plasmid with BC tags |
| SEQ ID NO: 47 | DNA sequence encoding flavonoid-3′5′- |
| | hydroxylase from Solanum lycopersicum |
| SEQ ID NO: 48 | DNA sequence encoding cytochrome |
| | P450 reductase (ATR1) from |
| | Arabidopsis thaliana |
| SEQ ID NO: 49 | DNA sequence of pEVE191-Closing |
| | linker EZ for 3 gene HRT plasmid |
| SEQ ID NO: 50 | DNA sequence of pEVE2177-empty |
| | HRT plasmid with CD tags |
| SEQ ID NO: 51 | DNA sequence encoding anthocyanin |
| | 3-O-glucoside: 6″-O-p- |
| | coumaroyltransferase, Arabidopsis |
| | thaliana |
| SEQ ID NO: 52 | DNA sequence encoding anthocyanin 3- |
| | O-glucoside-6″-O-malonyltransferase, |
| | Dahlia variabilis |
| SEQ ID NO: 53 | DNA sequence of pEVE1918-Closing |
| | linker GZ for 5 gene plasmid |
| SEQ ID NO: 54 | Protein sequence of anthocyanin-5-O- |
| | glycosyl transferase of Vitis amurensis |
| SEQ ID NO: 55 | Protein sequence of flavonoid-3′5′- |
| | hydroxylase of Solanum lycopersicum |
| SEQ ID NO: 56 | Protein sequence of cytochrome P450 |
| | reductase (ATR1) from Arabidopsis |
| | thaliana |
| SEQ ID NO: 57 | Protein sequence of anthocyanin 3-O- |
| | glucoside: 6″-O-p-coumaroyltransferase |
| | of Arabidopsis thaliana |
| SEQ ID NO: 58 | Protein sequence of anthocyanin 3-O- |
| | glucoside-6″-O malonyltransferase of |
| | Dahlia variabilis |
| SEQ ID NO: 1 | ATGACGACACAAGATGTGATAGTCAATGATCAGAATGATCAGAAACAGT |
| | GTAGTAATGACGTCATTTTCCGATCGAGATTGCCTGATATATACATCCCT |
| | AACCACCTCCCACTCCACGACTACATCTTCGAAAATATCTCAGAGTTCG |
| | CCGCTAAGCCATGCTTGATCAACGGTCCCACCGGCGAAGTATACACCT |
| | ACGCCGATGTCCACGTAACATCTCGGAAACTCGCCGCCGGTCTTCATAA |
| | CCTCGGCGTGAAGCAACACGACGTTGTAATGATCCTCCTCCCGAACTCT |
| | CCTGAAGTAGTCCTCACTTTCCTTGCCGCCTCCTTCATCGGCGCAATCA |
| | CCACCTCCGCGAACCCGTTCTTCACTCCGGCGGAGATTTCTAAACAAGC |
| | CAAAGCCTCCGCGGCGAAACTCATCGTCACTCAATCCCGTTACGTCGAT |
| | AAAATCAAGAACCTCCAAAACGACGGCGTTTTGATCGTCACCACCGACT |
| | CCGACGCCATCCCCGAAAACTGCCTCCGTTTCTCCGAGTTAACTCAGTC |
| | CGAAGAACCACGAGTGGACTCAATACCGGAGAAGATTTCGCCAGAAGA |
| | CGTCGTGGCGCTTCCTTTCTCATCCGGCACGACGGGTCTCCCCAAAGG |
| | AGTGATGCTAACACACAAAGGTCTAGTCACGAGCGTGGCGCAGCAAGT |
| | CGACGGCGAGAATCCGAATCTTTACTTCAACAGAGACGACGTGATCCTC |
| | TGTGTCTTGCCTATGTTCCATATATACGCTCTCAACTCCATCATGCTCTG |
| | TAGTCTCAGAGTTGGTGCCACGATCTTGATAATGCCTAAGTTCGAAATC |
| | ACTCTCTTGTTAGAGCAGATACAAAGGTGTAAAGTCACGGTGGCTATGG |
| | TCGTGCCACCGATCGTTTTAGCTATCGCGAAGTCGCCGGAGACGGAGA |
| | AGTATGATCTGAGCTCGGTTAGGATGGTTAAGTCTGGAGCAGCTCCTCT |
| | TGGTAAGGAGCTTGAAGATGCTATTAGTGCTAAGTTTCCTAACGCCAAG |
| | CTTGGTCAGGGCTATGGGATGACAGAAGCAGGTCCGGTGCTAGCAATG |
| | TCGTTAGGGTTTGCTAAAGAGCCGTTTCCAGTGAAGTCAGGAGCATGTG |
| | GTACGGTGGTGAGGAACGCCGAGATGAAGATACTTGATCCAGACACAG |
| | GAGATTCTTTGCCTAGGAACAAACCCGGCGAAATATGCATCCGTGGCAA |
| | CCAAATCATGAAAGGCTATCTCAATGACCCCTTGGCCACGGCATCGACG |
| | ATCGATAAAGATGGTTGGCTTCACACTGGAGACGTCGGATTTATCGATG |
| | ATGACGACGAGCTTTTCATTGTGGATAGATTGAAAGAACTCATCAAGTA |
| | CAAAGGATTTCAAGTGGCTCCAGCTGAGCTAGAGTCTCTCCTCATAGGT |
| | CATCCAGAAATCAATGATGTTGCTGTCGTCGCCATGAAGGAAGAAGATG |
| | CTGGTGAGGTTCCTGTTGCGTTTGTGGTGAGATCGAAAGATTCAAATAT |
| | ATCCGAAGATGAAATCAAGCAATTCGTGTCAAAACAGGTTGTGTTTTATA |
| | AGAGAATCAACAAAGTGTTCTTCACTGACTCTATTCCTAAAGCTCCATCA |
| | GGGAAGATATTGAGGAAGGATCTAAGAGCAAGACTAGCAAATGGATTAA |
| | TGAACTAG |
| SEQ ID NO: 2 | MTTQDVIVNDQNDQKQCSNDVIFRSRLPDIYIPNHLPLHDYIFENISEFAAKP |
| | CLINGPTGEVYTYADVHVTSRKLAAGLHNLGVKQHDVVMILLPNSPEVVLTF |
| | LAASFIGAITTSANPFFTPAEISKQAKASAAKLIVTQSRYVDKIKNLQNDGVLI |
| | VITDSDAIPENCLRFSELTQSEEPRVDSIPEKISPEDVVALPFSSGTTGLPK |
| | GVMLTHKGLVTSVAQQVDGENPNLYFNRDDVILCVLPMFHIYALNSIMLCSL |
| | RVGATILIMPKFEITLLLEQIQRCKVTVAMVVPPIVLAIAKSPETEKYDLSSVR |
| | MVKSGAAPLGKELEDAISAKFPNAKLGQGYGMTEAGPVLAMSLGFAKEPF |
| | PVKSGACGTVVRNAEMKILDPDTGDSLPRNKPGEICIRGNQIMKGYLNDPL |
| | ATASTIDKDGWLHTGDVGFIDDDDELFIVDRLKELIKYKGFQVAPAELESLLI |
| | GHPEINDVAVVAMKEEDAGEVPVAFVVRSKDSNISEDEIKQFVSKQVVFYK |
| | RINKVFFTDSIPKAPSGKILRKDLRARLANGLMN |
| SEQ ID NO: 3 | ATGGCTCCAGCCACTACCTTAACCTCTATTGCACATGAAAAGACATTACA |
| | GCAGAAGTTCGTTAGAGATGAGGATGAAAGGCCTAAGGTTGCCTATAAC |
| | GACTTTTCTAATGAAATTCCAATAATCTCTTTGGCTGGTATAGACGAAGT |
| | AGAAGGTAGAAGGGGAGAAATATGTAAGAAGATTGTTGCAGCTTGCGAA |
| | GATTGGGGCATTTTCCAGATCGTAGACCATGGTGTAGATGCCGAATTGA |
| | TATCAGAAATGACAGGTTTGGCTAGAGAATTCTTCGCATTGCCTTCAGA |
| | AGAGAAGTTAAGGTTTGATATGTCCGGTGGTAAGAAAGGTGGTTTTATA |
| | GTCTCTAGTCATTTACAGGGTGAAGCCGTTCAAGATTGGAGAGAAATCG |
| | TAACATATTTCTCATACCCAATTAGACACAGAGATTACTCCAGGTGGCCT |
| | GATAAGCCAGAAGCCTGGAGGGAAGTTACTAAGAAATACTCAGATGAGT |
| | TGATGGGATTAGCTTGTAAATTGTTGGGCGTGTTGTCAGAAGCCATGGG |
| | ATTGGATACAGAGGCCTTGACCAAAGCATGTGTTGATATGGACCAAAAG |
| | GTAGTTGTCAACTTCTACCCTAAATGCCCTCAACCAGACTTGACATTAG |
| | GCTTGAAAAGACATACCGACCCCGGCACTATCACTTTATTATTACAAGA |
| | CCAAGTCGGTGGTTTGCAGGCTACTAGAGACGACGGTAAAACCTGGAT |
| | CACTGTTCAACCCGTTGAAGGAGCATTCGTCGTTAATTTGGGCGATCAT |
| | GGACACTTATTGTCCAATGGTAGATTTAAGAATGCTGATCACCAAGCTG |
| | TGGTCAACTCTAATAGTAGTAGATTATCCATTGCTACATTTCAGAACCCA |
| | GCACAAGAAGCAATTGTTTATCCTTTATCTGTGAGAGAAGGAGAGAAGC |
| | CTATTTTAGAGGCACCAATTACATATACTGAGATGTATAAGAAGAAGATG |
| | TCTAAAGATTTGGAGTTAGCAAGATTGAAGAAATTAGCTAAAGAGCAACA |
| | AAGTCAAGATTTAGAGAAGGCTAAAGTGGATACTAAACCAGTGGATGAT |
| | ATCTTCGCTTAA |
| SEQ ID NO: 4 | MAPATTLTSIAHEKTLQQKFVRDEDERPKVAYNDFSNEIPIISLAGIDEVEGR |
| | RGEICKKIVAACEDWGIFQIVDHGVDAELISEMTGLAREFFALPSEEKLRFD |
| | MSGGKKGGFIVSSHLQGEAVQDWREIVTYFSYPIRHRDYSRWPDKPEAW |
| | REVTKKYSDELMGLACKLLGVLSEAMGLDTEALTKACVDMDQKVVVNFYP |
| | KCPQPDLTLGLKRHTDPGTITLLLQDQVGGLQATRDDGKTWITVQPVEGAF |
| | VVNLGDHGHLLSNGRFKNADHQAVVNSNSSRLSIATFQNPAQEAIVYPLSV |
| | REGEKPILEAPITYTEMYKKKMSKDLELARLKKLAKEQQSQDLEKAKVDTKP |
| | VDDIFA |
| SEQ ID NO: 5 | ATGATGCACAAAGGTACAGTTTGTGTTACTGGTGCTGCCGGCTTCGTAG |
| | GTAGTTGGTTAATCATGAGGTTATTAGAACAAGGTTACTCCGTTAAGGCT |
| | ACAGTGAGAGATCCTTCTAACATGAAGAAAGTTAAGCATTTGTTGGATTT |
| | ACCCGGAGCAGCAAATAGGTTGACTTTGTGGAAGGCAGATTTAGTTGAT |
| | GAAGGTTCCTTTGATGAACCTATTCAAGGTTGCACAGGTGTATTCCATG |
| | TCGCAACTCCAATGGATTTCGAGTCTAAAGATCCTGAGAGTGAGATGAT |
| | TAAACCTACAATCGAGGGCATGTTAAACGTTTTGAGGTCATGTGCAAGA |
| | GCATCCAGTACTGTCAGAAGGGTAGTTTTCACTTCCTCTGCCGGTACTG |
| | TTAGTATCCATGAAGGCAGAAGACACTTATACGATGAAACCAGTTGGTC |
| | AGACGTCGATTTCTGCAGGGCCAAGAAGATGACAGGTTGGATGTATTTC |
| | GTCTCTAAAACCTTAGCAGAAAAGGCCGCCTGGGATTTCGCAGAAAAGA |
| | ATAACATTGACTTCATTTCTATTATACCCACTTTAGTCAATGGTCCCTTTG |
| | TTATGCCAACTATGCCACCATCAATGTTGTCAGCTTTGGCTTTAATTACC |
| | AGAAATGAACCTCATTACTCAATTTTGAACCCTGTGCAATTTGTACATTT |
| | GGATGATTTATGCAATGCTCATATTTTCTTGTTTGAATGTCCAGATGCTA |
| | AGGGTAGATACATCTGTTCTTCACACGATGTAACAATCGCCGGTTTAGC |
| | TCAAATATTGAGACAAAGATATCCAGAGTTTGACGTGCCAACAGAATTTG |
| | GAGAAATGGAGGTGTTTGACATTATATCATATTCTTCTAAGAAGTTAACT |
| | GACTTGGGATTTGAATTTAAATATTCTTTAGAGGACATGTTTGACGGCGC |
| | TATACAGTCTTGTAGAGAAAAGGGCTTGTTGCCTCCAGCTACAAAAGAA |
| | CCATCCTATGCTACCGAACAATTGATAGCTACCGGACAGGACAATGGAC |
| | ACTAA |
| SEQ ID NO: 6 | MMHKGTVCVTGAAGFVGSWLIMRLLEQGYSVKATVRDPSNMKKVKHLLDL |
| | PGAANRLTLWKADLVDEGSFDEPIQGCTGVFHVATPMDFESKDPESEMIK |
| | PTIEGMLNVLRSCARASSTVRRVVFTSSAGTVSIHEGRRHLYDETSWSDVD |
| | FCRAKKMTGWMYFVSKTLAEKAAWDFAEKNNIDFISIIPTLVNGPFVMPTM |
| | PPSMLSALALITRNEPHYSILNPVQFVHLDDLCNAHIFLFECPDAKGRYICSS |
| | HDVTIAGLAQILRQRYPEFDVPTEFGEMEVFDIISYSSKKLTDLGFEFKYSLE |
| | DMFDGAIQSCREKGLLPPATKEPSYATEQLIATGQDNGH |
| SEQ ID NO: 7 | ATGGGTACTGAAGCTGAAACCGTTTGTGTTACTGGTGCTTCTGGTTTTAT |
| | TGGTTCCTGGTTGATCATGAGATTATTGGAAAAAGGTTACGCTGTTAGA |
| | GCCACTGTTAGAGATCCAGATAATATGAAGAAGGTCACCCACTTGTTGG |
| | AATTGCCAAAGGCTTCTACTCATTTGACTTTGTGGAAAGCCGATTTGTCT |
| | GTTGAAGGTTCTTACGATGAAGCTATTCAAGGTTGTACTGGTGTTTTCCA |
| | TGTTGCTACTCCAATGGATTTCGAATCTAAGGATCCAGAAAACGAAGTTA |
| | TCAAGCCAACCATTAACGGTGTTTTGGATATTATGAGAGCTTGCGCTAA |
| | CTCTAAGACCGTTAGAAAGATCGTTTTCACTTCTTCTGCTGGTACTGTTG |
| | ATGTCGAAGAAAAAAGAAAGCCAGTCTACGATGAATCTTGCTGGTCTGA |
| | TTTGGATTTCGTCCAATCTATTAAGATGACCGGTTGGATGTACTTCGTTT |
| | CTAAAACTTTGGCTGAACAAGCTGCTTGGAAGTTCGCTAAAGAAAACAA |
| | CTTGGACTTCATCTCCATTATCCCAACTTTGGTTGTTGGTCCATTCATCA |
| | TGCAATCTATGCCACCATCTTTGTTGACTGCCTTGTCTTTGATTACTGGT |
| | AACGAAGCTCATTACGGTATCTTGAAACAAGGTCATTACGTTCACTTGG |
| | ATGACTTGTGTATGTCCCATATCTTCTTGTACGAAAACCCAAAAGCTGAA |
| | GGTAGATATATCTGCAACTCTGATGATGCCAACATTCATGATTTGGCTAA |
| | GTTGTTGAGAGAAAAGTACCCAGAATACAACGTTCCAGCTAAGTTCAAG |
| | GATATCGACGAAAATTTGGCTTGCGTTGCTTTCTCATCTAAGAAGTTGAC |
| | AGATTTGGGTTTCGAATTCAAGTACTCCTTGGAAGATATGTTTGCTGGTG |
| | CAGTTGAAACCTGTAGAGAAAAGGGTTTGATTCCATTGTCCCACAGAAA |
| | ACAAGTCGTCGAAGAATGCAAAGAAAATGAAGTTGTTCCAGCTTCTTAA |
| SEQ ID NO: 8 | MGTEAETVCVTGASGFIGSWLIMRLLEKGYAVRATVRDPDNMKKVTHLLEL |
| | PKASTHLTLWKADLSVEGSYDEAIQGCTGVFHVATPMDFESKDPENEVIKP |
| | TINGVLDIMRACANSKTVRKIVFTSSAGTVDVEEKRKPVYDESCWSDLDFV |
| | QSIKMTGWMYFVSKTLAEQAAWKFAKENNLDFISIIPTLVVGPFIMQSMPPS |
| | LLTALSLITGNEAHYGILKQGHYVHLDDLCMSHIFLYENPKAEGRYICNSDD |
| | ANIHDLAKLLREKYPEYNVPAKFKDIDENLACVAFSSKKLTDLGFEFKYSLE |
| | DMFAGAVETCREKGLIPLSHRKQVVEECKENEVVPAS |
| SEQ ID NO: 9 | ATGGTTAACGCCGTTGTTACTACCCCATCTAGAGTTGAATCTTTGGCTAA |
| | GTCTGGTATTCAAGCCATCCCAAAAGAATACGTTAGACCACAAGAAGAA |
| | TTGAACGGTATCGGTAACATTTTCGAAGAAGAAAAGAAAGACGAAGGTC |
| | CACAAGTTCCAACCATCGATTTGAAAGAAATCGACTCCGAAGACAAAGA |
| | AATCAGAGAAAAGTGCCACCAATTGAAAAAGGCTGCTATGGAATGGGGT |
| | GTTATGCATTTGGTTAATCACGGTATCTCCGACGAATTGATCAACAGAGT |
| | TAAGGTTGCTGGTGAAACCTTTTTCGATCAACCAGTCGAAGAAAAAGAA |
| | AAGTACGCTAACGATCAAGCCAACGGTAATGTTCAAGGTTACGGTTCTA |
| | AATTGGCTAACTCTGCTTGTGGTCAATTGGAATGGGAAGATTACTTTTTC |
| | CATTGCGCTTTCCCAGAAGATAAGAGAGATTTGTCTATCTGGCCAAAGA |
| | ACCCAACTGATTATACTCCAGCTACTTCTGAATACGCCAAGCAAATTAGA |
| | GCTTTGGCTACTAAGATTTTGACCGTCTTGTCTATTGGTTTGGGTTTGGA |
| | AGAAGGTAGATTGGAAAAAGAAGTTGGTGGTATGGAAGATTTGTTGTTG |
| | CAAATGAAGATCAACTACTACCCAAAGTGTCCACAACCAGAATTGGCTT |
| | TGGGTGTTGAAGCTCATACTGATGTTTCTGCTTTGACCTTCATCTTGCAT |
| | AATATGGTCCCAGGTTTACAATTATTCTACGAAGGTCAATGGGTTACCG |
| | CTAAGTGTGTTCCAAATTCCATTATCATGCATATCGGTGACACCATCGAA |
| | ATCTTGTCTAACGGTAAATACAAGTCCATCTTGCACAGAGGTGTTGTCAA |
| | CAAAGAAAAGGTTAGATTCTCCTGGGCTATTTTCTGTGAACCACCTAAA |
| | GAAAAGATCATCTTGAAGCCATTGCCAGAAACTGTTACTGAAGCTGAAC |
| | CACCAAGATTTCCACCAAGAACTTTTGCTCAACATATGGCCCATAAGTTG |
| | TTCAGAAAGGATGATAAGGATGCTGCCGTTGAACATAAGGTTTTCAACG |
| | AAGATGAATTGGATACTGCTGCTGAACACAAAGTCTTGAAGAAGGATAA |
| | TCAAGACGCTGTTGCTGAAAACAAGGACATCAAAGAAGATGAACAATGT |
| | GGTCCAGCAGAACACAAAGATATCAAAGAAGATGGTCAAGGTGCTGCT |
| | GCAGAAAACAAGGTTTTCAAAGAAAACAATCAAGATGTCGCCGCCGAAG |
| | AATCTAAGTAA |
| SEQ ID NO: 10 | MVNAVVTTPSRVESLAKSGIQAIPKEYVRPQEELNGIGNIFEEEKKDEGPQV |
| | PTIDLKEIDSEDKEIREKCHQLKKAAMEWGVMHLVNHGISDELINRVKVAGE |
| | TFFDQPVEEKEKYANDQANGNVQGYGSKLANSACGQLEWEDYFFHCAFP |
| | EDKRDLSIWPKNPTDYTPATSEYAKQIRALATKILTVLSIGLGLEEGRLEKEV |
| | GGMEDLLLQMKINYYPKCPQPELALGVEAHTDVSALTFILHNMVPGLQLFY |
| | EGQWVTAKCVPNSIIMHIGDTIEILSNGKYKSILHRGVVNKEKVRFSWAIFCE |
| | PPKEKIILKPLPETVTEAEPPRFPPRTFAQHMAHKLFRKDDKDAAVEHKVFN |
| | EDELDTAAEHKVLKKDNQDAVAENKDIKEDEQCGPAEHKDIKEDGQGAAA |
| | ENKVFKENNQDVAAEESK* |
| SEQ ID NO: 11 | ATGTCAGCAAATTCTAACTACATGAACAAAAGTCGTCTCCATGTCGCTGT |
| | GTTTCCATTCCCTTTTGGAACACACGCGACTCCACTTTTCAACATAACCC |
| | AAAAACTAGCATCATTTATGCCTGATGTCGTCTTCTCCTTCTTCAACATC |
| | CCACAATCCAACGCTAAGATATCTTCTGATTTTAAAAACGATACCATAAA |
| | CATGTATGATGTGTGGGACGGGGTGCCGGAAGGATATGTCTTCAAGGG |
| | TAAGCCTCAAGAAGACATCGAGCTCTTCATGCTGGCTGCACCTCCCACA |
| | TTGACAGAGGCGTTGGCTAAAGCCGAGGTGGAAACAGGGACCAAGGTG |
| | AGCTGCATACTTGGCGATGCCTTTTTATGGTTCCTGGAGGAACTCGCCC |
| | AACAAAAACAAGTTCCCTGGATTACTACTTATATGTCTGAGGAGCATTCT |
| | CTTTTGGCTCATATTTGCACTGATCTTATCAGACAAACTATTGGCATTCA |
| | TGAGAAAGCAGAAGAGCGGAAAGATGAAGAGCTAGATTTCATTCCAGG |
| | ATTGTCCAAGATTAGAGTCCAAGACTTACCAGAGGGAATCGTGATGGGA |
| | AATTTGGATTCGTATTTTGCGAGAATGCTTCACCAAATGGGGCGGGCAT |
| | TACCGCGTGCATCAGCAGTTTGCATTAGTTCATGTCAAGAACTAGACCC |
| | TGTTGCGACTAATGAGCTTAACAGAAAATTGAATAAATTGATTAATGTTG |
| | GACCTCTAAGTCTAATTACGCAATCAAACTCATTACCTTCAGGCACAAAC |
| | AAGAGTCTGGGTTGGCTTGATAAACAAGAATCTGAAAACAGTGTTGCGT |
| | ACGTTAGTTTTGGGTCAGTTGCACGCCCTGATGCAACCGAGATTACAGC |
| | CCTGGCTCAAGCATTGGAGGCAAGTCAGGTCAAATTTATCTGGTCGATT |
| | AGAGACAATCTTAAGGTACATTTGCCAGGTGGATTTATTGAGAATACAAA |
| | GGATAAAGGGATGGTGGTGTCGTGGGTGCCACAGACAGCTGTGTTGGC |
| | TCACAAGGCAGTTGGTGTTTTCATAACCCATTTCGGTCACAATTCCATCA |
| | TGGAAAGTATTGCAAGTGAGGTTCCAATGATAGGGCGACCATTCATCGG |
| | GGAACAAAAGTTGAACGGTAGAATAGTGGAAGCCAAATGGTGTATCGGT |
| | TTGGTTGTGGAAGGTGGAGTTTTCACTAAAGATGGTGTACTGAGAAGCT |
| | TGAACAAAATACTAGGTAGCACACAAGGTGAAGAAATGAGGAGAAATAT |
| | AAGAGACCTACGACTCATGGTTGACAAGGCACTCAGTCCTGACGGAAG |
| | CTGCAATACAAACTTGAAACATTTGGTCGACATGATCGTCACTTCTAACT |
| | AA |
| SEQ ID NO: 12 | MSANSNYMNKSRLHVAVFPFPFGTHATPLFNITQKLASFMPDVVFSFFNIP |
| | QSNAKISSDFKNDTINMYDVWDGVPEGYVFKGKPQEDIELFMLAAPPTLTE |
| | ALAKAEVETGTKVSCILGDAFLWFLEELAQQKQVPWITTYMSEEHSLLAHIC |
| | TDLIRQTIGIHEKAEERKDEELDFIPGLSKIRVQDLPEGIVMGNLDSYFARML |
| | HQMGRALPRASAVCISSCQELDPVATNELNRKLNKLINVGPLSLITQSNSLP |
| | SGTNKSLGWLDKQESENSVAYVSFGSVARPDATEITALAQALEASQVKFIW |
| | SIRDNLKVHLPGGFIENTKDKGMVVSWVPQTAVLAHKAVGVFITHFGHNSI |
| | MESIASEVPMIGRPFIGEQKLNGRIVEAKWCIGLVVEGGVFTKDGVLRSLNK |
| | ILGSTQGEEMRRNIRDLRLMVDKALSPDGSCNTNLKHLVDMIVTSN |
| SEQ ID NO: 13 | ATGGCTGCTTCCATTACCGCTATTACCGTTGAAAATTTGGAATACCCAG |
| | CTGTTGTTACTTCTCCAGTTACTGGTAAGTCTTACTTTTTGGGTGGTGCT |
| | GGTGAAAGAGGTTTGACTATTGAAGGTAACTTCATTAAGTTCACCGCCA |
| | TCGGTGTTTACTTGGAAGATATTGCTGTTGCTTCTTTGGCTGCTAAATGG |
| | AAGGGTAAATCCTCCGAAGAATTATTGGAAACCTTGGACTTCTACAGAG |
| | ACATTATTTCTGGTCCATTCGAAAAGTTGATCAGAGGTTCCAAGATCAGA |
| | GAATTGTCTGGTCCAGAATACTCCAGAAAGGTTATGGAAAATTGCGTTG |
| | CCCATTTGAAGTCTGTTGGTACTTATGGTGATGCTGAAGCTGAAGCTAT |
| | GCAAAAATTTGCTGAAGCCTTTAAGCCAGTTAATTTTCCACCAGGTGCTT |
| | CCGTTTTTTACAGACAATCTCCAGATGGTATCTTGGGTTTGTCTTTTTCA |
| | CCAGATACCTCCATCCCAGAAAAAGAAGCTGCTTTGATTGAAAACAAGG |
| | CTGTTTCTTCTGCTGTCTTGGAAACTATGATTGGTGAACATGCTGTTTCC |
| | CCAGATTTGAAAAGATGTTTAGCTGCTAGATTGCCTGCCTTGTTGAATGA |
| | AGGTGCTTTTAAGATTGGTAACTAA |
| SEQ ID NO: 14 | MAASITAITVENLEYPAVVTSPVTGKSYFLGGAGERGLTIEGNFIKFTAIGVY |
| | LEDIAVASLAAKWKGKSSEELLETLDFYRDIISGPFEKLIRGSKIRELSGPEYS |
| | RKVMENCVAHLKSVGTYGDAEAEAMQKFAEAFKPVNFPPGASVFYRQSP |
| | DGILGLSFSPDTSIPEKEAALIENKAVSSAVLETMIGEHAVSPDLKRCLAARL |
| | PALLNEGAFKIGN |
| SEQ ID NO: 15 | ATGGCGGGCAACGGCGCCATCGTGGAGAGCGACCCGCTGAACTGGGG |
| | CGCGGCGGCGGCGGAGCTGGCCGGGAGCCACCTGGACGAGGTGAAG |
| | CGCATGGTGGCGCAGGCCCGGCAGCCCGTGGTCAAGATCGAGGGCTC |
| | CACCCTCCGCGTCGGCCAGGTGGCCGCCGTCGCCTCCGCCAAGGACG |
| | CGTCCGGCGTCGCCGTCGAGCTCGACGAGGAGGCCCGCCCCCGCGTC |
| | AAGGCCAGCAGCGAGTGGATCCTCGACTGCATCGCCCACGGCGGCGA |
| | CATCTACGGCGTCACCACCGGCTTCGGCGGCACCTCCCACCGCCGCA |
| | CCAAGGACGGGCCCGCGCTCCAGGTCGAGCTGCTCAGGCATCTCAAC |
| | GCCGGAATCTTCGGCACCGGCAGCGACGGGCACACGCTGCCGTCGGA |
| | GGTCACCCGCGCGGCGATGCTGGTGCGCATCAACACCCTCCTCCAGG |
| | GCTACTCCGGCATCCGCTTCGAGATCCTCGAGGCCATCACGAAGCTGC |
| | TCAACACCGGTGTCAGCCCCTGCCTGCCGCTCCGGGGCACCATCACCG |
| | CGTCGGGCGACCTGGTCCCGCTCTCCTACATCGCCGGCCTCATCACGG |
| | GCCGCCCCAACGCGCAGGCCGTCACCGTCGACGGAAGGAAGGTGGAC |
| | GCCGCCGAGGCGTTCAAGATCGCCGGCATCGAGGGCGGCTTCTTCAA |
| | GCTCAACCCCAAGGAGGGCCTCGCCATCGTCAACGGCACGTCCGTGG |
| | GCTCCGCGCTCGCGGCCACCGTGATGTACGACGCCAACGTCCTGGCC |
| | GTCCTGTCGGAGGTCCTGTCCGCCGTCTTTTGCGAGGTCATGAACGGC |
| | AAGCCCGAGTACACGGACCACCTGACCCACAAGCTGAAGCACCACCCG |
| | GGGTCCATCGAGGCCGCGGCCATCATGGAGCACATCCTGGATGGCAG |
| | CTCCTTCATGAAGCAGGCCAAGAAGGTGAACGAGCTGGACCCGCTGCT |
| | GAAGCCCAAGCAGGACAGGTACGCGCTCCGCACGTCGCCGCAGTGGC |
| | TGGGCCCCCAGATCGAGGTCATCCGCGCCGCCACCAAGTCCATCGAG |
| | CGCGAGGTCAACTCCGTGAACGACAACCCGGTCATCGACGTCCACCGC |
| | GGCAAGGCGCTGCACGGCGGCAACTTCCAGGGCACCCCCATCGGCGT |
| | GTCCATGGACAACGCCCGCCTCGCCATCGCCAACATCGGCAAGCTCAT |
| | GTTCGCGCAGTTCTCCGAGCTCGTCAACGAGTTCTACAACAACGGGCT |
| | CACCTCCAACCTGGCCGGCAGCCGCAACCCCAGCCTGGACTACGGCTT |
| | CAAGGGCACCGAGATCGCCATGGCCTCCTACTGCTCCGAGCTCCAGTA |
| | CCTGGGCAACCCCATCACCAACCACGTGCAGAGCGCGGACGAGCACA |
| | ACCAGGACGTGAACTCCCTGGGCCTCGTCTCGGCCAGGAAGACCGCC |
| | GAGGCGATCGACATCCTGAAGCTCATGTCGTCCACCTACATCGTGGCG |
| | CTGTGCCAGGCCGTGGACCTGCGCCACCTCGAGGAGAACATCAAGGC |
| | GTCGGTGAAGAACACCGTGACCCAGGTGGCCAAGAAGGTGCTGACCAT |
| | GAACCCCTCGGGCGAGCTCTCCAGCGCCCGCTTCAGCGAGAAGGAGC |
| | TGATCAGCGCCATCGACCGCGAGGCCGTGTTCACGTACGCGGAGGAC |
| | GCGGCCAGCGCCAGCCTGCCGCTGATGCAGAAGCTGCGCGCCGTGCT |
| | GGTGGACCACGCCCTCAGCAGCGGCGAGCGCGGAGCGGGAGCCCTC |
| | CGTGTTCTCCAAGATCACCAGGTTCGAGGAGGAGCTCCGCGCGGTGCT |
| | GCCCCAGGAGGTGGAGGCCGCCCGCGTGGCGTCGCCGAGGGCACCG |
| | CCCCCGTGGCGAACCGGATCGCGGACAGCCGGTCGTTCCCGCTGTAC |
| | CGCTTCGTGCGCGAGGAGCTCGGCTGCGTGTTCCTGACCGGCGAGAG |
| | GCTCAAGTCCCCCGGCGAGGAGTGCAACAAGGTGTTCGTCGGCATCAG |
| | CCAGGGCAAGCTCGTGGACCCCATGCTCGAGTGCCTCAAGGAGTGGG |
| | ACGGCAAGCCGCTGCCCATCAACATCAAGTAA |
| SEQ ID NO: 16 | MAGNGAIVESDPLNWGAAAAELAGSHLDEVKRMVAQARQPVVKIEGSTLR |
| | VGQVAAVASAKDASGVAVELDEEARPRVKASSEWILDCIANGGDIYGVTTG |
| | FGGTSHRRTKDGPALQVELLRHLNAGIFGTGSDGHTLPSEVTRAAMLVRIN |
| | TLLQGYSGIRFEILEAITKLLNTGVSPCLPLRGTITASGDLVPLSYIAGLITGRP |
| | NAQAVTVDGRKVDAAEAFKIAGIEGGFFKLNPKEGLAIVNGTSVGSALAATV |
| | MYDANVLAVLSEVLSAVFCEVMNGKPEYTDHLTHKLKHHPGSIEAAAIMEHI |
| | LDGSSFMKQAKKVNELDPLLKPKQDRYALRTSPQWLGPQIEVIRAATKSIE |
| | REVNSVNDNPVIDVHRGKALHGGNFQGTPIGVSMDNARLAIANIGKLMFAQ |
| | FSELVNEFYNNGLTSNLAGSRNPSLDYGFKGTEIAMASYCSELQYLGNPIT |
| | NHVQSADEHNQDVNSLGLVSARKTAEAIDILKLMSSTYIVALCQAVDLRHLE |
| | ENIKASVKNTVTQVAKKVLTMNPSGELSSARFSEKELISAIDREAVFTYAED |
| | AASASLPLMQKLRAVLVDHALSSGERGAGALRVLQDHQVRGGAPRGAAP |
| | GGGGRPRGVAEGTAPVANRIADSRSFPLYRFVREELGCVFLTGERLKSPG |
| | EECNKVFVGISQGKLVDPMLECLKEWDGKPLPINIK |
| SEQ ID NO: 17 | ATGGACCAAATTGAAGCAATGCTATGCGGTGGTGGTGAAAAGACCAAG |
| | GTGGCCGTAACGACAAAAACTCTTGCAGATCCTTTGAATTGGGGTCTGG |
| | CAGCTGACCAGATGAAAGGTAGCCATCTGGATGAAGTTAAGAAGATGGT |
| | TGAGGAATACAGAAGACCAGTCGTAAATCTAGGCGGCGAGACATTGAC |
| | GATAGGACAGGTAGCTGCTATTTCGACCGTTGGCGGTTCAGTGAAGGT |
| | AGAACTTGCAGAAACAAGTAGAGCCGGAGTTAAGGCTTCATCAGATTGG |
| | GTCATGGAAAGTATGAACAAGGGCACAGATTCCTATGGCGTTACCACAG |
| | GCTTTGGTGCTACCTCTCATAGAAGAACTAAAAATGGCACTGCTTTGCA |
| | AACAGAACTGATCAGATTCCTTAACGCCGGTATTTTCGGTAATACAAAG |
| | GAAACTTGCCATACATTACCCCAATCGGCAACAAGAGCTGCTATGCTTG |
| | TTAGGGTGAACACTTTGTTGCAAGGTTACTCTGGAATAAGGTTTGAAATT |
| | CTTGAGGCCATCACTTCACTATTGAACCACAACATTTCTCCTTCGTTGCC |
| | CTTAAGAGGAACAATAACTGCCAGCGGTGATTTGGTTCCCCTTTCATAT |
| | ATCGCAGGCTTATTAACGGGAAGACCTAATTCAAAGGCCACTGGTCCAG |
| | ACGGAGAATCCTTAACCGCTAAGGAAGCATTTGAGAAAGCTGGTATTTC |
| | AACTGGTTTCTTTGATTTgCAACCCAAGGAAGGTTTAGCCCTGGTGAATG |
| | GCACCGCTGTCGGCAGCGGTATGGCATCCATGGTGTTGTTTGAAGCTA |
| | ACGTACAAGCAGTTTTGGCCGAAGTTTTGTCCGCAATTTTTGCCGAAGT |
| | CATGAGTGGAAAACCTGAGTTTACTGATCACTTGACCCACAGGTTAAAA |
| | CATCACCCAGGACAAATTGAAGCAGCAGCTATCATGGAGCACATTTTGG |
| | ACGGCTCTAGCTACATGAAGTTAGCCCAGAAGGTTCATGAAATGGACCC |
| | TTTGCAAAAACCCAAACAAGATAGATATGCTTTAAGGACATCCCCACAAT |
| | GGCTTGGCCCTCAAATTGAAGTAATTAGACAAGCTACAAAGTCTATAGA |
| | AAGAGAGATCAACTCTGTTAACGATAATCCACTTATTGATGTGTCGAGG |
| | AATAAGGCAATACATGGAGGCAATTTCCAGGGTACACCCATAGGAGTCA |
| | GTATGGATAATACCAGGCTTGCCATAGCCGCAATTGGCAAATTAATGTT |
| | TGCCCAATTTTCTGAATTGGTCAATGACTTCTACAATAACGGTTTGCCTT |
| | CGAATCTGACCGCATCTTCTAACCCTAGTCTTGATTATGGTTTCAAAGGT |
| | GCTGAGATAGCAATGGCAAGCTATTGTTCAGAGCTGCAATATCTAGCCA |
| | ACCCAGTAACCTCTCATGTACAATCAGCCGAACAACACAATCAGGATGT |
| | TAATTCTTTGGGCCTGATTTCATCAAGAAAAACAAGCGAGGCCGTTGAT |
| | ATCCTTAAATTAATGTCCACAACATTTTTAGTGGGTATATGCCAGGCCGT |
| | AGATTTgAGACACTTGGAAGAGAATTTGAGACAGACAGTGAAAAATACC |
| | GTATCACAGGTTGCAAAAAAGGTTCTAACTACAGGTATCAATGGTGAATT |
| | GCACCCATCAAGATTCTGTGAAAAAGATTTATTAAAAGTTGTAGATAGAG |
| | AACAAGTATTTACTTACGTTGACGATCCATGTAGCGCTACTTATCCATTG |
| | ATGCAGAGATTGAGACAAGTTATTGTAGATCACGCTTTATCCAATGGTG |
| | AAACTGAGAAAAATGCCGTTACTTCAATATTCCAAAAGATAGGTGCCTTT |
| | GAAGAAGAACTGAAGGCAGTTTTACCAAAGGAAGTCGAAGCTGCTAGA |
| | GCCGCATACGGAAATGGTACTGCCCCTATACCAAATAGAATCAAAGAGT |
| | GTAGGTCGTACCCTTTGTACAGATTCGTTAGAGAAGAGTTGGGAACCAA |
| | ATTACTAACTGGTGAAAAAGTCGTTAGCCCAGGTGAAGAATTTGACAAG |
| | GTATTCACAGCTATGTGCGAGGGAAAGTTGATAGATCCACTTATGGATT |
| | GCTTGAAAGAGTGGAATGGTGCACCTATTCCAATCTGCTAA |
| SEQ ID NO: 18 | MDQIEAMLCGGGEKTKVAVTIKTLADPLNWGLAADQMKGSHLDEVKKMV |
| | EEYRRPVVNLGGETLTIGQVAAISTVGGSVKVELAETSRAGVKASSDWVME |
| | SMNKGTDSYGVTTGFGATSHRRTKNGTALQTELIRFLNAGIFGNTKETCHT |
| | LPQSATRAAMLVRVNTLLQGYSGIRFEILEAITSLLNHNISPSLPLRGTITASG |
| | DLVPLSYIAGLLTGRPNSKATGPDGESLTAKEAFEKAGISTGFFDLQPKEGL |
| | ALVNGTAVGSGMASMVLFEANVQAVLAEVLSAIFAEVMSGKPEFTDHLTHR |
| | LKHHPGQIEAAAIMEHILDGSSYMKLAQKVHEMDPLQKPKQDRYALRTSPQ |
| | WLGPQIEVIRQATKSIEREINSVNDNPLIDVSRNKAIHGGNFQGTPIGVSMD |
| | NTRLAIAAIGKLMFAQFSELVNDFYNNGLPSNLTASSNPSLDYGFKGAEIAM |
| | ASYCSELQYLANPVTSHVQSAEQHNQDVNSLGLISSRKTSEAVDILKLMST |
| | TFLVGICQAVDLRHLEENLRQTVKNTVSQVAKKVLTTGINGELHPSRFCEKD |
| | LLKVVDREQVFTYVDDPCSATYPLMQRLRQVIVDHALSNGETEKNAVTSIF |
| | QKIGAFEEELKAVLPKEVEAARAAYGNGTAPIPNRIKECRSYPLYRFVREEL |
| | GTKLLTGEKVVSPGEEFDKVFTAMCEGKLIDPLMDCLKEWNGAPIPIC |
| SEQ ID NO: 19 | ATGATGGATTTTGTTTTGTTAGAAAAAGCTCTTCTTGGTTTGTTCATTGCA |
| | ACTATAGTAGCCATCACAATCTCTAAGCTAAGGGGAAAGAAACTTAAGTT |
| | GCCTCCAGGCCCAATCCCTGTCCCAGTGTTTGGTAATTGGTTACAAGTT |
| | GGCGACGACTTAAACCAGAGGAATTTGGTAGAGTATGCTAAAAAGTTCG |
| | GCGACTTATTTCTACTTAGGATGGGTCAAAGAAACTTGGTCGTGGTTTC |
| | ATCCCCTGACTTAGCAAAAGACGTACTACATACCCAGGGTGTCGAGTTC |
| | GGAAGTAGAACTAGAAATGTTGTGTTTGATATTTTCACAGGCAAAGGTC |
| | AAGATATGGTTTTTACCGTATACAGCGAGCACTGGAGGAAAATGAGAAG |
| | AATAATGACTGTCCCATTCTTTACAAACAAAGTGGTTCAACAGTATAGGT |
| | TCGGATGGGAGGACGAAGCCGCTAGAGTAGTCGAGGATGTTAAGGCAA |
| | ATCCTGAAGCCGCTACCAACGGTATTGTGTTGAGGAATAGATTACAACT |
| | TTTGATGTACAACAATATGTATAGAATAATGTTTGACAGGAGATTTGAAT |
| | CTGTTGATGATCCATTATTCCTAAAACTTAAGGCATTGAATGGCGAGAGA |
| | TCAAGGTTAGCTCAATCCTTTGAATACAACTTCGGTGACTTCATTCCTAT |
| | ATTGAGGCCATTCTTGAGAGGATATCTTAAGTTGTGTCAGGAAATCAAG |
| | GACAAAAGGTTAAAGCTATTCAAGGACTACTTCGTCGACGAGAGAAAAA |
| | AGTTGGAGAGTATCAAGAGCGTAGGTAATAACTCCTTAAAGTGCGCCAT |
| | AGATCATATTATCGAGGCACAAGAAAAAGGCGAGATAAACGAGGATAAC |
| | GTGTTATACATCGTCGAGAATATCAACGTGGCTGCCATTGAAACTACAC |
| | TTTGGTCTATTGAATGGGGTATAGCAGAACTAGTGAATAACCCTGAAAT |
| | CCAGAAAAAATTGAGACACGAATTAGACACCGTACTTGGAGCTGGTGTT |
| | CAAATTTGTGAACCAGATGTTCAAAAATTGCCTTATCTACAGGCCGTGAT |
| | AAAAGAGACTTTAAGGTACAGGATGGCAATTCCATTGTTAGTCCCACAT |
| | ATGAATCTTCACGAAGCCAAATTGGCCGGCTATGATATCCCTGCAGAGA |
| | GCAAAATTTTGGTAAACGCTTGGTGGTTAGCCAATAATCCAGCACATTG |
| | GAACAAACCTGATGAGTTTAGACCAGAAAGATTTTTGGAGGAAGAATCC |
| | AAGGTCGAGGCTAATGGAAACGACTTTAAGTACATCCCTTTCGGTGTTG |
| | GCAGAAGATCTTGCCCAGGTATAATTCTTGCTTTACCAATCCTTGGAATA |
| | GTAATTGGTAGGTTGGTTCAAAACTTCGAGTTACTTCCACCTCCAGGCC |
| | AAAGCAAAATAGATACAGCCGAAAAAGGTGGACAGTTTTCATTGCAAAT |
| | CCTAAAGCATTCCACTATTGTGTGTAAACCTAGAAGTTCTTAA |
| SEQ ID NO: 20 | MMDFVLLEKALLGLFIATIVAITISKLRGKKLKLPPGPIPVPVFGNWLQVGDD |
| | LNQRNLVEYAKKFGDLFLLRMGQRNLVVVSSPDLAKDVLHTQGVEFGSRT |
| | RNVVFDIFTGKGQDMVFTVYSEHWRKMRRIMTVPFFTNKVVQQYRFGWE |
| | DEAARVVEDVKANPEAATNGIVLRNRLQLLMYNNMYRIMFDRRFESVDDPL |
| | FLKLKALNGERSRLAQSFEYNFGDFIPILRPFLRGYLKLCQEIKDKRLKLFKD |
| | YFVDERKKLESIKSVGNNSLKCAIDHIlEAQEKGEINEDNVLYIVENINVAAIET |
| | TLWSIEWGIAELVNNPEIQKKLRHELDTVLGAGVQICEPDVQKLPYLQAVIK |
| | ETLRYRMAIPLLVPHMNLHEAKLAGYDIPAESKILVNAWWLANNPAHWNKP |
| | DEFRPERFLEEESKVEANGNDFKYIPFGVGRRSCPGIILALPILGIVIGRLVQ |
| | NFELLPPPGQSKIDTAEKGGQFSLQILKHSTIVCKPRSS |
| SEQ ID NO: 21 | ATGGCTGCAGTAAGATTGAAAGAAGTTAGAATGGCACAGAGGGCTGAA |
| | GGTTTAGCTACAGTTTTAGCAATCGGTACTGCCGTTCCAGCTAATTGTG |
| | TTTATCAAGCTACCTATCCAGATTATTATTTTAGGGTTACTAAAAGTGAG |
| | CACTTGGCAGATTTAAAGGAGAAGTTTCAAAGAATGTGTGACAAATCAAT |
| | GATTAGAAAGAGACACATGCACTTGACCGAGGAAATATTGATCAAGAAC |
| | CCAAAGATCTGTGCACACATGGAGACCTCATTGGATGCTAGACACGCCA |
| | TCGCATTAGTTGAAGTTCCCAAATTGGGCCAAGGTGCAGCTGAGAAGG |
| | CCATTAAGGAGTGGGGCCAACCCTTGTCTAAGATTACTCATTTGGTATTT |
| | TGCACAACATCCGGCGTTGACATGCCCGGTGCTGATTACCAATTAACAA |
| | AGTTGTTAGGTTTGTCCCCTACAGTCAAAAGGTTAATGATGTACCAACAA |
| | GGTTGCTTTGGTGGTGCAACTGTTTTGAGATTGGCAAAAGATATCGCTG |
| | AAAATAATAGAGGTGCCAGAGTGTTAGTCGTTTGTTCCGAGATAACTGC |
| | TATGGCCTTCAGAGGTCCATGCAAGAGTCATTTAGATTCCTTGGTAGGT |
| | CATGCCTTGTTCGGTGATGGTGCCGCTGCTGCAATTATAGGCGCTGAC |
| | CCAGACCAATTAGACGAACAACCAGTTTTCCAGTTGGTATCAGCTTCTC |
| | AGACTATATTACCAGAATCAGAAGGTGCCATAGATGGCCATTTAACAGA |
| | AGCTGGTTTAACTATACATTTATTAAAAGATGTTCCTGGTTTAATTTCAGA |
| | GAACATTGAACAGGCTTTGGAGGATGCCTTTGAACCTTTAGGTATTCAT |
| | AACTGGAATTCAATTTTCTGGATTGCACATCCTGGTGGCCCTGCCATTTT |
| | AGACAGAGTTGAAGATAGAGTAGGATTGGATAAGAAGAGAATGAGGGC |
| | TTCTAGGGAAGTGTTATCTGAATACGGAAATATGTCTAGTGCCTCTGTGT |
| | TGTTTGTGTTAGATGTCATGAGGAAAAGTTCTGCTAAAGACGGATTGGC |
| | AACCACAGGAGAAGGAAAAGATTGGGGAGTGTTGTTTGGATTCGGACC |
| | AGGCTTGACTGTAGAAACCTTAGTGTTGCATAGTGTCCCAGTCCCTGTC |
| | CCTACTGCAGCTTCTGCATGA |
| SEQ ID NO: 22 | MAAVRLKEVRMAQRAEGLATVLAIGTAVPANCVYQATYPDYYFRVTKSEHL |
| | ADLKEKFQRMCDKSMIRKRHMHLTEEILIKNPKICAHMETSLDARHAIALVE |
| | VPKLGQGAAEKAIKEWGQPLSKITHLVFCTTSGVDMPGADYQLTKLLGLSP |
| | TVKRLMMYQQGCFGGATVLRLAKDIAENNRGARVLVVCSEITAMAFRGPC |
| | KSHLDSLVGHALFGDGAAAAIIGADPDQLDEQPVFQLVSASQTILPESEGAI |
| | DGHLTEAGLTIHLLKDVPGLISENIEQALEDAFEPLGIHNWNSIFWIAHPGGP |
| | AILDRVEDRVGLDKKRMRASREVLSEYGNMSSASVLFVLDVMRKSSAKDG |
| | LATTGEGKDWGVLFGFGPGLTVETLVLHSVPVPVPTAASA |
| SEQ ID NO: 23 | ATGCCGTTTGGAATAGACAACACCGACTTCACTGTCCTGGCGGGGCTA |
| | GTGCTTGCCGTGCTACTGTACGTAAAGAGAAACTCCATCAAGGAACTGC |
| | TGATGTCCGATGACGGAGATATCACAGCTGTCAGCTCGGGCAACAGAG |
| | ACATTGCTCAGGTGGTGACCGAAAACAACAAGAACTACTTGGTGTTGTA |
| | TGCGTCGCAGACTGGGACTGCCGAGGATTACGCCAAAAAGTTTTCCAA |
| | GGAGCTGGTGGCCAAGTTCAACCTAAACGTGATGTGCGCAGATGTTGA |
| | GAACTACGACTTTGAGTCGCTAAACGATGTGCCCGTCATAGTCTCGATT |
| | TTTATCTCTACATATGGTGAAGGAGACTTCCCCGACGGGGCGGTCAACT |
| | TTGAAGACTTTATTTGTAATGCGGAAGCGGGTGCACTATCGAACCTGAG |
| | GTATAATATGTTTGGTCTGGGAAATTCTACTTATGAATTCTTTAATGGTG |
| | CCGCCAAGAAGGCCGAGAAGCATCTCTCCGCTGCGGGCGCTATCAGAC |
| | TAGGCAAGCTCGGTGAAGCTGATGATGGTGCAGGAACTACAGAGAAG |
| | ATTACATGGCCTGGAAGGACTCCATCCTGGAGGTTTTGAAAGACGAACT |
| | GCATTTGGACGAACAGGAAGCCAAGTTCACCTCTCAATTCCAGTACACT |
| | GTGTTGAACGAAATCACTGACTCCATGTCGCTTGGTGAACCCTCTGCTC |
| | ACTATTTGCCCTCGCATCAGTTGAACCGCAACGCAGACGGCATCCAATT |
| | GGGTCCCTTCGATTTGTCTCAACCGTATATTGCACCCATCGTGAAATCT |
| | CGCGAACTGTTCTCTTCCAATGACCGTAATTGCATCCACTCTGAATTTGA |
| | CTTGTCCGGCTCTAACATCAAGTACTCCACTGGTGACCATCTTGCTGTT |
| | TGGCCTTCCAACCCATTGGAAAAGGTCGAACAGTTCTTATCCATATTCAA |
| | CCTGGACCCTGAAACCATTTTTGACTTGAAGCCCCTGGATCCCACCGTC |
| | AAAGTGCCCTTCCCAACGCCAACTACTATTGGCGCTGCTATTAAACACT |
| | ATTTGGAAATTACAGGACCTGTCTCCAGACAATTGTTTTCATCTTTGATT |
| | CAGTTCGCCCCCAACGCTGACGTCAAGGAAAAATTGACTCTGCTTTCGA |
| | AAGACAAGGACCAATTCGCCGTCGAGATAACCTCCAAATATTTCAACAT |
| | CGCAGATGCTCTGAAATATTTGTCTGATGGCGCCAAATGGGACACCGTA |
| | CCCATGCAATTCTTGGTCGAATCAGTTCCCCAAATGACTCCTCPTTACTA |
| | CTCTATCTCTTCCTCTTCTCTGTCTGAAAAGCAAACCGTCCATGTCACCT |
| | CCATTGTGGAAAACTTTCCTAACCCAGAATTGCCTGATGCTCCTCCAGT |
| | TGTTGGTGTTACGACTAACTTGTTAAGAAACATTCAATTGGCTCAAAACA |
| | ATGTTAACATTGCCGAAACTAACCTACCTGTTCACTACGATTTAAATGGC |
| | CCACGTAAACTTTTCGCCAATTACAAATTGCCCGTCCACGTTCGTCGTT |
| | CTAACTTCAGATTGCCTTCCAACCCTTCCACCCCAGTTATCATGATCGGT |
| | CCAGGTACCGGTGTTGCCCCATTCCGTGGGTTTATCAGAGAGCGTGTC |
| | GCGTTCCTCGAATCACAAAAGAAGGGCGGTAACAACGTTTCGCTAGGTA |
| | AGCATATACTGTTTTATGGATCCCGTAACACTGATGATTTCTTGTACCAG |
| | GACGAATGGCCAGAATACGCCAAAAAATTGGATGGTTCGTTCGAAATGG |
| | TCGTGGCCCATTCCAGGTTGCCAAACACCAAAAAAGTTTATGTTCAAGA |
| | TAAATTAAAGGATTACGAAGACCAAGTATTTGAAATGATTAACAACGGTG |
| | CATTTATCTACGTCTGTGGTGATGCAAAGGGTATGGCCAAGGGTGTGTC |
| | AACCGCATTGGTTGGCATCTTATCCCGTGGTAAATCCATTACCACTGAT |
| | GAAGCAACAGAGCTAATCAAGATGCTCAAGACTTCAGGTAGATACCAAG |
| | AAGATGTCTGGTAA |
| SEQ ID NO: 24 | MPFGIDNTDFTVLAGLVLAVLLYVKRNSIKELLMSDDGDITAVSSGNRDIAQ |
| | VVTENNKNYLVLYASQTGTAEDYAKKFSKELVAKFNLNVMCADVENYDFES |
| | LNDVPVIVSIFISTYGEGDFPDGAVNFEDFICNAEAGALSNLRYNMFGLGNS |
| | TYEFFNGAAKKAEKHLSAAGAIRLGKLGEADDGAGTTDEDYMAWKDSILEV |
| | LKDELHLDEQEAKFTSQFQYTVLNEITDSMSLGEPSAHYLPSHQLNRNADG |
| | IQLGPFDLSQPYIAPIVKSRELFSSNDRNCIHSEFDLSGSNIKYSTGDHLAVW |
| | PSNPLEKVEQFLSIFNLDPETIFDLKPLDPTVKVPFPTPTTIGAAIKHYLEITGP |
| | VSRQLFSSLIQFAPNADVKEKLTLLSKDKDQFAVEITSKYFNIADALKYLSDG |
| | AKWDTVPMQFLVESVPQMTPRYYSISSSSLSEKQTVHVTSNENFPNPELP |
| | DAPPVVGVTTNLLRNIQLAQNNVNIAETNLPVHYDLNGPRKLFANYKLPVHV |
| | RRSNFRLPSNPSTPVIMIGPGTGVAPFRGFIRERVAFLESQKKGGNNVSLG |
| | KHILFYGSRNTDDFLYQDEWPEYAKKLDGSFEMVVAHSRLPNTKKVYVQD |
| | KLKDYEDQVFEMINNGAFIYVCGDAKGMAKGVSTALVGILSRGKSITTDEAT |
| | ELIKMLKTSGRYQEDVW |
| SEQ ID NO: 25 | ATGACCAAGCCATCTGATCCAACCAGAGATTCTCATGTTGCTGTTTTGG |
| | CTTTTCCATTTGGTACTCATGCTGCTCCATTATTGACTGTTACTAGAAGA |
| | TTGGCTTCTGCTTCTCCATCTACCGTTTTTTCTTTTTTCAACACCGCCCA |
| | ATCCAACTCCTCTTTGTTTTCATCTGGTGATGAAGCTGATAGACCAGCCA |
| | ATATTAGAGTTTACGATATTGCTGATGGTGTCCCAGAAGGTTACGTTTTT |
| | TCAGGTAGACCACAAGAAGCCATCGAATTATTCTTGCAAGCTGCTCCAG |
| | AAAACTTCAGAAGAGAAATTGCTAAGGCTGAAACCGAAGTTGGTACTGA |
| | AGTTAAGTGTTTGATGACCGATGCTTTTTTTTGGTTCGCTGCTGATATGG |
| | CTACTGAAATCAATGCTTCTTGGATTGCTTTTTGGACTGCTGGTGCTAAT |
| | TCTTTGTCTGCTCACTTGTACACCGATTTGATTAGAGAAACCATCGGTGT |
| | CAAAGAAGTCGGTGAAAGAATGGAAGAAACTATTGGTGTTATTTCCGGT |
| | ATGGAAAAGATCAGAGTTAAGGATACTCCAGAAGGTGTTGTTTTCGGTA |
| | ACTTGGATTCTGTTTTCTCCAAGATGTTGCACCAAATGGGTTTGGCTTTG |
| | CCAAGAGCTACTGCTGTTTTTATCAACTCCTTCGAAGATTTGGATCCTAC |
| | CTTGACTAACAACTTGAGATCCAGATTCAAGAGATACTTGAACATTGGTC |
| | CATTGGGTTTGTTGTCCTCTACATTGCAACAATTGGTTCAAGATCCACAT |
| | GGTTGTTTGGCTTGGATGGAAAAAAGATCATCTGGTTCCGTTGCCTACA |
| | TTTCTTTTGGTACTGTTATGACTCCACCACCAGGTGAATTGGCTGCTATT |
| | GCTGAAGGTTTGGAATCTTCTAAGGTTCCATTTGTTTGGTCCTTGAAAGA |
| | AAAGTCCTTGGTCCAATTGCCAAAGGGTTTTTTGGATAGPACTAGAGAA |
| | CAAGGTATCGTTGTTCCATGGGCTCCACAAGTTGAATTATTGAAACATG |
| | AAGCTACCGGTGTTTTCGTTACTCATTGTGGTTGGAATTCTGTCTTGGAA |
| | TCAGTTTCTGGTGGTGTTCCAATGATCTGTAGACCATTTTTTGGTGACCA |
| | AAGATTGAACGGTAGAGCCGTTGAAGTTGTTTGGGAAATTGGTATGACC |
| | ATCATCAATGGTGTTTTCACCAAGGATGGTTTCGAAAAGTGTTTGGATAA |
| | GGTTTTGGTCCAAGACGACGGTAAAAAGATGAAGTGTAATGCCAAGAAG |
| | TTGAAAGAATTGGCTTACGAAGCTGTCTCCTCTAAAGGTAGATCATCCG |
| | AAAATTTCAGAGGTTTGTTGGATGCCGTTGTCAACATTATCTGA |
| SEQ ID NO: 26 | MTKPSDPTRDSHVAVLAFPFGTHAAPLLTVTRRLASASPSTVFSFFNTAQS |
| | NSSLFSSGDEADRPANIRVYDIADGVPEGYVFSGRPQEAIELFLQAAPENF |
| | RREIAKAETEVGTEVKCLMTDAFFWFAADMATEINASWIAFWTAGANSLSA |
| | HLYTDLIRETIGVKEVGERMEETIGVISGMEKIRVKDTPEGVVFGNLDSVFSK |
| | MLHQMGLALPRATAVFINSFEDLDPILTNNLRSRFKRYLNIGPLGLLSSTLQ |
| | QLVQDPHGCLAWMEKRSSGSVAYISFGTVMTPPPGELAAIAEGLESSKVPF |
| | VWSLKEKSLVQLPKGFLDRTREQGIVVPWAPQVELLKHEATGVFVTHCGW |
| | NSVLESVSGGVPMICRPFFGDQRLNGRAVEVVWEIGMTIINGVFTKDGFEK |
| | CLDKVLVQDDGKKMKCNAKKLKELAYEAVSSKGRSSENFRGLLDAVVNII |
| SEQ ID NO: 27 | ATGGAGATTTTAAGTTTAATTTTGTATACAGTTATCTTCAGTTTCTTATTG |
| | CAATTTATTTTGAGATCTTTCTTTAGGAAAAGATATCCATTACCATTACCT |
| | CCAGGTCCAAAACCATGGCCAATAATAGGCAACTTAGTACACTTGGGAC |
| | CCAAACCACACCAGTCTACCGCCGCTATGGCCCAAACATATGGTCCATT |
| | GATGTACTTAAAGATGGGCTTCGTAGACGTCGTTGTCGCTGCATCTGCA |
| | AGTGTTGCTGCACAATTCTTGAAGACTCACGATGCTAACTTCTCTTCTAG |
| | ACCTCCAAATAGTGGCGCTGAGCATATGGCCTATAATTACCAAGACTTG |
| | GTTTTCGCCCCATACGGCCCTAGGTGGAGAATGTTAAGGAAAATATGTT |
| | CTGTGCACTTGTTCTCTACAAAAGCATTGGATGATTTCAGACATGTCAGA |
| | CAAGACGAAGTAAAGACTTTAACCAGAGCATTAGCTTCAGCAGGTCAGA |
| | AGCCCGTGAAGTTAGGCCAATTATTAAACGTCTGTACTACTAATGCTTTA |
| | GCCAGAGTAATGTTAGGTAAAAGAGTCTTCGCTGACGGTTCAGGCGAT |
| | GTTGACCCACAAGCCGCAGAATTCAAATCTATGGTAGTTGAGATGATGG |
| | TCGTCGCCGGTGTATTTAACATAGGAGATTTCATTCCTCAATTAAATTGG |
| | TTGGACATTCAAGGTGTGGCCGCTAAAATGAAGAAGTTACATGCTAGAT |
| | TCGATGCTTTCTTGACAGACATATTGGAAGAACATAAAGGTAAAATCTTT |
| | GGTGAAATGAAGGATTTATTAAGTACCTTAATCTCCTTGAAGAATGATGA |
| | TGCCGACAATGATGGTGGAAAATTGACAGATAGAGAGATTAAAGCATTA |
| | TTATTAAACTTGTTTGTTGCAGGAACTGATACTTCATCCTCAACTGTTGA |
| | ATGGGCAATTGCCGAATTGATCAGAAATCCAAAGATTTTGGCTCAGGCT |
| | CAACAAGAGATCGACAAAGTGGTAGGTAGAGACAGGTTGGTGGGCGAA |
| | TTAGATTTAGCACAATTAACCTACTTGGAAGCAATTGTTAAGGAAACCTT |
| | TAGATTGCATCCCTCCACTCCATTATCATTGCCAAGAATAGCATCAGAAT |
| | CATGTGAAATCAACGGTTACTTTATCCCAAAAGGATCCACTTTATTATTG |
| | AATGTTTGGGCTATAGCCAGGGATCCTAATGCTTGGGCCGATCCTTTAG |
| | AATTTAGACCTGAAAGATTCTTGCCTGGTGGTGAAAAGCCTAAGGTGGA |
| | TGTAAGGGGAAATGATTTTGAGGTGATTCCCTTTGGAGCAGGTAGGAG |
| | GATTTGCGCTGGAATGAATTTGGGTATTAGGATGGTTCAGTTAATGATC |
| | GCAACATTGATACATGCATTTAACTGGGATTTGGTTTCCGGTCAGTTGC |
| | CTGAAATGTTGAACATGGAAGAGGCTTATGGTTTGACATTGCAGAGAGC |
| | TGATCCTTTGGTTGTTCATCCCAGACCCAGATTGGAAGCTCAGGCTTAT |
| | ATCGGTTGA |
| SEQ ID No. 28 | MEILSLILYTVIFSFLLQFILRSFFRKRYPLPLPPGPKPWPIIGNLVHLGPKPH |
| | QSTAAMAQTYGPLMYLKMGFVDVVVAASASVAAQFLKTHDANFSSRPPNS |
| | GAEHMAYNYQDLVFAPYGPRWRMLRKICSVHLFSTKALDDFRHVRQDEVK |
| | TLTRALASAGQKPVKLGQLLNVCITNALARVMLGKRVFADGSGDVDPQAA |
| | EFKSMVVEMMVVAGVFNIGDFIPQLNWLDIQGVAAKMKKLHARFDAFLTDIL |
| | EEHKGKIFGEMKDLLSTLISLKNDDADNDGGKLTDTEIKALLLNLFVAGTDTS |
| | SSTVEWAIAELIRNPKILAQAQQEIDKVVGRDRLVGELDLAQLTYLEAIVKET |
| | FRLHPSTPLSLPRIASESCEINGYFIPKGSTLLLNVWAIARDPNAWADPLEFR |
| | PERFLPGGEKPKVDVRGNDFEVIPFGAGRRICAGMNLGIRMVQLMIATLIHA |
| | FNWDLVSGQLPEMLNMEEAYGLTLQRADPLVVHPRPRLEAQAYIG |
| SEQ ID NO: 29 | ATGACTGTTAGTCCATCTATCGCTAGTGCAGCCAAATCTGGCAGAGTAT |
| | TAATTATCGGTGCCACCGGCTTTATAGGTAAATTTGTTGCTGAAGCATCT |
| | TTGGATAGTGGCTTGCCAACATATGTCTTAGTAAGACCAGGTCCTTCAA |
| | GACCAAGTAAAAGTGATACAATTAAATCTTTAAAAGACAGGGGCGCAAT |
| | AATTTTACACGGTGTCATGTCTGATAAACCATTGATGGAAAAATTGTTAA |
| | AGGAGCATGAAATCGAGATTGTTATTTCAGCTGTGGGTGGTGCTACTAT |
| | TTTAGATCAAATCACCTTGGTAGAAGCTATCACCTCAGTAGGAACAGTC |
| | AAGAGATTTTTGCCCTCCGAATTTGGCCATGACGTAGATAGAGCCGACC |
| | CTGTTGAACCCGGTTTGACCATGTATTTGGAAAAGAGAAAGGTCAGAAG |
| | GGCCATAGAAAAGTCTGGTGTACCATACACTTACATATGCTGTAACTCA |
| | ATCGCCTCATGGCCATACTATGATAATAAGCACCCTTCTGAAGTGGTGC |
| | CACCTTTGGATCAATTCCAGATCTATGGCGATGGAACCGTTAAGGCATA |
| | CTTTGTGGATGGACCTGATATTGGTAAATTTACTATGAAGACTGTCGATG |
| | ATATCAGGACTATGAACAAAAACGTTCATTTCAGACCATCCTCCAATTTA |
| | TATGATATTAATGGATTGGCCTCATTGTGGGAAAAGAAGATTGGAAGAA |
| | CTTTGCCAAAGGTGACTATAACCGAGAATGACTTGTTAACAATGGCAGC |
| | TGAAAACAGAATTCCTGAATCTATAGTTGCATCCTTCACACATGATATTT |
| | TCATAAAAGGTTGCCAAACTAATTTTCCCATAGAAGGTCCTAATGACGTT |
| | GACATTGGAACATTATATCCTGAGGAATCCTTTAGGACTTTAGACGAATG |
| | TTTCAATGATTTCTTAGTTAAAGTTGGTGGTAAATTAGAGACAGACAAAT |
| | TAGCAGCTAAAAACAAAGCAGCAGTTGGTGTCGAGCCCATGGCTATTAC |
| | AGCTACATGTGCTTAA |
| SEQ ID NO: 30 | MTVSPSIASAAKSGRVLIIGATGFIGKFVAEASLDSGLPTYVLVRPGPSRPSK |
| | SDTIKSLKDRGAIILHGVMSDKPLMEKLLKEHEIEIVISAVGGATILDQITLVEAI |
| | TSVGTVKRFLPSEFGHDVDRADPVEPGLTMYLEKRKVRRAIEKSGVPYTYI |
| | CCNSIASWPYYDNKHPSEVVPPLDQFQIYGDGTVKAYFVDGPDIGKFTMKT |
| | VDDIRTMNKNVHFRPSSNLYDINGLASLWEKKIGRTLPKVTITENDLLTMAA |
| | ENRIPESIVASFTHDIFIKGCQTNFPIEGPNDVDIGTLYPEESFRTLDECFNDF |
| | LVKVGGKLETDKLAAKNKAAVGVEPMAITATCA |
| SEQ ID NO: 31 | ATGACTTCTGCACTTTATGCCTCCGATCTTTTCAAACAATTGAAAAGTAT |
| | CATGGGAACGGATTCTTTGTCCGATGATGTTGTATTAGTTATTGCTACAA |
| | CTTCTCTGGCACTGGTTGCTGGTTTCGTTGTCTTATTGTGGAAAAAGAC |
| | CACGGCAGATCGTTCCGGCGAGCTAAAGCCACTAATGATCCCTAAGTCT |
| | CTGATGGCGAAAGATGAGGATGATGACTTAGATCTAGGTTCTGGAAAAA |
| | CGAGAGTCTCTATCTTCTTCGGCACACAAACCGGAACAGCCGAAGGATT |
| | CGCTAAAGCACTTTCAGAAGAGATCAAAGCAAGATACGAAAAGGCGGCT |
| | GTAAAAGTAATCGATTTGGATGATTACGCTGCCGATGATGACCAATATG |
| | AGGAAAAGTTGAAAAAGGAAACATTGGCTTTCTTTTGTGTAGCCACGTAT |
| | GGTGATGGTGAACCAACCGATAACGCCGCAAGATTCTACAAGTGGTTTA |
| | CTGAAGAGAACGAAAGAGATATCAAGTTGCAGCAACTTGCTTACGGCGT |
| | TTTTGCCTTAGGTAACAGACAATACGAGCACTTTAACAAGATAGGTATTG |
| | TCTTAGATGAAGAGTTATGCAAAAAGGGTGCGAAGAGATTGATTGAAGT |
| | CGGTTTAGGAGATGATGATCAATCTATCGAGGATGACTTTAATGCATGG |
| | AAGGAATCTTTGTGGTCTGAATTAGATAAGTTACTTAAGGACGAAGATGA |
| | TAAATCCGTTGCCACTCCATACACAGCCGTCATTCCAGAATATAGAGTA |
| | GTTACTCATGATCCAAGATTCACAACACAGAAATCAATGGAAAGTAATGT |
| | GGCTAATGGTAATACTACCATCGATATTCATCATCCATGTAGAGTAGAC |
| | GTTGCAGTTCAAAAGGAATTGCACACTCATGAATCAGACAGATCTTGCA |
| | TACATCTTGAATTTGATATATCACGTACTGGTATCACTTACGAAACAGGT |
| | GATCACGTGGGTGTCTACGCTGAAAACCATGTTGAAATTGTAGAGGAAG |
| | CTGGAAAGTTGTTGGGCCATAGTTTAGATCTTGTTTTCTCAATTCATGCC |
| | GATAAAGAGGATGGCTCACCACTAGAAAGTGCAGTGCCTCCACCATTTC |
| | CAGGACCATGCACCCTAGGTACCGGTTTAGCTCGTTACGCGGATCTGTT |
| | AAATCCTCCACGTAAATCAGCTCTAGTGGCCTTGGCTGCGTACGCCACA |
| | GAACCTTCTGAGGCAGAAAAACTGAAACATCTAACTTCACCAGATGGTA |
| | AGGATGAATACTCACAATGGATAGTAGCTAGTCAACGTTCTTTACTAGAA |
| | GTTATGGCTGCTTTCCCATCCGCTAAACCTCCTTTGGGTGTTTTCTTCGC |
| | CGCAATAGCGCCTAGACTGCAACCAAGATACTATTCAATTTCATCCTCA |
| | CCTAGACTGGCACCATCAAGAGTTCATGTCACATCCGCTTTAGTGTACG |
| | GTCCAACTCCTACTGGTAGAATCCATAAGGGCGTTTGTTCAACATGGAT |
| | GAAAAACGCGGTTCCAGCAGAGAAGTCTCACGAATGTTCTGGTGCTCC |
| | AATCTTTATCAGAGCCTCCAACTTCAAACTGCCTTCCAATCCTTCTACTC |
| | CTATTGTCATGGTCGGTCCTGGTACAGGTCTTGCTCCATTCAGAGGTTT |
| | CTTACAAGAGAGAATGGCCTTAAAGGAGGATGGTGAAGAGTTGGGATC |
| | TTCTTTGTTGTTTTTCGGCTGTAGAAACAGACAAATGGATTTCATCTACG |
| | AAGATGAACTGAATAACTTTGTAGATCAAGGAGTTATTTCAGAGTTGATA |
| | ATGGCTTTTTCTAGAGAAGGTGCTCAGAAGGAGTACGTCCAACACAAAA |
| | TGATGGAAAAGGCCGCACAAGTTTGGGACTTAATCAAAGAGGAAGGCT |
| | ATCTATATGTCTGTGGTGATGCAAAGGGTATGGCAAGAGATGTTCACAG |
| | AACACTTCATACTATAGTCCAGGAACAGGAAGGCGTTAGTTCTTCTGAA |
| | GCGGAAGCAATTGTGAAAAAGTTACAAACAGAGGGAAGATACTTGAGAG |
| | ATGTGTGGTAA |
| SEQ ID NO: 32 | MTSALYASDLFKQLKSIMGTDSLSDDVVLVIATTSLALVAGFVVLLWKKTTA |
| | DRSGELKPLMIPKSLMAKDEDDDLDLGSGKTRVSIFFGTQTGTAEGFAKAL |
| | SEEIKARYEKAAVKVIDLDDYAADDDQYEEKLKKETLAFFCVATYGDGEPTD |
| | NAARFYKWFTEENERDIKLQQLAYGVFALGNRQYEHFNKIGIVLDEELCKK |
| | GAKRLIEVGLGDDDQSIEDDFNAWKESLWSELDKLLKDEDDKSVATPYTAV |
| | IPEYRVVTHDPRFTTQKSMESNVANGNTTIDIHHPCRVDVAVQKELHTHES |
| | DRSCIHLEFDISRTGITYETGDHVGVYAENHVEIVEEAGKLLGHSLDLVFSIH |
| | ADKEDGSPLESAVPPPFPGPCTLGTGLARYADLLNPPRKSALVALAAYATE |
| | PSEAEKLKHLTSPDGKDEYSQWIVASQRSLLEVMAAFPSAKPPLGVFFAAI |
| | APRLQPRYYSISSSPRLAPSRVHVTSALVYGPTPTGRIHKGVCSTWMKNAV |
| | PAEKSHECSGAPIFIRASNFKLPSNPSTPIVMVGPGTGLAPFRGFLQERMAL |
| | KEDGEELGSSLLFFGCRNRQMDFIYEDELNNFVDQGVISELIMAFSREGAQ |
| | KEYVQHKMMEKAAQVWDLIKEEGYLYVCGDAKGMARDVHRTLHTIVQEQE |
| | GVSSSEAEAIVKKLQTEGRYLRDVW |
| SEQ ID NO: 33 | ATGGCAATTCTAGTCACCGACTTCGTTGTCGCGGCTATAATTTTCTTGAT |
| | CACTCGGTTCTTAGTTCGTTCTCTTTTCAAGAAACCAACCCGACCGCTC |
| | CCCCCGGGTCCTCTCGGTTGGCCCTTGGTGGGCGCCCTCCCTCTCCTA |
| | GGCGCCATGCCTCACGTCGCACTAGCCAAACTCGCTAAGAAGTATGGT |
| | CCGATCATGCACCTAAAAATGGGCACGTGCGACATGGTGGTCGCGTCC |
| | ACCCCCGAGTCGGCTCGAGCCTTCCTCAAAACGCTAGACCTCAACTTCT |
| | CCAACCGCCCACCCAACGCGGGCGCATCCCACCTAGCGTACGGCGCG |
| | CAGGACTTAGTCTTCGCCAAGTACGGTCCGAGGTGGAAGACTTTAAGAA |
| | AATTGAGCAACCTCCACATGCTAGGCGGGAAGGCGTTGGATGATTGGG |
| | CAAATGTGAGGGTCACCGAGCTAGGCCACATGCTTAAAGCCATGTGCG |
| | AGGCGAGCCGGTGCGGGGAGCCCGTGGTGCTGGCCGAGATGCTCACG |
| | TACGCCATGGCGAACATGATCGGTCAAGTGATACTCAGCCGGCGCGTG |
| | TTCGTGACCAAAGGGACCGAGTCTAACGAGTTCAAAGACATGGTGGTC |
| | GAGTTGATGACGTCCGCCGGGTACTTCAACATCGGTGACTTCATACCCT |
| | CGATCGCTTGGATGGATTTGCAAGGGATCGAGCGAGGGATGAAGAAGC |
| | TGCACACGAAGTTTGATGTGTTATTGACGAAGATGGTGAAGGAGCATAG |
| | AGCGACGAGTCATGAGCGCAAAGGGAAGGCAGATTTCCTCGACGTTCT |
| | CTTGGAAGAATGCGACAATACAAATGGGGAGAAGCTTAGTATTACCAAT |
| | ATCAAAGCTGTCCTTTTGAATCTATTCACGGCGGGCACGGACACATCTT |
| | CGAGCATAATCGAATGGGCGTTAACGGAGATGATCAAGAATCCGACGA |
| | TCTTAAAAAAGGCGCAAGAGGAGATGGATCGAGTCATCGGTCGTGATC |
| | GGAGGCTGCTCGAATCGGACATATCGAGCCTCCCGTACCTACAAGCCA |
| | TTGCTAAAGAAACGTATCGCAAACACCCGTCGACGCCTCTCAACTTGCC |
| | GAGGATTGCGATCCAAGCATGTGAAGTTGATGGCTACTACATCCCTAAG |
| | GACGCGAGGCTTAGCGTGAACATTTGGGCGATCGGTCGGGACCCGAAT |
| | GTTTGGGAGAATCCGTTGGAGTTCTTGCCGGAAAGATTCTTGTCTGAAG |
| | AGAATGGGAAGATCAATCCCGGTGGGAATGATTTTGAGCTGATTCCGTT |
| | TGGAGCCGGGAGGAGAATTTGTGCGGGGACAAGGATGGGAATGGTCC |
| | TTGTAAGTTATATTTTGGGCACTTTGGTCCATTCTTTTGATTGGAAATTAC |
| | CAAATGGTGTCGCTGAGCTTAATATGGATGAAAGTTTTGGGCTTGCATT |
| | GCAAAAGGCCGTGCCGCTCTCGGCCTTGGTCAGCCCACGGTTGGCCTC |
| | AAACGCGTACGCAACCTGA |
| SEQ ID NO: 34 | MAILVTDFVVAAIIFLITRFLVRSLFKKPTRPLPPGPLGWPLVGALPLLGAMP |
| | HVALAKLAKKYGPIMHLKMGTCDMVVASTPESARAFLKTLDLNFSNRPPNA |
| | GASHLAYGAQDLVFAKYGPRWKTLRKLSNLHMLGGKALDDWANVRVTEL |
| | GHMLKAMCEASRCGEPVVLAEMLTYAMANMIGQVILSRRVFVTKGTESNE |
| | FKDMVVELMTSAGYFNIGDFIPSIAWMDLQGIERGMKKLHTKFDVLLTKMV |
| | KEHRATSHERKGKADFLDVLLEECDNTNGEKLSITNIKAVLLNLFTAGTDTS |
| | SSIIEWALTEMIKNPTILKKAQEEMDRVIGRDRRLLESDISSLPYLQAIAKETY |
| | RKHPSTPLNLPRIAIQACEVDGYYIPKDARLSVNIWAIGRDPNVWENPLEFL |
| | PERFLSEENGKINPGGNDFELIPFGAGRRICAGTRMGMVLVSYILGTLVHSF |
| | DWKLPNGVAELNMDESFGLALQKAVPLSALVSPRLASNAYAT |
| SEQ ID NO: 35 | CTAAATTGTAAGCGTTAATATTTTGTTAAAATTCGCGTTAAATTTTT |
| | GTTAAATCAGCTCATTTTTTAACCAATAGGCCGAAATCGGCAAAAT |
| | CCCTTATAAATCAAAAGAATAGACCGAGATAGGGTTGAGTGGCCG |
| | CTACAGGGCGCTCCCATTCGCCATTCAGGCTGCGCAACTGTTGGGA |
| | AGGGCGTTTCGGTGCGGGCCTCTTCGCTATTACGCCAGCTGGCGAA |
| | AGGGGGATGTGCTGCAAGGCGATTAAGTTGGGTAACGCCAGGGTT |
| | TTCCCAGTCACGACGTTGTAAAACGACGGCCAGTGAGCGCGACGT |
| | AATACGACTCACTATAGGGCGAATTGAAGGAAGGCCGTCAAGGCC |
| | GCATGTCGACGGCGCGCCAGTTACTTGCTCTATGCGTTTGCGCATC |
| | CTCTTTTTACTTTTTTTTTTTCAGTAAAGCCTAAGCATAAATCGTTT |
| | TATACGTACGACACGTTCAACTTTTCTTGGTTAGTAGTGGCAATCT |
| | CTGCAATACATACAGGGAGTCATGGTCTATCATCTTGTCCAATCAA |
| | AGAAGCATCGGTTCAGATCGAGCAAACTGTAGGGAGAAAGGAAA |
| | GTAGAAATGCAGAGTGTGCTATATGTCCAATCTCGGTTTTGTAGTT |
| | TGGATGTCATTAGAGATCTACCACCCAACCGGCTGCTTTCATGTGG |
| | AACAGAAAAGAAATCGGGGCGCTTCCTCTTCTGTATTCCTTTAATT |
| | AACGTTTTTATTCAGCCATCTAACCATCATACCCCCATACGGTAAC |
| | AAAACCTCTTCTAAGAAAAGAAGTCTCTGCTCCTCCGCCATCTTAT |
| | TTTTATTCGCTGCGCGCGTTTATTGTCGCATCGCTAGCCAGCAAAA |
| | AGTTGGTTGCCTTTTTTTACCTAAAAAAGACACATCTAACTGATTA |
| | GTTTTCCGTTTTAGGATATTGACGCCAAGCGTGCGTCTGATTCCCG |
| | GGTCATCGTCCACCTCCGGAGAACAGGCCACCATCACGCATCTGT |
| | GTCTGAATTTCATCACGAGGCGCGCCTTTTCCCGTCTTTCAGTGCCT |
| | TGTTCAGTTCTTCCTGACGGGCGGTATATTTCTCCAGCTTACTAGTT |
| | TACGTGGATTGAGCCAGCAATACAGATCATTATTAAACTGTTTTGT |
| | ACATGATGTTAGTATATAATCGTAAAGCTTTTCTAATATGTATACC |
| | TTATACATGGAACTCCACAGAACTTGCAAACATACCAAAAATCCTT |
| | TATTCTTGTTCACTCATTTTACATCAAAAAATAATATTTCAGTTATT |
| | AAGGAAAATAAAAAAATAGATTAGAGAAGCATTTTGAAGAAATA |
| | GTATATTCTTTTATTGAACCTAAGAGCGTGATATTTTTACTCGAAA |
| | TAAAATACGAAAAATCTATACACTCATCTTTCCGACTACTATTGGC |
| | TCCTGCTCAAAAAAAGAGGGAAAAAAAGCTCCAAAATTCTATCTT |
| | TTCCTATCGCTCCTGTCCTATCCTTATTACGTTCATTACTATTTTAA |
| | TACTATCCATTCTTTTATTTTCAGTCTAAAAAAAACATTTCTCATAA |
| | CGGGAAAAGCAAAAAAATGTCAAGCTTATACATCAAAACACCACT |
| | GCATGCATTATCTGCTGGTCCGGATTCTCAGGCGCGCCCCTGCAGG |
| | CTGGGCCTCATGGGCCTTCCTTTCACTGCCCGCTTTCCAGTCGGGA |
| | AACCTGTCGTGCCAGCTGCATTAACATGGTCATAGCTGTTTCCTTG |
| | CGTATTGGGCGCTCTCCGCTTCCTCGCTCACTGACTCGCTGCGCTC |
| | GGTCGTTCGGGTAAAGCCTGGGGTGCCTAATGAGCAAAAGGCCAG |
| | CAAAAGGCCAGGAACCGTAAAAAGGCCGCGTTGCTGGCGTTTTTC |
| | CATAGGCTCCGCCCCCCTGACGAGCATCACAAAAATCGACGCTCA |
| | AGTCAGAGGTGGCGAAACCCGACAGGACTATAAAGATACCAGGC |
| | GTTTCCCCCTGGAAGCTCCCTCGTGCGCTCTCCTGTTCCGACCCTGC |
| | CGCTTACCGGATACCTGTCCGCCTTTCTCCCTTCGGGAAGCGTGGC |
| | GCTTTCTCATAGCTCACGCTGTAGGTATCTCAGTTCGGTGTAGGTC |
| | GTTCGCTCCAAGCTGGGCTGTGTGCACGAACCCCCCGTTCAGCCCG |
| | ACCGCTGCGCCTTATCCGGTAACTATCGTCTTGAGTCCAACCCGGT |
| | AAGACACGACTTATCGCCACTGGCAGCAGCCACTGGTAACAGGAT |
| | TAGCAGAGCGAGGTATGTAGGCGGTGCTACAGAGTTCTTGAAGTG |
| | GTGGCCTAACTACGGCTACACTAGAAGAACAGTATTTGGTATCTGC |
| | GCTCTGCTGAAGCCAGTTACCTTCGGAAAAAGAGTTGGTAGCTCTT |
| | GATCCGGCAAACAAACCACCGCTGGTAGCGGTGGTTTTTTTGTTTG |
| | CAAGCAGCAGATTACGCGCAGAAAAAAAGGATCTCAAGAAGATC |
| | CTTTGATCTTTTCTACGGGGTCTGACGCTCAGTGGAACGAAAACTC |
| | ACGTTAAGGGATTTTGGTCATGAGATTATCAAAAAGGATCTTCACC |
| | TAGATCCTTTTAAATTAAAAATGAAGTTTTAAATCAATCTAAAGTA |
| | TATATGAGTAAACTTGGTCTGACAGTTATTAGAAAAATTCATCCAG |
| | CAGACGATAAAACGCAATACGCTGGCTATCCGGTGCCGCAATGCC |
| | ATACAGCACCAGAAAACGATCCGCCCATTCGCCGCCCAGTTCTTCC |
| | GCAATATCACGGGTGGCCAGCGCAATATCCTGATAACGATCCGCC |
| | ACGCCCAGACGGCCGCAATCAATAAAGCCGCTAAAACGGCCATTT |
| | TCCACCATAATGTTCGGCAGGCACGCATCACCATGGGTCACCACC |
| | AGATCTTCGCCATCCGGCATGCTCGCTTTCAGACGCGCAAACAGCT |
| | CTGCCGGTGCCAGGCCCTGATGTTCTTCATCCAGATCATCCTGATC |
| | CACCAGGCCCGCTTCCATACGGGTACGCGCACGTTCAATACGATGT |
| | TTCGCCTGATGATCAAACGGACAGGTCGCCGGGTCCAGGGTATGC |
| | AGACGACGCATGGCATCCGCCATAATGCTCACTTTTTCTGCCGGCG |
| | CCAGATGGCTAGACAGCAGATCCTGACCCGGCACTTCGCCCAGCA |
| | GCAGCCAATCACGGCCCGCTTCGGTCACCACATCCAGCACCGCCG |
| | CACACGGAACACCGGTGGTGGCCAGCCAGCTCAGACGCGCCGCTT |
| | CATCCTGCAGCTCGTTCAGCGCACCGCTCAGATCGGTTTTCACAAA |
| | CAGCACCGGACGACCCTGCGCGCTCAGACGAAACACCGCCGCATC |
| | AGAGCAGCCAATGGTCTGCTGCGCCCAATCATAGCCAAACAGACG |
| | TTCCACCCACGCTGCCGGGCTACCCGCATGCAGGCCATCCTGTTCA |
| | ATCATACTCTTCCTTTTTCAATATTATTGAAGCATTTATCAGGGTTA |
| | TTGTCTCATGAGCGGATACATATTTGAATGTATTTAGAAAAATAAA |
| | CAAATAGGGGTTCCGCGCACATTTCCCCGAAAAGTGCCAC |
| SEQ ID NO: 36 | CGGTGCGGGCCTCTTCGCTATTACGCCAGCTGGCGAAAGGGGGAT |
| | GTGCTGCAAGGCGATTAAGTTGGGTAACGCCAGGGTTTTCCCAGTC |
| | ACGACGTTGTAAAACGACGGCCAGTGAATTGTAATACGACTCACT |
| | ATAGGGCGACCCTTAGGATCCTATGGCGCGCCTCATCGTCCACCTC |
| | CGGAGAACAGGCCACCATCACGCATCTGTGTCTGAATTTCATCACG |
| | ACGCGCCGCTGCAGGTCGACAACCCTTAATATAACTTCGTATAATG |
| | TATGCTATACGAAGTTATTAGGTCTAGAGATCCCAATACAACAGAT |
| | CACGTGATCTTTTGTAAGATGAAGTTGAAGTGAGTGTTGCACCGTG |
| | CCAATGCAGGTGGCTATTAGATTAAATATGTGATTTGTTCTATTAA |
| | GTTTCCTGTATAATTAATGGGGAGCGCTGATTCTCTTTTGGTACGC |
| | TTCCCATCCAGCATTTCTGTATCTTTCACCTTCAACCTTAGGATCTC |
| | TACCCTTGGCGAAAAGTCCTCTGCCAACAATGATGATATCTGATCC |
| | ACCACTTACAACTTCGTCGACGGTTCTGTACTGCTGACCCAATGCA |
| | TCGCCTTTGTCGTCTAAACCTACACCTGGGGTCATGATTAGCCAAT |
| | CAAACCCTTCTTCTCTTCCTCCCATATCGTTCTGAGCAATGAACCC |
| | AATAACGAAATCTTTATCACTCTTTGCAATATCAACGGTACCCTTA |
| | GTATATTCACCGTGTGCTAGAGAACCCTTGGAAGACAATTCAGCA |
| | AGCATCAATAATCCCCTTGGTTCTTTGGTGACCTCTTGCGCACCTT |
| | GTTTCAAGCCAGCAACAATACCAGCACCAGTAACCCCGTGGGCGT |
| | TGGTGATATCAGACCATTCTGCGATACGGTAAACGCCCGATGTATA |
| | TTGTAATTTGACTGTGTTACCGATATCGGCGAATTTTCTGTCCTCAA |
| | ATATCAAGAACTTGTATTTCTCTGCCAATGCTTTCAATGGAACGAC |
| | AGTACCCTCATAACTGAAATCATCCAAGATATCAACGTGTGTTTTC |
| | AAAAGGCAAATGTATGGACCCAACGTTTCAACAAGTTTCAATAGC |
| | TCATCAGTCGAACGAACGTCAAGAGAAGCACACAAATTGGTCTTC |
| | TTTTCATCCATTAAACGTAAAAGTTTCGATGCAACCGGACTTGCAT |
| | GAGTCTCAGCTCTACTGGTATATGATTTTGTGGACATGGTGCAACT |
| | AATTGACGGGAGTGTATTGACGCTGGCGTACTGGCTTTCACAAAAT |
| | GGCCCAATCACAACCACATCTTAGATAGTTGAAATGACTTTAGATA |
| | ACATCAATTGAGATGAGCTTAATCATGTCAAAGCTAAAAGTGTCA |
| | CCATGAACGACAATTCTTAAGCAAATCACGTGATATAGATCCACG |
| | AATAACCACCATTTGATGCTCGAGGCAAGTAATGTGTGTAAAAAA |
| | ATGCGTTACCACCATCCAATGCAGACCGATCTTCTACCCAGAATCA |
| | CATATATTTATGTACCGAGTACCTTTTTTCTATCTTCCAATTGCTTC |
| | TCCCATATGATTGTCTCCGTAAGCTCGAAATTTCTAAGTTGGATTTT |
| | AATCTTCACGCAGGATGACAGTTCGATGAGCTTCTGAGGAGTGTTT |
| | AGAACATAATCAGTTTATCCATGGTCTATCTCTTCTTGTCGCTTTTT |
| | CTCCTCGATAGAACCTAAATAAAACGAGCTCTCGAGAACCCTTAA |
| | TATAACTTCGTATAATGTATGCTATACGAAGTTATTAGGTGATATC |
| | AGATCCGGCGCGTGGCACCCTTGCGGGCCATGTCATACACCGCCTT |
| | CAGAGCAGCCGGACCTATCTGCCCGTTGGCGCGCCTATTGAAAGA |
| | TCTTAAGGGGATATCCTCGAGGTTCCCTTTAGTGAGGGTTAATTGC |
| | GAGCTTGGCGTAATCATGGTCATAGCTGTTTCCTGTGTGAAATTGT |
| | TATCCGCTCACAATTCCACACAACATACGAGCCGGAAGCATAAAG |
| | TGTAAAGCCTGGGGTGCCTAATGAGTGAGCTAACTCACATTAATTG |
| | CGTTGCGCTCACTGCCCGCTTTCCAGTCGGGAAACCTGTCGTGCCA |
| | GCTGCATTAATGAATCGGCCAACGCGCGGGGAGAGGCGGTTTGCG |
| | TATTGGGCGCTCTTCCGCTTCCTCGCTCACTGACTCGCTGCGCTCG |
| | GTCGTTCGGCTGCGGCGAGCGGTATCAGCTCACTCAAAGGCGGTA |
| | ATACGGTTATCCACAGAATCAGGGGATAACGCAGGAAAGAACATG |
| | TGAGCAAAAGGCCAGCAAAAGGCCAGGAACCGTAAAAAGGCCGC |
| | GTTGCTGGCGTTTTTCCATAGGCTCCGCCCCCCTGACGAGCATCAC |
| | AAAAATCGACGCTCAAGTCAGAGGTGGCGAAACCCGACAGGACTA |
| | TAAAGATACCAGGCGTTTCCCCCTGGAAGCTCCCTCGTGCGCTCTC |
| | CTGTTCCGACCCTGCCGCTTACCGGATACCTGTCCGCCTTTCTCCCT |
| | TCGGGAAGCGTGGCGCTTTCTCATAGCTCACGCTGTAGGTATCTCA |
| | GTTCGGTGTAGGTCGTTCGCTCCAAGCTGGGCTGTGTGCACGAACC |
| | CCCCGTTCAGCCCGACCGCTGCGCCTTATCCGGTAACTATCGTCTT |
| | GAGTCCAACCCGGTAAGACACGACTTATCGCCACTGGCAGCAGCC |
| | ACTGGTAACAGGATTAGCAGAGCGAGGTATGTAGGCGGTGCTACA |
| | GAGTTCTTGAAGTGGTGGCCTAACTACGGCTACACTAGAAGAACA |
| | GTATTTGGTATCTGCGCTCTGCTGAAGCCAGTTACCTTCGGAAAAA |
| | GAGTTGGTAGCTCTTGATCCGGCAAACAAACCACCGCTGGTAGCG |
| | GTGGTTTTTTTGTTTGCAAGCAGCAGATTACGCGCAGAAAAAAAG |
| | GATCTCAAGAAGATCCTTTGATCTTTTCTACGGGGTCTGACGCTCA |
| | GTGGAACGAAAACTCACGTTAAGGGATTTTGGTCATGAGATTATC |
| | AAAAAGGATCTTCACCTAGATCCTTTTAAATTAAAAATGAAGTTTT |
| | AAATCAATCTAAAGTATATATGAGTAAACTTGGTCTGACAGTTACC |
| | AATGCTTAATCAGTGAGGCACCTATCTCAGCGATCTGTCTATTTCG |
| | TTCATCCATAGTTGCCTGACTCCCCGTCGTGTAGATAACTACGATA |
| | CGGGAGGGCTTACCATCTGGCCCCAGTGCTGCAATGATACCGCGA |
| | GACCCACGCTCACCGGCTCCAGATTTATCAGCAATAAACCAGCCA |
| | GCCGGAAGGGCCGAGCGCAGAAGTGGTCCTCAACTTTATCCGCC |
| | TCCATCCAGTCTATTAATTGTTGCCGGGAAGCTAGAGTAAGTAGTT |
| | CGCCAGTTAATAGTTTGCGCAACGTTGTTGCCATTGCTACAGGCAT |
| | CGTGGTGTCACGCTCGTCGTTTGGTATGGCTTCATTCAGCTCCGGT |
| | TCCCAACGATCAAGGCGAGTTACATGATCCCCCATGTTGTGCAAA |
| | AAAGCGGTTAGCTCCTTCGGTCCTCCGATCGTTGTCAGAAGTAAGT |
| | TGGCCGCAGTGTTATCACTCATGGTTATGGCAGCACTGCATAATTC |
| | TCTTACTGTCATGCCATCCGTAAGATGCTTTTCTGTGACTGGTGAG |
| | TACTCAACCAAGTCATTCTGAGAATAGTGTATGCGGCGACCGAGTT |
| | GCTCTTGCCCGGCGTCAATACGGGATAATACCGCGCCACATAGCA |
| | GAACTTTAAAAGTGCTCATCATTGGAAAACGTTCTTCGGGGCGAA |
| | AACTCTCAAGGATCTTACCGCTGTTGAGATCCAGTTCGATGTAACC |
| | CACTCGTGCACCCAACTGATCTTCAGCATCTTTTACTTTCACCAGC |
| | GTTTCTGGGTGAGCAAAAACAGGAAGGCAAAATGCCGCAAAAAA |
| | GGGAATAAGGGCGACACGGAAATGTTGAATACTCATACTCTTCCT |
| | TTTTCAATATTATTGAAGCATTTATCAGGGTTATTGTCTCATGAGC |
| | GGATACATATTTGAATGTATTTAGAAAAATAAACAAATAGGGGTT |
| | CCGCGCACATTTCCCCGAAAAGTGCCACCTGACGCGCCCTGTAGC |
| | GGCGCATTAAGCGCGGCGGGTGTGGTGGTTACGCGCAGCGTGACC |
| | GCTACACTTGCCAGCGCCCTAGCGCCCGCTCCTTTCGCTTTCTTCCC |
| | TTCCTTTCTCGCCACGTTCGCCGGCTTTCCCCGTCAAGCTCTAAATC |
| | GGGGGCTCCCTTTAGGGTTCCGATTTAGTGCTTTACGGCACCTCGA |
| | CCCCAAAAAACTTGATTAGGGTGATGGTTCACGTAGTGGGCCATC |
| | GCCCTGATAGACGGTTTTTCGCCCTTTGACGTTGGAGTCCACGTTC |
| | TTTAATAGTGGACTCTTGTTCCAAACTGGAACAACACTCAACCCTA |
| | TCTCGGTCTATTCTTTTGATTTATAAGGGATTTTGCCGATTTCGGCC |
| | TATTGGTTAAAAAATGAGCTGATTTAACAAAAATTTAACGCGAATT |
| | TTAACAAAATATTAACGCTTACAATTTGCCATTCGCCATTCAGGCT |
| | GCGCAACTGTTGGGAAGGGCGAT |
| SEQ ID NO: 37 | CGGTGCGGGCCTCTTCGCTATTACGCCAGCTGGCGAAAGGGGGAT |
| | GTGCTGCAAGGCGATTAAGTTGGGTAACGCCAGGGTTTTCCCAGTC |
| | ACGACGTTGTAAAACGACGGCCAGTGAATTGTAATACGACTCACT |
| | ATAGGGCGACCCTAGGATCCTATGGCGCGCCGCCACCAACAGCCC |
| | CGCCAATGGCGCTGCCGATACTCCCGACAATCCCCACCATTGCCTG |
| | ACGCGTCCAGTATCCCAGCAGATACGGGATATCGACATTTCTGCAC |
| | CATTCCGGCGGGTATAGGTTTTATTGATGGCCTCATCCACACGCAG |
| | CAGCGTCTGTTCATCGTCGTGGCGGCCCATAATAATCTGCCGGTCA |
| | ATCAGCCAGCTTTCCTCACCCGGCCCCCATCCCCATACGCGCATTT |
| | CGTAGCGGTCCAGCTGGGAGTCGATACCGGCGGTCAGGTAAGCCA |
| | CACGGTCAGGAACGGGCGCTGAATAATGCTCTTTCCGCTCTGCCAT |
| | CACTTCAGCATCCGGACGTTCGCCAATTTTCGCCTCCCACGTCTCA |
| | CCGAGCGTGGTGTTTACGAAGGTTTTACGTTTTCCCGTATCCCCTTT |
| | CGTTTTCATCCAGTCTTTGACAATCTGCACCCAGGTGGTGAACGGG |
| | CTGTACGCTGTCCAGATGTGAAAGGTCACACTGTCAGGTGGCTCA |
| | ATCTCTTCACCGGATGACGAAAACCAGAGAATGCCATCACGGGTC |
| | CAGATCCCGGTCTTTTCGCAGATATAACGGGCATCAGTAAAGTCCA |
| | GCTCCTGCTGGCGGATGACGCAGGCATTATGCTCGCAGAGATAAA |
| | ACACGCTGGAGACGCGTTTTCCCGTCTTTCAGTGCCTTGTTCAGTT |
| | CTTCCTGACGGGCGGTATATTTCTCCAGCTTGGCGCGCCTAAGACT |
| | TAGATCTTAAGGGGATATCCTCGAGGTTCCCTTTAGTGAGGGTTAA |
| | TTGCGAGCTTGGCGTAATCATGGTCATAGCTGTTTCCTGTGTGAAA |
| | TTGTTATCCGCTCACAATTCCACACAACATACGAGCCGGAAGCATA |
| | AAGTGTAAAGCCTGGGGTGCCTAATGAGTGAGCTAACTCACATTA |
| | ATTGCGTTGCGCTCACTGCCCGCTTTCCAGTCGGGAAACCTGTCGT |
| | GCCAGCTGCATTAATGAATCGGCCAACGCGCGGGGAGAGGCGGTT |
| | TGCGTATTGGGCGCTCTTCCGCTTCCTCGCTCACTGACTCGCTGCG |
| | CTCGGTCGTTCGGCTGCGGCGAGCGGTATCAGCTCACTCAAAGGC |
| | GGTAATACGGTTATCCACAGAATCAGGGGATAACGCAGGAAAGAA |
| | CATGTGAGCAAAAGGCCAGCAAAAGGCCAGGAACCGTAAAAAGG |
| | CCGCGTTGCTGGCGTTTTTCCATAGGCTCCGCCCCCCTGACGAGCA |
| | TCACAAAAATCGACGCTCAAGTCAGAGGTGGCGAAACCCGACAGG |
| | ACTATAAAGATACCAGGCGTTTCCCCCTGGAAGCTCCCTCGTGCGC |
| | TCTCCTGTTCCGACCCTGCCGCTTACCGGATACCTGTCCGCCTTTCT |
| | CCCTTCGGGAAGCGTGGCGCTTTCTCATAGCTCACGCTGTAGGTAT |
| | CTCAGTTCGGTGTAGGTCGTTCGCTCCAAGCTGGGCTGTGTGCACG |
| | AACCCCCCGTTCAGCCCGACCGCTGCGCCTTATCCGGTAACTATCG |
| | TCTTGAGTCCAACCCGGTAAGACACGACTTATCGCCACTGGCAGC |
| | AGCCACTGGTAACAGGATTAGCAGAGCGAGGTATGTAGGCGGTGC |
| | TACAGAGTTCTTGAAGTGGTGGCCTAACTACGGCTACACTAGAAG |
| | AACAGTATTTGGTATCTGCGCTCTGCTGAAGCCAGTTACCTTCGGA |
| | AAAAGAGTTGGTAGCTCTTGATCCGGCAAACAAACCACCGCTGGT |
| | AGCGGTGGTTTTTTTGTTTGCAAGCAGCAGATTACGCGCAGAAAA |
| | AAAGGATCTCAAGAAGATCCTTTGATCTTTTCTACGGGGTCTGACG |
| | CTCAGTGGAACGAAAACTCACGTTAAGGGATTTTGGTCATGAGAT |
| | TATCAAAAAGGATCTTCACCTAGATCCTTTTAAATTAAAAATGAAG |
| | TTTTAAATCAATCTAAAGTATATATGAGTAAACTTGGTCTGACAGT |
| | TACCAATGCTTAATCAGTGAGGCACCTATCTCAGCGATCTGTCTAT |
| | TTCGTTCATCCATAGTTGCCTGACTCCCCGTCGTGTAGATAACTAC |
| | GATACGGGAGGGCTTACCATCTGGCCCCAGTGCTGCAATGATACC |
| | GCGAGACCCACGCTCACCGGCTCCAGATTTATCAGCAATAAACCA |
| | GCCAGCCGGAAGGGCCGAGCGCAGAAGTGGTCCTGCAACTTTATC |
| | CGCCTCCATCCAGTCTATTAATTGTTGCCGGGAAGCTAGAGTAAGT |
| | AGTTCGCCAGTTAATAGTTTGCGCAACGTTGTTGCCATTGCTACAG |
| | GCATCGTGGTGTCACGCTCGTCGTTTGGTATGGCTTCATTCAGCTC |
| | CGGTTCCCAACGATCAAGGCGAGTTACATGATCCCCCATGTTGTGC |
| | AAAAAAGCGGTTAGCTCCTTCGGTCCTCCGATCGTTGTCAGAAGTA |
| | AGTTGGCCGCAGTGTTATCACTCATGGTTATGGCAGCACTGCATAA |
| | TTCTCTTACTGTCATGCCATCCGTAAGATGCTTTTCTGTGACTGGTG |
| | AGTACTCAACCAAGTCATTCTGAGAATAGTGTATGCGGCGACCGA |
| | GTTGCTCTTGCCCGGCGTCAATACGGGATAATACCGCGCCACATAG |
| | CAGAACTTTAAAAGTGCTCATCATTGGAAAACGTTCTTCGGGGCG |
| | AAAACTCTCAAGGATCTTACCGCTGTTGAGATCCAGTTCGATGTAA |
| | CCCACTCGTGCACCCAACTGATCTTCAGCATCTTTTACTTTCACCA |
| | GCGTTTCTGGGTGAGCAAAAACAGGAAGGCAAAATGCCGCAAAA |
| | AAGGGAATAAGGGCGACACGGAAATGTTGAATACTCATACTCTTC |
| | CTTTTTCAATATTATTGAAGCATTTATCAGGGTTATTGTCTCATGAG |
| | CGGATACATATTTGAATGTATTTAGAAAAATAAACAAATAGGGGT |
| | TCCGCGCACATTTCCCCGAAAAGTGCCACCTGACGCGCCCTGTAGC |
| | GGCGCATTAAGCGCGGCGGGTGTGGTGGTTACGCGCAGCGTGACC |
| | GCTACACTTGCCAGCGCCCTAGCGCCCGCTCCTTTCGCTTTCTTCCC |
| | TTCCTTTCTCGCCACGTTCGCCGGCTTTCCCCGTCAAGCTCTAAATC |
| | GGGGGCTCCCTTTAGGGTTCCGATTTAGTGCTTTACGGCACCTCGA |
| | CCCCAAAAAACTTGATTAGGGTGATGGTTCACGTAGTGGGCCATC |
| | GCCCTGATAGACGGTTTTTCGCCCTTTGACGTTGGAGTCCACGTTC |
| | TTTAATAGTGGACTCTTGTTCCAAACTGGAACAACACTCAACCCTA |
| | TCTCGGTCTATTCTTTTGATTTATAAGGGATTTTGCCGATTTCGGCC |
| | TATTGGTTAAAAAATGAGCTGATTTAACAAAAATTTAACGCGAATT |
| | TTAACAAAATATTAACGCTTACAATTTGCCATTCGCCATTCAGGCT |
| | GCGCAACTGTTGGGAAGGGCGAT |
| SEQ ID NO: 38 | CGGCCGCCTGCACGGTCCTGTTCCCTAGCATGTACGTGAGCGTATT |
| | TCCTTTTAAACCACGACGCTTTGTCTTCATTCAACGTTTCCCATTGT |
| | TTTTTTCTACTATTGCTTTGCTGTGGGAAAAACTTATCGAAAGATG |
| | ACGACTTTTTCTTAATTCTCGTTTTAAGAGCTTGGTGAGCGCTAGG |
| | AGTCACTGCCAGGTATCGTTTGAACACGGCATTAGTCAGGGAAGT |
| | CATAACACAGTCCTTTCCCGCAATTTTCTTTTTCTATTACTCTTGGC |
| | CTCCTCTAGTACACTCTATATTTTTTTATGCCTCGGTAATGATTTTC |
| | ATTTTTTTTTTTCCACCTAGCGGATGACTCTTTTTTTTTCTTAGCGAT |
| | TGGCATTATCACATAATGAATTATACATTATATAAAGTAATGTGAT |
| | TTCTTCGAAGAATATACTAAAAAATGAGCAGGCAAGATAAACGAA |
| | GGCAAAGATGACAGAGCAGAAAGCCCTAGTAAAGCGTATTACAA |
| | ATGAAACCAAGATTCAGATTGCGATCTCTTTAAAGGGTGGTCCCCT |
| | AGCGATAGAGCACTCGATCTTCCCAGAAAAAGAGGCAGAAGCAGT |
| | AGCAGAACAGGCCACACAATCGCAAGTGATTAACGTCCACACAGG |
| | TATAGGGTTTCTGGACCATATGATACATGCTCTGGCCAAGCATTCC |
| | GGCTGGTCGCTAATCGTTGAGTGCATTGGTGACTTACACATAGACG |
| | ACCATCACACCACTGAAGACTGCGGGATTGCTCTCGGTCAAGCTTT |
| | TAAAGAGGCCCTAGGGGCCGTGCGTGGAGTAAAAAGGTTTGGATC |
| | AGGATTTGCGCCTTTGGATGAGGCACTTTCCAGAGCGGTGGTAGAT |
| | CTTTCGAACAGGCCGTACGCAGTTGTCGAACTTGGTTTGCAAAGGG |
| | AGAAAGTAGGAGATCTCTCTTGCGAGATGATCCCGCATTTTCTTGA |
| | AAGCTTTGCAGAGGCTAGCAGAATTACCCTCCACGTTGATTGTCTG |
| | CGAGGCAAGAATGATCATCACCGTAGTGAGAGTGCGTTCAAGGCT |
| | CTTGCGGTTGCCATAAGAGAAGCCACCTCGCCCAATGGTACCAAC |
| | GATGTTCCCTCCACCAAAGGTGTTCTTATGTAGTGACACCGATTAT |
| | TTAAAGCTGCAGCATACGATATATATACATGTGTATATATGTATAC |
| | CTATGAATGTCAGTAAGTATGTATACGAACAGTATGATACTGAAG |
| | ATGACAAGGTAATGCATCATTCTATACGTGTCATTCTGAACGAGGC |
| | GCGCTTTCCTTTTTTCTTTTTGCTTTTTCTTTTTTTTTCTCTTGAACTC |
| | GATCGAGAAAAAAAATATAAAAGAGATGGAGGAACGGGAAAAAG |
| | TTAGTTGTGGTGATAGGTGGCAAGTGGTATTCCGTAAGAACAACA |
| | AGAAAAGCATTTCATATTATGGCTGAACTGAGCGAACAAGTGCAA |
| | AATTTAAGCATCAACGACAACAACGAGAATGGTTATGTTCCTCCTC |
| | ACTTAAGAGGAAAACCAAGAAGTGCCAGAAATAACAGTAGCAAC |
| | TACAATAACAACAACGGCGGCTACAACGGTGGCCGTGGCGGTGGC |
| | AGCTTCTTTAGCAACAACCGTCGTGGTGGTTACGGCAACGGTGGTT |
| | TCTTCGGTGGAAACAACGGTGGCAGCAGATCTAACGGCCGTTCTG |
| | GTGGTAGATGGATCGATGGCAAACATGTCCCAGCTCCAAGAAACG |
| | AAAAGGCCGAGATCGCCATATTTGGTGTGGCGGCCGCACGCGTTC |
| | ATCGTCCACCTCCGGAGAACAGGCCACCATCACGCATCTGTGTCTG |
| | AATTTCATCACGGGCGCGCCCTGGGCCTCATGGGCCTTCCGCTCAC |
| | TGCCCGCTTTCCAGTCGGGAAACCTGTCGTGCCAGCTGCATTAACA |
| | TGGTCATAGCTGTTTCCTTGCGTATTGGGCGCTCTCCGCTTCCTCGC |
| | TCACTGACTCGCTGCGCTCGGTCGTTCGGGTAAAGCCTGGGGTGCC |
| | TAATGAGCAAAAGGCCAGCAAAAGGCCAGGAACCGTAAAAAGGC |
| | CGCGTTGCTGGCGTTTTTCCATAGGCTCCGCCCCCCTGACGAGCAT |
| | CACAAAAATCGACGCTCAAGTCAGAGGTGGCGAAACCCGACAGG |
| | ACTATAAAGATACCAGGCGTTTCCCCCTGGAAGCTCCCTCGTGCGC |
| | TCTCCTGTTCCGACCCTGCCGCTTACCGGATACCTGTCCGCCTTTCT |
| | CCCTTCGGGAAGCGTGGCGCTTTCTCATAGCTCACGCTGTAGGTAT |
| | CTCAGTTCGGTGTAGGTCGTTCGCTCCAAGCTGGGCTGTGTGCACG |
| | AACCCCCCGTTCAGCCCGACCGCTGCGCCTTATCCGGTAACTATCG |
| | TCTTGAGTCCAACCCGGTAAGACACGACTTATCGCCACTGGCAGC |
| | AGCCACTGGTAACAGGATTAGCAGAGCGAGGTATGTAGGCGGTGC |
| | TACAGAGTTCTTGAAGTGGTGGCCTAACTACGGCTACACTAGAAG |
| | AACAGTATTTGGTATCTGCGCTCTGCTGAAGCCAGTTACCTTCGGA |
| | AAAAGAGTTGGTAGCTCTTGATCCGGCAAACAAACCACCGCTGGT |
| | AGCGGTGGTTTTTTTGTTTGCAAGCAGCAGATTACGCGCAGAAAA |
| | AAAGGATCTCAAGAAGATCCTTTGATCTTTTCTACGGGGTCTGACG |
| | CTCAGTGGAACGAAAACTCACGTTAAGGGATTTTGGTCATGAGAT |
| | TATCAAAAAGGATCTTCACCTAGATCCTTTTAAATTAAAAATGAAG |
| | TTTTAAATCAATCTAAAGTATATATGAGTAAACTTGGTCTGACAGC |
| | TACCAATGCTTAATCAGTGAGGCACCTATCTCAGCGATCTGTCTAT |
| | TTCGTTCATCCATAGTTGCCTGACTCCCCGTCGTGTAGATAACTAC |
| | GATACGGGAGGGCTTACCATCTGGCCCCAGTGCTGCAATGATACC |
| | GCGAGAACCACGCTCACCGGCTCCAGATTTATCAGCAATAAACCA |
| | GCCAGCCGGAAGGGCCGAGCGCAGAAGTGGTCCTGCAACTTTATC |
| | CGCCTCCATCCAGTCTATTAATTGTTGCCGGGAAGCTAGAGTAAGT |
| | AGTTCGCCAGTTAATAGTTTGCGCAACGTTGTTGCCATTGCTACAG |
| | GCATCGTGGTGTCACGCTCGTCGTTTGGTATGGCTTCATTCAGCTC |
| | CGGTTCCCAACGATCAAGGCGAGTTACATGATCCCCCATGTTGTGC |
| | AAAAAAGCGGTTAGCTCCTTCGGTCCTCCGATCGTTGTCAGAAGTA |
| | AGTTGGCCGCAGTGTTATCACTCATGGTTATGGCAGCACTGCATAA |
| | TTCTCTTACTGTCATGCCATCCGTAAGATGCTTTTCTGTGACTGGTG |
| | AGTACTCAACCAAGTCATTCTGAGAATAGTGTATGCGGCGACCGA |
| | GTTGCTCTTGCCCGGCGTCAATACGGGATAATACCGCGCCACATAG |
| | CAGAACTTTAAAAGTGCTCATCATTGGAAAACGTTCTTCGGGGCG |
| | AAAACTCTCAAGGATCTTACCGCTGTTGAGATCCAGTTCGATGTAA |
| | CCCACTCGTGCACCCAACTGATCTTCAGCATCTTTTACTTTCACCA |
| | GCGTTTCTGGGTGAGCAAAAACAGGAAGGCAAAATGCCGCAAAA |
| | AAGGGAATAAGGGCGACACGGAAATGTTGAATACTCATACTCTTC |
| | CTTTTTCAATATTATTGAAGCATTTATCAGGGTTATTGTCTCATGAG |
| | CGGATACATATTTGAATGTATTTAGAAAAATAAACAAATAGGGGT |
| | TCCGCGCACATTTCCCCGAAAAGTGCCACCTAAATTGTAAGCGTTA |
| | ATATTTTGTTAAAATTCGCGTTAAATTTTTGTTAAATCAGCTCATTT |
| | TTTAACCAATAGGCCGAAATCGGCAAAATCCCTTATAAATCAAAA |
| | GAATAGACCGAGATAGGGTTGAGTGGCCGCTACAGGGCGCTCCCA |
| | TTCGCCATTCAGGCTGCGCAACTGTTGGGAAGGGCGTTTCGGTGCG |
| | GGCCTCTTCGCTATTACGCCAGCTGGCGAAAGGGGGATGTGCTGC |
| | AAGGCGATTAAGTTGGGTAACGCCAGGGTTTTCCCAGTCACGACG |
| | TTGTAAAACGACGGCCAGTGAGCGCGACGTAATACGACTCACTAT |
| | AGGGCGAATTGGCGGAAGGCCGTCAAGGCCGCATGGCGCGCCTTT |
| | CCCGTCTTTCAGTGCCTTGTTCAGTTCTTCCTGACGGGCGGTATATT |
| | TCTCCAGCTTGGCCTATGCGGCCCTGTCAGACCAAGTTTACGAGCT |
| | CGCTTGGACTCCTGTTGATAGATCCAGTAATGACCTCAGAACTCCA |
| | TCTGGATTTGTTCAGAACGCTCGGTTGCCGCCGGGCGTTTTTTATT |
| | GGTGAGAATCCAAGCACTAGGGACAGTAAGACGGGTAAGCCTGTT |
| | GATGATACCGCTGCCTTACTGGGTGCATTAGCCAGTCTGAATGACC |
| | TGTCACGGGATAATCCGAAGTGGTCAGACTGGAAAATCAGAGGGC |
| | AGGAACTGCTGAACAGCAAAAAGTCAGATAGCACCACATAGCAG |
| | ACCCGCCATAAAACGCCCTGAGAAGCCCGTGACGGGCTTTTCTTGT |
| | ATTATGGGTAGTTTCCTTGCATGAATCCATAAAAGGCGCCTGTAGT |
| | GCCATTTACCCCCATTCACTGCCAGAGCCGTGAGCGCAGCGAACT |
| | GAATGTCACGAAAAAGACAGCGACTCAGGTGCCTGATGGTCGGAG |
| | ACAAAAGGAATATTCAGCGATTTGCCCGAGCTTGCGAGGGTGCTA |
| | CTTAAGCCTTTAGGGTTTTAAGGTCTGTTTTGTAGAGGAGCAAACA |
| | GCGTTTGCGACATCCTTTTGTAATACTGCGGAACTGACTAAAGTAG |
| | TGAGTTATACACAGGGCTGGGATCTATTCTTTTTATCTTTTTTTATT |
| | CTTTCTTTATTCTATAAATTATAACCACTTGAATATAAACAAAAAA |
| | AACACACAAAGGTCTAGCGGAATTTACAGAGGGTCTAGCAGAATT |
| | TACAAGTTTTCCAGCAAAGGTCTAGCAGAATTTACAGATACCCAC |
| | AACTCAAAGGAAAAGGACATGTAATTATCATTGACTAGCCCATCT |
| | CAATTGGTATAGTGATTAAAATCACCTAGACCAATTGAGATGTATG |
| | TCTGAATTAGTTGTTTTCAAAGCAAATGAACTAGCGATTAGTCGCT |
| | ATGACTTAACGGAGCATGAAACCAAGCTAATTTTATGCTGTGTGGC |
| | ACTACTCAACCCCACGATTGAAAACCCTACAAGGAAAGAACGGAC |
| | GGTATCGTTCACTTATAACCAATACGCTCAGATGATGAACATCAGT |
| | AGGGAAAATGCTTATGGTGTATTAGCTAAAGCAACCAGAGAGCTG |
| | ATGACGAGAACTGTGGAAATCAGGAATCCTTTGGTTAAAGGCTTT |
| | GAGATTTTCCAGTGGACAAACTATGCCAAGTTCTCAAGCGAAAAA |
| | TTAGAATTAGTTTTTAGTGAAGAGATATTGCCTTATCTTTTCCAGTT |
| | AAAAAATTCATAAAATATAATCTGGAACATGTTAAGTCTTTTGAA |
| | AACAAATACTCTATGAGGATTTATGAGTGGTTATTAAAAGAACTA |
| | ACACAAAAGAAAACTCACAAGGCAAATATAGAGATTAGCCTTGAT |
| | GAATTTAAGTTCATGTTAATGCTTGAAAATAACTACCATGAGTTTA |
| | AAAGGCTTAACCAATGGGTTTTGAAACCAATAAGTAAAGATTTAA |
| | ACACTTACAGCAATATGAAATTGGTGGTTGATAAGCGAGGCCGCC |
| | CGACTGATACGTTGATTTTCCAAGTTGAACTAGATAGACAAATGG |
| | ATCTCGTAACCGAACTTGAGAACAACCAGATAAAAATGAATGGTG |
| | ACAAAATACCAACAACCATTACATCAGATTCCTACCTACATAACG |
| | GACTAAGAAAAACACTACACGATGCTTTAACTGCAAAAATTCAGC |
| | TCACCAGTTTTGAGGCAAAATTTTTGAGTGACATGCAAAGTAAGTA |
| | TGATCTCAATGGTTCGTTCTCATGGCTCACGCAAAAACAACGAACC |
| | ACACTAGAGAACATACTGGCTAAATACGGAAGGATCTGAGGTTCT |
| | TATGGCTCTTGTATCTATCAGTGAAGCATCAAGACTAACAAACAA |
| | AAGTAGAACAACTGTTCACCGTTACATATCAAAGGGAAAACTGTC |
| | CATATGCACAGATGAAAACGGTGTAAAAAAGATAGATACATCAGA |
| | GCTTTTACGAGTTTTTGGTGCATTCAAAGCTGTTCACCATGAACAG |
| | ATCGACAATGTAACG |
| SEQ ID NO: 39 | CGGTGCGGGCCTCTTCGCTATTACGCCAGCTGGCGAAAGGGGGAT |
| | GTGCTGCAAGGCGATTAAGTTGGGTAACGCCAGGGTTTTCCCAGTC |
| | ACGACGTTGTAAAACGACGGCCAGTGAATTGTAATACGACTCACT |
| | ATAGGGCGACCCTTAGGATCCTATGGCGCGCCTCATCGTCCACCTC |
| | CGGAGAACAGGCCACCATCACGCATCTGTGTCTGAATTTCATCACG |
| | ACGCGCCTTAAGGGCACCAATAACTGCCTTAAAAAAATTACGCCC |
| | CGCCCTGCCACTCATCGCAGTACTGTTGTAATTCATTAAGCATTCT |
| | GCCGACATGGAAGCCATCACAGACGGCATGATGAACCTGAATCGC |
| | CAGCGGCATCAGCACCTTGTCGCCTTGCGTATAATATTTGCCCATG |
| | GTGAAAACGGGGGCGAAGAAGTTGTCCATATTGGCCACGTTTAAA |
| | TCAAAACTGGTGAAACTCACCCAGGGATTGGCTGAGACGAAAAAC |
| | ATATTCTCAATAAACCCTTTAGGGAAATAGGCCAGGTTTTCACCGT |
| | AACACGCCACATCTTGCGAATATATGTGTAGAAACTGCCGGAAAT |
| | CGTCGTGGTATTCACTCCAGAGCGATGAAAACGTTTCAGTTTGCTC |
| | ATGGAAAACGGTGTAACAAGGGTGAACACTATCCCATATCACCAG |
| | CTCACCGTCTTTCATTGCCATACGGAATTCCGGATGAGCATTCATC |
| | AGGCGGGCAAGAATGTGAATAAAGGCCGGATAAAACTTGTGCTTA |
| | TTTTTCTTTACGGTCTTTAAAAAGGCCGTAATATCCAGCTGAACGG |
| | TCTGGTTATAGGTACATTGAGCAACTGACTGAAATGCCTCAAAATG |
| | TTCTTTACGATGCCATTGGGATATATCAACGGTGGTATATCCAGTG |
| | ATTTTTTTCTCCATTTTAGCTTCCTTAGCTCCTGAAAATCTCGATAA |
| | CTCAAAAAATACGCCCGGTAGTGATCTTATTTCATTATGGTGAAAG |
| | TTGGAACCTCTTACGTGCCGATCAACGTCTCATTTTCGCCAAAAGT |
| | TGGCCCAGGGCTTCCCGGTATCAACAGGGACACCAGGATTTATTTA |
| | TTCTGCGAAGTGATCTTCCGTCACAGGTATTGGACCACCCTGTGGG |
| | TTTATAAGCGCGCTGCTGGCGTGTAAGGCGGTGACGGCGAAGGAA |
| | GGGTCCTTTTCATCACGTGCTATAAAAATAATTATAATTTAAATTT |
| | TTTAATATAAATATATAAATTAAAAATAGAAAGTAAAAAAAGAAA |
| | TTAAAGAAAAAATAGTTTTTGTTTTCCGAAGATGTAAAAGACTCTA |
| | GGGGGATCGCCAACAAATACTACCTTTTATCTTGCTCTTCCTGCTC |
| | TCAGGTATTAATGCCGAATTGTTTCATCTTGTCTGTGTAGAAGACC |
| | ACACACGAAAATCCTGTGATTTTACATTTTACTTATCGTTAATCGA |
| | ATGTATATCTATTTAATCTGCTTTTCTTGTCTAATAAATATATATGT |
| | AAAGTACGCTTTTTGTTGAAATTTTTTAAACCTTTGTTTATTTTTTTT |
| | TCTTCATTCCGTAACTCTTCTACCTTCTTTATTTACTTTCTAAAATCC |
| | AAATACAAAACATAAAAATAAATAAACACAGAGTAAATTCCCAAA |
| | TTATTCCATCATTAAAAGATACGAGGCGCGTGTAAGTTACAGGCA |
| | AGCGATCCGTCCTAAGAAACCATTATTATCATGACATTAACCTATA |
| | AAAATAGGCGTATCACGAGGCCCTTTCGTCTCGCGCGTTTCGGTGA |
| | TGACGGTGAAAACCTCTGACACATGCAGCTCCCGGAGACGGTCAC |
| | AGCTTGTCTGTAAGCGGATGCCGGGAGCAGACAAGCCCGTCAGGG |
| | CGCGTCAGCGGGTGTTGGCGGGTGTCGGGGCTGGCTTAACTATGC |
| | GGCATCAGAGCAGATTGTACTGAGAGTGCACCACGGCGCGTGGCA |
| | CCCTTGCGGGCCATGTCATACACCGCCTTCAGAGCAGCCGGACCTA |
| | TCTGCCCGTTGGCGCGCCTATTGAAAGATCTTAAGGGGATATCCTC |
| | GAGGTTCCCTTTAGTGAGGGTTAATTGCGAGCTTGGCGTAATCATG |
| | GTCATAGCTGTTTCCTGTGTGAAATTGTTATCCGCTCACAATTCCA |
| | CACAACATACGAGCCGGAAGCATAAAGTGTAAAGCCTGGGGTGCC |
| | TAATGAGTGAGCTAACTCACATTAATTGCGTTGCGCTCACTGCCCG |
| | CTTTCCAGTCGGGAAACCTGTCGTGCCAGCTGCATTAATGAATCGG |
| | CCAACGCGCGGGGAGAGGCGGTTTGCGTATTGGGCGCTCTTCCGC |
| | TTCCTCGCTCACTGACTCGCTGCGCTCGGTCGTTCGGCTGCGGCGA |
| | GCGGTATCAGCTCACTCAAAGGCGGTAATACGGTTATCCACAGAA |
| | TCAGGGGATAACGCAGGAAAGAACATGTGAGCAAAAGGCCAGCA |
| | AAAGGCCAGGAACCGTAAAAAGGCCGCGTTGCTGGCGTTTTTCCA |
| | TAGGCTCCGCCCCCCTGACGAGCATCACAAAAATCGACGCTCAAG |
| | TCAGAGGTGGCGAAACCCGACAGGACTATAAAGATACCAGGCGTT |
| | TCCCCCTGGAAGCTCCCTCGTGCGCTCTCCTGTTCCGACCCTGCCG |
| | CTTACCGGATACCTGTCCGCCTTTCTCCCTTCGGGAAGCGTGGCGC |
| | TTTCTCATAGCTCACGCTGTAGGTATCTCAGTTCGGTGTAGGTCGT |
| | TCGCTCCAAGCTGGGCTGTGTGCACGAACCCCCCGTTCAGCCCGAC |
| | CGCTGCGCCTTATCCGGTAACTATCGTCTTGAGTCCAACCCGGTAA |
| | GACACGACTTATCGCCACTGGCAGCAGCCACTGGTAACAGGATTA |
| | GCAGAGCGAGGTATGTAGGCGGTGCTACAGAGTTCTTGAAGTGGT |
| | GGCCTAACTACGGCTACACTAGAAGAACAGTATTTGGTATCTGCG |
| | CTCTGCTGAAGCCAGTTACCTTCGGAAAAAGAGTTGGTAGCTCTTG |
| | ATCCGGCAAACAAACCACCGCTGGTAGCGGTGGTTTTTTTGTTTGC |
| | AAGCAGCAGATTACGCGCAGAAAAAAAGGATCTCAAGAAGATCCT |
| | TTGATCTTTTCTACGGGGTCTGACGCTCAGTGGAACGAAAACTCAC |
| | GTTAAGGGATTTTGGTCATGAGATTATCAAAAAGGATCTTCACCTA |
| | GATCCTTTTAAATTAAAAATGAAGTTTTAAATCAATCTAAAGTATA |
| | TATGAGTAAACTTGGTCTGACAGTTACCAATGCTTAATCAGTGAGG |
| | CACCTATCTCAGCGATCTGTCTATTTCGTTCATCCATAGTTGCCTGA |
| | CTCCCCGTCGTGTAGATAACTACGATACGGGAGGGCTTACCATCTG |
| | GCCCCAGTGCTGCAATGATACCGCGAGACCCACGCTCACCGGCTC |
| | CAGATTTATCAGCAATAAACCAGCCAGCCGGAAGGGCCGAGCGCA |
| | GAAGTGGTCCTGCAACTTTATCCGCCTCCATCCAGTCTATTAATTG |
| | TTGCCGGGAAGCTAGAGTAAGTAGTTCGCCAGTTAATAGTTTGCGC |
| | AACGTTGTTGCCATTGCTACAGGCATCGTGGTGTCACGCTCGTCGT |
| | TTGGTATGGCTTCATTCAGCTCCGGTTCCCAACGATCAAGGCGAGT |
| | TACATGATCCCCCATGTTGTGCAAAAAAGCGGTTAGCTCCTTCGGT |
| | CCTCCGATCGTTGTCAGAAGTAAGTTGGCCGCAGTGTTATCACTCA |
| | TGGTTATGGCAGCACTGCATAATTCTCTTACTGTCATGCCATCCGT |
| | AAGATGCTTTTCTGTGACTGGTGAGTACTCAACCAAGTCATTCTGA |
| | GAATAGTGTATGCGGCGACCGAGTTGCTCTTGCCCGGCGTCAATAC |
| | GGGATAATACCGCGCCACATAGCAGAACTTTAAAAGTGCTCATCA |
| | TTGGAAAACGTTCTTCGGGGCGAAAACTCTCAAGGATCTTACCGCT |
| | GTTGAGATCCAGTTCGATGTAACCCACTCGTGCACCCAACTGATCT |
| | TCAGCATCTTTTACTTTCACCAGCGTTTCTGGGTGAGCAAAAACAG |
| | GAAGGCAAAATGCCGCAAAAAAGGGAATAAGGGCGACACGGAAA |
| | TGTTGAATACTCATACTCTTCCTTTTTCAATATTATTGAAGCATTTA |
| | TCAGGGTTATTGTCTCATGAGCGGATACATATTTGAATGTATTTAG |
| | AAAAATAAACAAATAGGGGTTCCGCGCACATTTCCCCGAAAAGTG |
| | CCACCTGACGCGCCCTGTAGCGGCGCATTAAGCGCGGCGGGTGTG |
| | GTGGTTACGCGCAGCGTGACCGCTACACTTGCCAGCGCCCTAGCG |
| | CCCGCTCCTTTCGCTTTCTTCCCTTCCTTTCTCGCCACGTTCGCCGG |
| | CTTTCCCCGTCAAGCTCTAAATCGGGGGCTCCCTTTAGGGTTCCGA |
| | TTTAGTGCTTTACGGCACCTCGACCCCAAAAAACTTGATTAGGGTG |
| | ATGGTTCACGTAGTGGGCCATCGCCCTGATAGACGGTTTTTCGCCC |
| | TTTGACGTTGGAGTCCACGTTCTTTAATAGTGGACTCTTGTTCCAA |
| | ACTGGAACAACACTCAACCCTATCTCGGTCTATTCTTTTGATTTAT |
| | AAGGGATTTTGCCGATTTCGGCCTATTGGTTAAAAAATGAGCTGAT |
| | TTAACAAAAATTTAACGCGAATTTTAACAAAATATTAACGCTTACA |
| | ATTTGCCATTCGCCATTCAGGCTGCGCAACTGTTGGGAAGGGCGAT |
| SEQ ID NO: 40 | CGGTGCGGGCCTCTTCGCTATTACGCCAGCTGGCGAAAGGGGGAT |
| | GTGCTGCAAGGCGATTAAGTTGGGTAACGCCAGGGTTTTCCCAGTC |
| | ACGACGTTGTAAAACGACGGCCAGTGAATTGTAATACGACTCACT |
| | ATAGGGCGACCCTTAGGATCCTATGGCGCGCCACCACGGTGAACA |
| | ATCCCCGCTGGCTCATATTTGCCGCCGGTTCCCGTAAATCCTCCGG |
| | TACGCGTCCAGTATCCCAGCAGATACGGGATATCGACATTTCTGCA |
| | CCATTCCGGCGGGTATAGGTTTTATTGATGGCCTCATCCACACGCA |
| | GCAGCGTCTGTTCATCGTCGTGGCGGCCCATAATAATCTGCCGGTC |
| | AATCAGCCAGCTTTCCTCACCCGGCCCCCATCCCCATACGCGCATT |
| | TCGTAGCGGTCCAGCTGGGAGTCGATACCGGCGGTCAGGTAAGCC |
| | ACACGGTCAGGAACGGGCGCTGAATAATGCTCTTTCCGCTCTGCCA |
| | TCACTTCAGCATCCGGACGTTCGCCAATTTTCGCCTCCCACGTCTC |
| | ACCGAGCGTGGTGTTTACGAAGGTTTTACGTTTTCCCGTATCCCCT |
| | TTCGTTTTCATCCAGTCTTTGACAATCTGCACCCAGGTGGTGAACG |
| | GGCTGTACGCTGTCCAGATGTGAAAGGTCACACTGTCAGGTGGCT |
| | CAATCTCTTCACCGGATGACGAAAACCAGAGAATGCCATCACGGG |
| | TCCAGATCCCGGTCTTTTCGCAGATATAACGGGCATCAGTAAAGTC |
| | CAGCTCCTGCTGGCGGATGACGCAGGCATTATGCTCGCAGAGATA |
| | AAACACGCTGGAGACGCGTTTTCCCGTCTTTCAGTGCCTTGTTCAG |
| | TTCTTCCTGACGGGCGGTATATTTCTCCAGCTTGGCGCGCCTAAGA |
| | CTTAGATCTTAAGGGGATATCCTCGAGGTTCCCTTTAGTGAGGGTT |
| | AATTGCGAGCTTGGCGTAATCATGGTCATAGCTGTTTCCTGTGTGA |
| | AATTGTTATCCGCTCACAATTCCACACAACATACGAGCCGGAAGC |
| | ATAAAGTGTAAAGCCTGGGGTGCCTAATGAGTGAGCTAACTCACA |
| | TTAATTGCGTTGCGCTCACTGCCCGCTTTCCAGTCGGGAAACCTGT |
| | CGTGCCAGCTGCATTAATGAATCGGCCAACGCGCGGGGAGAGGCG |
| | GTTTGCGTATTGGGCGCTCTTCCGCTTCCTCGCTCACTGACTCGCTG |
| | CGCTCGGTCGTTCGGCTGCGGCGAGCGGTATCAGCTCACTCAAAG |
| | GCGGTAATACGGTTATCCACAGAATCAGGGGATAACGCAGGAAAG |
| | AACATGTGAGCAAAAGGCCAGCAAAAGGCCAGGAACCGTAAAAA |
| | GGCCGCGTTGCTGGCGTTTTTCCATAGGCTCCGCCCCCCTGACGAG |
| | CATCACAAAAATCGACGCTCAAGTCAGAGGTGGCGAAACCCGACA |
| | GGACTATAAAGATACCAGGCGTTTCCCCCTGGAAGCTCCCTCGTGC |
| | GCTCTCCTGTTCCGACCCTGCCGCTTACCGGATACCTGTCCGCCTTT |
| | CTCCCTTCGGGAAGCGTGGCGCTTTCTCATAGCTCACGCTGTAGGT |
| | ATCTCAGTTCGGTGTAGGTCGTTCGCTCCAAGCTGGGCTGTGTGCA |
| | CGAACCCCCCGTTCAGCCCGACCGCTGCGCCTTATCCGGTAACTAT |
| | CGTCTTGAGTCCAACCCGGTAAGACACGACTTATCGCCACTGGCA |
| | GCAGCCACTGGTAACAGGATTAGCAGAGCGAGGTATGTAGGCGGT |
| | GCTACAGAGTTCTTGAAGTGGTGGCCTAACTACGGCTACACTAGA |
| | AGAACAGTATTTGGTATCTGCGCTCTGCTGAAGCCAGTTACCTTCG |
| | GAAAAAGAGTTGGTAGCTCTTGATCCGGCAAACAAACCACCGCTG |
| | GTAGCGGTGGTTTTTTTGTTTGCAAGCAGCAGATTACGCGCAGAAA |
| | AAAAGGATCTCAAGAAGATCCTTTGATCTTTTCTACGGGGTCTGAC |
| | GCTCAGTGGAACGAAAACTCACGTTAAGGGATTTTGGTCATGAGA |
| | TTATCAAAAAGGATCTTCACCTAGATCCTTTTAAATTAAAAATGAA |
| | GTTTTAAATCAATCTAAAGTATATATGAGTAAACTTGGTCTGACAG |
| | TTACCAATGCTTAATCAGTGAGGCACCTATCTCAGCGATCTGTCTA |
| | TTTCGTTCATCCATAGTTGCCTGACTCCCCGTCGTGTAGATAACTA |
| | CGATACGGGAGGGCTTACCATCTGGCCCCAGTGCTGCAATGATAC |
| | CGCGAGACCCACGCTCACCGGCTCCAGATTTATCAGCAATAAACC |
| | AGCCAGCCGGAAGGGCCGAGCGCAGAAGTGGTCCTGCAACTTTAT |
| | CCGCCTCCATCCAGTCTATTAATTGTTGCCGGGAAGCTAGAGTAAG |
| | TAGTTCGCCAGTTAATAGTTTGCGCAACGTTGTTGCCATTGCTACA |
| | GGCATCGTGGTGTCACGCTCGTCGTTTGGTATGGCTTCATTCAGCT |
| | CCGGTTCCCAACGATCAAGGCGAGTTACATGATCCCCCATGTTGTG |
| | CAAAAAAGCGGTTAGCTCCTTCGGTCCTCCGATCGTTGTCAGAAGT |
| | AAGTTGGCCGCAGTGTTATCACTCATGGTTATGGCAGCACTGCATA |
| | ATTCTCTTACTGTCATGCCATCCGTAAGATGCTTTTCTGTGACTGGT |
| | GAGTACTCAACCAAGTCATTCTGAGAATAGTGTATGCGGCGACCG |
| | AGTTGCTCTTGCCCGGCGTCAATACGGGATAATACCGCGCCACATA |
| | GCAGAACTTTAAAAGTGCTCATCATTGGAAAACGTTCTTCGGGGC |
| | GAAAACTCTCAAGGATCTACCGCTGTTGAGATCCAGTTCGATGTA |
| | ACCCACTCGTGCACCCAACTGATCTTCAGCATCTTTTACTTTCACC |
| | AGCGTTTCTGGGTGAGCAAAAACAGGAAGGCAAAATGCCGCAAA |
| | AAAGGGAATAAGGGCGACACGGAAATGTTGAATACTCATACTCTT |
| | CCTTTTTCAATATTATTGAAGCATTTATCAGGGTTATTGTCTCATGA |
| | GCGGATACATATTTGAATGTATTTAGAAAAATAAACAAATAGGGG |
| | TTCCGCGCACATTTCCCCGAAAAGTGCCACCTGACGCGCCCTGTAG |
| | CGGCGCATTAAGCGCGGCGGGTGTGGTGGTTACGCGCAGCGTGAC |
| | CGCTACACTTGCCAGCGCCCTAGCGCCCGCTCCTTTCGCTTTCTTCC |
| | CTTCCTTTCTCGCCACGTTCGCCGGCTTTCCCCGTCAAGCTCTAAAT |
| | CGGGGGCTCCCTTTAGGGTTCCGATTTAGTGCTTTACGGCACCTCG |
| | ACCCCAAAAAACTTGATTAGGGTGATGGTTCACGTAGTGGGCCAT |
| | CGCCCTGATAGACGGTTTTTCGCCCTTTGACGTTGGAGTCCACGTT |
| | CTTTAATAGTGGACTCTTGTTCCAAACTGGAACAACACTCAACCCT |
| | ATCTCGGTCTATTCTTTTGATTTATAAGGGATTTTGCCGATTTCGGC |
| | CTATTGGTTAAAAAATGAGCTGATTTAACAAAAATTTAACGCGAA |
| | TTTTAACAAAATATTAACGCTTACAATTTGCCATTCGCCATTCAGG |
| | CTGCGCAACTGTTGGGAAGGGCGAT |
| SEQ ID NO: 41 | CGGTGCGGGCCTCTTCGCTATTACGCCAGCTGGCGAAAGGGGGAT |
| | GTGCTGCAAGGCGATTAAGTTGGGTAACGCCAGGGTTTTCCCAGTC |
| | ACGACGTTGTAAAACGACGGCCAGTGAATTGTAATACGACTCACT |
| | ATAGGGCGACCCTTAGGCGCGCCTTTCCCGTCTTTCAGTGCCTTGT |
| | TCAGTTCTTCCTGACGGGCGGTATATTTCTCCAGCTTACGCGCCAT |
| | GCAGGGATATCAGATCTTCGAGGAGAACTTCTAGTATATCCACAT |
| | ACCTAATATTATTGCCTTATTAAAAATGGAATCCCAACAATTACAT |
| | CAAAATCCACATTCTCTTCAAAATCAATTGTCCTGTACTTCCTTGTT |
| | CATGTGTGTTCAAAAACGTTATATTTATAGGATAATTATACTCTAT |
| | TTCTCAACAAGTAATTGGTTGTTTGGCCGAGCGGTCTAAGGCGCCT |
| | GATTCAAGAAATATCTTGACCGCAGTTAACTGTGGGAATACTCAG |
| | GTATCGTAAGATGCAAGAGTTCGAATCTCTTAGCAACCATTATTTT |
| | TTTCCTCAACATAACGAGAACACACAGGGGCGCTATCGCACAGAA |
| | TCAAATTCGATGATTGGAAATTTTTTGTTAATTTCAGAGGTCGCCT |
| | GACGCATATACCTTTTTCAACTGAAAAATTGGGAGAAAAAGGAAA |
| | GGTGAGAGGCCGGAACCGGCTTTTCATATAGAATAGAGAAGCGTT |
| | CATGACTAAATGCTTGCATCACAATACTTGAAGTTGACAATATTAT |
| | TTAAGGACCTATTGTTTTTTCCAATAGGTGGTTAGCAATCGTCTTA |
| | CTTTCTAACTTTTCTTACCTTTTACATTTCAGCAATATATATATATA |
| | TTTCAAGGATATACCATTCTAATGTCTGCCCCTATGTCTGCCCCTA |
| | AGAAGATCGTCGTTTTGCCAGGTGACCACGTTGGTCAAGAAATCA |
| | CAGCCGAAGCCATTAAGGTTCTTAAAGCTATTTCTGATGTTCGTTC |
| | CAATGTCAAGTTCGATTTCGAAAATCATTTAATTGGTGGTGCTGCT |
| | ATCGATGCTACAGGTGTCCCACTTCCAGATGAGGCGCTGGAAGCC |
| | TCCAAGAAGGTTGATGCCGTTTTGTTAGGTGCTGTGGCTGGTCCTA |
| | AATGGGGTACCGGTAGTGTTAGACCTGAACAAGGTTTACTAAAAA |
| | TCCGTAAAGAACTTCAATTGTACGCCAACTTAAGACCATGTAACTT |
| | TGCATCCGACTCTCTTTTAGACTTATCTCCAATCAAGCCACAATTT |
| | GCTAAAGGTACTGACTTCGTTGTTGTCAGAGAATTAGTGGGAGGT |
| | ATTTACTTTGGTAAGAGAAAGGAAGACGATGGTGATGGTGTCGCT |
| | TGGGATAGTGAACAATACACCGTTCCAGAAGTGCAAAGAATCACA |
| | AGAATGGCCGCTTTCATGGCCCTACAACATGAGCCACCATTGCCTA |
| | TTTGGTCCTTGGATAAAGCTAATCTTTTGGCCTCTTCAAGATTATG |
| | GAGAAAAACTGTGGAGGAAACCATCAAGAACGAATTCCCTACATT |
| | GAAGGTTCAACATCAATTGATTGATTCTGCCGCCATGATCCTAGTT |
| | AAGAACCCAACCCACCTAAATGGTATTATAATCACCAGCAACATG |
| | TTTGGTGATATCATCTCCGATGAAGCCTCCGTTATCCCAGGTTCCTT |
| | GGGTTTGTTGCCATCTGCGTCCTTGGCCTCTTTGCCAGACAAGAAC |
| | ACCGCATTTGGTTTGTACGAACCATGCCACGGTTCTGCTCCAGATT |
| | TGCCAAAGAATAAGGTTGACCCTATCGCCACTATCTTGTCTGCTGC |
| | AATGATGTTGAAATTGTCATTGAACTTGCCTGAAGAAGGTAAGGC |
| | CATTGAAGATGCAGTTAAAAAGGTTTTGGATGCAGGTATCAGAAC |
| | TGGTGATTTAGGTGGTTCCAACAGTACCACCGAAGTCGGTGATGCT |
| | GTCGCCGAAGAAGTTAAGAAAATCCTTGCTTAAAAAGATTCTCTTT |
| | TTTTATGATATTTGTACATAAACTTTATAAATGAAATTCATAATAG |
| | AAACGACACGAAATTACAAAATGGAATATGTTCATAGGGTAGACG |
| | AAACTATATACGCAATCTACATACATTTATCAAGAAGGAGAAAAA |
| | GGAGGATAGTAAAGGAATACAGGTAAGCAAATTGATACTAATGGC |
| | TCAACGTGATAAGGAAAAAGAATTGCACTTTAACATTAATATTGA |
| | CAAGGAGGAGGGCACCACACAAAAAGTTAGGTGTAACAGAAAAT |
| | CATGAAACTACGATTCCTAATTTGATATTGGAGGATTTTCTCTAAA |
| | AAAAAAAAAATACAACAAATAAAAAACACTCAATGACCTGACCAT |
| | TTGATGGAGTTTAAGTCAATACCTTCTTGAAGCATTTCCCATAATG |
| | GTGAAAGTTCCCTCAAGAATTTTACTCTGTCAGAAACGGCCTTACG |
| | ACGTAGTCGAGCATGCGTATTGGGCGCTCTTCCGCTTCCTCGCTCA |
| | CTGACTCGCTGCGCTCGGTCGTTCGGCTGCGGCGAGCGGTATCAGC |
| | TCACTCAAAGGCGGTAATACGGTTATCCACAGAATCAGGGGATAA |
| | CGCAGGAAAGAACATGTGAGCAAAAGGCCAGCAAAAGGCCAGGA |
| | ACCGTAAAAAGGCCGCGTTGCTGGCGTTTTTCCATAGGCTCCGCCC |
| | CCCTGACGAGCATCACAAAAATCGACGCTCAAGTCAGAGGTGGCG |
| | AAACCCGACAGGACTATAAAGATACCAGGCGTTTCCCCCTGGAAG |
| | CTCCCTCGTGCGCTCTCCTGTTCCGACCCTGCCGCTTACCGGATAC |
| | CTGTCCGCCTTTCTCCCTTCGGGAAGCGTGGCGCTTTCTCATAGCTC |
| | ACGCTGTAGGTATCTCAGTTCGGTGTAGGTCGTTCGCTCCAAGCTG |
| | GGCTGTGTGCACGAACCCCCCGTTCAGCCCGACCGCTGCGCCTTAT |
| | CCGGTAACTATCGTCTTGAGTCCAACCCGGTAAGACACGACTTATC |
| | GCCACTGGCAGCAGCCACTGGTAACAGGATTAGCAGAGCGAGGTA |
| | TGTAGGCGGTGCTACAGAGTTCTTGAAGTGGTGGCCTAACTACGG |
| | CTACACTAGAAGGACAGTATTTGGTATCTGCGCTCTGCTGAAGCCA |
| | GTTACCTTCGGAAAAAGAGTTGGTAGCTCTTGATCCGGCAAACAA |
| | ACCACCGCTGGTAGCGGTGGTTTTTTTGTTTGCAAGCAGCAGATTA |
| | CGCGCAGAAAAAAAGGATCTCAAGAAGATCCTTTGATCTTTTCTAC |
| | GGGGTCTGACGCTCAGTGGAACGAAAACTCACGTTAAGGGATTTT |
| | GGTCATGAGATTATCAAAAAGGATCTTCACCTAGATCCTTTTAAAT |
| | TAAAAATGAAGTTTTAAATCAATCTAAAGTATATATGAGTATACTT |
| | GGTCTGACAGTTAACGGCGCGTTCATCGTCCACCTCCGGAGAACA |
| | GGCCACCATCACGCATCTGTGTCTGAATTTCATCACGGGCGCGCCT |
| | AAGGGGATATCCTCGAGGTTCCCTTTAGTGAGGGTTAATTGCGAGC |
| | TTGGCGTAATCATGGTCATAGCTGTTTCCTGTGTGAAATTGTTATC |
| | CGCTCACAATTCCACACAACATACGAGCCGGAAGCATAAAGTGTA |
| | AAGCCTGGGGTGCCTAATGAGTGAGCTAACTCACATTAATTGCGTT |
| | GCGCTCACTGCCCGCTTTCCAGTCGGGAAACCTGTCGTGCCAGCTG |
| | CATTAACATCATACCGTATAGGCTATCCAATGCTTAATCAGTGAGG |
| | CACCTATCTCAGCGATCTGTCTATTTCGTTCATCCATAGTTGCCTGA |
| | CTCCCCGTCGTGTAGATAACTACGATACGGGAGGGCTTACCATCTG |
| | GCCCCAGTGCTGCAATGATACCGCGAGACCCACGCTCACCGGCTC |
| | CAGATTTATCAGCAATAAACCAGCCAGCCGGAAGGGCCGAGCGCA |
| | GAAGTGGTCCTGCAACTTTATCCGCCTCCATCCAGTCTATTAATTG |
| | TTGCCGGGAAGCTAGAGTAAGTAGTTCGCCAGTTAATAGTTTGCGC |
| | AACGTTGTTGCCATTGCTACAGGCATCGTGGTGTCACGCTCGTCGT |
| | TTGGTATGGCTTCATTCAGCTCCGGTTCCCAACGATCAAGGCGAGT |
| | TACATGATCCCCCATGTTGTGCAAAAAAGCGGTTAGCTCCTTCGGT |
| | CCTCCGATCGTTGTCAGAAGTAAGTTGGCCGCAGTGTTATCACTCA |
| | TGGTTATGGCAGCACTGCATAATTCTCTTACTGTCATGCCATCCGT |
| | AAGATGCTTTTCTGTGACTGGTGAGTACTCAACCAAGTCATTCTGA |
| | GAATAGTGTATGCGGCGACCGAGTTGCTCTTGCCCGGCGTCAATAC |
| | GGGATAATACCGCGCCACATAGCAGAACTTTAAAAGTGCTCATCA |
| | TTGGAAAACGTTCTTCGGGGCGAAAACTCTCAAGGATCTTACCGCT |
| | GTTGAGATCCAGTTCGATGTAACCCACTCGTGCACCCAACTGATCT |
| | TCAGCATCTTTTACTTTCACCAGCGTTTCTGGGTGAGCAAAAACAG |
| | GAAGGCAAAATGCCGCAAAAAAGGGAATAAGGGCGACACGGAAA |
| | TGTTGAATACTCATACTCTTCCTTTTTCAATATTATTGAAGCATTTA |
| | TCAGGGTTATTGTCTCATGAGCGGATACATATTTGAATGTATTTAG |
| | AAAAATAAACAAATAGGGGTTCCGCGCACATTTCCCCGAAAAGTG |
| | CCACCTGACGCGCCCTGTAGCGGCGCATTAAGCGCGGCGGGTGTG |
| | GTGGTTACGCGCAGCGTGACCGCTACACTTGCCAGCGCCCTAGCG |
| | CCCGCTCCTTTCGCTTTCTTCCCTTCCTTTCTCGCCACGTTCGCCGG |
| | CTTTCCCCGTCAAGCTCTAAATCGGGGGCTCCCTTTAGGGTTCCGA |
| | TTTAGTGCTTTACGGCACCTCGACCCCAAAAAACTTGATTAGGGTG |
| | ATGGTTCACGTAGTGGGCCATCGCCCTGATAGACGGTTTTTCGCCC |
| | TTTGACGTTGGAGTCCACGTTCTTTAATAGTGGACTCTTGTTCCAA |
| | ACTGGAACAACACTCAACCCTATCTCGGTCTATTCTTTTGATTTAT |
| | AAGGGATTTTGCCGATTTCGGCCTATTGGTTAAAAAATGAGCTGAT |
| | TTAACAAAAATTTAACGCGAATTTTAACAAAATATTAACGCTTACA |
| | ATTTGCCATTCGCCATTCAGGCTGCGCAACTGTTGGGAAGGGCGAT |
| SEQ ID NO: 42 | CGGTGCGGGCCTCTTCGCTATTACGCCAGCTGGCGAAAGGGGGAT |
| | GTGCTGCAAGGCGATTAAGTTGGGTAACGCCAGGGTTTTCCCAGTC |
| | ACGACGTTGTAAAACGACGGCCAGTGAATTGTAATACGACTCACT |
| | ATAGGGCGACCCTTAGGATCTAAGCATTGGCGCGCCCCGGCTGTCT |
| | GCCATGCTGCCCGGTGTACCGACATAACCGCCGGTGGCATAGCCG |
| | CGLATACGCGTCTCCAGCGTGTTTTATCTCTGCGAGCATAATGCCT |
| | GCGTCATCCGCCAGCAGGAGCTGGACTTTACTGATGCCCGTTATAT |
| | CTGCGAAAAGACCGGGATCTGGACCCGTGATGGCATTCTCTGGTTT |
| | TCGTCATCCGGTGAAGAGATTGAGCCACCTGACAGTGTGACCTTTC |
| | ACATCTGGACAGCGTACAGCCCGTTCACCACCTGGGTGCAGATTGT |
| | CAAAGACTGGATGAAAACGAAAGGGGATACGGGAAAACGTAAAA |
| | CCTTCGTAAACACCACGCTCGGTGAGACGTGGGAGGCGAAAATTG |
| | GCGAACGTCCGGATGCTGAAGTGATGGCAGAGCGGAAAGAGCATT |
| | ATTCAGCGCCCGTTCCTGACCGTGTGGCTTACCTGACCGCCGGTAT |
| | CGACTCCCAGCTGGACCGCTACGAAATGCGCGTATGGGGATGGGG |
| | GCCGGGTGAGGAAAGCTGGCTGATTGACCGGCAGATTATTATGGG |
| | CCGCCACGACGATGAACAGACGCTGCTGCGTGTGGATGAGGCCAT |
| | CAATAAAACCTATACCCGCCGGAATGGTGCAGAAATGTCGATATC |
| | CCGTATCTGCTGGGATACTGGACGCGTTTTCCCGTCTTTCAGTGCC |
| | TTGTTCAGTTCTTCCTGACGGGCGGTATATTTCTCCAGCTTGGCGC |
| | GCCTAAGACTTAGATCTTAAGGGGATATCCTCGAGGTTCCCTTTAG |
| | TGAGGGTTAATTGCGAGCTTGGCGTAATCATGGTCATAGCTGTTTC |
| | CTGTGTGAAATTGTTATCCGCTCACAATTCCACACAACATACGAGC |
| | CGGAAGCATAAAGTGTAAAGCCTGGGGTGCCTAATGAGTGAGCTA |
| | ACTCACATTAATTGCGTTGCGCTCACTGCCCGCTTTCCAGTCGGGA |
| | AACCTGTCGTGCCAGCTGCATTAATGAATCGGCCAACGCGCGGGG |
| | AGAGGCGGTTTGCGTATTGGGCGCTCTTCCGCTTCCTCGCTCACTG |
| | ACTCGCTGCGCTCGGTCGTTCGGCTGCGGCGAGCGGTATCAGCTCA |
| | CTCAAAGGCGGTAATACGGTTATCCACAGAATCAGGGGATAACGC |
| | AGGAAAGAACATGTGAGCAAAAGGCCAGCAAAAGGCCAGGAACC |
| | GTAAAAAGGCCGCGTTGCTGGCGTTTTTCCATAGGCTCCGCCCCCC |
| | TGACGAGCATCACAAAAATCGACGCTCAAGTCAGAGGTGGCGAAA |
| | CCCGACAGGACTATAAAGATACCAGGCGTTTCCCCCTGGAAGCTC |
| | CCTCGTGCGCTCTCCTGTTCCGACCCTGCCGCTTACCGGATACCTG |
| | TCCGCCTTTCTCCCTTCGGGAAGCGTGGCGCTTTCTCATAGCTCAC |
| | GCTGTAGGTATCTCAGTTCGGTGTAGGTCGTTCGCTCCAAGCTGGG |
| | CTGTGTGCACGAACCCCCCGTTCAGCCCGACCGCTGCGCCTTATCC |
| | GGTAACTATCGTCTTGAGTCCAACCCGGTAAGACACGACTTATCGC |
| | CACTGGCAGCAGCCACTGGTAACAGGATTAGCAGAGCGAGGTATG |
| | TAGGCGGTGCTACAGAGTTCTTGAAGTGGTGGCCTAACTACGGCT |
| | ACACTAGAAGAACAGTATTTGGTATCTGCGCTCTGCTGAAGCCAGT |
| | TACCTTCGGAAAAAGAGTTGGTAGCTCTTGATCCGGCAAACAAAC |
| | CACCGCTGGTAGCGGTGGTTTTTTTGTTTGCAAGCAGCAGATTACG |
| | CGCAGAAAAAAAGGATCTCAAGAAGATCCTTTGATCTTTTCTACG |
| | GGGTCTGACGCTCAGTGGAACGAAAACTCACGTTAAGGGATTTTG |
| | GTCATGAGATTATCAAAAAGGATCTTCACCTAGATCCTTTTAAATT |
| | AAAAATGAAGTTTTAAATCAATCTAAAGTATATATGAGTAAACTT |
| | GGTCTGACAGTTACCAATGCTTAATCAGTGAGGCACCTATCTCAGC |
| | GATCTGTCTATTTCGTTCATCCATAGTTGCCTGACTCCCCGTCGTGT |
| | AGATAACTACGATACGGGAGGGCTTACCATCTGGCCCCAGTGCTG |
| | CAATGATACCGCGAGACCCACGCTCACCGGCTCCAGATTTATCAG |
| | CAATAAACCAGCCAGCCGGAAGGGCCGAGGuCAGAAGTGGTCCTG |
| | CAACTTTATCCGCCTCCATCCAGTCTATTAATTGTTGCCGGGAAGC |
| | TAGAGTAAGTAGTTCGCCAGTTAATAGTTTGCGCAACGTTGTTGCC |
| | ATTGCTACAGGCATCGTGGTGTCACGCTCGTCGTTTGGTATGGCTT |
| | CATTCAGCTCCGGTTCCCAACGATCAAGGCGAGTTACATGATCCCC |
| | CATGTTGTGCAAAAAAGCGGTTAGCTCCTTCGGTCCTCCGATCGTT |
| | GTCAGAAGTAAGTTGGCCGCAGTGTTATCACTCATGGTTATGGCAG |
| | CACTGCATAATTCTCTTACTGTCATGCCATCCGTAAGATGCTTTTCT |
| | GTGACTGGTGAGTACTCAACCAAGTCATTCTGAGAATAGTGTATGC |
| | GGCGACCGAGTTGCTCTTGCCCGGCGTCAATACGGGATAATACCG |
| | CGCCACATAGCAGAACTTTAAAAGTGCTCATCATTGGAAAACGTT |
| | CTTCGGGGCGAAAACTCTCAAGGATCTTACCGCTGTTGAGATCCAG |
| | TTCGATGTAACCCACTCGTGCACCCAACTGATCTTCAGCATCTTTT |
| | ACTTTCACCAGCGTTTCTGGGTGAGCAAAAACAGGAAGGCAAAAT |
| | GCCGCAAAAAAGGGAATAAGGGCGACACGGAAATGTTGAATACT |
| | CATACTCTTCCTTTTTCAATATTATTGAAGCATTTATCAGGGTTATT |
| | GTCTCATGAGCGGATACATATTTGAATGTATTTAGAAAAATAAAC |
| | AAATAGGGGTTCCGCGCACATTTCCCCGAAAAGTGCCACCTGACG |
| | CGCCCTGTAGCGGCGCATTAAGCGCGGCGGGTGTGGTGGTTACGC |
| | GCAGCGTGACCGCTACACTTGCCAGCGCCCTAGCGCCCGCTCCTTT |
| | CGCTTTCTTCCCTTCCTTTCTCGCCACGTTCGCCGGCTTTCCCCGTC |
| | AAGCTCTAAATCGGGGGCTCCCTTTAGGGTTCCGATTTAGTGCTTT |
| | ACGGCACCTCGACCCCAAAAAACTTGATTAGGGTGATGGTTCACG |
| | TAGTGGGCCATCGCCCTGATAGACGGTTTTTCGCCCTTTGACGTTG |
| | GAGTCCACGTTCTTTAATAGTGGACTCTTGTTCCAAACTGGAACAA |
| | CACTCAACCCTATCTCGGTCTATTCTTTTGATTTATAAGGGATTTTG |
| | CCGATTTCGGCCTATTGGTTAAAAAATGAGCTGATTTAACAAAAAT |
| | TTAACGCGAATTTTAACAAAATATTAACGCTTACAATTTGCCATTC |
| | GCCATTCAGGCTGCGCAACTGTTGGGAAGGGCGAT |
| SEQ ID NO: 43 | AAGCTTAAA |
| SEQ ID NO: 44 | CCGCGG |
| SEQ ID NO: 45 | CGGTGCGGGCCTCTTCGCTATTACGCCAGCTGGCGAAAGGGGG |
| | ATGTGCTGCAAGGCGATTAAGTTGGGTAACGCCAGGGTTTTCCC |
| | AGTCACGACGTTGTAAAACGACGGCCAGTGAATTGTAATACGACT |
| | CACTATAGGGCGACCCTTAGGATCCTATGGCGCGCCACCACGGT |
| | GAACAATCCCCGCTGGCTCATATTTGCCGCCGGTTCCCGTAAATC |
| | CTCCGGTACGCGCCGGGCCGTATACTTACATATAGTAGATGTCAA |
| | GCGTAGGCGCTTCCCCTGCCGGCTGTGAGGGCGCCATAACCAA |
| | GGTATCTATAGACCGCCAATCAGCAAACTACCTCCGTACATTCAT |
| | GTTGCACCCACACATTTATACACCCAGACCGCGACAAATTACCCA |
| | TAAGGTTGTTTGTGACGGCGTCGTACAAGAGAACGTGGGAACTTT |
| | TTAGGCTCACCAAAAAAGAAAGAAAAAATACGAGTTGCTGACAGA |
| | AGCCTCAAGAAAAAAATTCTTCTTCGACTATGCTGGAGGCAG |
| | AGATGATCGAGCCGGTAGTTAACTATATATAGCTAAATTGGTTCC |
| | ATCACCTTCTTTTCTGGTGTCGCTCCTTCTAGTGCTATTTCTGGCT |
| | TTTCCTATTTTTTTTTTTCCATTTTTCTTTCTCTCTTTCTAATATATA |
| | AATTCTCTTGCATTTTCTATTTTTCTCTCTATCTATTCTACTTGTTTA |
| | TTCCCTTCAAGGTTTTTTTTTAAGGAGTACTTGTTTTTAGAATATAC |
| | GGTCAACGAACTATAATTAACTAAACAAGCTTAAAATGGCTAACCC |
| | ACACCCACATTTCTTGATTATTACTTTTCCAGCCCAAGGTCATATT |
| | AACCCAGCTTTGGAATTGGCCAAAAGATTGATTGGTGTTGGTGCT |
| | GATGTTACTTTCGCTACTACTATTCATGCCAAGTCCAGATTGGTTA |
| | AGAACCCAACTGTTGATGGTTTGAGATTCTCTACTTTCTCCGATG |
| | GTCAAGAAGAAGGTGTTAAGAGAGGTCCAAACGAATTGCCAGTTT |
| | TTCAAAGATTGGCCTCCGAAAACTTGTCCGAATTGATTATGGCTT |
| | CTGCTAATGAAGGTAGACCAATCTCTTGTTTGATCTACTCCATTTT |
| | GATTCCAGGTGCTGCTGAATTGGCTAGATCATTCAATATTCCATCT |
| | GCTTTCTTGTGGATTCAACCAGCTACTGTTTTGGACATCTATTACT |
| | ACTACTTCAACGGTTTCGGTGACTTGATCAGATCCAAATCTTCTGA |
| | TCCATCCTTCTCCATTGAATTACCAGGTTTGCCATCTTTGTCCAGA |
| | CAAGATTTGCCATCCTTTTTCGTTGGTTCCGACCAAAATCAAGAAA |
| | ACCATGCTTTGGCTGCCTTTCAAAAGCACTTGGAAATTTTGGAAC |
| | AAGAAGAAAACCCAAAGGTCTTGGTTAACACTTTCGATGCTTTAG |
| | AACCAGAAGCCTTGAGAGCTGTTGAAAAGTTGAAATTGACTGCTG |
| | TTGGTCCATTGGTTCCATCTGGTTTTTCTGATGGTAAAGATGCTTC |
| | TGATACACCATCTGGTGGTGATTTGTCTGATGGTTCTAGAGATTAT |
| | ATGGAATGGTTGAAGTCCAAGCCAGAATCTACTGTTGTTTACGTT |
| | TCCTTCGGTTCCATCAGTATGTTCTCTATGCAACAAATGGAAGAAA |
| | TCGCCAGAGGTTTGTTGGAATCTGGTAGACCATTTTTGTGGGTTA |
| | TCAGAGCTAAAGAAAACGGTGAAGAAAACAAAGAAGAAGATAAGT |
| | TGTCCTGCCAAGAAGAATTGGAAAAGCAAGGTATGTTGATCCAAT |
| | GGTGCTCTCAAATGGAAGTTTTGTCTCATCCATCTTTGGGTTGTTT |
| | CGTTACTCATTGTGGTTGGAACTCCTCTATTGAATCTTTAGCTTCT |
| | GGTGTTCCAATGATTGCATTTCCACAATGGGCTGATCAAGGTACT |
| | AATACCAAGTTGATTAAGGACGTTTGGAAAACCGGTGTTAGATTG |
| | ATGGTTAACGAAGAAGAAATTGTCACCTCCGACGAATTGAGAAGA |
| | TGCTTGGAATTAGTTATGGGTGATGGTGAAAAGGGTCAAGAAATG |
| | AGAAAGAATGCTAAGAAGTGGAAGATTTTGGCTAAAGAAGCCTTA |
| | AAAGAAGGTGGTTCCTCTCACAAGAATTTGAAGAACTTCGTTGAC |
| | GAAGTCATCCAAGGTTACTGACCGCGGACAAATCGCTCTTAAATA |
| | TATACCTAAAGAACATTAAAGCTATATTATAAGCAAAGATACGTAA |
| | ATTTTGCTTATATTATTATACACATATCATATTTCTATATTTTTAAGA |
| | TTTGGTTATATAATGTACGTAATGCAAAGGAAATAAATTTTATACAT |
| | TATTGAACAGCGTCCAAGTAACTACATTATGTGCACTAATAGTTTA |
| | GCGTCGTGAAGACTTTATTGTGTCGCGAAAAGTAAAAATTTTAAAA |
| | ATTAGAGCACCTTGAACTTGCGAAAAAGGTTCTCATCAACTGTTTA |
| | AAAGGAGGATATCAGGTCCTATTTCTGACAAACAATATACAAATTT |
| | AGTTTCAAAGGCGCGTTGCAAAATGGAATTTCGCCGCAGCGGCC |
| | TGAATGGCTGTACCGCCTGACGCGGATGCGCCGGCGCGCCTATT |
| | GAAAGATCTTAAGGGGATATCCTCGAGGTTCCCTTTAGTGAGGGT |
| | TAATTGCGAGCTTGGCGTAATCATGGTCATAGCTGTTTCCTGTGT |
| | GAAATTGTTATCCGCTCACAATTCCACACAACATACGAGCCGGAA |
| | GCATAAAGTGTAAAGCCTGGGGTGCCTAATGAGTGAGCTAACTCA |
| | CATTAATTGCGTTGCGCTCACTGCCCGCTTTCCAGTCGGGAAACC |
| | TGTCGTGCCAGCTGCATTAATGAATCGGCCAACGCGCGGGGAGA |
| | GGCGGTTTGCGTATTGGGCGCTCTTCCGCTTCCTCGCTCACTGA |
| | CTCGCTGCGCTCGGTCGTTCGGCTGCGGCGAGCGGTATCAGCT |
| | CACTCAAAGGCGGTAATACGGTTATCCACAGAATCAGGGGATAAC |
| | GCAGGAAAGAACATGTGAGCAAAAGGCCAGCAAAAGGCCAGGAA |
| | CCGTAAAAAGGCCGCGTTGCTGGCGTTTTTCCATAGGCTCCGCC |
| | CCCCTGACGAGCATCACAAAAATCGACGCTCAAGTCAGAGGTGG |
| | CGAAACCCGACAGGACTATAAAGATACCAGGCGTTTCCCCCTGG |
| | AAGCTCCCTCGTGCGCTCTCCTGTTCCGACCCTGCCGCTTACCG |
| | GATACCTGTCCGCCTTTCTCCCTTCGGGAAGCGTGGCGCTTTCTC |
| | ATAGCTCACGCTGTAGGTATCTCAGTTCGGTGTAGGTCGTTCGCT |
| | CCAAGCTGGGCTGTGTGCACGAACCCCCCGTTCAGCCCGACCG |
| | CTGCGCCTTATCCGGTAACTATCGTCTTGAGTCCAACCCGGTAAG |
| | ACACGACTTATCGCCACTGGCAGCAGCCACTGGTAACAGGATTA |
| | GCAGAGCGAGGTATGTAGGCGGTGCTACAGAGTTCTTGAAGTGG |
| | TGGCCTAACTACGGCTACACTAGAAGAACAGTATTTGGTATCTGC |
| | GCTCTGCTGAAGCCAGTTACCTTCGGAAAAAGAGTTGGTAGCTCT |
| | TGATCCGGCAAACAAACCACCGCTGGTAGCGGTGGTTTTTTTGTT |
| | TGCAAGCAGCAGATTACGCGCAGAAAAAAAGGATCTCAAGAAGAT |
| | CCTTTGATCTTTTCTACGGGGTCTGACGCTCAGTGGAACGAAAAC |
| | TCACGTTAAGGGATTTTGGTCATGAGATTATCAAAAAGGATCTTCA |
| | CCTAGATCCTTTTAAATTAAAAATGAAGTTTTAAATCAATCTAAAGT |
| | ATATATGAGTAAACTTGGTCTGACAGTTACCAATGCTTAATCAGTG |
| | AGGCACCTATCTCAGCGATCTGTCTATTTCGTTCATCCATAGTTG |
| | CCTGACTCCCCGTCGTGTAGATAACTACGATACGGGAGGGCTTA |
| | CCATCTGGCCCCAGTGCTGCAATGATACCGCGAGACCCACGCTC |
| | ACCGGCTCCAGATTTATCAGCAATAAACCAGCCAGCCGGAAGGG |
| | CCGAGCGCAGAAGTGGTCCTGCAACTTTATCCGCCTCCATCCAG |
| | TCTATTAATTGTTGCCGGGAAGCTAGAGTAAGTAGTTCGCCAGTT |
| | AATAGTTTGCGCAACGTTGTTGCCATTGCTACAGGCATCGTGGTG |
| | TCACGCTCGTCGTTTGGTATGGCTTCATTCAGCTCCGGTTCCCAA |
| | CGATCAAGGCGAGTTACATGATCCCCCATGTTGTGCAAAAAAGCG |
| | GTTAGCTCCTTCGGTCCTCCGATCGTTGTCAGAAGTAAGTTGGCC |
| | GCAGTGTTATCACTCATGGTTATGGCAGCACTGCATAATTCTCTTA |
| | CTGTCATGCCATCCGTAAGATGCTTTTCTGTGACTGGTGAGTACT |
| | CAACCAAGTCATTCTGAGAATAGTGTATGCGGCGACCGAGTTGCT |
| | CTTGCCCGGCGTCAATACGGGATAATACCGCGCCACATAGCAGA |
| | ACTTTAAAAGTGCTCATCATTGGAAAACGTTCTTCGGGGCGAAAA |
| | CTCTCAAGGATCTTACCGCTGTTGAGATCCAGTTCGATGTAACCC |
| | ACTCGTGCACCCAACTGATCTTCAGCATCTTTTACTTTCACCAGC |
| | GTTTCTGGGTGAGCAAAAACAGGAAGGCAAAATGCCGCAAAAAA |
| | GGGAATAAGGGCGAAACGGAAATGTTGAATACTCATACTCTTCCT |
| | TTTTCAATATTATTGAAGCATTTATCAGGGTTATTGTCTCATGAGC |
| | GGATACATATTTGAATGTATTTAGAAAAATAAACAAATAGGGGTTC |
| | CGCGCACATTTCCCCGAAAAGTGCCACCTGACGCGCCCTGTAGC |
| | GGCGCATTAAGCGCGGCGGGTGTGGTGGTTACGCGCAGCGTGA |
| | CCGCTACACTTGCCAGCGCCCTAGCGCCCGCTCCTTTCGCTTTC |
| | TTCCCTTCCTTTCTCGCCACGTTCGCCGGCTTTCCCCGTCAAGCT |
| | CTAAATCGGGGGCTCCCTTTAGGGTTCCGATTTAGTGCTTTACGG |
| | CACCTCGACCCCAAAAAACTTGATTAGGGTGATGGTTCACGTAGT |
| | GGGCCATCGCCCTGATAGACGGTTTTTCGCCCTTTGACGTTGGA |
| | GTCCACGTTCTTTAATAGTGGACTCTTGTTCCAAACTGGAACAAC |
| | ACTCAACCCTATCTCGGTCTATTCTTTTGATTTATAAGGGATTTTG |
| | CCGATTTCGGCCTATTGGTTAAAAAATGAGCTGATTTAACAAAAAT |
| | TTAACGCGAATTTTAACAAAATATTAACGCTTACAATTTGCCATTC |
| | GCCATTCAGGCTGCGCAACTGTTGGGAAGGGCGAT |
| SEQ ID NO: 46 | CGGTGCGGGCCTCTTCGCTATTACGCCAGCTGGCGAAAGGGGG |
| | ATGTGCTGCAAGGCGATTAAGTTGGGTAACGCCAGGGTTTTCCC |
| | AGTCACGACGTTGTAAAACGACGGCCAGTGAATTGTAATACGACT |
| | CACTATAGGGCGACCCTTAGGATCCTATGGCGCGCCGGCACCCT |
| | TGCGGGCCATGTCATACACCGCCTTCAGAGCAGCCGGACCTATC |
| | TGCCCGTTACGCGCCAGCTTGCAAATTAAAGCCTTCGAGCGTCC |
| | CAAAACCTTCTCAAGCAAGGTTTTCAGTATAATGTTACATGCGTAC |
| | ACGCGTCTGTACAGAAAAAAAAGAAAAATTTGAAATATAAATAACG |
| | TTCTTAATACTAACATAACTATAAAAAAATAAATAGGGACCTAGAC |
| | TTCAGGTTGTCTAACTCCTTCCTTTTCGGTTAGAGCGGATGTGGG |
| | GGGAGGGCGTGAATGTAAGCGTGACATAACTAATTACATGATATC |
| | GACAAAGGAAAAGGGGGACGGATCTCCGAGGCCTCGGACCCGT |
| | CGGGCCGCCGTCGGACGTGCCGCGGATCCCCGGGTCGAGCCTG |
| | AACGGCCTCGAGGCCTGAACGGCCTCGACGAATTCATTATTTGTA |
| | GAGCTCATCCATGCCATGTGTAATCCCAGCAGCAGTTACAAACTC |
| | AAGAAGGACCATGTGGTCACGCTTTTCGTTGGGATCTTTCGAAAG |
| | GGCAGATTGTGTCGACAGGTAATGGTTGTCTGGTAAAAGGACAG |
| | GGCCATCGCCAATTGGAGTATTTTGTTGATAATGGTCTGCTAGTT |
| | GAACGGATCCATCTTCAATGTTGTGGCGAATTTTGAAGTTAGCTTT |
| | GATTCCATTCTTTTGTTTGTCTGCCGTGATGTATACATTGTGTGAG |
| | TTATAGTTGTACTCGAGTTTGTGTCCGAGAATGTTTCCATCTTCTT |
| | TAAAATCAATACCTTTTAACTCGATACGATTAACAAGGGTATCACC |
| | TTCAAACTTGACTTCAGCACGCGTCTTGTAGTTCCCGTCATCTTTG |
| | AAAGATATAGTGCGTTCCTGTACATAACCTTCGGGCATGGCACTC |
| | TTGAAAAAGTCATGCCGTTTCATATGATCCGGATAACGGGAAAAG |
| | CATTGAACACCATAAGAGAAAGTAGTGACAAGTGTTGGCCATGGA |
| | ACAGGTAGTTTTCCAGTAGTGCAAATAAATTTAAGGGTAAGCTGG |
| | CCCTGCAGGCCAAGCTTTTTGTTTGTTTATGTGTGTTTATTCGAAA |
| | CTAAGTTCTTGGTGTTTTAAAACTAAAAAAAAGACTAACTATAAAA |
| | GTAGAATTTAAGAAGTTTAAGAAATAGATTTACAGAATTACAATCA |
| | ATACCTACCGTCTTTATATACTTATTAGTCAAGTAGGGGAATAATT |
| | TCAGGGAACTGGTTTCAACCTTTTTTTTCAGCTTTTTCCAAATCAG |
| | AGAGAGCAGAAGGTAATAGAAGGTGTAAGAAAATGAGATAGATAC |
| | ATGCGTGGGTCAATTGCCTTGTGTCATCATTTACTCCAGGCAGGT |
| | TGCATCACTCCATTGAGGTTGTGTCCGTTTTTTGCCTGTTTGTGC |
| | CCCTGTTCTCTGTAGTTGCGCTAAGAGAATGGACCTATGAACTGA |
| | TGGTTGGTGAAGAAAACAATATTTTGGTGCTGGGATTCTTTTTTTT |
| | TCTGGATGCCAGCTTAAAAAGCGGGCTCCATTATATTTAGTGGAT |
| | GCCAGGAATAAACTGTTCACCCAGACACCTACGATGTTATATATT |
| | CTGTGTAACCCGCCCCCTATTTTGGGCATGTACGGGTTACAGCA |
| | GAATTAAAAGGCTAATTTTTTGACTAAATAAAGTTAGGAAAATCAC |
| | TACTATTAATTATTTACGTATTCTTTGAAATGGCAGTATTGATAATG |
| | ATAAACTCGAACTGGGCGCGTCGTGCCGTCGTTGTTAATCACCAC |
| | ATGGTTATTCTGCTCAAACGTCCCGGACGCCTGCGAGGCGCGCC |
| | TATTGAAAGATCTTAAGGGGATATCCTCGAGGTTCCCTTTAGTGA |
| | GGGTTAATTGCGAGCTTGGCGTAATCATGGTCATAGCTGTTTCCT |
| | GTGTGAAATTGTTATCCGCTCACAATTCCACACAACATACGAGCC |
| | GGAAGCATAAAGTGTAAAGCCTGGGGTGCCTAATGAGTGAGCTA |
| | ACTCACATTAATTGCGTTGCGCTCACTGCCCGCTTTCCAGTCGGG |
| | AAACCTGTCGTGCCAGCTGCATTAATGAATCGGCCAACGCGCGG |
| | GGAGAGGCGGTTTGCGTATTGGGCGCTCTTCCGCTTCCTCGCTC |
| | ACTGACTCGCTGCGCTCGGTCGTTCGGCTGCGGCGAGCGGTATC |
| | AGCTCACTCAAAGGCGGTAATACGGTTATCCACAGAATCAGGGG |
| | ATAACGCAGGAAAGAACATGTGAGCAAAAGGCCAGCAAAAGGCC |
| | AGGAACCGTAAAAAGGCCGCGTTGCTGGCGTTTTTCCATAGGCT |
| | CCGCCCCCCTGACGAGCATCACAAAAATCGACGCTCAAGTCAGA |
| | GGTGGCGAAACCCGACAGGACTATAAAGATACCAGGCGTTTCCC |
| | CCTGGAAGCTCCCTCGTGCGCTCTCCTGTTCCGACCCTGCCGCT |
| | TACCGGATACCTGTCCGCCTTTCTCCCTTCGGGAAGCGTGGCGC |
| | TTTCTCATAGCTCACGCTGTAGGTATCTCAGTTCGGTGTAGGTCG |
| | TTCGCTCCAAGCTGGGCTGTGTGCACGAACCCCCCGTTCAGCCC |
| | GACCGCTGCGCCTTATCCGGTAACTATCGTCTTGAGTCCAACCC |
| | GGTAAGACACGACTTATCGCCACTGGCAGCAGCCACTGGTAACA |
| | GGATTAGCAGAGCGAGGTATGTAGGCGGTGCTACAGAGTTCTTG |
| | AAGTGGTGGCCTAACTACGGCTACACTAGAAGAACAGTATTTGGT |
| | ATCTGCGCTCTGCTGAAGCCAGTTACCTTCGGAAAAAGAGTTGGT |
| | AGCTCTTGATCCGGCAAACAAACCACCGCTGGTAGCGGTGGTTT |
| | TTTTGTTTGCAAGCAGCAGATTACGCGCAGAAAAAAAGGATCTCA |
| | AGAAGATCCTTTGATCTTTTCTACGGGGTCTGACGCTCAGTGGAA |
| | CGAAAACTCACGTTAAGGGATTTTGGTCATGAGATTATCAAAAAG |
| | GATCTTCACCTAGATCCTTTTAAATTAAAAATGAAGTTTTAAATCAA |
| | TCTAAAGTATATATGAGTAAACTTGGTCTGACAGTTACCAATGCTT |
| | AATCAGTGAGGCACCTATCTCAGCGATCTGTCTATTTCGTTCATC |
| | CATAGTTGCCTGACTCCCCGTCGTGTAGATAACTACGATACGGGA |
| | GGGCTTACCATCTGGCCCCAGTGCTGCAATGATACCGCGAGACC |
| | CACGCTCACCGGCTCCAGATTTATCAGCAATAAACCAGCCAGCC |
| | GGAAGGGCCGAGCGCAGAAGTGGTCCTGCAACTTTATCCGCCTC |
| | CATCCAGTCTATTAATTGTTGCCGGGAAGCTAGAGTAAGTAGTTC |
| | GCCAGTTAATAGTTTGCGCAACGTTGTTGCCATTGCTACAGGCAT |
| | CGTGGTGTCACGCTCGTCGTTTGGTATGGCTTCATTCAGCTCCG |
| | GTTCCCAACGATCAAGGCGAGTTACATGATCCCCCATGTTGTGCA |
| | AAAAAGCGGTTAGCTCCTTCGGTCCTCCGATCGTTGTCAGAAGTA |
| | AGTTGGCCGCAGTGTTATCACTCATGGTTATGGCAGCACTGCATA |
| | ATTCTCTTACTGTCATGCCATCCGTAAGATGCTTTTCTGTGACTGG |
| | TGAGTACTCAACCAAGTCATTCTGAGAATAGTGTATGCGGCGACC |
| | GAGTTGCTCTTGCCCGGCGTCAATACGGGATAATACCGCGCCAC |
| | ATAGCAGAACTTTAAAAGTGCTCATCATTGGAAAACGTTCTTCGG |
| | GGCGAAAACTCTCAAGGATCTTACCGCTGTTGAGATCCAGTTCGA |
| | TGTAACCCACTCGTGCACCCAACTGATCTTCAGCATCTTTTACTTT |
| | CACCAGCGTTTCTGGGTGAGCAAAAACAGGAAGGCAAAATGCCG |
| | CAAAAAAGGGAATAAGGGCGACACGGAAATGTTGAATACTCATAC |
| | TCTTCCTTTTTCAATATTATTGAAGCATTTATCAGGGTTATTGTCTC |
| | ATGAGCGGATACATATTTGAATGTATTTAGAAAAATAAACAAATAG |
| | GGGTTCCGCGCACATTTCCCCGAAAAGTGCCACCTGACGCGCCC |
| | TGTAGCGGCGCATTAAGCGCGGCGGGTGTGGTGGTTACGCGCA |
| | GCGTGACCGCTACACTTGCCAGCGCCCTAGCGCCCGCTCCTTTC |
| | GCTTTCTTCCCTTCCTTTCTCGCCACGTTCGCCGGCTTTCCCCGT |
| | CAAGCTCTAAATCGGGGGCTCCCTTTAGGGTTCCGATTTAGTGCT |
| | TTACGGCACCTCGACCCCAAAAAACTTGATTAGGGTGATGGTTCA |
| | CGTAGTGGGCCATCGCCCTGATAGACGGTTTTTCGCCCTTTGAC |
| | GTTGGAGTCCACGTTCTTTAATAGTGGACTCTTGTTCCAAACTGG |
| | AACAACACTCAACCCTATCTCGGTCTATTCTTTTGATTTATAAGGG |
| | ATTTTGCCGATTTCGGCCTATTGGTTAAAAAATGAGCTGATTTAAC |
| | AAAAATTTAACGCGAATTTTAACAAAATATTAACGCTTACAATTTGC |
| | CATTCGCCATTCAGGCTGCGCAACTGTTGGGAAGGGCGAT |
| SEQ ID NO: 47 | CGGTGCGGGCCTCTTCGCTATTACGCCAGCTGGCGAAAGGGGG |
| | ATGTGCTGCAAGGCGATTAAGTTGGGTAACGCCAGGGTTTTCCC |
| | AGTCACGACGTTGTAAAACGACGGCCAGTGAATTGTAATACGACT |
| | CACTATAGGGCGACCCTTAAGATCTGTAATGGCGCGCCATGCGC |
| | GGCTATGCCACCGGCGGTTATGTCGGTACACCGGGCAGCATGG |
| | CAGACAGCCGGACGCGCCACGCACAGATATTATAACATCTGCAT |
| | AATAGGCATTTGCAAGAATTACTCGTGAGTAAGGAAAGAGTGAGG |
| | AACTATCGCATACCTGCATTTAAAGATGCCGATTTGGGCGCGAAT |
| | CCTTTATTTTGGCTTCACCCTCATACTATTATCAGGGCCAGAAAAA |
| | GGAAGTGTTTCCCTCCTTCTTGAATTGATGTTACCCTCATAAAGCA |
| | CGTGGCCTCTTATCGAGAAAGAAATTACCGTCGCTCGTGATTTGT |
| | TTGCAAAAAGAACAAAACTGAAAAAACCCAGACACGCTCGACTTC |
| | CTGTCTTCCTATTGATTGCAGCTTCCAATTTCGTCACACAACAAGG |
| | TCCTAGCGACGGCTCACAGGTTTTGTAACAAGCAATCGAAGGTTC |
| | TGGAATGGCGGGAAAGGGTTTAGTACCACATGCTATGATGCCCA |
| | CTGTGATCTCCAGAGCAAAGTTCGTTCGATCGTACTGTTACTCTC |
| | TCTCTTTCAAACAGAATTGTCCGAATCGTGTGACAACAACAGCCT |
| | GTTCTCACACACTCTTTTCTTCTAACCAAGGGGGTGGTTTAGTTTA |
| | GTAGAACCTCGTGAAACTTACATTTACATATATATAAACTTGCATA |
| | AATTGGTCAATGCAAGAAATACATATTTGGTCTTTTCTAATTCGTA |
| | GTTTTTCAAGTTCTTAGATGCTTTCTTTTTCTCTTTTTTACAGATCA |
| | TCAAGGAAGTAATTATCTACTTTTTACAACAAATATAAAACAAAGC |
| | TTAAAATGGCCTTGAGAATCAACGAATTATTCGTCGCTGCCATCAT |
| | CTACATCATCGTTCATATTATCATCTCCAAGTTGATCACCACCGTT |
| | AGAGAAAGAGGTAGAAGATTGCCATTGCCACCAGGTCCAACTGG |
| | TTGGCCAGTTATTGGTGCTTTGCCATTATTGGGTTCTATGCCACAT |
| | GTTGCTTTGGCTAAAATGGCTAAGAAATACGGTCCAATCATGTAC |
| | TTGAAGGTTGGTACTTGTGGTATGGTTGTTGCTTCTACTCCAAAT |
| | GCTGCTAAGGCTTTCTTGAAAACCTTGGACATTAACTTCTCTAACA |
| | GACCACCTAATGCTGGTGCTACTCATTTGGCTTATAATGCCCAAG |
| | ATATGGTTTTTGCTCCATATGGTCCAAGATGGAAGTTGTTGAGAA |
| | AGTTGTCTAACTTGCATATGTTGGGTGGTAAGGCTTTGGAAAATT |
| | GGGCTAATGTTAGAGCTAACGAATTGGGTCATATGTTGAAGTCTA |
| | TGTTCGATGCTTCTCAAGATGGTGAATGCGTTGTTATTGCTGATG |
| | TTTTGACTTTCGCTATGGCTAACATGATCGGTCAAGTTATGTTGTC |
| | CAAGAGAGTTTTCGTTGAAAAGGGTGTCGAAGTTAACGAATTCAA |
| | GAACATGGTTGTCGAATTGATGACTGTTGCTGGTTACTTTAACATC |
| | GGTGATTTCATTCCAAAGTTGGCCTGGATGGATATTCAAGGTATT |
| | GAAAAAGGTATGAAGAACTTGCACAAGAAGTTCGACGATTTGTTG |
| | ACCAAGATGTTTGATGAACATGAAGCCACCTCCAACGAAAGAAAA |
| | GAAAATCCAGATTTCTTGGATGTCGTCATGGCCAATAGAGATAAT |
| | TCTGAAGGTGAAAGATTGTCCACCACCAATATTAAGGCCTTGTTG |
| | TTGAATTTGTTCACCGCTGGTACTGATACCTCCTCTTCTGTTATTG |
| | AATGGGCTTTAGCTGAAATGATGAAGAACCCAAAAATCTTCAAAA |
| | AGGCCCAACAAGAAATGGACCAAGTTATCGGTAAAAACAGAAGAT |
| | TGATCGAATCCGACATTCCAAACTTGCCATATTTGAGAGCTATCT |
| | GCAAAGAAACTTTCAGAAAGCACCCATCTACTCCATTGAATTTGC |
| | CAAGAGTTTCTTCTGAACCATGTACCGTTGATGGTTACTACATCC |
| | CAAAAAACACTAGATTGTCCGTTAACATTTGGGCCATTGGTAGAG |
| | ATCCAGATGTTTGGGAAAATCCATTGGAATTCACTCCAGAAAGAT |
| | TCTTGTCTGGTAAGAACGCTAAGATTGAACCTAGAGGTAACGACT |
| | TTGAATTGATTCCATTTGGTGCCGGTAGAAGAATTTGTGCTGGTA |
| | CTAGAATGGGTATCGTTGTCGTTGAATATATCTTAGGTACTTTGGT |
| | CCACTCCTTCGATTGGAAATTGCCAAACAACGTTATCGACATCAA |
| | CATGGAAGAATCATTTGGTTTGGCCTTGCAAAAAGCTGTTCCATT |
| | AGAAGCTATGGTTACCCCAAGATTGTCTTTGGATGTTTACAGATG |
| | CTAACCGCGGATCTCTTATGTCTTTACGATTTATAGTTTTCATTAT |
| | CAAGTATGCCTATATTAGTATATAGCATCTTTAGATGACAGTGTTC |
| | GAAGTTTCACGAATAAAAGATAATATTCTACTTTTTGCTCCCACCG |
| | CGTTTGCTAGCACGAGTGAACACCATCCCTCGCCTGTGAGTTGTA |
| | CCCATTCCTCTAAACTGTAGACATGGTAGCTTCAGCAGTGTTCGT |
| | TATGTACGGCATCCTCCAACAAACAGTCGGTTATAGTTTGTCCTG |
| | CTCCTCTGAATCGTCTCCCTCGATATTTCTCATTTTCCTTCGGCGC |
| | GTTCGCAGGCGTCCGGGACGTTTGAGCAGAATAACCATGTGGTG |
| | ATTAACAACGACGGCACGGGCGCGCCAATGCTTAGATCTTAAGG |
| | GGATATCCTCGAGGTTCCCTTTAGTGAGGGTTAATTGCGAGCTTG |
| | GCGTAATCATGGTCATAGCTGTTTCCTGTGTGAAATTGTTATCCG |
| | CTCACAATTCCACACAACATACGAGCCGGAAGCATAAAGTGTAAA |
| | GCCTGGGGTGCCTAATGAGTGAGCTAACTCACATTAATTGCGTTG |
| | CGCTCACTGCCCGCTTTCCAGTCGGGAAACCTGTCGTGCCAGCT |
| | GCATTAATGAATCGGCCAACGCGCGGGGAGAGGCGGTTTGCGTA |
| | TTGGGCGCTCTTCCGCTTCCTCGCTCACTGACTCGCTGCGCTCG |
| | GTCGTTCGGCTGCGGCGAGCGGTATCAGCTCACTCAAAGGCGGT |
| | AATACGGTTATCCACAGAATCAGGGGATAACGCAGGAAAGAACAT |
| | GTGAGCAAAAGGCCAGCAAAAGGCCAGGAACCGTAAAAAGGCCG |
| | CGTTGCTGGCGTTTTTCCATAGGCTCCGCCCCCCTGACGAGCAT |
| | CACAAAAATCGACGCTCAAGTCAGAGGTGGCGAAACCCGACAGG |
| | ACTATAAAGATACCAGGCGTTTCCCCCTGGAAGCTCCCTCGTGC |
| | GCTCTCCTGTTCCGACCCTGCCGCTTACCGGATACCTGTCCGCC |
| | TTTCTCCCTTCGGGAAGCGTGGCGCTTTCTCATAGCTCACGCTGT |
| | AGGTATCTCAGTTCGGTGTAGGTCGTTCGCTCCAAGCTGGGCTG |
| | TGTGCACGAACCCCCCGTTCAGCCCGACCGCTGCGCCTTATCCG |
| | GTAACTATCGTCTTGAGTCCAACCCGGTAAGACACGACTTATCGC |
| | CACTGGCAGCAGCCACTGGTAACAGGATTAGCAGAGCGAGGTAT |
| | GTAGGCGGTGCTACAGAGTTCTTGAAGTGGTGGCCTAACTACGG |
| | CTACACTAGAAGAACAGTATTTGGTATCTGCGCTCTGCTGAAGCC |
| | AGTTACCTTCGGAAAAAGAGTTGGTAGCTCTTGATCCGGCAAACA |
| | AACCACCGCTGGTAGCGGTGGTTTTTTTGTTTGCAAGCAGCAGAT |
| | TACGCGCAGAAAAAAAGGATCTCAAGAAGATCCTTTGATCTTTTCT |
| | ACGGGGTCTGACGCTCAGTGGAACGAAAACTCACGTTAAGGGAT |
| | TTTGGTCATGAGATTATCAAAAAGGATCTTCACCTAGATCCTTTTA |
| | AATTAAAAATGAAGTTTTAAATCAATCTAAAGTATATATGAGTAAAC |
| | TTGGTCTGACAGTTACCAATGCTTAATCAGTGAGGCACCTATCTC |
| | AGCGATCTGTCTATTTCGTTCATCCATAGTTGCCTGACTCCCCGT |
| | CGTGTAGATAACTACGATACGGGAGGGCTTACCATCTGGCCCCA |
| | GTGCTGCAATGATACCGCGAGACCCACGCTCACCGGCTCCAGAT |
| | TTATCAGCAATAAACCAGCCAGCCGGAAGGGCCGAGCGCAGAAG |
| | TGGTCCTGCAACTTTATCCGCCTCCATCCAGTCTATTAATTGTTGC |
| | CGGGAAGCTAGAGTAAGTAGTTCGCCAGTTAATAGTTTGCGCAAC |
| | GTTGTTGCCATTGCTACAGGCATCGTGGTGTCACGCTCGTCGTTT |
| | GGTATGGCTTCATTCAGCTCCGGTTCCCAACGATCAAGGCGAGTT |
| | ACATGATCCCCCATGTTGTGCAAAAAAGCGGTTAGCTCCTTCGGT |
| | CCTCCGATCGTTGTCAGAAGTAAGTTGGCCGCAGTGTTATCACTC |
| | ATGGTTATGGCAGCACTGCATAATTCTCTTACTGTCATGCCATCC |
| | GTAAGATGCTTTTCTGTGACTGGTGAGTACTCAACCAAGTCATTC |
| | TGAGAATAGTGTATGCGGCGACCGAGTTGCTCTTGCCCGGCGTC |
| | AATACGGGATAATACCGCGCCACATAGCAGAACTTTAAAAGTGCT |
| | CATCATTGGAAAACGTTCTTCGGGGCGAAAACTCTCAAGGATCTT |
| | ACCGCTGTTGAGATCCAGTTCGATGTAACCCACTCGTGCACCCAA |
| | CTGATCTTCAGCATCTTTTACTTTCACCAGCGTTTCTGGGTGAGC |
| | AAAAACAGGAAGGCAAAATGCCGCAAAAAAGGGAATAAGGGCGA |
| | CACGGAAATGTTGAATACTCATACTCTTCCTTTTTCAATATTATTGA |
| | AGCATTTATCAGGGTTATTGTCTCATGAGCGGATACATATTTGAAT |
| | GTATTTAGAAAAATAAACAAATAGGGGTTCCGCGCACATTTCCCC |
| | GAAAAGTGCCACCTGACGCGCCCTGTAGCGGCGCATTAAGCGCG |
| | GCGGGTGTGGTGGTTACGCGCAGCGTGACCGCTACACTTGCCA |
| | GCGCCCTAGCGCCCGCTCCTTTCGCTTTCTTCCCTTCCTTTCTCG |
| | CCACGTTCGCCGGCTTTCCCCGTCAAGCTCTAAATCGGGGGCTC |
| | CCTTTAGGGTTCCGATTTAGTGCTTTACGGCACCTCGACCCCAAA |
| | AAACTTGATTAGGGTGATGGTTCACGTAGTGGGCCATCGCCCTG |
| | ATAGACGGTTTTTCGCCCTTTGACGTTGGAGTCCACGTTCTTTAAT |
| | AGTGGACTCTTGTTCCAAACTGGAACAACACTCAACCCTATCTCG |
| | GTCTATTCTTTTGATTTATAAGGGATTTTGCCGATTTCGGCCTATT |
| | GGTTAAAAAATGAGCTGATTTAACAAAAATTTAACGCGAATTTTAA |
| | CAAAATATTAACGCTTACAATTTGCCATTCGCCATTCAGGCTGCG |
| | CAACTGTTGGGAAGGGCGAT |
| SEQ ID NO: 48 | CGGTGCGGGCCTCTTCGCTATTACGCCAGCTGGCGAAAGGGGG |
| | ATGTGCTGCAAGGCGATTAAGTTGGGTAACGCCAGGGTTTTCCC |
| | AGTCACGACGTTGTAAAACGACGGCCAGTGAATTGTAATACGACT |
| | CACTATAGGGCGACCCTTAGGATCTAAGCATTGGCGCGCCCCGG |
| | CTGTCTGCCATGCTGCCCGGTGTACCGACATAACCGCCGGTGGC |
| | ATAGCCGCGCATACGCGCCATTTCCTTCCATCTTGTGATTCATGC |
| | TATCCATCTTTTTTGAGTATCCAATTAACGAAGACGTTACCAGCTG |
| | ATTGAAGGTTCTCAAAGTGACTGTACTCCATGTTTTCTTATCATCC |
| | ATGTAGTTATTTTTCAAACTGCAAATTCAAGAAAAAGCCACGCGTG |
| | TGCACCTTTTTTTTCCCCTTCCAGTGCATTATGCAATAGACAGCAC |
| | GAGTCTTTGAAAAAGTAACTTATAAAACTGTATCAATTTTTAAACCT |
| | AAATAGATTCATAAACTATTCGTTAATATAAAGTGTTCTAAACTATG |
| | ATGAAAAAATAAGCAGAAAAGACTAATAATTCTTAGTTAAAAGCAC |
| | TCCGCGGTTACCACACATCTCTCAAGTATCTTCCCTCTGTTTGTAA |
| | CTTTTTCACAATTGCTTCCGCTTCAGAAGAACTAACGCCTTCCTGT |
| | TCCTGGACTATAGTATGAAGTGTTCTGTGAACATCTCTTGCCATAC |
| | CCTTTGCATCACCACAGACATATAGATAGCCTTCCTCTTTGATTAA |
| | GTCCCAAACTTGTGCGGCCTTTTCCATCATTTTGTGTTGGACGTA |
| | CTCCTTCTGAGCACCTTCTCTAGAAAAAGCCATTATCAACTCTGAA |
| | ATAACTCCTTGATCTACAAAGTTATTCAGTTCATCTTCGTAGATGA |
| | AATCCATTTGTCTGTTTCTACAGCCGAAAAACAACAAAGAAGATCC |
| | CAACTCTTCACCATCCTCCTTTAAGGCCATTCTCTCTTGTAAGAAA |
| | CCTCTGAATGGAGCAAGACCTGTACCAGGACCGACCATGACAAT |
| | AGGAGTAGAAGGATTGGAAGGCAGTTTGAAGTTGGAGGCTCTGA |
| | TAAAGATTGGAGCACCAGAACATTCGTGAGACTTCTCTGCTGGAA |
| | CCGCGTTTTTCATCCATGTTGAACAAACGCCCTTATGGATTCTAC |
| | CAGTAGGAGTTGGACCGTACACTAAAGCGGATGTGACATGAACT |
| | CTTGATGGTGCCAGTCTAGGTGAGGATGAAATTGAATAGTATCTT |
| | GGTTGCAGTCTAGGCGCTATTGCGGCGAAGAAAACACCCAAAGG |
| | AGGTTTAGCGGATGGGAAAGCAGCCATAACTTCTAGTAAAGAACG |
| | TTGACTAGCTACTATCCATTGTGAGTATTCATCCTTACCATCTGGT |
| | GAAGTTAGATGTTTCAGTTTTTCTGCCTCAGAAGGTTCTGTGGCG |
| | TACGCAGCCAAGGCCACTAGAGCTGATTTACGTGGAGGATTTAAC |
| | AGATCCGCGTAACGAGCTAAACCGGTACCTAGGGTGCATGGTCC |
| | TGGAAATGGTGGAGGCACTGCACTTTCTAGTGGTGAGCCATCCT |
| | CTTTATCGGCATGAATTGAGAAAACAAGATCTAAACTATGGCCCA |
| | ACAACTTTCCAGCTTCCTCTACAATTTCAACATGGTTTTCAGCGTA |
| | GACACCCACGTGATCACCTGTTTCGTAAGTGATACCAGTACGTGA |
| | TATATCAAATTCAAGATGTATGCAAGATCTGTCTGATTCATGAGTG |
| | TGCAATTCCTTTTGAACTGCAACGTCTACTCTACATGGATGATGAA |
| | TATCGATGGTAGTATTACCATTAGCCACATTACTTTCCATTGATTT |
| | CTGTGTTGTGAATCTTGGATCATGAGTAACTACTCTATATTCTGGA |
| | ATGACGGCTGTGTATGGAGTGGCAACGGATTTATCATCTTCGTCC |
| | TTAAGTAACTTATCTAATTCAGACCACAAAGATTCCTTCCATGCAT |
| | TAAAGTCATCCTCGATAGATTGATCATCATCTCCTAAACCGACTTC |
| | AATCAATCTCTTCGCACCCTTTTTGCATAACTCTTCATCTAAGACA |
| | ATACCTATCTTGTTAAAGTGCTCGTATTGTCTGTTACCTAAGGCAA |
| | AAACGCCGTAAGCAAGTTGCTGCAACTTGATATCTCTTTCGTTCT |
| | CTTCAGTAAACCACTTGTAGAATCTTGCGGCGTTATCGGTTGGTT |
| | CACCATCACCATACGTGGCTACACAAAAGAAAGCCAATGTTTCCT |
| | TTTTCAACTTTTCCTCATATTGGTCATCATCGGCAGCGTAATCATC |
| | CAAATCGATTACTTTTACAGCCGCCTTTTCGTATCTTGCTTTGATC |
| | TCTTCTGAAAGTGCTTTAGCGAATCCTTCGGCTGTTCCGGTTTGT |
| | GTGCCGAAGAAGATAGAGACTCTCGTTTTTCCAGAACCTAGATCT |
| | AAGTCATCATCCTCATCTTTCGCCATCAGAGACTTAGGGATCATTA |
| | GTGGCTTTAGCTCGCCGGAACGATCTGCCGTGGTCTTTTTCCACA |
| | ATAAGACAACGAAACCAGCAACCAGTGCCAGAGAAGTTGTAGCA |
| | ATAACTAATACAACATCATCGGACAAAGAATCCGTTCCCATGATAC |
| | TTTTCAATTGTTTGAAAAGATCGGAGGCATAAAGTGCAGAAGTCA |
| | TTTTAAGCTTTTTGTAATTAAAACTTAGATTAGATTGCTATGCTTTC |
| | TTTCTAATGAGCAAGAAGTAAAAAAAGTTGTAATAGAACAAGAAAA |
| | ATGAAACTGAAACTTGAGAAATTGAAGACCGTTTATTAACTTAAAT |
| | ATCAATGGGAGGTCATCGAAAGAGAAAAAAATCAAAAAAAAAAAT |
| | TTTCAAGAAAAAGAAACGTGATAAAAATTTTTATTGCCTTTTTCGA |
| | CGAAGAAAAAGAAACGAGGCGGTCTCTTTTTTCTTTTCCAAACCTT |
| | TAGTACGGGTAATTAACGACACCCTAGAGGAAGAAAGAGGGGAA |
| | ATTTAGTATGCTGTGCTTGGGTGTTTTGAAGTGGTACGGCGATGC |
| | GCGGAGTCCGAGAAAATCTGGAAGAGTAAAAAAGGAGTAGAAAC |
| | ATTTTGAAGCTAGGCGCGTCAGCCGGTAAAGATTCCCCACGCCA |
| | ATCCGGCTGGTTGCCTCCTTCGTGAAGACAAACTCGGCGCGCCA |
| | TTACAGATCTTAAGGGGATATCCTCGAGGTTCCCTTTAGTGAGGG |
| | TTAATTGCGAGCTTGGCGTAATCATGGTCATAGCTGTTTCCTGTG |
| | TGAAATTGTTATCCGCTCACAATTCCACACAACATACGAGCCGGA |
| | AGCATAAAGTGTAAAGCCTGGGGTGCCTAATGAGTGAGCTAACTC |
| | ACATTAATTGCGTTGCGCTCACTGCCCGCTTTCCAGTCGGGAAAC |
| | CTGTCGTGCCAGCTGCATTAATGAATCGGCCAACGCGCGGGGAG |
| | AGGCGGTTTGCGTATTGGGCGCTCTTCCGCTTCCTCGCTCACTG |
| | ACTCGCTGCGCTCGGTCGTTCGGCTGCGGCGAGCGGTATCAGCT |
| | CACTCAAAGGCGGTAATACGGTTATCCACAGAATCAGGGGATAAC |
| | GCAGGAAAGAACATGTGAGCAAAAGGCCAGCAAAAGGCCAGGAA |
| | CCGTAAAAAGGCCGCGTTGCTGGCGTTTTTCCATAGGCTCCGCC |
| | CCCCTGACGAGCATCACAAAAATCGACGCTCAAGTCAGAGGTGG |
| | CGAAACCCGACAGGACTATAAAGATACCAGGCGTTTCCCCCTGG |
| | AAGCTCCCTCGTGCGCTCTCCTGTTCCGACCCTGCCGCTTACCG |
| | GATACCTGTCCGCCTTTCTCCCTTCGGGAAGCGTGGCGCTTTCTC |
| | ATAGCTCACGCTGTAGGTATCTCAGTTCGGTGTAGGTCGTTCGCT |
| | CCAAGCTGGGCTGTGTGCACGAACCCCCCGTTCAGCCCGACCG |
| | CTGCGCCTTATCCGGTAACTATCGTCTTGAGTCCAACCCGGTAAG |
| | ACACGACTTATCGCCACTGGCAGCAGCCACTGGTAACAGGATTA |
| | GCAGAGCGAGGTATGTAGGCGGTGCTACAGAGTTCTTGAAGTGG |
| | TGGCCTAACTACGGCTACACTAGAAGAACAGTATTTGGTATCTGC |
| | GCTCTGCTGAAGCCAGTTACCTTCGGAAAAAGAGTTGGTAGCTCT |
| | TGATCCGGCAAACAAACCACCGCTGGTAGCGGTGGTTTTTTTGTT |
| | TGCAAGCAGCAGATTACGCGCAGAAAAAAAGGATCTCAAGAAGAT |
| | CCTTTGATCTTTTCTACGGGGTCTGACGCTCAGTGGAACGAAAAC |
| | TCACGTTAAGGGATTTTGGTCATGAGATTATCAAAAAGGATCTTCA |
| | CCTAGATCCTTTTAAATTAAAAATGAAGTTTTAAATCAATCTAAAGT |
| | ATATATGAGTAAACTTGGTCTGACAGTTACCAATGCTTAATCAGTG |
| | AGGCACCTATCTCAGCGATCTGTCTATTTCGTTCATCCATAGTTG |
| | CCTGACTCCCCGTCGTGTAGATAACTACGATACGGGAGGGCTTA |
| | CCATCTGGCCCCAGTGCTGCAATGATACCGCGAGACCCACGCTC |
| | ACCGGCTCCAGATTTATCAGCAATAAACCAGCCAGCCGGAAGGG |
| | CCGAGCGCAGAAGTGGTCCTGCAACTTTATCCGCCTCCATCCAG |
| | TCTATTAATTGTTGCCGGGAAGCTAGAGTAAGTAGTTCGCCAGTT |
| | AATAGTTTGCGCAACGTTGTTGCCATTGCTACAGGCATCGTGGTG |
| | TCACGCTCGTCGTTTGGTATGGCTTCATTCAGCTCCGGTTCCCAA |
| | CGATCAAGGCGAGTTACATGATCCCCCATGTTGTGCAAAAAAGCG |
| | GTTAGCTCCTTCGGTCCTCCGATCGTTGTCAGAAGTAAGTTGGCC |
| | GCAGTGTTATCACTCATGGTTATGGCAGCACTGCATAATTCTCTTA |
| | CTGTCATGCCATCCGTAAGATGCTTTTCTGTGACTGGTGAGTACT |
| | CAACCAAGTCATTCTGAGAATAGTGTATGCGGCGACCGAGTTGCT |
| | CTTGCCCGGCGTCAATACGGGATAATACCGCGCCACATAGCAGA |
| | ACTTTAAAAGTGCTCATCATTGGAAAACGTTCTTCGGGGCGAAAA |
| | CTCTCAAGGATCTTACCGCTGTTGAGATCCAGTTCGATGTAACCC |
| | ACTCGTGCACCCAACTGATCTTCAGCATCTTTTACTTTCACCAGC |
| | GTTTCTGGGTGAGCAAAAACAGGAAGGCAAAATGCCGCAAAAAA |
| | GGGAATAAGGGCGACACGGAAATGTTGAATACTCATACTCTTCCT |
| | TTTTCAATATTATTGAAGCATTTATCAGGGTTATTGTCTCATGAGC |
| | GGATACATATTTGAATGTATTTAGAAAAATAAACAAATAGGGGTTC |
| | CGCGCACATTTCCCCGAAAAGTGCCACCTGACGCGCCCTGTAGC |
| | GGCGCATTAAGCGCGGCGGGTGTGGTGGTTACGCGCAGCGTGA |
| | CCGCTACACTTGCCAGCGCCCTAGCGCCCGCTCCTTTCGCTTTC |
| | TTCCCTTCCTTTCTCGCCACGTTCGCCGGCTTTCCCCGTCAAGCT |
| | CTAAATCGGGGGCTCCCTTTAGGGTTCCGATTTAGTGCTTTACGG |
| | CACCTCGACCCCAAAAAACTTGATTAGGGTGATGGTTCACGTAGT |
| | GGGCCATCGCCCTGATAGACGGTTTTTCGCCCTTTGACGTTGGA |
| | GTCCACGTTCTTTAATAGTGGACTCTTGTTCCAAACTGGAACAAC |
| | ACTCAACCCTATCTCGGTCTATTCTTTTGATTTATAAGGGATTTTG |
| | CCGATTTCGGCCTATTGGTTAAAAAATGAGCTGATTTAACAAAAAT |
| | TTAACGCGAATTTTAACAAAATATTAACGCTTACAATTTGCCATTC |
| | GCCATTCAGGCTGCGCAACTGTTGGGAAGGGCGAT |
| SEQ ID NO: 49 | CGGTGCGGGCCTCTTCGCTATTACGCCAGCTGGCGAAAGGGGG |
| | ATGTGCTGCAAGGCGATTAAGTTGGGTAACGCCAGGGTTTTCCC |
| | AGTCACGACGTTGTAAAACGACGGCCAGTGAATTGTAATACGACT |
| | CACTATAGGGCGACCCTTAGGATCCTATGGCGCGCCCAGCCGGT |
| | AAAGATTCCCCACGCCAATCCGGCTGGTTGCCTCCTTCGTGAAG |
| | ACAAACTCACGCGTCCAGTATCCCAGCAGATACGGGATATCGAC |
| | ATTTCTGCACCATTCCGGCGGGTATAGGTTTTATTGATGGCCTCA |
| | TCCACACGCAGCAGCGTCTGTTCATCGTCGTGGCGGCCCATAAT |
| | AATCTGCCGGTCAATCAGCCAGCTTTCCTCACCCGGCCCCCATC |
| | CCCATACGCGCATTTCGTAGCGGTCCAGCTGGGAGTCGATACCG |
| | GCGGTCAGGTAAGCCACACGGTCAGGAACGGGCGCTGAATAATG |
| | CTCTTTCCGCTCTGCCATCACTTCAGCATCCGGACGTTCGCCAAT |
| | TTTCGCCTCCCACGTCTCACCGAGCGTGGTGTTTACGAAGGTTTT |
| | ACGTTTTCCCGTATCCCCTTTCGTTTTCATCCAGTCTTTGACAATC |
| | TGCACCCAGGTGGTGAACGGGCTGTACGCTGTCCAGATGTGAAA |
| | GGTCACACTGTCAGGTGGCTCAATCTCTTCACCGGATGACGAAAA |
| | CCAGAGAATGCCATCACGGGTCCAGATCCCGGTCTTTTCGCAGA |
| | TATAACGGGCATCAGTAAAGTCCAGCTCCTGCTGGCGGATGACG |
| | CAGGCATTATGCTCGCAGAGATAAAACACGCTGGAGACGCGTTTT |
| | CCCGTCTTTCAGTGCCTTGTTCAGTTCTTCCTGACGGGCGGTATA |
| | TTTCTCCAGCTTGGCGCGCCTAAGACTTAGATCTTAAGGGGATAT |
| | CCTCGAGGTTCCCTTTAGTGAGGGTTAATTGCGAGCTTGGCGTAA |
| | TCATGGTCATAGCTGTTTCCTGTGTGAAATTGTTATCCGCTCACAA |
| | TTCCACACAACATACGAGCCGGAAGCATAAAGTGTAAAGCCTGG |
| | GGTGCCTAATGAGTGAGCTAACTCACATTAATTGCGTTGCGCTCA |
| | CTGCCCGCTTTCCAGTCGGGAAACCTGTCGTGCCAGCTGCATTA |
| | ATGAATCGGCCAACGCGCGGGGAGAGGCGGTTTGCGTATTGGG |
| | CGCTCTTCCGCTTCCTCGCTCACTGACTCGCTGCGCTCGGTCGT |
| | TCGGCTGCGGCGAGCGGTATCAGCTCACTCAAAGGCGGTAATAC |
| | GGTTATCCACAGAATCAGGGGATAACGCAGGAAAGAACATGTGA |
| | GCAAAAGGCCAGCAAAAGGCCAGGAACCGTAAAAAGGCCGCGTT |
| | GCTGGCGTTTTTCCATAGGCTCCGCCCCCCTGACGAGCATCACA |
| | AAAATCGACGCTCAAGTCAGAGGTGGCGAAACCCGACAGGACTA |
| | TAAAGATACCAGGCGTTTCCCCCTGGAAGCTCCCTCGTGCGCTC |
| | TCCTGTTCCGACCCTGCCGCTTACCGGATACCTGTCCGCCTTTCT |
| | CCCTTCGGGAAGCGTGGCGCTTTCTCATAGCTCACGCTGTAGGT |
| | ATCTCAGTTCGGTGTAGGTCGTTCGCTCCAAGCTGGGCTGTGTG |
| | CACGAACCCCCCGTTCAGCCCGACCGCTGCGCCTTATCCGGTAA |
| | CTATCGTCTTGAGTCCAACCCGGTAAGACACGACTTATCGCCACT |
| | GGCAGCAGCCACTGGTAACAGGATTAGCAGAGCGAGGTATGTAG |
| | GCGGTGCTACAGAGTTCTTGAAGTGGTGGCCTAACTACGGCTAC |
| | ACTAGAAGAACAGTATTTGGTATCTGCGCTCTGCTGAAGCCAGTT |
| | ACCTTCGGAAAAAGAGTTGGTAGCTCTTGATCCGGCAAACAAACC |
| | ACCGCTGGTAGCGGTGGTTTTTTTGTTTGCAAGCAGCAGATTACG |
| | CGCAGAAAAAAAGGATCTCAAGAAGATCCTTTGATCTTTTCTACG |
| | GGGTCTGACGCTCAGTGGAACGAAAACTCACGTTAAGGGATTTT |
| | GGTCATGAGATTATCAAAAAGGATCTTCACCTAGATCCTTTTAAAT |
| | TAAAAATGAAGTTTTAAATCAATCTAAAGTATATATGAGTAAACTTG |
| | GTCTGACAGTTACCAATGCTTAATCAGTGAGGCACCTATCTCAGC |
| | GATCTGTCTATTTCGTTCATCCATAGTTGCCTGACTCCCCGTCGT |
| | GTAGATAACTACGATACGGGAGGGCTTACCATCTGGCCCCAGTG |
| | CTGCAATGATACCGCGAGACCCACGCTCACCGGCTCCAGATTTA |
| | TCAGCAATAAACCAGCCAGCCGGAAGGGCCGAGCGCAGAAGTG |
| | GTCCTGCAACTTTATCCGCCTCCATCCAGTCTATTAATTGTTGCC |
| | GGGAAGCTAGAGTAAGTAGTTCGCCAGTTAATAGTTTGCGCAACG |
| | TTGTTGCCATTGCTACAGGCATCGTGGTGTCACGCTCGTCGTTTG |
| | GTATGGCTTCATTCAGCTCCGGTTCCCAACGATCAAGGCGAGTTA |
| | CATGATCCCCCATGTTGTGCAAAAAAGCGGTTAGCTCCTTCGGTC |
| | CTCCGATCGTTGTCAGAAGTAAGTTGGCCGCAGTGTTATCACTCA |
| | TGGTTATGGCAGCACTGCATAATTCTCTTACTGTCATGCCATCCG |
| | TAAGATGCTTTTCTGTGACTGGTGAGTACTCAACCAAGTCATTCT |
| | GAGAATAGTGTATGCGGCGACCGAGTTGCTCTTGCCCGGCGTCA |
| | ATACGGGATAATACCGCGCCACATAGCAGAACTTTAAAAGTGCTC |
| | ATCATTGGAAAACGTTCTTCGGGGCGAAAACTCTCAAGGATCTTA |
| | CCGCTGTTGAGATCCAGTTCGATGTAACCCACTCGTGCACCCAAC |
| | TGATCTTCAGCATCTTTTACTTTCACCAGCGTTTCTGGGTGAGCAA |
| | AAACAGGAAGGCAAAATGCCGCAAAAAAGGGAATAAGGGCGACA |
| | CGGAAATGTTGAATACTCATACTCTTCCTTTTTCAATATTATTGAA |
| | GCATTTATCAGGGTTATTGTCTCATGAGCGGATACATATTTGAATG |
| | TATTTAGAAAAATAAACAAATAGGGGTTCCGCGCACATTTCCCCG |
| | AAAAGTGCCACCTGACGCGCCCTGTAGCGGCGCATTAAGCGCGG |
| | CGGGTGTGGTGGTTACGCGCAGCGTGACCGCTACACTTGCCAGC |
| | GCCCTAGCGCCCGCTCCTTTCGCTTTCTTCCCTTCCTTTCTCGCC |
| | ACGTTCGCCGGCTTTCCCCGTCAAGCTCTAAATCGGGGGCTCCC |
| | TTTAGGGTTCCGATTTAGTGCTTTACGGCACCTCGACCCCAAAAA |
| | ACTTGATTAGGGTGATGGTTCACGTAGTGGGCCATCGCCCTGATA |
| | GACGGTTTTTCGCCCTTTGACGTTGGAGTCCACGTTCTTTAATAG |
| | TGGACTCTTGTTCCAAACTGGAACAACACTCAACCCTATCTCGGT |
| | CTATTCTTTTGATTTATAAGGGATTTTGCCGATTTCGGCCTATTGG |
| | TTAAAAAATGAGCTGATTTAACAAAAATTTAACGCGAATTTTAACA |
| | AAATATTAACGCTTACAATTTGCCATTCGCCATTCAGGCTGCGCA |
| | ACTGTTGGGAAGGGCGAT |
| SEQ ID NO: 50 | TTTCCCAGTCACGACGTTGTAAAACGACGGCCAGTGAATTGTAAT |
| | ACGACTCACTATAGGGCGACCCTTAAGATCTGTAATGGCGCGCC |
| | ATGCGCGGCTATGCCACCGGCGGTTATGTCGGTACACCGGGCA |
| | GCATGGCAGACAGCCGGACGCGCCACGCACAGATATTATAACAT |
| | CTGCATAATAGGCATTTGCAAGAATTACTCGTGAGTAAGGAAAGA |
| | GTGAGGAACTATCGCATACCTGCATTTAAAGATGCCGATTTGGGC |
| | GCGAATCCTTTATTTTGGCTTCACCCTCATACTATTATCAGGGCCA |
| | GAAAAAGGAAGTGTTTCCCTCCTTCTTGAATTGATGTTACCCTCAT |
| | AAAGCACGTGGCCTCTTATCGAGAAAGAAATTACCGTCGCTCGTG |
| | ATTTGTTTGCAAAAAGAACAAAACTGAAAAAACCCAGACACGCTC |
| | GACTTCCTGTCTTCCTATTGATTGCAGCTTCCAATTTCGTCACACA |
| | ACAAGGTCCTAGCGACGGCTCACAGGTTTTGTAACAAGCAATCGA |
| | AGGTTCTGGAATGGCGGGAAAGGGTTTAGTACCACATGCTATGAT |
| | GCCCACTGTGATCTCCAGAGCAAAGTTCGTTCGATCGTACTGTTA |
| | CTCTCTCTCTTTCAAACAGAATTGTCCGAATCGTGTGACAACAACA |
| | GCCTGTTCTCACACACTCTTTTCTTCTAACCAAGGGGGTGGTTTA |
| | GTTTAGTAGAACCTCGTGAAACTTACATTTACATATATATAAACTT |
| | GCATAAATTGGTCAATGCAAGAAATACATATTTGGTCTTTTCTAAT |
| | TCGTAGTTTTTCAAGTTCTTAGATGCTTTCTTTTTCTCTTTTTTACA |
| | GATCATCAAGGAAGTAATTATCTACTTTTTACAACAAATATAAAAC |
| | AAAGCTTGGCCTGCAGGGCCAGCTTACCCTTAAATTTATTTGCAC |
| | TACTGGAAAACTACCTGTTCCATGGCCAACACTTGTCACTACTTTC |
| | TCTTATGGTGTTCAATGCTTTTCCCGTTATCCGGATCATATGAAAC |
| | GGCATGACTTTTTCAAGAGTGCCATGCCCGAAGGTTATGTACAGG |
| | AACGCACTATATCTTTCAAAGATGACGGGAACTACAAGACGCGTG |
| | CTGAAGTCAAGTTTGAAGGTGATACCCTTGTTAATCGTATCGAGT |
| | TAAAAGGTATTGATTTTAAAGAAGATGGAAACATTCTCGGACACAA |
| | ACTCGAGTACAACTATAACTCACACAATGTATACATCACGGCAGA |
| | CAAACAAAAGAATGGAATCAAAGCTAACTTCAAAATTCGCCACAA |
| | CATTGAAGATGGATCCGTTCAACTAGCAGACCATTATCAACAAAA |
| | TACTCCAATTGGCGATGGCCCTGTCCTTTTACCAGACAACCATTA |
| | CCTGTCGACACAATCTGCCCTTTCGAAAGATCCCAACGAAAAGCG |
| | TGACCACATGGTCCTTCTTGAGTTTGTAACTGCTGCTGGGATTAC |
| | ACATGGCATGGATGAGCTCTACAAATAATGAATTCGTCGAGGCCG |
| | TTCAGGCCTCGAGGCCGTTCAGGCTCGACCCGGGGATCCGCGG |
| | ATCTCTTATGTCTTTACGATTTATAGTTTTCATTATCAAGTATGCCT |
| | ATATTAGTATATAGCATCTTTAGATGACAGTGTTCGAAGTTTCACG |
| | AATAAAAGATAATATTCTACTTTTTGCTCCCACCGCGTTTGCTAGC |
| | ACGAGTGAACACCATCCCTCGCCTGTGAGTTGTACCCATTCCTCT |
| | AAACTGTAGACATGGTAGCTTCAGCAGTGTTCGTTATGTACGGCA |
| | TCCTCCAACAAACAGTCGGTTATAGTTTGTCCTGCTCCTCTGAAT |
| | CGTCTCCCTCGATATTTCTCATTTTCCTTCGGCGCGTTCGCAGGC |
| | GTCCGGGACGTTTGAGCAGAATAACCATGTGGTGATTAACAACGA |
| | CGGCACGGGCGCGCCAATGCTTAGATCTTAAGGGGATATCCTCG |
| | AGGTTCCCTTTAGTGAGGGTTAATTGCGAGCTTGGCGTAATCATG |
| | GTCATAGCTGTTTCCTGTGTGAAATTGTTATCCGCTCACAATTCCA |
| | CACAACATACGAGCCGGAAGCATAAAGTGTAAAGCCTGGGGTGC |
| | CTAATGAGTGAGCTAACTCACATTAATTGCGTTGCGCTCACTGCC |
| | CGCTTTCCAGTCGGGAAACCTGTCGTGCCAGCTGCATTAATGAAT |
| | CGGCCAACGCGCGGGGAGAGGCGGTTTGCGTATTGGGCGCTCT |
| | TCCGCTTCCTCGCTCACTGACTCGCTGCGCTCGGTCGTTCGGCT |
| | GCGGCGAGCGGTATCAGCTCACTCAAAGGCGGTAATACGGTTAT |
| | CCACAGAATCAGGGGATAACGCAGGAAAGAACATGTGAGCAAAA |
| | GGCCAGCAAAAGGCCAGGAACCGTAAAAAGGCCGCGTTGCTGG |
| | CGTTTTTCCATAGGCTCCGCCCCCCTGACGAGCATCACAAAAATC |
| | GACGCTCAAGTCAGAGGTGGCGAAACCCGACAGGACTATAAAGA |
| | TACCAGGCGTTTCCCCCTGGAAGCTCCCTCGTGCGCTCTCCTGT |
| | TCCGACCCTGCCGCTTACCGGATACCTGTCCGCCTTTCTCCCTTC |
| | GGGAAGCGTGGCGCTTTCTCATAGCTCACGCTGTAGGTATCTCA |
| | GTTCGGTGTAGGTCGTTCGCTCCAAGCTGGGCTGTGTGCACGAA |
| | CCCCCCGTTCAGCCCGACCGCTGCGCCTTATCCGGTAACTATCG |
| | TCTTGAGTCCAACCCGGTAAGACACGACTTATCGCCACTGGCAG |
| | CAGCCACTGGTAACAGGATTAGCAGAGCGAGGTATGTAGGCGGT |
| | GCTACAGAGTTCTTGAAGTGGTGGCCTAACTACGGCTACACTAGA |
| | AGAACAGTATTTGGTATCTGCGCTCTGCTGAAGCCAGTTACCTTC |
| | GGAAAAAGAGTTGGTAGCTCTTGATCCGGCAAACAAACCACCGC |
| | TGGTAGCGGTGGTTTTTTTGTTTGCAAGCAGCAGATTACGCGCAG |
| | AAAAAAAGGATCTCAAGAAGATCCTTTGATCTTTTCTACGGGGTCT |
| | GACGCTCAGTGGAACGAAAACTCACGTTAAGGGATTTTGGTCATG |
| | AGATTATCAAAAAGGATCTTCACCTAGATCCTTTTAAATTAAAAAT |
| | GAAGTTTTAAATCAATCTAAAGTATATATGAGTAAACTTGGTCTGA |
| | CAGTTACCAATGCTTAATCAGTGAGGCACCTATCTCAGCGATCTG |
| | TCTATTTCGTTCATCCATAGTTGCCTGACTCCCCGTCGTGTAGATA |
| | ACTACGATACGGGAGGGCTTACCATCTGGCCCCAGTGCTGCAAT |
| | GATACCGCGAGACCCACGCTCACCGGCTCCAGATTTATCAGCAA |
| | TAAACCAGCCAGCCGGAAGGGCCGAGCGCAGAAGTGGTCCTGC |
| | AACTTTATCCGCCTCCATCCAGTCTATTAATTGTTGCCGGGAAGC |
| | TAGAGTAAGTAGTTCGCCAGTTAATAGTTTGCGCAACGTTGTTGC |
| | CATTGCTACAGGCATCGTGGTGTCACGCTCGTCGTTTGGTATGG |
| | CTTCATTCAGCTCCGGTTCCCAACGATCAAGGCGAGTTACATGAT |
| | CCCCCATGTTGTGCAAAAAAGCGGTTAGCTCCTTCGGTCCTCCGA |
| | TCGTTGTCAGAAGTAAGTTGGCCGCAGTGTTATCACTCATGGTTA |
| | TGGCAGCACTGCATAATTCTCTTACTGTCATGCCATCCGTAAGAT |
| | GCTTTTCTGTGACTGGTGAGTACTCAACCAAGTCATTCTGAGAAT |
| | AGTGTATGCGGCGACCGAGTTGCTCTTGCCCGGCGTCAATACGG |
| | GATAATACCGCGCCACATAGCAGAACTTTAAAAGTGCTCATCATT |
| | GGAAAACGTTCTTCGGGGCGAAAACTCTCAAGGATCTTACCGCT |
| | GTTGAGATCCAGTTCGATGTAACCCACTCGTGCACCCAACTGATC |
| | TTCAGCATCTTTTACTTTCACCAGCGTTTCTGGGTGAGCAAAAACA |
| | GGAAGGCAAAATGCCGCAAAAAAGGGAATAAGGGCGACACGGAA |
| | ATGTTGAATACTCATACTCTTCCTTTTTCAATATTATTGAAGCATTT |
| | ATCAGGGTTATTGTCTCATGAGCGGATACATATTTGAATGTATTTA |
| | GAAAAATAAACAAATAGGGGTTCCGCGCACATTTCCCCGAAAAGT |
| | GCCACCTGACGCGCCCTGTAGCGGCGCATTAAGCGCGGCGGGT |
| | GTGGTGGTTACGCGCAGCGTGACCGCTACACTTGCCAGCGCCCT |
| | AGCGCCCGCTCCTTTCGCTTTCTTCCCTTCCTTTCTCGCCACGTT |
| | CGCCGGCTTTCCCCGTCAAGCTCTAAATCGGGGGCTCCCTTTAG |
| | GGTTCCGATTTAGTGCTTTACGGCACCTCGACCCCAAAAAACTTG |
| | ATTAGGGTGATGGTTCACGTAGTGGGCCATCGCCCTGATAGACG |
| | GTTTTTCGCCCTTTGACGTTGGAGTCCACGTTCTTTAATAGTGGA |
| | CTCTTGTTCCAAACTGGAACAACACTCAACCCTATCTCGGTCTATT |
| | CTTTTGATTTATAAGGGATTTTGCCGATTTCGGCCTATTGGTTAAA |
| | AAATGAGCTGATTTAACAAAAATTTAACGCGAATTTTAACAAAATA |
| | TTAACGCTTACAATTTGCCATTCGCCATTCAGGCTGCGCAACTGT |
| | TGGGAAGGGCGAT |
| SEQ ID NO: 51 | CGGTGCGGGCCTCTTCGCTATTACGCCAGCTGGCGAAAGGGGG |
| | ATGTGCTGCAAGGCGATTAAGTTGGGTAACGCCAGGGTTTTCCC |
| | AGTCACGACGTTGTAAAACGACGGCCAGTGAATTGTAATACGACT |
| | CACTATAGGGCGACCCTTAGGATCCTATGGCGCGCCGGCACCCT |
| | TGCGGGCCATGTCATACACCGCCTTCAGAGCAGCCGGACCTATC |
| | TGCCCGTTACGCGCCAGCTTGCAAATTAAAGCCTTCGAGCGTCC |
| | CAAAACCTTCTCAAGCAAGGTTTTCAGTATAATGTTACATGCGTAC |
| | ACGCGTCTGTACAGAAAAAAAAGAAAAATTTGAAATATAAATAACG |
| | TTCTTAATACTAACATAACTATAAAAAAATAAATAGGGACCTAGAC |
| | TTCAGGTTGTCTAACTCCTTCCTTTTCGGTTAGAGCGGATGTGGG |
| | GGGAGGGCGTGAATGTAAGCGTGACATAACTAATTACATGATATC |
| | GACAAAGGAAAAGGGGGACGGATCTCCGAGGCCTCGGACCCGT |
| | CGGGCCGCCGTCGGACGTGCCGCGGTCAGGTGGCGAACTTCTT |
| | AATACCTTGTTGCAAGATAGAGTCGAAAACGTCCATCTTTTTCTTT |
| | TCCAAGGCAATACCAATTTCAACACCGTTAGAACCATCTCTAGATT |
| | CAGAGAAGGCAATGGAACCACCAGTTTCAATATGAACGATTTCCA |
| | TCTTGCATGGCTTACCCAAACCAAAATCCATATCGTACAAACCCA |
| | ATTTTGGAGCACCAGCAATAGAGGTTGGGTAATGAGACATAACCC |
| | ATTTTCTAACACCTTGACCCCATCTTGGAGCAGTTTTCAACAAATC |
| | GGAGGACAACATATCCTTGATTCTAGCAGTAATAGCATCAGAAGC |
| | AGCCAAAACGCACTTTTCACCCAACAAATCATGTTTTTTGACAGAG |
| | ACTATACCTGGAGCCATACAGTTACCGAAGTAAGTTTGTGGAATA |
| | GGTTGGGTGTACTTCAATCTGTTTCTACAGTCAACGTTAATCATCA |
| | AGTGGAAAACTTCGTCCTTATCTTCTTCGTTAGCCTTAGTTTCAGA |
| | ATCTTGGACCAAGGTCTTAATCAAGGAAACCCAGATAAAAGCCAA |
| | GGTAACAACGAAGGTAGAAACTGGAGATTGATTTTCGGATTGTTC |
| | GGTGACCCAAGACTTCAAGTTATCGATTTGCTTTCTGGACAAGGT |
| | GAAAGTAGCTCTAACCATGTTTTCTGGAGTAACATGAGAAGAGTG |
| | CTTGGCGGAATTTTGTGACCAAAATCTTTCCAAATGACCAGCACC |
| | AACTTCACCTGGATCCTTGATCATGTTTCTGCAAGAATGAATTGG |
| | CAAAGATGGCAACAAAACAGTAGCTGGATCTTTACCAGAAGATTT |
| | GGTCAAGGACATCCAGTACTTCATGAAATGTGAGAAAGTAACACC |
| | ATCAGCAACAACATGAGTAGCAGAGTTACCAATACAGATACCAGC |
| | ACCTGGAAAAATAGTGACTTGCATAGCCATAATTGGTCTCATTTGA |
| | ATACCTTCAGGTGAAACATGTGGTGGTGGCAATTTTGGCAAAACA |
| | CCATGTAAAACGGAAATATCCTTTGGGGAATCGGACTTCAATTGA |
| | TCGAAATCGGTTTCAGTAGATTCAGCAACGGTGAAAACCAAAGAG |
| | TCTTGACCATCATTGTAATGCAAGTATGGTGGATCTGGTCTTGGT |
| | GGAATAATCAACTTACCGGCGTATGGAAAAAAATGTTGCAAGGTA |
| | ATAGACAAGGAGTGCTTCAAGTTTGGGACGAAATCTTGTAAGAAA |
| | GATTCGGTGGAGTTTTGGTAGGAGAAGAAGAACAAAGAATCAGC |
| | CAATGGTAAAGACAACCATGGGGCATCAAAAAAAGTCAATGGCAA |
| | AGTAGTAGATGGAACAGTACCCTTTGGTGGAGAAATATGGCAGGT |
| | TTCAATAATCTTTGGTGGTTGCAAGTGAGCAACCATTTTAAGCTTT |
| | TTGTTTGTTTATGTGTGTTTATTCGAAACTAAGTTCTTGGTGTTTTA |
| | AAACTAAAAAAAAGACTAACTATAAAAGTAGAATTTAAGAAGTTTA |
| | AGAAATAGATTTACAGAATTACAATCAATACCTACCGTCTTTATAT |
| | ACTTATTAGTCAAGTAGGGGAATAATTTCAGGGAACTGGTTTCAA |
| | CCTTTTTTTTCAGCTTTTTCCAAATCAGAGAGAGCAGAAGGTAATA |
| | GAAGGTGTAAGAAAATGAGATAGATACATGCGTGGGTCAATTGCC |
| | TTGTGTCATCATTTACTCCAGGCAGGTTGCATCACTCCATTGAGG |
| | TTGTGTCCGTTTTTTGCCTGTTTGTGCCCCTGTTCTCTGTAGTTGC |
| | GCTAAGAGAATGGACCTATGAACTGATGGTTGGTGAAGAAAACAA |
| | TATTTTGGTGCTGGGATTCTTTTTTTTTCTGGATGCCAGCTTAAAA |
| | AGCGGGCTCCATTATATTTAGTGGATGCCAGGAATAAACTGTTCA |
| | CCCAGACACCTACGATGTTATATATTCTGTGTAACCCGCCCCCTA |
| | TTTTGGGCATGTACGGGTTACAGCAGAATTAAAAGGCTAATTTTTT |
| | GACTAAATAAAGTTAGGAAAATCACTACTATTAATTATTTACGTATT |
| | CTTTGAAATGGCAGTATTGATAATGATAAACTCGAACTGGGCGCG |
| | TCGTGCCGTCGTTGTTAATCACCACATGGTTATTCTGCTCAAACG |
| | TCCCGGACGCCTGCGAGGCGCGCCTATTGAAAGATCTTAAGGGG |
| | ATATCCTCGAGGTTCCCTTTAGTGAGGGTTAATTGCGAGCTTGGC |
| | GTAATCATGGTCATAGCTGTTTCCTGTGTGAAATTGTTATCCGCTC |
| | ACAATTCCACACAACATACGAGCCGGAAGCATAAAGTGTAAAGCC |
| | TGGGGTGCCTAATGAGTGAGCTAACTCACATTAATTGCGTTGCGC |
| | TCACTGCCCGCTTTCCAGTCGGGAAACCTGTCGTGCCAGCTGCA |
| | TTAATGAATCGGCCAACGCGCGGGGAGAGGCGGTTTGCGTATTG |
| | GGCGCTCTTCCGCTTCCTCGCTCACTGACTCGCTGCGCTCGGTC |
| | GTTCGGCTGCGGCGAGCGGTATCAGCTCACTCAAAGGCGGTAAT |
| | ACGGTTATCCACAGAATCAGGGGATAACGCAGGAAAGAACATGT |
| | GAGCAAAAGGCCAGCAAAAGGCCAGGAACCGTAAAAAGGCCGC |
| | GTTGCTGGCGTTTTTCCATAGGCTCCGCCCCCCTGACGAGCATC |
| | ACAAAAATCGACGCTCAAGTCAGAGGTGGCGAAACCCGACAGGA |
| | CTATAAAGATACCAGGCGTTTCCCCCTGGAAGCTCCCTCGTGCG |
| | CTCTCCTGTTCCGACCCTGCCGCTTACCGGATACCTGTCCGCCTT |
| | TCTCCCTTCGGGAAGCGTGGCGCTTTCTCATAGCTCACGCTGTA |
| | GGTATCTCAGTTCGGTGTAGGTCGTTCGCTCCAAGCTGGGCTGT |
| | GTGCACGAACCCCCCGTTCAGCCCGACCGCTGCGCCTTATCCGG |
| | TAACTATCGTCTTGAGTCCAACCCGGTAAGACACGACTTATCGCC |
| | ACTGGCAGCAGCCACTGGTAACAGGATTAGCAGAGCGAGGTATG |
| | TAGGCGGTGCTACAGAGTTCTTGAAGTGGTGGCCTAACTACGGC |
| | TACACTAGAAGAACAGTATTTGGTATCTGCGCTCTGCTGAAGCCA |
| | GTTACCTTCGGAAAAAGAGTTGGTAGCTCTTGATCCGGCAAACAA |
| | ACCACCGCTGGTAGCGGTGGTTTTTTTGTTTGCAAGCAGCAGATT |
| | ACGCGCAGAAAAAAAGGATCTCAAGAAGATCCTTTGATCTTTTCT |
| | ACGGGGTCTGACGCTCAGTGGAACGAAAACTCACGTTAAGGGAT |
| | TTTGGTCATGAGATTATCAAAAAGGATCTTCACCTAGATCCTTTTA |
| | AATTAAAAATGAAGTTTTAAATCAATCTAAAGTATATATGAGTAAAC |
| | TTGGTCTGACAGTTACCAATGCTTAATCAGTGAGGCACCTATCTC |
| | AGCGATCTGTCTATTTCGTTCATCCATAGTTGCCTGACTCCCCGT |
| | CGTGTAGATAACTACGATACGGGAGGGCTTACCATCTGGCCCCA |
| | GTGCTGCAATGATACCGCGAGACCCACGCTCACCGGCTCCAGAT |
| | TTATCAGCAATAAACCAGCCAGCCGGAAGGGCCGAGCGCAGAAG |
| | TGGTCCTGCAACTTTATCCGCCTCCATCCAGTCTATTAATTGTTGC |
| | CGGGAAGCTAGAGTAAGTAGTTCGCCAGTTAATAGTTTGCGCAAC |
| | GTTGTTGCCATTGCTACAGGCATCGTGGTGTCACGCTCGTCGTTT |
| | GGTATGGCTTCATTCAGCTCCGGTTCCCAACGATCAAGGCGAGTT |
| | ACATGATCCCCCATGTTGTGCAAAAAAGCGGTTAGCTCCTTCGGT |
| | CCTCCGATCGTTGTCAGAAGTAAGTTGGCCGCAGTGTTATCACTC |
| | ATGGTTATGGCAGCACTGCATAATTCTCTTACTGTCATGCCATCC |
| | GTAAGATGCTTTTCTGTGACTGGTGAGTACTCAACCAAGTCATTC |
| | TGAGAATAGTGTATGCGGCGACCGAGTTGCTCTTGCCCGGCGTC |
| | AATACGGGATAATACCGCGCCACATAGCAGAACTTTAAAAGTGCT |
| | CATCATTGGAAAACGTTCTTCGGGGCGAAAACTCTCAAGGATCTT |
| | ACCGCTGTTGAGATCCAGTTCGATGTAACCCACTCGTGCACCCAA |
| | CTGATCTTCAGCATCTTTTACTTTCACCAGCGTTTCTGGGTGAGC |
| | AAAAACAGGAAGGCAAAATGCCGCAAAAAAGGGAATAAGGGCGA |
| | CACGGAAATGTTGAATACTCATACTCTTCCTTTTTCAATATTATTGA |
| | AGCATTTATCAGGGTTATTGTCTCATGAGCGGATACATATTTGAAT |
| | GTATTTAGAAAAATAAACAAATAGGGGTTCCGCGCACATTTCCCC |
| | GAAAAGTGCCACCTGACGCGCCCTGTAGCGGCGCATTAAGCGCG |
| | GCGGGTGTGGTGGTTACGCGCAGCGTGACCGCTACACTTGCCA |
| | GCGCCCTAGCGCCCGCTCCTTTCGCTTTCTTCCCTTCCTTTCTCG |
| | CCACGTTCGCCGGCTTTCCCCGTCAAGCTCTAAATCGGGGGCTC |
| | CCTTTAGGGTTCCGATTTAGTGCTTTACGGCACCTCGACCCCAAA |
| | AAACTTGATTAGGGTGATGGTTCACGTAGTGGGCCATCGCCCTG |
| | ATAGACGGTTTTTCGCCCTTTGACGTTGGAGTCCACGTTCTTTAAT |
| | AGTGGACTCTTGTTCCAAACTGGAACAACACTCAACCCTATCTCG |
| | GTCTATTCTTTTGATTTATAAGGGATTTTGCCGATTTCGGCCTATT |
| | GGTTAAAAAATGAGCTGATTTAACAAAAATTTAACGCGAATTTTAA |
| | CAAAATATTAACGCTTACAATTTGCCATTCGCCATTCAGGCTGCG |
| | CAACTGTTGGGAAGGGCGAT |
| SEQ ID NO: 52 | CGGTGCGGGCCTCTTCGCTATTACGCCAGCTGGCGAAAGGGGG |
| | ATGTGCTGCAAGGCGATTAAGTTGGGTAACGCCAGGGTTTTCCC |
| | AGTCACGACGTTGTAAAACGACGGCCAGTGAATTGTAATACGACT |
| | CACTATAGGGCGACCCTTAGGATCCTATGGCGCGCCGGCACCCT |
| | TGCGGGCCATGTCATACACCGCCTTCAGAGCAGCCGGACCTATC |
| | TGCCCGTTACGCGCCAGCTTGCAAATTAAAGCCTTCGAGCGTCC |
| | CAAAACCTTCTCAAGCAAGGTTTTCAGTATAATGTTACATGCGTAC |
| | ACGCGTCTGTACAGAAAGAAAAATTTGAAATATAAATAACG |
| | TTCTTAATACTAACATAACTATAAAAAAATAAATAGGGACCTAGAC |
| | TTCAGGTTGTCTAACTCCTTCCTTTTCGGTTAGAGCGGATGTGGG |
| | GGGAGGGCGTGAATGTAAGCGTGACATAACTAATTACATGATATC |
| | GACAAAGGAAAAGGGGGACGGATCTCCGAGGCCTCGGACCCGT |
| | CGGGCCGCCGTCGGACGTGCCGCGGTTAAGAAGCAATAGCGGA |
| | TTCCAAACCGTCGTTAAAGATTTTACCAAAGGCTTCCATTTGCATG |
| | GATGGGAAACAAACACCAATTTCAAAATCTTGGGCGGATTCTTTA |
| | CAAGCTGACAAAGAAACAGAGGCGGAGTAGTCAATAGAAACAAC |
| | TTCGTACTTCATAGCCTTACCCCAACCGAAATCAATATCGTAGAA |
| | GTTCAACTTTGGAGTACCAGAAATACCCATCTTTCTAGCTGGAAT |
| | CTTAAAACCATCGTACCATCTATCAGCGTATTCCAAAATACCACCC |
| | TTCTTGTTAACCATCTTAGAGATACCTTCACCAATCAACTTAGCAG |
| | CCATAACAAAACCGTTTTCACCCTTCAAGACACCGTTCTTAATAGT |
| | GACAATACATGGAGCAGAACAGTTACCGAAGTAGTTTTCTGGTAA |
| | TGGTGGATCTAATCTTGATCTGCAACCGACAGAAACGATGAATTG |
| | TTCCAATTCATCTTCACCCTTTTTTTCACCCATGTTGACCAAGGAC |
| | TTAACGATACAAGACCAAATGTAACCGCAGGTAACAGTGAAAGAA |
| | GAAGTGTATTCCAACATTGGCAATTGAGTCAAGACTTGCTTCTTCA |
| | AACCGGAAATATGAGTTCTGGCCAAAACGAAAGTAGCTCTAACTC |
| | TATCAGATGAAGAACCAACCAAAGAAGGAGCTTGGTAGAAAGTAC |
| | CCAATCTGGTTTGATTCAATCTGTTTTCGTATAATTGTGGGTTAAC |
| | AACAACTCTATCGAAAACTGGTGGGGAACCATTTTTCAAGAATGG |
| | TTGATCTTCACCAGTTTCACAAACAGAAGCCCAAGCCTTCAAAAA |
| | ACCGAATCTAGTGTTAGCATCAGACAAAGAGTGATGGTTGGTCAA |
| | ACCAATAGAAATACCGGAGTTTGGGAAGTAAGTAACTTGAACAGA |
| | GAAAACTGGCAAGGTAACGTAATCAGATTCTTTTACAGCGTTACC |
| | CAATGGTGGAACCAATGGATAGAAATTTTCGCACTTTCTTGGATG |
| | GTTAGCAGACAAATCGTTGAAATCCAAGGTAGTTTCAGCGAAAGT |
| | CAAAGCAACAGAATCACCTTCAACATGTCTGATTTCTGGCTTTCTG |
| | GTAGAATCATGTGGATTTGGGTAAACGATCAACTTACCGACGAAT |
| | GGAAAGTAATGTTGCAAGGTAATGGACAAGGAGTGCTTCAAATTT |
| | GGGATAACAGTTTCGGTGAAATGGGACTTGGAGTATGGAAAATG |
| | GTAGAAGTACAAGTGATGAACTGGTGGAAACAACAACCAGGCAAT |
| | ATCGAAGAAAGTCAATGGCAATGATCTATGACCAATAGTAGATGG |
| | TGGTGGAGAAATTCTAGAGTGTTCCAAGATGGTCAAGTTTGGGAT |
| | GTTGTCCATTTTAAGCTTTTTGTTTGTTTATGTGTGTTTATTCGAAA |
| | CTAAGTTCTTGGTGTTTTAAAACTAAAAAAAAGACTAACTATAAAA |
| | GTAGAATTTAAGAAGTTTAAGAAATAGATTTACAGAATTACAATCA |
| | ATACCTACCGTCTTTATATACTTATTAGTCAAGTAGGGGAATAATT |
| | TCAGGGAACTGGTTTCAACCTTTTTTTTCAGCTTTTTCCAAATCAG |
| | AGAGAGCAGAAGGTAATAGAAGGTGTAAGAAAATGAGATAGATAC |
| | ATGCGTGGGTCAATTGCCTTGTGTCATCATTTACTCCAGGCAGGT |
| | TGCATCACTCCATTGAGGTTGTGTCCGTTTTTGCCTGTTTGTGC |
| | CCCTGTTCTCTGTAGTTGCGCTAAGAGAATGGACCTATGAACTGA |
| | TGGTTGGTGAAGAAAACAATATTTTGGTGCTGGGATTCTTTTTTTT |
| | TCTGGATGCCAGCTTAAAAAGCGGGCTCCATTATATTTAGTGGAT |
| | GCCAGGAATAAACTGTTCACCCAGACACCTACGATGTTATATATT |
| | CTGTGTAACCCGCCCCCTATTTTGGGCATGTACGGGTTACAGCA |
| | GAATTAAAAGGCTAATTTTTTGACTAAATAAAGTTAGGAAAATCAC |
| | TACTATTAATTATTTACGTATTCTTTGAAATGGCAGTATTGATAATG |
| | ATAAACTCGAACTGGGCGCGTCGTGCCGTCGTTGTTAATCACCAC |
| | ATGGTTATTCTGCTCAAACGTCCCGGACGCCTGCGAGGCGCGCC |
| | TATTGAAAGATCTTAAGGGGATATCCTCGAGGTTCCCTTTAGTGA |
| | GGGTTAATTGCGAGCTTGGCGTAATCATGGTCATAGCTGTTTCCT |
| | GTGTGAAATTGTTATCCGCTCACAATTCCACACAACATACGAGCC |
| | GGAAGCATAAAGTGTAAAGCCTGGGGTGCCTAATGAGTGAGCTA |
| | ACTCACATTAATTGCGTTGCGCTCACTGCCCGCTTTCCAGTCGGG |
| | AAACCTGTCGTGCCAGCTGCATTAATGAATCGGCCAACGCGCGG |
| | GGAGAGGCGGTTTGCGTATTGGGCGCTCTTCCGCTTCCTCGCTC |
| | ACTGACTCGCTGCGCTCGGTCGTTCGGCTGCGGCGAGCGGTATC |
| | AGCTCACTCAAAGGCGGTAATACGGTTATCCACAGAATCAGGGG |
| | ATAACGCAGGAAAGAACATGTGAGCAAAAGGCCAGCAAAAGGCC |
| | AGGAACCGTAAAAAGGCCGCGTTGCTGGCGTTTTTCCATAGGCT |
| | CCGCCCCCCTGACGAGCATCACAAAAATCGACGCTCAAGTCAGA |
| | GGTGGCGAAACCCGACAGGACTATAAAGATACCAGGCGTTTCCC |
| | CCTGGAAGCTCCCTCGTGCGCTCTCCTGTTCCGACCCTGCCGCT |
| | TACCGGATACCTGTCCGCCTTTCTCCCTTCGGGAAGCGTGGCGC |
| | TTTCTCATAGCTCACGCTGTAGGTATCTCAGTTCGGTGTAGGTCG |
| | TTCGCTCCAAGCTGGGCTGTGTGCACGAACCCCCCGTTCAGCCC |
| | GACCGCTGCGCCTTATCCGGTAACTATCGTCTTGAGTCCAACCC |
| | GGTAAGACACGACTTATCGCCACTGGCAGCAGCCACTGGTAACA |
| | GGATTAGCAGAGCGAGGTATGTAGGCGGTGCTACAGAGTTCTTG |
| | AAGTGGTGGCCTAACTACGGCTACACTAGAAGAACAGTATTTGGT |
| | ATCTGCGCTCTGCTGAAGCCAGTTACCTTCGGAAAAAGAGTTGGT |
| | AGCTCTTGATCCGGCAAACAAACCACCGCTGGTAGCGGTGGTTT |
| | TTTTGTTTGCAAGCAGCAGATTACGCGCAGAAAAAAAGGATCTCA |
| | AGAAGATCCTTTGATCTTTTCTACGGGGTCTGACGCTCAGTGGAA |
| | CGAAAACTCACGTTAAGGGATTTTGGTCATGAGATTATCAAAAAG |
| | GATCTTCACCTAGATCCTTTTAAATTAAAAATGAAGTTTTAAATCAA |
| | TCTAAAGTATATATGAGTAAACTTGGTCTGACAGTTACCAATGCTT |
| | AATCAGTGAGGCACCTATCTCAGCGATCTGTCTATTTCGTTCATC |
| | CATAGTTGCCTGACTCCCCGTCGTGTAGATAACTACGATACGGGA |
| | GGGCTTACCATCTGGCCCCAGTGCTGCAATGATACCGCGAGACC |
| | CACGCTCACCGGCTCCAGATTTATCAGCAATAAACCAGCCAGCC |
| | GGAAGGGCCGAGCGCAGAAGTGGTCCTGCAACTTTATCCGCCTC |
| | CATCCAGTCTATTAATTGTTGCCGGGAAGCTAGAGTAAGTAGTTC |
| | GCCAGTTAATAGTTTGCGCAACGTTGTTGCCATTGCTACAGGCAT |
| | CGTGGTGTCACGCTCGTCGTTTGGTATGGCTTCATTCAGCTCCG |
| | GTTCCCAACGATCAAGGCGAGTTACATGATCCCCCATGTTGTGCA |
| | AAAAAGCGGTTAGCTCCTTCGGTCCTCCGATCGTTGTCAGAAGTA |
| | AGTTGGCCGCAGTGTTATCACTCATGGTTATGGCAGCACTGCATA |
| | ATTCTCTTACTGTCATGCCATCCGTAAGATGCTTTTCTGTGACTGG |
| | TGAGTACTCAACCAAGTCATTCTGAGAATAGTGTATGCGGCGACC |
| | GAGTTGCTCTTGCCCGGCGTCAATACGGGATAATACCGCGCCAC |
| | ATAGCAGAACTTTAAAAGTGCTCATCATTGGAAAACGTTCTTCGG |
| | GGCGAAAACTCTCAAGGATCTTACCGCTGTTGAGATCCAGTTCGA |
| | TGTAACCCACTCGTGCACCCAACTGATCTTCAGCATCTTTTACTTT |
| | CACCAGCGTTTCTGGGTGAGCAAAAACAGGAAGGCAAAATGCCG |
| | CAAAAAAGGGAATAAGGGCGACACGGAAATGTTGAATACTCATAC |
| | TCTTCCTTTTTCAATATTATTGAAGCATTTATCAGGGTTATTGTCTC |
| | ATGAGCGGATACATATTTGAATGTATTTAGAAAAATAAACAAATAG |
| | GGGTTCCGCGCACATTTCCCCGAAAAGTGCCACCTGACGCGCCC |
| | TGTAGCGGCGCATTAAGCGCGGCGGGTGTGGTGGTTACGCGCA |
| | GCGTGACCGCTACACTTGCCAGCGCCCTAGCGCCCGCTCCTTTC |
| | GCTTTCTTCCCTTCCTTTCTCGCCACGTTCGCCGGCTTTCCCCGT |
| | CAAGCTCTAAATCGGGGGCTCCCTTTAGGGTTCCGATTTAGTGCT |
| | TTACGGCACCTCGACCCCAAAAAACTTGATTAGGGTGATGGTTCA |
| | CGTAGTGGGCCATCGCCCTGATAGACGGTTTTTCGCCCTTTGAC |
| | GTTGGAGTCCACGTTCTTTAATAGTGGACTCTTGTTCCAAACTGG |
| | AACAACACTCAACCCTATCTCGGTCTATTCTTTTGATTTATAAGGG |
| | ATTTTGCCGATTTCGGCCTATTGGTTAAAAAATGAGCTGATTTAAC |
| | AAAAATTTAACGCGAATTTTAACAAAATATTAACGCTTACAATTTGC |
| | CATTCGCCATTCAGGCTGCGCAACTGTTGGGAAGGGCGAT |
| SEQ ID NO: 53 | CGGTGCGGGCCTCTTCGCTATTACGCCAGCTGGCGAAAGGGGG |
| | ATGTGCTGCAAGGCGATTAAGTTGGGTAACGCCAGGGTTTTCCC |
| | AGTCACGACGTTGTAAAACGACGGCCAGTGAATTGTAATACGACT |
| | CACTATAGGGCGACCCTTAAGATCTAAGTCTTAGGCGCGCCAAG |
| | CTGGAGAAATATACCGCCCGTCAGGAAGAACTGAACAAGGCACT |
| | GAAAGACGGGAAAACGCGTCCAGTATCCCAGCAGATACGGGATA |
| | TCGACATTTCTGCACCATTCCGGCGGGTATAGGTTTTATTGATGG |
| | CCTCATCCACACGCAGCAGCGTCTGTTCATCGTCGTGGCGGCCC |
| | ATAATAATCTGCCGGTCAATCAGCCAGCTTTCCTCACCCGGCCCC |
| | CATCCCCATACGCGCATTTCGTAGCGGTCCAGCTGGGAGTCGAT |
| | ACCGGCGGTCAGGTAAGCCACACGGTCAGGAACGGGCGCTGAA |
| | TAATGCTCTTTCCGCTCTGCCATCACTTCAGCATCCGGACGTTCG |
| | CCAATTTTCGCCTCCCACGTCTCACCGAGCGTGGTGTTTACGAAG |
| | GTTTTACGTTTTCCCGTATCCCCTTTCGTTTTCATCCAGTCTTTGA |
| | CAATCTGCACCCAGGTGGTGAACGGGCTGTACGCTGTCCAGATG |
| | TGAAAGGTCACACTGTCAGGTGGCTCAATCTCTTCACCGGATGAC |
| | GAAAACCAGAGAATGCCATCACGGGTCCAGATCCCGGTCTTTTC |
| | GCAGATATAACGGGCATCAGTAAAGTCCAGCTCCTGCTGGCGGA |
| | TGACGCAGGCATTATGCTCGCAGAGATAAAACACGCTGGAGACG |
| | CGTGGCGCATCCGCGTCAGGCGGTACAGCCATTCAGGCCGCTG |
| | CGGCGAAATTCCATTTTGCAGGCGCGCCAATGCTTAGATCCTAAG |
| | GGGATATCCTCGAGGTTCCCTTTAGTGAGGGTTAATTGCGAGCTT |
| | GGCGTAATCATGGTCATAGCTGTTTCCTGTGTGAAATTGTTATCC |
| | GCTCACAATTCCACACAACATACGAGCCGGAAGCATAAAGTGTAA |
| | AGCCTGGGGTGCCTAATGAGTGAGCTAACTCACATTAATTGCGTT |
| | GCGCTCACTGCCCGCTTTCCAGTCGGGAAACCTGTCGTGCCAGC |
| | TGCATTAATGAATCGGCCAACGCGCGGGGAGAGGCGGTTTGCGT |
| | ATTGGGCGCTCTTCCGCTTCCTCGCTCACTGACTCGCTGCGCTC |
| | GGTCGTTCGGCTGCGGCGAGCGGTATCAGCTCACTCAAAGGCG |
| | GTAATACGGTTATCCACAGAATCAGGGGATAACGCAGGAAAGAA |
| | CATGTGAGCAAAAGGCCAGCAAAAGGCCAGGAACCGTAAAAAGG |
| | CCGCGTTGCTGGCGTTTTTCCATAGGCTCCGCCCCCCTGACGAG |
| | CATCACAAAAATCGACGCTCAAGTCAGAGGTGGCGAAACCCGAC |
| | AGGACTATAAAGATACCAGGCGTTTCCCCCTGGAAGCTCCCTCGT |
| | GCGCTCTCCTGTTCCGACCCTGCCGCTTACCGGATACCTGTCCG |
| | CCTTTCTCCCTTCGGGAAGCGTGGCGCTTTCTCATAGCTCACGCT |
| | GTAGGTATCTCAGTTCGGTGTAGGTCGTTCGCTCCAAGCTGGGC |
| | TGTGTGCACGAACCCCCCGTTCAGCCCGACCGCTGCGCCTTATC |
| | CGGTAACTATCGTCTTGAGTCCAACCCGGTAAGACACGACTTATC |
| | GCCACTGGCAGCAGCCACTGGTAACAGGATTAGCAGAGCGAGGT |
| | ATGTAGGCGGTGCTACAGAGTTCTTGAAGTGGTGGCCTAACTAC |
| | GGCTACACTAGAAGAACAGTATTTGGTATCTGCGCTCTGCTGAAG |
| | CCAGTTACCTTCGGAAAAAGAGTTGGTAGCTCTTGATCCGGCAAA |
| | CAAACCACCGCTGGTAGCGGTGGTTTTTTTGTTTGCAAGCAGCAG |
| | ATTACGCGCAGAAAAAAAGGATCTCAAGAAGATCCTTTGATCTTTT |
| | CTACGGGGTCTGACGCTCAGTGGAACGAAAACTCACGTTAAGGG |
| | ATTTTGGTCATGAGATTATCAAAAAGGATCTTCACCTAGATCCTTT |
| | TAAATTAAAAATGAAGTTTTAAATCAATCTAAAGTATATATGAGTAA |
| | ACTTGGTCTGACAGTTACCAATGCTTAATCAGTGAGGCACCTATC |
| | TCAGCGATCTGTCTATTTCGTTCATCCATAGTTGCCTGACTCCCC |
| | GTCGTGTAGATAACTACGATACGGGAGGGCTTACCATCTGGCCC |
| | CAGTGCTGCAATGATACCGCGAGACCCACGCTCACCGGCTCCAG |
| | ATTTATCAGCAATAAACCAGCCAGCCGGAAGGGCCGAGCGCAGA |
| | AGTGGTCCTGCAACTTTATCCGCCTCCATCCAGTCTATTAATTGTT |
| | GCCGGGAAGCTAGAGTAAGTAGTTCGCCAGTTAATAGTTTGCGC |
| | AACGTTGTTGCCATTGCTACAGGCATCGTGGTGTCACGCTCGTC |
| | GTTTGGTATGGCTTCATTCAGCTCCGGTTCCCAACGATCAAGGCG |
| | AGTTACATGATCCCCCATGTTGTGCAAAAAAGCGGTTAGCTCCTT |
| | CGGTCCTCCGATCGTTGTCAGAAGTAAGTTGGCCGCAGTGTTATC |
| | ACTCATGGTTATGGCAGCACTGCATAATTCTCTTACTGTCATGCC |
| | ATCCGTAAGATGCTTTTCTGTGACTGGTGAGTACTCAACCAAGTC |
| | ATTCTGAGAATAGTGTATGCGGCGACCGAGTTGCTCTTGCCCGG |
| | CGTCAATACGGGATAATACCGCGCCACATAGCAGAACTTTAAAAG |
| | TGCTCATCATTGGAAAACGTTCTTCGGGGCGAAAACTCTCAAGGA |
| | TCTTACCGCTGTTGAGATCCAGTTCGATGTAACCCACTCGTGCAC |
| | CCAACTGATCTTCAGCATCTTTTACTTTCACCAGCGTTTCTGGGTG |
| | AGCAAAAACAGGAAGGCAAAATGCCGCAAAAAAGGGAATAAGGG |
| | CGACACGGAAATGTTGAATACTCATACTCTTCCTTTTTCAATATTA |
| | TTGAAGCATTTATCAGGGTTATTGTCTCATGAGCGGATACATATTT |
| | GAATGTATTTAGAAAAATAAACAAATAGGGGTTCCGCGCACATTT |
| | CCCCGAAAAGTGCCACCTGACGCGCCCTGTAGCGGCGCATTAAG |
| | CGCGGCGGGTGTGGTGGTTACGCGCAGCGTGACCGCTACACTT |
| | GCCAGCGCCCTAGCGCCCGCTCCTTTCGCTTTCTTCCCTTCCTTT |
| | CTCGCCACGTTCGCCGGCTTTCCCCGTCAAGCTCTAAATCGGGG |
| | GCTCCCTTTAGGGTTCCGATTTAGTGCTTTACGGCACCTCGACCC |
| | CAAAAAACTTGATTAGGGTGATGGTTCACGTAGTGGGCCATCGCC |
| | CTGATAGACGGTTTTTCGCCCTTTGACGTTGGAGTCCACGTTCTT |
| | TAATAGTGGACTCTTGTTCCAAACTGGAACAACACTCAACCCTAT |
| | CTCGGTCTATTCTTTTGATTTATAAGGGATTTTGCCGATTTCGGCC |
| | TATTGGTTAAAAAATGAGCTGATTTAACAAAAATTTAACGCGAATT |
| | TTAACAAAATATTAACGCTTACAATTTGCCATTCGCCATTCAGGCT |
| | GCGCAACTGTTGGGAAGGGCGAT |
| SEQ ID NO: 54 | MANPHPHFLIITFPAQGHINPALELAKRLIGVGADVTFATTIHAKSRLV |
| | KNPTVDGLRFSTFSDGQEEGVKRGPNELPVFQRLASENLSELIMAS |
| | ANEGRPISCLIYSILIPGAAELARSFNIPSAFLWIQPATVLDIYYYYFNG |
| | FGDLIRSKSSDPSFSIELPGLPSLSRQDLPSFFVGSDQNQENHALAA |
| | FQKHLEILEQEENPKVLVNTFDALEPEALRAVEKLKLTAVGPLVPSGF |
| | SDGKDASDTPSGGDLSDGSRDYMEWLKSKPESTVVYVSFGSISMF |
| | SMQQMEEIARGLLESGRPFLWVIRAKENGEENKEEDKLSCQEELEK |
| | QGMLIQWCSQMEVLSHPSLGCFVTHCGWNSSIESLASGVPMIAFPQ |
| | WADQGTNTKLIKDVWKTGVRLMVNEEEIVTSDELRRCLELVMGDGE |
| | KGQEMRKNAKKWKILAKEALKEGGSSHKNLKNFVDEVIQGY |
| SEQ ID NO: 55 | MALRINELFVAAIIYIIVHIIISKLITTVRERGRRLPLPPGPTGWPVIGALP |
| | LLGSMPHVALAKMAKKYGPIMYLKVGTCGMVVASTPNAAKAFLKTL |
| | DINFSNRPPNAGATHLAYNAQDMVFAPYGPRWKLLRKLSNLHMLGG |
| | KALENWANVRANELGHMLKSMFDASQDGECVVIADVLTFAMANMIG |
| | QVMLSKRVFVEKGVEVNEFKNMVVELMTVAGYFNIGDFIPKLAWMDI |
| | QGIEKGMKNLHKKFDDLLTKMFDEHEATSNERKENPDFLDVVMANR |
| | DNSEGERLSTTNIKALLLNLFTAGTDTSSSVIEWALAEMMKNPKIFKK |
| | AQQEMDQVIGKNRRLIESDIPNLPYLRAICKETFRKHPSTPLNLPRVS |
| | SEPCTVDGYYIPKNTRLSVNIWAIGRDPDVWENPLEFTPERFLSGKN |
| | AKIEPRGNDFELIPFGAGRRICAGTRMGIVVVEYILGTLVHSFDWKLP |
| | NNVIDINMEESFGLALQKAVPLEAMVTPRLSLDVYRC |
| SEQ ID NO: 56 | MTSALYASDLFKQLKSIMGTDSLSDDVVLVIATTSLALVAGFVVLLWK |
| | KTTADRSGELKPLMIPKSLMAKDEDDDLDLGSGKTRVSIFFGTQTGT |
| | AEGFAKALSEEIKARYEKAAVKVIDLDDYAADDDQYEEKLKKETLAFF |
| | CVATYGDGEPTDNAARFYKWFTEENERDIKLQQLAYGVFALGNRQY |
| | EHFNKIGIVLDEELCKKGAKRLIEVGLGDDDQSIEDDFNAWKESLWS |
| | ELDKLLKDEDDKSVATPYTAVIPEYRVVTHDPRFTTQKSMESNVANG |
| | NTTIDIHHPCRVDVAVQKELHTHESDRSCIHLEFDISRTGITYETGDH |
| | VGVYAENHVEIVEEAGKLLGHSLDLVFSIHADKEDGSPLESAVPPPF |
| | PGPCTLGTGLARYADLLNPPRKSALVALAAYATEPSEAEKLKHLTSP |
| | DGKDEYSQWIVASQRSLLEVMAAFPSAKPPLGVFFAAIAPRLQPRYY |
| | SISSSPRLAPSRVHVTSALVYGPTPTGRIHKGVCSTWMKNAVPAEKS |
| | HECSGAPIFIRASNFKLPSNPSTPIVMVGPGTGLAPFRGFLQERMAL |
| | KEDGEELGSSLLFFGCRNRQMDFIYEDELNNFVDQGVISELIMAFSR |
| | EGAQKEYVQHKMMEKAAQVWDLIKEEGYLYVCGDAKGMARDVHRT |
| | LHTIVQEQEGVSSSEAEAIVKKLQTEGRYLRDVW |
| SEQ ID NO: 57 | MVAHLQPPKIIETCHISPPKGTVPSTTLPLTFFDAPWLSLPLADSLFFF |
| | SYQNSTESFLQDFVPNLKHSLSITLQHFFPYAGKLIIPPRPDPPYLHY |
| | NDGQDSLVFTVAESTETDFDQLKSDSPKDISVLHGVLPKLPPPHVSP |
| | EGIQMRPIMAMQVTIFPGAGICIGNSATHVVADGVTFSHFMKYWMSL |
| | TKSSGKDPATVLLPSLPIHSCRNMIKDPGEVGAGHLERFWSQNSAK |
| | HSSHVTPENMVRATFTLSRKQIDNLKSWVTEQSENQSPVSTFVVTL |
| | AFIWVSLIKTLVQDSETKANEEDKDEVFHLMINVDCRNRLKYTQPIPQ |
| | TYFGNCMAPGIVSVKKHDLLGEKCVLAASDAITARIKDMLSSDLLKTA |
| | PRWGQGVRKWVMSHYPTSIAGAPKLGLYDMDFGLGKPCKMEIVHIE |
| | TGGSIAFSESRDGSNGVEIGIALEKKKMDVFDSILQQGIKKFAT |
| SEQ ID NO: 58 | MDNIPNLTILEHSRISPPPSTIGHRSLPLTFFDIAWLLFPPVHHLYFYHF |
| | PYSKSHFTETVIPNLKHSLSITLQHYFPFVGKLIVYPNPHDSTRKPEIR |
| | HVEGDSVALTFAETTLDFNDLSANHPRKCENFYPLVPPLGNAVKESD |
| | YVTLPVFSVQVTYFPNSGISIGLTNHHSLSDANTRFGFLKAWASVCE |
| | TGEDQPFLKNGSPPVFDRVVVNPQLYENRLNQTRLGTFYQAPSLVG |
| | SSSDRVRATFVLARTHISGLKKQVLTQLPMLEYTSSFTVTCGYIWSCI |
| | VKSLVNMGEKKGEDELEQFIVSVGCRSRLDPPLPENYFGNCSAPCIV |
| | TIKNGVLKGENGFVMAAKLIGEGISKMVNKKGGILEYADRWYDGFKI |
| | PARKMGISGTPKLNFYDIDFGWGKAMKYEVVSIDYSASVSLSACKES |
| | AQDFEIGVCFPSMQMEAFGKIFNDGLESAIAS |

1. A microorganism, comprising an operative metabolic pathway capable of producing an anthocyanin from a simple sugar, the operative metabolic pathway comprising:
a 4-coumaric acid-CoA ligase (4CL);
a chalcone synthase (CHS);
a flavanone 3-hydroxylase (F3H);
a dihydroflavonol-4-reductase (DFR);
an anthocyanidin synthase (ANS);
an anthocyanidin 3-O-glycosyltransferase (A3GT);
a chalcone isomerase (CHI); and
at least one of
a) a tyrosine ammonia lyase (TAL); or
b) a phenylalanine ammonia lyase (PAL) and a trans-cinnamate 4-monooxygenase (C4H),
wherein at least one enzyme of the operative metabolic pathway is encoded by a gene heterologous to the microorganism.

2. The microorganism of claim 1, wherein the metabolic pathway further comprises:
a tyrosine ammonia lyase (TAL);
a phenylalanine ammonia lyase (PAL); and
a trans-cinnamate 4-monooxygenase (C4H).

3. The microorganism of claim 1, wherein the metabolic pathway further comprises one or more of:
a flavonoid 3′-hydroxylase (F3′H);
a flavonoid 3′-5′-hydroxylase (F3′5′H);
a leucoanthocyanidin reductase (LAR); or
a CYP450 reductase (CPR).

4. The microorganism of claim 3, wherein the anthocyanin is pelargonidin-3-O-glucoside (P3G), cyanidin-3-O-glucoside (C3G), or delphinidin-3-O-glucoside (D3G).

5. The microorganism of claim 1, wherein the microorganism is a yeast or a bacteria.

6. (canceled)

7. (canceled)

8. (canceled)

9. (canceled)

10. The microorganism of claim 1, wherein a plurality of enzymes comprising the operative metabolic pathway are encoded by genes that are heterologous to the microorganism.

11. (canceled)

12. (canceled)

13. (canceled)

14. The microorganism of claim 1, wherein the operative metabolic pathway comprises:
a 4-coumaric acid-CoA ligase (4CL) encoded by the nucleic acid sequence set forth in SEQ ID NO: 1;
a chalcone synthase (CHS) encoded by the nucleic acid sequence set forth in SEQ ID NO: 21;
a flavanone 3-hydroxylase (F3H) encoded by the nucleic acid sequence set forth in SEQ ID NO: 3;
a dihydroflavonol-4-reductase (DFR) encoded by the nucleic acid sequence set forth in SEQ ID NO: 5 or SEQ ID NO: 7;
an anthocyanidin synthase (ANS) encoded by the nucleic acid sequence set forth in SEQ ID NO: 9;
an anthocyanidin 3-O-glycosyltransferase (A3GT) encoded by the nucleic acid sequence set forth in SEQ ID NO: 11;
a chalcone isomerase (CHI) encoded by the nucleic acid sequence set forth in SEQ ID NO: 13; and
at least one of
a) a tyrosine ammonia lyase (TAL) encoded by the nucleic acid sequence set forth in SEQ ID NO: 15, or
b) a phenylalanine ammonia lyase (PAL) encoded by the nucleic acid sequence set forth in SEQ ID NO: 17 and a trans-cinnamate 4-monooxygenase (C4H) encoded by the nucleic acid sequence set forth in SEQ ID NO: 19.

15. The microorganism of claim 14 further comprising a flavonoid 3′-5′-hydroxylase (F3'S′H) encoded by the nucleic acid sequence set forth in SEQ ID NO: 33.

16. A method of producing an anthocyanin, comprising the steps of:
a) culturing the microorganism of claim 1 in a culture medium, wherein the anthocyanin is produced by the microorganism; and
b) optionally isolating the anthocyanin.

17. The method of claim 16, wherein the anthocyanin is pelargonidin-3-O-glucoside (P3G), cyanidin-3-O-glucoside (C3G), and/or delphinidin-3-O-glucoside (D3G).

18. (canceled)

19. (canceled)

20. (canceled)

21. (canceled)

22. (canceled)

23. (canceled)

24. (canceled)

25. (canceled)

26. (canceled)

27. (canceled)

28. (canceled)

29. The method of claim 18, wherein the simple sugar comprises glucose, glycerol, ethanol, or easily fermentable raw materials.

30. A microorganism, comprising an operative metabolic pathway capable of producing an anthocyanin from a simple sugar, the operative metabolic pathway comprising:
a 4-coumaric acid-CoA ligase (4CL);
a chalcone synthase (CHS);
a flavanone 3-hydroxylase (F3H);
a dihydroflavonol-4-reductase (DFR);
an anthocyanidin synthase (ANS);
an anthocyanidin 3-O-glycosyltransferase (A3GT);
a chalcone isomerase (CHI);
at least one of
a) a tyrosine ammonia lyase (TAL); or
b) a phenylalanine ammonia lyase (PAL) and a trans-cinnamate 4-monooxygenase (C4H); and
an anthocyanin-5-O-glycosyl transferase (A5GT), an anthocyanin-3-O-aromatic acyl transferase (A3AAT), or an anthocyanin-3-O-malonyl acyl transferase (A3MAT),
wherein at least one enzyme of the operative metabolic pathway is encoded by a gene heterologous to the microorganism.

31. The microorganism of claim 30, wherein the anthocyanin is pelargonidin-3,5-O-diglucoside, cyanidin-3,5-O-diglucoside, delphinidin-3,5-O-diglucoside, pelargonidin-3-O-coumaroyl-glucoside, pelargonidin-3-O-coumaroyl glucoside-5-O-glucoside, pelargonidin-3-O-malonyl glucoside, or pelargonidin-3-O-malonyl glucoside-5-O-glucoside.

32. A method of producing an anthocyanin, comprising the steps of:
a) culturing the microorganism of claim 30;
b) producing an anthocyanin by the microorganism; and
c) optionally isolating the anthocyanin.

33. (canceled)

34. A method of producing an anthocyanin, comprising the steps of:
a) culturing the microorganism of claim 1;
b) producing an anthocyanin by the microorganism; and
c) optionally isolating the anthocyanin.
